SEQUENCE LISTING (233 sequences)

SEQ ID NO: 1 - protein, 387 aa, Catenibacterium sp.
MRKLLKSFKTEINPTEEQKVRIRKTIGTCRFIYNFYLAHNKELHESGKKFMSSSQFRVWLNNEYLPSHPEYSWIKEAYSKSVTQAVNDGQTAFTRFFNHKSAFPKFKKKGKSDVKMYFVRNNPKDCSCERHRIKIPSLGWVRIKEKGYIPTTKDGYVIKSGHVSIKADRYYVSVLIEIPDKTSASNSSEGIGIDLGLKDFAIASNGKTYKNINKSAKLKKLEKKLIREQRSLSRKYENIKKGGSTQKRNIQKQRLKIQKLHHRIDNIRTDYINKIIAEIVKTKPSYITIEDLNISGMMKNRHLSKAVASQKFYEFKTKLQAKCRENGIELRVVDRWFPSSKTCHCCKSIKKDLKLSDRLFRCDCGYIEDRDFNAALNLRDATTYEVA

SEQ ID NO: 2 - protein, 402 aa, Mitsuokella sp.
MKRLLKSFKTELRLTESQTDKVRRSLGICRFLANQYIARNIWLCRLYQRGVLDEKQPHFITANDFDKYVNHVLKKKFPWIDQCGSKARKKVLVNAEQAFRRFFKGPARFPRFKRKNQQNVKLYFPKNNKGDWTIWRHKIQIPTIGRVRLKEFGYLPQGVTIKNGHVSCEAGRYYVSVTAEIEETSRYNNDLEASYHPQSDGIGVDLGVKSLAIVSDGRIFENINTSSRARRLEKRLRREQRRLSRKYEIRKKKGGSTATASANIEKKRLSVQKLHQRLTNLRRNYENQVIHALVEQKPRFITMEDLNVTGMMKNRHLSKSVAQQRFYSLREKLRRKAEIIGIEFRIADRFYPSSKTCHACGHVHGGLKLKDRIYVCPVCGYTEDRDLNAALNLRDTKNYRIA

SEQ ID NO: 3 - protein, 387 aa, Blautia sp.
MRKLQKSFKTEINPTDEQKLKIRRTIGTCRYVYNFYLDQNRKRYEKGEKFMSGKSFSVWLNNEYLPQNPEYAWIKEVSSKSVKRSLENACTAFSRFFKGQSHFPRYKKKNKSNVKMYFVKNNRTDCFCERHRVNIPTLGWVRIKEKGYLPTTKDGYVIKSGTVSMRAGRYYVSVLVEVPDPPKEENRNEGIGIDLGIKSFAVLSDGTVYQNINKSAKIKKLEKQLRREQRSLSRKYENLKKGVSTQRANIQKQKLKVQRLYQRIDNIRTDYIKKTIAGIVKTKPSFVTIEDLNVKGMMKNRHLSKATASQKFYEFRTRLQGKCAEKGIELRIADRWYPSSRRCHNCGQIRKDLKLSNRVYQCSCGYCEDRDLNAALNLRDVKTYQSA

SEQ ID NO: 4 - protein, 387 aa, Ruminococcus sp.
MRRLLKSFKTEIDPTEEQKIKIHKTIGTCRYIYNFYLFHNKERYDAGERFMSGKSFSVWLNNEYLPGHPEYFWIREVSSKSVKNTIENACTAFSRFFHHQSSFPRYKKKGRSDVKMYFVKNNPKDCLCERHRIKIPTLGWVRLKEKGYIPASKDGFVIRSGAVSMKAGRYYVSALVDIPAPEADGNFTDGIGIDLGLKDFAVVSNGTVYRNINKTARIRKLEKQLRRAQRKLSRKYENLKKGESTQRANICKQKLKVQKLHHRIENIRTDHINKVISEIVKTKPSHITIEDLNISGMMKNRHLSKAVASQKFYEFRTKLKIKCAEYGIELRVVDRWYPSSRICHCCSCIKKDLKLSDRIYRCTCGYIEDRDLNAARNLRDAETYEVA

SEQ ID NO: 5 - protein, 387 aa, Clostridium sp.
MWKLLKSFKTEINPTEEQKVKIRKTIGTCRYIYNFYLAHNKELHDNGKKFMSGKSFSVWLNNEYLPQNPDKLWIKEVSSKSVKRSIENGCVAFTRFFKHQSAFPNFKKKGKSDVKMYFVKNNPKDCRCERHRINIPSLGWVRIKEKGYIPTTKDGYVIKSGTVSIKAGRYYVSVLVEIPDMLIDSSNNDGIGIDLGLKNFAIVSNGKTYKNINKSARLKKLEKQLRREQKCLSRKYEYLKKGESTQRANIQKQKLKVQKLYHRLDNIHTDYINKIIAEIVKTKPSYITIEDLNVSGMMKNRHLSKAVASQKFYEFRTTLKSKCDDNNIELRVVDRFYPSSKLCHCCGSIKKDLKLSDRIYRCDCSYVADRDLNAALNLKDAKTYKIA

SEQ ID NO: 6 - protein, 387 aa, unidentified
MWKLLKSFKTEINPSEEQKVKIHKTIGTCRFIYNFYLAHNKELYDKGEKFMSGKSFSVWLNNEYLPQNPDKLWIKEVSSKSVKHSIENGCIAFTRFFKHQSAFPNLKKKGKSDVKMYFVKNNPKDCRCERHRINIPSLGWVRIKEKGYIPTTKDGYVIKSGTVSMKADRYYVSVLVEISNNKIANNSNAGIGIDLGLKDFAIVSNGKTYKNINKSARLKKHEKQLIREQRSLSRKYENLKKGESTQKANIQKQRLKVQKLHHRMDNIRTDYINKTIAEIVKTKPSYITIEDLNVKGMMKNRHLSKAVASQKFYEFRTKLQAKCNENGIELRVVDRWYPSSKTCHCCGAVKKDLKLSDRIFKCSCGYVEDRDFNAALNLRDAITYEVA

SEQ ID NO: 7 - protein, 387 aa, Lachnospira sp.
MRKLLKSFKTEINPTEEQKAKIRKTIGTCRYIYNFYLAHNKELYNKGEKFMSSNQFRVWLNNEYLPKHPECFWIKEAYSKAVTQAVNNGQAAFIRFFKHQSSFPRFKKKNRSDVKMYFVKNNPKDCRCQRHRINIPSLGWVRIKEKGYIPTTKDGYVIKSGHVSIKAGRYYVSALVEIPNNKIANSFNEGIGIDLGIKDFAIVSNGKIYKNINRSVRLKKLGKQLIREQKNPSRKYENLKKGESTQKANIQKQRLKVQKLHHKIDNIRTDHINKTIAEIVKTKPSYITIEDLNISGMMKNKHLSKAVASQKFYEFRIKLQAKCNENGIELRVVDRWYPSSKTCHCCGAIKKDLKLSDRIFKCDCGYSEDRDFNAALNLRDATTYEIA

SEQ ID NO: 8 - protein, 387 aa, Dorea formicigenerans
MWKLLKSFKTEINPSEEQKVKIHKTIGTCRFIYNFYLAHNKELYDNGEKFMSSNKFRVWLNHEYLSEHPEYSWIKEVYSKAVTQAVNNGQTAFARFFNHESAFPNFKKKGKSDVKMYFVKNNPKDCRCERHRINIPSLGWVRIKEKGYIPTTKDGYVIKSGTVSMKADRYYVSVLVEILNNKIANNSNAGIGIDLGLKEFAIVSNGKTYKNINKSARLKKLEKQLIREQRSLSRKYENLKKGESTQKTNIQKQRLKVQKLHHRIDNIRTDYINKTIVEIVKTKPSYITIEDLNVKGMMKNRHLSKAVASQKFYEFRTKLQAKCNENGIELRVVDRWYPSSKTCHCCGAVKKDLKLSDRIFKCSCGYVEDRDFNAALNLRDAITYEVA

SEQ ID NO: 9 - protein, 387 aa, Eubacterium hallii
MARLLKSFKTEINPSEEQKVRIYKTIGTCRFIYNFYLSHNKELYDKGEKFMSSNKFRLWLNNEFLPDNPDKMWIKEAYSKAVTQAVNHAQTAFKKFFDHKTAFPRFKKKGKSDVKMYFVRNNPKDCRCERHRIHIPSLGWIRIKEKGYIPTTKDGYIIKSGTVSIKAGRFYVSALVEVPDRKITEPFNEGIGIDLGLKEFAVVSNGRIYKNINRSARIRKLEKQLIREQRRLSRKYEDLKKGETTQKANIQKQKRKVQKLHQRIQNIRTDYMNKIIAEIVKTKPSYITIEDLNVKGMMKNRHLSKAVASQKFYEFRRKLQAKCTEKGMELRIADRWYPSSKKCHCCGCINKELKLSDRIYRCGCGYVEDRDFNAALNLRDARTYKTA

SEQ ID NO: 10 - protein, 387 aa, unidentified
MRKLLKSFKTEINPTEEQKTKIRKTIGTCRFIYNFYLAHNKKLHEDGKKFMSSNKFRIWLNNEYLPQHPEYLWIKEAYSKAVTQSVNNGQTAFTRFFNHESAFPNFKKKGKSDVKMYFVKNNPKDCHCERHRIKIPSLGWVRIKEKGYIPTTKDGYVIKSGSVSIKADRYYVSVLVEVSDNKTINHFDEGIGIDLGLKDFAIVSNGRTYQNINKSAKLKKLERQLIREQRCLSRKYENLKKGDATQRANIQKQKLKVQKLHHRIDNIRTDYINKTIAEMVKIKPSYITIEDLNVKGMMKNRHLSKAVASQKFYEFRAKLLTKCNKSGIELRVVDRWYPSSKICHCCGSIKKDLKLSDRIFKCRCGYIEDRDFNASLNLRDAETYSIA

SEQ ID NO: 11 - protein, 386 aa, Roseburia inulinivorans
MRKLLKSFKTEINPTVEQKIKINKTIGTCRYVYNFYLGHNKALYDKGEKFMTGKSFSVWLNNEYIPNNPDKIWIKEAYSKAVKKSIEDGCTAFTRYFKHQSAFPNFKKKGKSDVKMYFVKNNPKDCRCERHKLNIPTLGWVRMKEKGYIPTTKDGWKIKSGTVSIKADRYYVSVLVEIPDAKITDNSNDGMGIDLGLKDLAIVSNGKTYKNINKSARVKKLEKKLRREQRCLSRKYENLKKGESTQKNIQKQKLKVQRLHHKIDNIRTDYINKSIAEIVKAKPSYITIEDLNVSGMMKNRHLSKAVASQKFYEFRTKLKAKCDENGIELRVVDRWYPSSKICHCCGAVKKDLKLSDRIYRCDCGYIEDRDFNAALNLRDALTYEVA

SEQ ID NO: 12 - protein, 387 aa, Anaerobutyricum hallii
MRKLLKSFKTEIDPTEEQKVRIHKTIGTCRFIYNFYLAHNKELHKNGERFMSSSKFRVWLNNEYLPNHPEYSWIKEAYSKAVTQAVNNGQTAFTRFFNHESNFPNFKKKGRSDVKMYFVKNNPKDCCCERHRIKIPSLGWVRIKEKGYIPTTKDEYVIKSGHVSIKADRYYVSVLVEIPDDKMVNNSNEGIGIDLGLKDLAIISNGKTYKNINKSAKLKKLEKQLIRGQRSLSRKYENSKKGESTQRANIQKQKIKVQKLYQKIDNIRTDYINKTIAEIVKTKPSFITIEDLNVKGMMKNRHLSKAVASQKFYEFRIKLQAKCNENGIELRVVDRWYPSSKTCHCCKNIKKDLRLSDRIFKCDCGYIEDRDLNAALNLRDAMTYEVA

SEQ ID NO: 13 - protein, 386 aa, Eubacterium rectale
MRKLLKSFKTEINPTIEQKIKINKTIGTCRYVYNFYLGHNKTLYDNGEKFMTGKSFSVWLNNEYIPNNPDKIWIKEAYSKAVKKSIEDGCTAFTRFFKHQSAFPNFKKKGKSDVKMYFVKNNTKDCRCERHRLNIPTLGWVRIKEKGYIPTTKDGWKIKSGTVSVKADRYYVSVLVEIPNVKIANNSNGGIGIDLGLKDLAIVSNGKTYKNINKSTRIKKLEKKLRREQRCLSRKYENLKKGESTQKNIQKQKLKVQRLHHKIDNIRTDYINKSIAEIVKTKPSYITIENLNVSGMMKNRHLSKAVASQKFYEFRTKLKAKCDENGIELRVVDRWYPSSKICHCCGAIKKDLKLSDRIYRCDCGYVEDRDFNAALNLRDALTYEVA

SEQ ID NO: 14 - protein, 385 aa, Bacillus cereus
MKRAYLIEIKPTTEQVSKIHQTIGVCRYVYNLYISKNKEAYESEGKFLSGYDFSKWLNNIHTKECDQWIKEVSSKAVKQAIMNGDKAFKRFFKGLSKFPRYKKKRKQDVKCYFPKNNKTDWTVERHRVKVPTIGWVRLKEYGYIPKNATVKSGTVYQRAGRYFVSVLCERAEIRVKPMLNKVGLGIDLGLKDFAVRSDGKTHKNINKTVQVKKIEKRLKREQRSLSRKFENRKKRGEQSVTNKRANIDKNILRVQKLHARLANIRLAYVKSIVNDVVKTKPTFITVEDLNVKGMMKSKHLSKTIAKQSFFAFKTWLLTKCEEYGIELRQVDRFYPSSKLCSCCSQKKVDLKLSDRVYTCECGNVMDRDLNAAINLLQAKEYTILT

SEQ ID NO: 15 - protein, 370 aa, Weizmannia coagulans
MILAKKVRLNPTEEQERQLWKSVGTARWVYNWTLSKQEESYKNDGKFISDGELRKELTQLKKTEEYSWLLDVSNNVAKQAVKDACDAYKKFFKKLANKPRFKSRKRSKPSFYNDNVKLKVKEMLVLIEKVGWVKTSEQLPINTKYVNPRVSFDGKYWYLSVGIQQKHVHNDLTDNIIGIDVGVKELAVCSNGMVFKNINKTKTVKKIEKRLRRLQRKVSRKYEMNKEGNRFVKTCNIIKTEKKIRLLHRRLMNIRTNHIHQTTNAIVKTKPSTVVMEKLNIKGMMKNKYLSKAVQNQKLYEFKRQMKYKCEFNGIKFIEADKWYPSSKTCSNCGQIKKDLKLSERIFRCDCGLEIDRDLNASINLAKLAN

SEQ ID NO: 16 - protein, 387 aa, Blautia sp.
MVKAIKVMLIPNNVQKTKMFQYAGASRFAYNWALAREKENYEKGGRFIPDAELRKEFTKLRHSDEYAWLLSISNNVTKQAIKDACTAYKNFFKGLQKFPRFKYRKRSMPKFYQDNVKIRFSNTHVKFEGFSSGRKANKQKMNWVRLAEHGRIPTDAKYMNPRISFDGLNWWISVCVELPDCRETPNDDGVGIDLGIKNLAVCSDAVKYKNINKSQKVKKLEKQKRGLQRSISRSYEKNKRGESYCKTNNVIKKKKLLLKRNHRLTNIRKNYLNQTISEIVNRKPRFICIEDLNVSGMMKNRHLSKAVQEQGFFRFRKQLEYKCSDKGIQLIVADRFYPSSKLCSCCGNIKKDLKLSDRIYRCECGNRIDRDFQASINLKIYGERFAS

SEQ ID NO: 17 - protein, 384 aa, Blautia sp.
MLKSFKTEIHPTEEQKVRIHKTIGTCRFIYNFYLAHNKELYQNGEKFMSSSKFRVWLNHEYLPQHPEYLWIKEAYSKAVTQAVNHGQTAFKKFFNHESAFPKFKKKGRSDVKMYFVKNNPKDCRCERHRVNIPSLGWVRIKEKGYIPTTKEGYIIKSGHVSMKAGRYYVSALIEIPDNKTADLSNEGIGIDLGLKDFAIVSNGKTYKNINKSARLKKLEKRLVREQRCLSRKYENLKKGESTQRANIQKQKLKVQKLHHKIDNIRTDYINKTIAEIVKTKPSYITIEDLNVSGMMKNKHLSRAVASQKFYEFRIKLKAKSRENGIELRVADRWYPSSKICHCCGTIKKDLKLSDRTFRCHCGYVEDRDFNAALNLRDVKTYKIA

SEQ ID NO: 18 - protein, 387 aa, Blautia sp.
MRRLLKSFKTEINPTEEQKIKIHKTIGTCRYIYNFYLFHNKERYDIGEKFMSGRAFSVWLNNEYLPKHPEYSWIREVSSKSVKNAIENACTAFSRFFHHQSGFPRYKKKGRSDVKMYFVRNNPKDCFCERHRIKIPTLGWVRLKEKGYIPTSRDGYVVRSGAVSMKAGRYYVSALIEVPTPKVDGNFSDGIGIDLGLKNFAVVSNGTVYRNINKTVQIRKLEKQLTRAQRKLSRKYENLRKGEFTQKANIQKQKLKVQKLYDRIENIRTDHINKTISEIVKTKPSHITIEDLNVSGMMKNRHLSKAVSSQKFYEFRTKLKAKCAEYGIELRVVDRWYPSSKTCHCCGCIKKDLKLSDRIYKCTCGYVEDRDFNAALNLRDARTYKVA

SEQ ID NO: 19 - protein, 386 aa, Butyricicoccus sp.
MRELLKSFKTEINPTAEQKVKINKTIGTCRFVYNFYLDHNKALHNKGEKFMTGKSFSVWLNNEYIPNNPDKAWIKEAYSKAVKKSIENGCIAFTRFFKHQSAFPNFKKKGKSDVKMYFVKNNPKDCRCERHRLNIPTLGWVRIKEKGYIPTTKDGWRIRSGTISIKAGRYYVSVLIEVPDVKNTNNNSKGIGIDLGLKDFAIISNGKIYKNINKSARVKKLENKLRREQRCFSRKYENLKKGESTQKNIQKQELKVQRLHHKIVNIRTDYINKTIAEIVKTKPSYITIEDLNVSGMMKNKHLSKAVASQKFYEFRTKLKAKCDENGIELRIVNRWYPSSKICHCCGAIKKDLKLSDRIYRCGCGYVEDRDFNAALNLRDALTYEIA

SEQ ID NO: 20 - protein, 386 aa, unidentified
MKKLLKSFKTEINPTVEQKIKINKTIGTCRYVYNFYLVHNRNLHDQCEKFMTGKSFSVWLNNEYIPNHPDKAWIKEAYSKAVKKSIEDGCTAFTRFFKHQSDFPNFKKKGKSDVKMYFVKNNPKDCRCERHRLNIPTLGWVRIKEKGYIPTTKDGWKIKSGTVSIRAGRYYVSALVEVPDATIYNNNNDGVGIDLGLKDLAIVSNGKTYKNINKSARVKKLEKQLRREQRCLSRKYESLKKGESTQKNIQKQKRKVQKLHHKIDNIRTDYINKTVAEIVKTKPSYITIEDLNISGMMKNRHLSKAIASQKFYEFRIKLKAKCDENGVELRVADRWYPSSKICHCCGTIKRDLKLSDRIYRCSCGYIEDRDFNASLNLRDAITYKIA

SEQ ID NO: 21 - protein, 387 aa, unidentified
MRKLLKSFKTEINPTEEQKTKIRKTIGTCRFIYNFYLAHNKKLHEDGKKFMSSNKFRIWLNNEYLPQHPEYSWIKEAYSKAVTQSVNNGQTAFTRFFNHESAFPNFKKKGKSDVKMYFVKNNPKDCHCERHRIKIPSLGWVRIKEKGYIPTTKDGYVIKSGSVSIKADRYYVSVLVEVSDNKTINHFDEGIGIDLGLKDFAIVSNGRTYQNINKSAKLKKLERQLIREQRCLSRKYENLKKGDATQRANIQKQKLKVQKLHHRIDNIRTDYINKTIAEMVKIKPSYITIEDLNVKGMMKNRHLSKAVASQKFYEFRAKLLAKCNESGIELRVVDRWYPSSKTCHCCGSIKKDLKLSDRIFKCRCGYIEDRDFNAALNLRDAETYSIA

SEQ ID NO: 22 - protein, 390 aa, Clostridium perfringens
MKKLKRAYKIEINPTAEQKSKIHQTIGVSRFIYNFYIAHNKEIYAREGKFVSGMDFSKWLNNKYIPNNKDMKWIKEVSSKATKQAIMNGDKAFRDFFKKAKGFPKFKKKKNQDVKAYFPKNNKTDWTLERHKVKIPTLGWVRLKEFGYIPVNSIVKSGTVSQKADKYYVSILVEEDDKKVYKSTNEGLGIDLGVKEFVVCSNGIKFKNINKTSTVKKVEKKLKREQRKLSRKYESLKIRNKNLKEGRATSQNIQKQLVKVQKLHQRLTNIRTDYINKIVSSIIKQKPSYITIEDLAVSNLMKNKHLSKAIASQKFFEFKTKLMFKCKENHIELRIVDRFYPSSKTCSSCGKVKKDLKLSDRVYKCDCGLTIDRDLNASINLKNAKEYKIA

SEQ ID NO: 23 - protein, 387 aa, Coprococcus catus
MVKAIKVMLIPNNVQKTKMFQYAGASRFAYNWALAREKESYEKGGRFISDSELRKEFTKLRHSDEYAWLLNISNNVTKQAIKDACTAYKNFFKGLQKFPRFKSRKRSMPKFYQDNVKIQFSNTHVKLEGFSSSRKANKQKKNWVRLAEHGRIPTDVKYMNPRISFDGLNWWISVCVEFPDCKETLNDDGVGIDLGIKDLAVCSDAVKYKNINKSQKVKKLEKQKRRLQRSISRSYEKNKKGESYCKTNNVIKKEKLLLKRNHRLTNIRKNYLNQTISEIVNRKPRFICIEDLNVSGMMKNRHLSKAVQEQGFFWFRKQLECKCNDKGIQLIVADRFYPSSKLCSCCGNIKEDLKLSDRVYRCECGNMIDRDFQASINLKAYGERFAS

SEQ ID NO: 24 - protein, 387 aa, Coprococcus comes
MVKAIKVMLIPNNVQKTKMFQYAGASRFAYNWALAREKENYEKGGKFISDSELRKEFTKLRHSDEYAWLLNISNNVTKQAIKDACTAYKNFFKGLQKFPRFKSKKRSMPKFYQDNVKIQFSNTHVKFEGFSSSRKANKQKMNWVRLAEHGRIPTDVKYMNPRISFDGLNWWISVCVEFPDCKETLNDDGVGIDLGIKDLAVCSDAVKYKNINKSQKVKKLEKQKRRLQRSISRSYEKNKKGESYCKTNNVIKKEKLLLKRNHRLTNIRKNYLNQTISEIVNRKPRFICIEDLNVSGMMKNRHLSKAVQEQGFFWLRKQLEYKCSDKGIQLIVADRFYPSSKLCSCCGNIKKDLKLSDREYRCECGNIIDRDFQASINLKGYGERFAS

SEQ ID NO: 25 - protein, 392 aa, Coprococcus sp.
MIKSIKVRLCPNNKQLTKLFQYAGCARFAYNWAIAREQENYKQGDKFLSDNELRKEFTQLKKQSEYQWLSEVSNNVTKQAIKDACNTYKKFFKGQCKYPKFKSRKHSSPSFYQDNIKIQFSDTHVKVEGFSMSKKKNKQKLNWIRLCEKGRIPTNCKYMNPRFTYDELHWYISVGIEVEDSTTIPLNNGIGIDLGIKDLAVCSDGNIYKNINKTTTVNKLEKRKRRLQRSISRRYEKNKKGGIYCKTCNIIKKEKELLKVTKRLTNIRQNYLHQTTSEIIKRKPSFICIEDLNVSGMMKNKHLSKAVQQQCFYEFRKQMEYKSAWNNIPVIIADRFFPSSKLCSCCESIKKDLKLSDRIYKCECGNVIDRDYQASLNLKMYGENVLKQQSVA

SEQ ID NO: 26 - protein, 387 aa, Dorea formicigenerans
MVKAIKVMLLPNNVQKTKMFQYAGASRFAYNWALAREKESYEKGGKFISDSELRKEFTKLRHSDEYAWLLNISNNVTKQAIKDACTAYRNFFKGLQKFPRFKSRKRSMPKFYQDNVKIQFSNTHVKLEGFSSSRKANKQKKNWVRLAEHGRIPTDVKYMNPRISFDGLNWWISVCVEFPDCKETLNDDGVGIDLGIKDLAVCSDAVKYKNINKSQKVKKLEKQKRRLQRSISRSYEKNKKGESYCKTNNVIKKEKLLLKRNHRLTNIRKNYLNQTISEIVNRKPRFICIEDLNVSGMMKNRHLSKAVQEQGFFGFRKQLEYKCNDKGIQLIVADRFYPSSKLCSCCGNIKKDLKLSDRVYRCECGNMINRDFQASINLKTYGERFAS

SEQ ID NO: 27 - protein, 311 aa, Dorea formicigenerans
MYSKAVKKSIEDGCAAFTRFFKHQSDFPKFKKKGKSDVKMYFVKNNPKDCQCERHRLKIPTLGWVRIKEKGYIPTTKAGYMIRSGTVSVKAGRFYVSVLVEIPDVNINNNLNEGIGIDLGLKDFAIISNGKTYRNINKSAGLKKLEKQLIREQRSLSRKYENLKKGESTQRANIQKQKLKVQKLHHKMDNIRTDYINKTIAEIVKTKPSYITIEDLNVKGMMKNRCLSKAVASQKFYEFRKRLKAKCDEKGIELRIADRFYPSSKTCHHCGSIRKNLKLSDRIYRCECGYVADRDLNAALNLKDAKTYKIA

SEQ ID NO: 28 - protein, 387 aa, Dorea sp.
MRKLLKSFKTEINPTEEQKVKIRKTIGTCRFIYNFYLAHNKELHKNGEKFMSSNKFRVWLNNEYLPNHPEYSWIKEAYSKAVTQAVNNGQTAFTRFFNHKSSFPNFKKKGKSDVKMYFVKNNPKDCRCERHRINIPSLGWVRIKEKGYIPTTKDGYVIKSGHVSIKADRYYVSVLIEIPNVKIANNSNAGIGIDLGLKDFAIVSNGKTYKNINKSAKLKKLEKQLIREQRSLSRKYENLKKGESAQKPNIQKQRLKVQKLHHRIDNIRTDYINKTIAEIVKTKPSYITIEDLNISGMMKNKHLSKAVASQKFNEFRTKLQAKCNENGIELRVVDRWFPSSKTCHCCKNIKKDLKLSDRIFKCDCGYIEDRDFNAALNLRDATTYEVA

SEQ ID NO: 29 - protein, 402 aa, unidentified
MIKSIKVRLNPNNKQSTKLFQYAGCARFAYNWAISREQDNYKQGNKFLSDSELRKEFTQLKKQSEYQWLNEVSNNVTKQAIKDACNTYKRFFKGQCKYPKFKSKKHSTPSFYQDNIKIQFTDTHVKVESFSMSKKQNKQKLNWIKLCEKGRIPTGCKYMNPRFTYDGLYWYVSIGIEVNDNTTLPSNEGIGIDLGIKHLAICSDGNTYKNINKTQTVKKLEKKKRRLQRSISRKYEKNKKGANYCKTSNIIKREKELLKLNHRLTNVRQNYLHQTTSEIVKREPSFICIEDLNVSGMMKNKHLSKAVQQQGFYEFRRQIEYKAMWNNIPVVIADRFFPSSKLCSCCGSIKKDLKLSDRIYKCECGNVIDRDYQAALNLKQYGENVLKQQSVAYHFQVITDMY

SEQ ID NO: 30 - protein, 387 aa, unidentified
MVKAIKVMLIPNNVQKTKLFQYAGASRFAYNWALAREKENYEKGGKFISDSELRKEFTRLRHSDEYAWLLNISNNVTKQAIKDACTAYQNFFKGLQKFPRFKSRKRSMPKFYQDNVKIQFSNTHVKFEGFSSSRKANKQKRNWVRLAEHGRIPTDAKYRNPRISFDGLNWWISVCVEFPDSKETLNEDGVGIDLGIKDLAVCSDAVKYKNINKSQKVKKLEKQKRRLQRSISRSYEKNKKGESYCKTNNVIKKEKFLLKRNHRLTNIRKNYLNQTISEIVNRKPRFICIEDLNVRGMMKNRHLSKAVQEQGFFGFRKQLEYKCSDKGIQFIVADRFYPSSKLCSCCGNIKKDLKLSDRIYRCECGNRIDRDFQASINLKAYGEQFAS

SEQ ID NO: 31 - protein, 343 aa, unidentified
MVKAIKVMLIPNNVQQTKLFQYAGASRFAYNWALAREKENYEKVGRFIPDTELRKEFTRPRNSDEYAWLLNVSNNVTKQAIKDACSAYKNFFKGLQRYPRFKAKKKSMPKFYQDNIKIQFRDTNVKFEGFSSSRKANKQKLNWVKLAEHGRIPTDAKYRNPRVSFDGLNWWISVCVEFPDCRDRSNHDGIGIDLGIKDLAICSDGNTYKNINKSQTAKKLEKCKRRLQRSVSRKYEHNKKGGSYCKTNNIVKKEKLLLKVNHRLANIRKDYLNQTTSEIANRKPRFICIEDLNVSGMIKNRHLSKAVQNQGFFEFRKQLEYKCNDRDFQASLNLKAYGEQFAS

SEQ ID NO: 32 - protein, 387 aa, unidentified
MVKAIKVMLIPNNVQKTKMFQYAGASRFAYNWALAREKENYEKGGRFISDSELRKEFTKLRHSDEYAWLLSISNNVTKQAIKDACIAYKNFFRGLQKFPRFKSRKRSMPKFYQDNVKIQFSNTHVKFEGFSSSRKANKQKMNWVRLAEHGRIPIDANYMNPRISFDGLNWWISVCVEFPDCRETLNDDGVGIDLGMKDFAVCSDAVKYKNINKSQKVKKLEKQKRRLQRSISRSYEKNKKGESYCKTNHVIKKEKLLLKRNHRLTNIRKNYLNQTVSEIVNRKPRFICIEDLNVSGMMKNRHLSKAVQEQGFFLFRKQLEYKCSDKGIQLIVADRFYPSSKLCSCCGNIKKDLKLSDRVYRCECGNMIDRDFQASINLKAYGERFAS

SEQ ID NO: 33 - protein, 375 aa, Megamonas funiformis
MILAKKVRIYPTKEQEQKLWQSVGTARFIYNYTLAKQEENYKNGGKFISDGIIRKELTQLKKSELIWLNEVSNNVTKQAVKDACNAYKRFFKGLAKKPKFKIRKKSKPSFYNDTSKLKVKEKKVLIEKVGWIKTNEQIPIDVKYNNPRVTYDNKYWYISVGVEVDKKQEELTDISLGIDLGLKDLAVCSDGKVFKNINKTKEVKNLEKRLKQKQRQISRKYEINKIKKGGSCQFIKTKNIEKLENTTKLMHRKLTNIRDNYIHQVTTSIVKTKPYRIVIEDLNVSGMIKNKHLSDSVRKQCFNKFRQYITYKAELYGIGLVIADRFYPSSKTCSECGSIKKDLKLKDRVYRCPHCGVVIDRDYNASLNLSMYKLA

SEQ ID NO: 34 - protein, 377 aa, Megamonas rupellensis
MILAKKVRIYPTKEQEQKLWQSVGTARFIYNYTLAKQEENYKNGGKFINDGVIRKELTQLKKSELTWLNEVSNNITKQSVKDACKAYKRFFKNLANKPKFKSRKKSKPSFYNDPIKLKIKEKKVLIEKVGWIKINEQIPINVKYNNPRITYDNKYWYLSVGIEVDKKQEGLTNISLGIDLGLKELAICSDGKVFKNINKTKKIKKLEKRLKQKQRQISRKYEINKIKKEGGKRYQFIKTKNIKKLENTTKLIYRKLANIRNNYIHQVTTSIVKTKPYRIVIEDLNVSGMMKNKHLSDSVKKQCFHKFRQYITYKSELNGIELVVADRFYPSSKTCSKCGFIKKDLKLKDRVYRCPHCGAVIDRDLNASLNLSMYKLA

SEQ ID NO: 35 - protein, 393 aa, Mitsuokella multacida
MLKAFKTEIAPTKEQRDKIIRSIGVARFLYNQYISYNRRLYKMYRRGMLDERQKYFVTAVDFDKYVNHKLKQELPWIDQCGSKARKKALVNAEQAFRRFFAGISRFPTFKKKTNQDVKLYFPRNNKGDWKIWRHKLMIPTLKQVKLKEYGYLPVGMTATSGTVSYVAGRFYVSVVVDIDAKSKYNKDLDSCYAIQTEGIGIDLGVKDLAVVSDGRVFENINKSSKVKRLEKRLRREQRRLSRKYVGKVDTVSANIEKQKLKVQKLHQRITRIREDYENKTVHEIVKQKPRFITVEDLNVKGMMKNRHLAKAVAAQRFNHLLSKLKRKAKIIGIELREVDRFYPSSKTCHACGHVHRGLKLKDRVYICPECGYRMDRDYNASLNLRDAKEYRVV

SEQ ID NO: 36 - protein, 402 aa, Mitsuokella multacida
MRNLLKSFKTELRLTEGQRDKVRRSLGICRFLANQYIARNIWLYRLYQRGVLDEKQRRFVTANDFDKYVNHVLKKKFPWIDQCGSKARKKILVNAEQAFRRFFRGQAHFPRFKRKNQQNVKLYFPKNNKGDWTIWRHKIQIPTIGKVRLKEFGYLPQGVTIKNGHVSYKAGRYYVSVTAEIEEMSRYNNDLEASYHPRSDGIGVDLGVKSLAIVSDGRIFENINRTSRARRLERRLRREQRRLSRKYEMRKKKGGSTATASANIEKKKLSVQRLHRRLANLRRNHENQVIHALVEQKPRFITVEDLNVTGMMKNRHLSKAVVQQRFYTFREKLRRKAEIIGIEVRIADRFYPSSKTCHACGHVHSGLKLKDRTYVCPACGYKKDRDFNAALNLRDTKNYRIA

SEQ ID NO: 37 - protein, 434 aa, Mitsuokella sp.
MIRSIRVQLLPNNKQKTKLFQFAGAARYAYNWALDKQMDNFREGRKLQTDHELRKAFTLLRHSEENKWLLDISNNVTKQAIKDLCIAYKNFFHKQKQPIYIKYSSKKITHYNRIGKSLTVYDMNGHPKFRSKKNGDFRFYQDNVKIQFTGTHVKLESLAGSRRKNRQRLNWVRLAERNRIPVRAKYTNPRIAFDGERWWLSVGIQTARKLKPLRGLSFVCKKRRYARSEGIGIDLGIKDLAVLSDATKVGNINKTRAIRKLKKKRRRLQRQVSQKYQMNKKGDSYCKTSNLIKSEKRLLRINHRLANIRQNHVHQATTSIIKRKPSFVCLEDLNVRGMMKNRYLSEKIQEQNLYEFRRQIEYKAHWAKIPVVIADRWYPSSKTCVICGHVKKELNLLERTYICPVCGNIIDRDFQAALNLKRYGEAEQVKLANY

SEQ ID NO: 38 - protein, 370 aa, Peptoclostridium sp.
MEKGFKVQIFPNKEQKELMFKSFGCARFAYNWALAKEKENYEKGGKFISDMDLRKEFTQLKKKEEFKWLNEINNNVTKQAIKDLCKAYKNFFKKKAEHPRFKSKKKSKKSFYNDVYKIKFDDYKVRIESIGWMDMAENRIPRNCKYQNPKISFDGVKFWLSITCIVEDEENNLPKTEPIGIDMGIKTLMTCSNGMNFNRVSTKKEKKKLKRLQKKASRHYDKMMKTKSSDKSKNLLKLETLIRKQYQRISNVRLNNIHQATTKLVKLNPKAIVVEDLNVKGMIKNKYLSEKIADCSFYEIYRQLEYKCENKGMEFIVADRFYPSSKICSNCGHKKEKLSLSERTYRCECCGNVIDRDFNASLNLKNLAYN

SEQ ID NO: 39 - protein, 387 aa, Ruminococcus sp.
MVKAIKVMLIPNNIQKTKMFQYAGASRFAYNWALAREIENYEKGGKFISDAELRKEFTKLRHSDEYAWLLSISNNVTKQAIKDACTAYKNFFKGLQKFPRFKPKKRSIPKFYQDNIKIQFSNTHVKFEGFSSSRKRNKQKLNWVRLAEHGRIPTDAKYMNPRISFDGLNWWISVCVEFPDCIEKLNDDGIGIDLGIKDLAICSDGAKYKNINKSQKVKKLEKQKRRLQRSISRSYEKNKKGESYCKTNNVIKKEKLLLKRNHRLTNIRKNYLNQTTSEIVSRKPRFICIEDLNVSGMMKNRHLSKAVQNQGFFEFRKQLEYKCSDKGIQLIVADWFYPSSKLCSCCGNIKKDLKLSDRIYKCDCGNVIDRDFQASINLKAYGERVAS

SEQ ID NO: 40 - protein, 387 aa, Ruminococcus sp.
MWKLLKSFKTEINPSEEQKVKIHKTIGTCRFIYNFYLAHNKELYNNGEKFMSSNRFRVWLNNEYLSEHPEYSWIKEAYSKAVTQSVNNGQTAFTRFFNHESAFPKFKKKGRSDVKMYFVKNNPKDCRCERHRINIPSLGWVRIKEKGYIPTTKDGYVVKSGTVSMKADRYYVSVLVEISDNKIADNSNAGIGIDLGLKDFAIVSNGKTYKNINKSARLKKLEKQFIREQRCLSRKYENLKKGEVTQRANIQKQKLKVQKLHHKIDNIRTDYINKTIAEIVKTKPSYITIEDLNVNGMMKNRHLSKAVASQKFYEFRTKLQIKCNENGIELRIVDRWYPSSKTCHCCGAIKKDLKLSDRIFKCSCGYVEDRDLNAALNLRDAITYEVA

SEQ ID NO: 41 - protein, 392 aa, Ruminococcus sp.
MLTTKKVRLKLTPEQEIQFRKSCGVARWAYNYLLSEKQRVYDEYISNGKTGKKTISEGEVRKYINNVLKPTTHQWLKEVGSNVMKQAVKDADNAYKNFFNGLSKRPKFKSKKSSKQSFYVNYESLTRINGGFKGEKLGFVKTSEPLPKLADGEKYANPRITFDGKYWYLSVVYNIEPKSVQLTNESLGIDLGVKNLAICSNGVTYKNINKSKRVKALKRTLKREQRKLSRKIECNIIGYANNRKPIFRTPLQGCKNIQKQIHYIKLINRKINSIRNNHLHQATAEIVKTKPFQIVMETLNITGMMKNKHLAKAIAEEKLFEFKRQIKYKAEMYGIKVVEVPTFYPSSKTCSVCGCINTNLKLSDRVYHCDSCGITLDRDLNAAINLANYKVI

SEQ ID NO: 42 - protein, 386 aa, unidentified
MRKLLKSFKTEINPTVEQKIKINKTIGTCRYVYNFYLGHNKALYDKGEKFMTGKSFSVWLNNEYVPDNPDKIWIKEAYSKAVKKSIEDGCTAFTRFFKHQSAFPNFKRKGKSDVKMYFVKNNPKDCRCERHRLNIPTLGWVRMKEKGYIPTTKDGWKIKSGTVSIKADRYYVSVLVEIPDAKIANNSNDGIGIDLGLKDLAIVSNGKTYKNINKSARVKKLEKKLRREQRGLSRKYENSKKGESTQKNIQKQKLKVQRLHHKIDNIRTDYINKSIAEIVKTKPSYITIEDLNVSGMMKNRHLSKAVASQKFYEFRTKLKAKCDENGIELRVVDRWYPSSKVCHCCGAIKKDLKLSDRIYRCDCGYVEDRDFNAALNLRDALTYEVA

SEQ ID NO: 43 - protein, 387 aa, unidentified
MWKLLKSFKTEINPSEEQKVKIHKTIGTCRFIYNFYLAHNKELYDNGEKFMSSNKFRVWLNHEYLSEHPEYSWIKEVYSKAVTQAVNNGQTAFARFFNHESAFPNFKKKGKSDVKMYFVKNNPKDCRCERHRINIPSLGWVRIKEKGYIPTTKDGYVIKSGTVSMKADRYYVSVLVEILNNKIANNSNAGIGIDLGLKDFAIVSNGKTYKNINKSARLKKLEKQLIREQRSLSRKYENLKKRESTQKTNIQKQRLKVQKLHHRIDNIRTDYINKTIAEIVKTKPSYITIEDLNVKGMMKNRHLSKAVASQKFYEFKTKLQTKCNENGIELRVVDRWYPSSKTCHCCGAVKKDLKLSDRMFKCSCGYVEDRDFNAALNLRDAITYEVA

SEQ ID NO: 44 - protein, 402 aa, Monoglobales sp.; identical to SEQ ID NO: 29

SEQ ID NO: 45 - protein, 380 aa, Eisenbergiella sp.
MLIPNNVQKTKLFQYAGASRFAYNWALAREKETYEKGGKFISDSELRKEFTRLRHSEEYAWLLNISNNVTKQAIKDACTAYQNFFKGLQKFPRFKSRKRSMPKFYQDNVKIQFSNTHVKFEGFSSSRKANKQKRNWVRLAEHGRIPTDVKYRNPRISFDGLNWWISVCVEFPDSKETLNEDGVGIDLGIKDLAVCSDAVKYKNINKSQKVKKLEKQKRRLQRSISRSYEKNKKGESYCKTNNVIKKEKLLLKRNHRLTNIRKNYLNQTISEIVNRKPRFICIEDLNVRGMMKNRHLSKAVQEQGFFLFRKQLEYKCSDKGVQLIVADRFYPSSKLCSCCGNIKKDLKLSDRVYRCECGNRIDRDFQASINLKAYGEQVAS

SEQ ID NO: 46 - protein, 337 aa, Anaerostipes hadrus
MSSNQFRVWLNNEYISKHPECFWIKEAYSKAVTQAVNNGQAAFTRFFKHQSSFPRFKKKNRSDVKMYFVKNNPKDCRCQRHRINIPSLGWVRIKEKGYIPTTKDGYVIKSGHVSIKAGRYYVSALVEIPNNKIANSSNEGIGVDLGIKDFAVVSNGKVYKNINRSARLKKLEKQLIREQRSLSRRYENLKKGESTQKANIQKQRLKVQKLHHRIDNIRTDHINKTIAEIVKTKPSYITIEDLNISGMMKNRYLSKAVASQKFYEFRIKLQAKCNENGIELRVVDRWYPSSKTCHCCGAIKKDLKLSDRIFKCDCGYTEDRDLNAALNLRDAMTYEVA

SEQ ID NO: 47 - protein, 383 aa, Eisenbergiella sp.
MLKSFKTEIDPTPEQKVKINKTIGTCRYVYNVYLSHNKALYDKGEKFMTGKSFSVWLNNEYIPNNPDKAWIKEVSSKAIKKSIEDGCTAFRRFFKHQSSFPSCKKKGRSDVKMYFVKNNPKDCKCERHRISIPTLGWMRMKEKGYIPTTKDGYAIKSGTVSMKAGRYYVSALVEVPNVNVRNNTNDGVGIDLGLKDLAIVSNGKIYKNINKSARLKKFEKRLRREQRCLSRKYDNRKKGESTQKNIQKQRLKVQRLHHKIENIRTDHINKTIAEIVKTKPSYITIEDLNVSGMMKNRHLSKAVASQKFYEFKIKLKAKCEDNGIELRVVDRFYPSSKLCHCCGRIKKDLKLSDRIYKCECGYIEDRDYNAALNLRDAVTYEVA

SEQ ID NO: 48 - protein, 336 aa, Mageeibacillus indolicus
MTGKSFSVWLNNEYIPNNPEKVWIKEAYSKAVKKSIEDGYTAFTRFFKHQSAFPNFKKKGKSDVKMYFVKNNPKDCRCERHRLNIPTLGWVRIKEKGYIPTTKDGWKIKSGTLSIKAGKYYVSVLVEIPDAKIANNSNDGIGIDLGLKDLAVVSNGKTYKNINKSARVKKLEKKLRREQRCLSRKYENIKEGESTQKNIQKQKLKVQRLHHKIDNIRTDYINKSIAKIVKTKPSYITIEDLNVSGMMKNRHLSKAVASQKFYEFRTKLKAKCDENGIELRVVDRWYPSSKICHCCGAIKKDLKLSDRIYRCDCGYIEDRDLNAALNLRDASTYEIA

SEQ ID NO: 49 - protein, 383 aa, Eubacterium rectale
MLKSFKTEISPTIEQKIKINKTIGTCRYVYNFYLDHNKTLYDNGEKFMTGKSFSVWLNNEYIPNNPDKIWIKEAYSKAVKKSIEDGCTAFTRFFKHQSAFPNFKKKGKSDVKMYFVKNNTKDCRCERHRLNIPTLGWVRIKEKGYIPTTKDGWKIKSGTVSVKADRYYVSVLVEIPDVKIANNSNGGIGIDLGLKDLAIVSNGKTYKNINKSTRIKKLEKKLRREQRCLSRKYENLKKGESTQKNIQKQKLKVQRLHHKIDNIRTDYINKSIAEIVKTKPSYITIENLNVSGMMKNRHLSKAVASQKFYEFRTKLKAKCDENGIELRVVDRWYPSSKICHCCGAIKKDLKLSDRIYHCDCGYVEDRDFNAALNLKDALTYEVA

SEQ ID NO: 50 - protein, 383 aa, unidentified
MLKSFRTEIHPTPEQKVKINKIIGTCRYIYNFYLSNNKALHDQGEAFMSGKAFSVWLNNEYIPDNPDKAWIKEVYSKAVKKSIEDGCLAFTRFFKHQSGFPKFKKKGKSDVKMYFVKNNPKDCICERHRIKIPALGWVRLKEKGYIPTTKDGWKIKSGTVSMKADRYYVSVVVEIPDTKPFCDSGEGIGIDLGLKDFAIVSNGKTYKNINKSAKLKKLEKQLRRAQRCLSRKYENLKKGESTQKNIQKQRLKVQKLYQRIDNIRTDYMNKTIAEIVKTKPSYITIEDLNVKGMMKNRHLSKAVASQKFYEFRTKLKNKCDENGIELRVVDRWYPSSKLCHCCGAIKKDLKLSDRIYKCNCGYIADRDYNASLNLRDAKTYKIA

SEQ ID NO: 51 - protein, 384 aa, Dorea formicigenerans
MLKSFKTEINPTEEQKVKIRKTIGTCRYIYNFYLAHNKELYDKGEKFMSGKSFSVWLNNEYLPQNPDKLWIKEVSSKSVKRSIENGCIAFTRFFKHQSAFPNFKKKGKSDVKMYFVKNNPKDCRCERHRINIPSLGWVRIKEKGYILTTKDGYAIKSGHVSIKADRYYVSVLVEIPNNKIANNSNEGIGIDLGLKDFAIVSNGKTYKNINKSAKLKKLEKQLIKEQRSLSRKYENLKKGESTQKTNIQKQRLKVQKLHHRIDNIRTDYINKTIAEIVKTKPSYITIEDLNISGMMKNKHLSKAVASQKFYEFRIKLQAKCKENGIELRVVDRWFPSSKTCHCCGAVKKDLKLSDRIFKCSCGYVEDRDFNAALNLRDAATYEVA

SEQ ID NO: 52 - protein, 384 aa, Agathobacter rectalis
MLRSFKTEINPSEEQKVKIHKTIGTCRFIYNFYLAHNKELYDNGEKFMSSNRFRVWLNNEYLSEHPEYLWIKEAYSKAVTQSVNNGQTAFTRFFNHESAFPKFKKKGRSDVKMYFVKNNPKDCRCERHRINIPSLGWVRIKEKGYIPTTKDGYVIKSGTVSIKADRYYVSVLVELPDNKTADNSNEGIGIDLGLKDFATVSNGKTYKNINKSAKLKKLEKQLIREQRSLSRKYENLQKGESTQRANIQKQKLKVQKLHHRIDNIRTDYINKTIAEIVKTKPSYITIEDLNVKGMMKNRHLSKAVASQKFYEFRIKLQAKCTENGIELRVADRWYPSSKTCHCCKNIRKDLKLSDRIFKCDCGYIEDRDLNAALNLRDAMTYKVA

SEQ ID NO: 53 - protein, 387 aa, Dorea sp.; identical to SEQ ID NO: 28

SEQ ID NO: 54 - protein, 384 aa, Hungatella sp.
MLKSFKTEINPTEEQKVKIRKTIGTCRYIYNFYLAHNKELHDNGEKFMSGKSFSVWLNNEYLPKNPDKLWIKEVSSKSVKRSIENGCVAFTRFFKHQSAFPNFKKKGKSDVKMYFVKNNPKDCRCERHRMNIPSLGWVRMKEKGYVPTTKDGYVIKSGHVSIKADRYYVSVLIEIPNSKTADNSNEGIGIDLGLKDFAIVSNGKTYKNINKSAKLKKLEKQLIREQRSLSRKYENLKEGESTQRANIQKQKLKVQKVHHKIDDIRTDYINKTIAEMVKTKPSYITIEDLNVSGMMKNKHLSKAVASQKFYEFRTKLQAKCKENGIELRVVDRWYPSSKTCHCCGAIRKDLKLSDRIFECDCGYIEDRDFNAALNLRDATTYEVA

SEQ ID NO: 55 - protein, 384 aa, Agathobacter faecis
MLKSFKTEINPTEEQKVKIRKTIGTCRYIYNFYLAHNKELHDNGEKFMSGKSFSVWLNNEYLPKNPDKLWIKEVSSKSVKRSIENGCVAFTRFFKHQSAFPNFKKKGKSDVKMYFVKNNPKDCRCERHRMNIPSLGWVRMKEKGYVPTTKDGYVIKSGHVSIKADRYYVSVLIEIPNNKIANHSKEGIGIDLGLKDFAIVSNGKTYKNINKSAKLKKLEKQLIREQRSLSRKYENLKKGESTQRANIQKQKLKVQKLHHKIDDIRTDYINKTIAEMVKTKPSYITIEDLNVSGMMKNKHLSKAVASQKFYEFRTKLQAKCKESGIELRVVDRWYPSSKTCHCCGAIKKNLKLSDRIFQCDCGYIEDRDFNAALNLRDATTYEVA

SEQ ID NO: 56 - protein, 384 aa, Clostridium sp.
MLKSFKTEINPTEEQKVKIRKTIGTCRYIYNFYLAHNKELHDNGEKFKSGKSFSVWLNNEYLPQNPDKLWIKEVSSKSVKHSIENGCVAFTRFFKHQSAFPNFKKKGRSDVKMYFVKNNPKDCRCERHRVNIPSLGWVRIKEKGYIPTTKEGYIIKSGHVSMKAGRYYVSALIEIPDNKTADLSNEGIGIDLGLKDFAIVSNGKTYKNINKSARLKKLEKRLVREQRCLSRKYENLKKGESTQRANIQKQKLKVQKLHHKIDNIRTDYINKTIAEIVKTKPSYITIEDLNVSGMMKNKHLSRAVASQKFYEFRIKLKAKSRENGIELRVADRWYPSSKICHCCGMIKKDLKLSDRIFRCHCGYVEDRDFNAALNLRGVKTYKIA

SEQ ID NO: 57 - protein, 387 aa, Fusicatenibacter saccharivorans
MRTLLKSFKTEINPTEEQKVKIRKTIGTCRYIYNFYLAHNKELHENGKKFMSGKRFSVWLNNEYLPKNPDKLWIKEVSSKSVKRSIENGCVAFTRFFKHQSDFPNFKKKGKSNVKMYFVKNNPKDCRCERHRINIPSLGWVRIKEKGYVPTTKDGYVIKSGHVSIKADRYYVSVLVELPDNKTADNSNEGIGIDLGLKDFAIVSNGKTYKNINKSTKLKKLEKQLIREQRSLSRKYENLKKGESTQRANIQKQKLKVQKLHHKIDNIRTDYINKTIAEIVKTKPSYITIEDLNVSGMMKNKHLSRAVASQKFYEFRIKLKAKSRENGIELRVADRWYPSSKICHCCGMIKKDLKLSDRIFRCHCGYVEDRDFNAALNLRGVKTYKIA

SEQ ID NO: 58 - RNA, 185 nt, synthetic construct
  source 1..185: mol_type = other RNA
atgaacgcaaacgtaagtatgtaccgaaggctatttcgggaatttacgactatggagtgcactagaacttgtgagtagatatgatttcggtcaatccaaagcatgcacagtgacatagtaagtaatgttcgtgagaacttacattatctcgatgtgagtatatttaatcacattttgagtggcag

SEQ ID NO: 59 - RNA, 207 nt, synthetic construct
  source 1..207: mol_type = other RNA
ttcacgttttcacgtcttgatacatgtaccgatggctagtcgggaacaggccgttgccttgtgaggcaaggaatacggccaacgcctttggagtgtacaagaacttgtgagtagtctcaggcttgcctggacaaaagcatacacgatgaataaggaatttttctaggcatagataatttctatgttattctatgtttttagtagcag

SEQ ID NO: 60 - RNA, 184 nt, synthetic construct
  source 1..184: mol_type = other RNA
gaaacgcagagatacatatgtaccgaaggccatttcgggaattaacgactgtggagtgtaccagaacttgtgagtagatacagctccggctggtcaaaagcatacacgtttgaaacagtaagtaggattcgtgagaactacaatatctcgatatgagtatatttgatcatattttgagtggcag

SEQ ID NO: 61 - RNA, 184 nt, synthetic construct
  source 1..184: mol_type = other RNA
tcccgcaaacgtaagtatgtaccgaaggctatttcgggaattaacgactgtggagtgtacttaggatctgtgagtagagataactccggttactcaaaagcatacacaaagaaacagtaagcggtattcgtgagaatccgcattatctcgatgtgagtatgtttaatcacattttgagtggcag

SEQ ID NO: 62 - RNA, 187 nt, synthetic construct
  source 1..187: mol_type = other RNA
tgaaaagcaaatgtaagtatgtaccgaaggctatttcgggaattaacgactgtggagtatacaagaacttgtgagtagcatatctgtttacagattgtaaaagcatatacgatgaagcagtaagtaatgttcgtgagaacttacaacatctcgatgtgagtatatttacacacattttgagtggcag

SEQ ID NO: 63 - RNA, 187 nt, synthetic construct
  source 1..187: mol_type = other RNA
ctaaaacgcaaacgtaagtatgtaccgaaggctattttcgggaatttacgactatggagtgtacaagaacttgtgagtagatatgatttcggtcaatccaaagcatacacgatgaaatagtaagtaatgttcgtgagaacttacattatctcgatgtgagcatatttaatcacattttgagtggcag

SEQ ID NO: 64 - RNA, 185 nt, synthetic construct
  source 1..185: mol_type = other RNA
ataaatgcaaacgtaggtatgtaccgaaggctatttcgggaatttacgactatggagtgtacaagaacttgtgagtagatatgatttcggtcaatccaaagcatacacgatgaagtagtaagtagaactcgtgagagtctacattatctcgatgtgagtatgtttaatcacattttgagtagcag

SEQ ID NO: 65 - RNA, 187 nt, synthetic construct
  source 1..187: mol_type = other RNA
ctaaaacgcaaacgtaagtatgtaccgaaggctattttcgggaatttacgactatggagtgtacaagaacttgcgagtagatatgatttcggtcaatccaaagcatacacgatgaaatagtaagtaatgttcgtgagaacttacattatctcgatgtgagcatatttaatcacattttgagtggcag

SEQ ID NO: 66 - RNA, 183 nt, synthetic construct
  source 1..183: mol_type = other RNA
aaaagcagatgtaagtatgtaccgaaggctatttcgggaatttacgactgtggagtgtacaagaacctgtgagtagatatgattttggtcaatcaaaagcatacacaaagaagcagtaagtagaactcgtgagagtttacattatctcgatgtgagtataattacacacattttgagtggcag

SEQ ID NO: 67 - RNA, 186 nt, synthetic construct
  source 1..186: mol_type = other RNA
ttaaaacgcaaatgtaagtatgtaccgaaggctatttcgggaatttacgactgtggagtgtacatgaacttgtgagtagatatgactacggtcaatcaaaaacatacacaatgaagcagtaagtaatgttcgtgagaacttacattatctcgatgtgggtatatttaatcacattttgagtggcag

SEQ ID NO: 68 - RNA, 185 nt, synthetic construct
  source 1..185: mol_type = other RNA
aacgcaaacgtaagtatgtactgcgggctatcgcaggaatttacgactgtggagtgtacaagaacttgtgagtagcgtattgtttacaatcgccaaagcatacacaatgaagcagtaagaagtatccgcaaggacttcaatttctcgatgtgtttgagtatatttcaacacattttgagtggcag

SEQ ID NO: 69 - RNA, 185 nt, synthetic construct
  source 1..185: mol_type = other RNA
ataaacgcaaacgtaggtatgtaccgaaggctatttcgggaatttacgactatggagtgtacaagaacttgtgagtagatatgatttcggtcaatccaaagcatacacgatgaaatagtaagtaaaactcgtgagagtttacattatctcgatgtgagtatatttaatcacattttgagtggcag

SEQ ID NO: 70 - RNA, 187 nt, synthetic construct
  source 1..187: mol_type = other RNA
taaacgcaaacgtaagtatgtactgcgggctatcgcaggaatttacgactgtggagtgtacacgaacttgtgagtagcgtattgtttacaatcgccaaagcatacacatcgaagcagtaagaagtatccgcaaggactttaatttctcgatgtgtttgagtatatttgaacacattttgagtggcag

SEQ ID NO: 71 - DNA, 149 bp, synthetic construct
  source 1..149: mol_type = other DNA
atagattgtatatatgtacggtgggctacatcggaatttacgcctgtggagtgtcacagcaaactgtagtagcaagtgattagcgaggcagggcacgtagaagcaggaatactctaagaatatacatttgtctatatttttagtagcag

SEQ ID NO: 72 - RNA, 155 nt, synthetic construct
  source 1..155: mol_type = other RNA
cacctaaaacgttaacgctaatatgtacgattcgttgcatcggaatttacgcctgtggagtgttacaccaaacgaaagtagcttcggcaaaatcggacacgatgaatcaggaagcaaacaagaatttataagtttttataatttcttggcaacgg

SEQ ID NO: 73 - RNA, 168 nt, synthetic construct
  source 1..168: mol_type = other RNA
cactgaaacgttaatgcaaatatgtacggatacgttagtccggaatttacgcctgtggagagtacaagaacttgtgagtagattgatatttatatcatcaaaagcatactcgttgaagcaggaatgaaacatggaagtttataactttttataagttttcagtaacgg

SEQ ID NO: 74 - RNA, 186 nt, synthetic construct
  source 1..186: mol_type = other RNA
ggaagcaaatgtaagtatgtaccgaaggctatttcgggaattaacgactgtggagtgtacatggacctacgagtagatatgatttcggtcaatcaaaagtatacacactgaagcagtaagtaagattcgtgagaacttacattatctcgatgtgataagtatatttaatcacattttgagtggcag

SEQ ID NO: 75 - RNA, 184 nt, synthetic construct
  source 1..184: mol_type = other RNA
tcctgcaaacgtaagtatgtaccgaaggctatttcgggaattaacgactgtggagtgtacttaggatctgtgagtagagataacgcaggttactcaaaaacatacacgaagaaacagtaagcggtattcgcgagaagccgcattatctcaatgtgagtatgtttaatcacattttgagtggcag

SEQ ID NO: 76 - RNA, 186 nt, synthetic construct
  source 1..186: mol_type = other RNA
ttaacgcaacgtaagtatgtaccgaaggctatttcgggaattaacgactgtggagtgtacaagaacttgtgagtagcgtattgcttgcaatcgccaaagcatacacattgaagcagtaagaagtatccgtgaggacttcagtttctcgatgtgtttgagtatatttaaacgcattttgagtggcag

SEQ ID NO: 77 - RNA, 187 nt, synthetic construct
  source 1..187: mol_type = other RNA
tgaacgcaaatgtaagtatgtaccgaaggctatttcgggaattaacgactgtggagtgtaccagaacttgtgagtagtgagttacttgtaaccacaaaagcatacatggtgaagcagtaagaagtatccgtgaggacttcaatttctcgatgtgattgagtatgttcaaacacattttgagtggcag

SEQ ID NO: 78 - RNA, 186 nt, synthetic construct
  source 1..186: mol_type = other RNA
ttaaaacgcaaatgtaagtatgtaccgaaggctatttcgggaatttacgactgtggagtgtacatgaacttgtgagtagatatgactacggtcaatcaaaaacatacacaatgaagcagtaagtaatgttcgcgagaacttacattatctcgatgtgggtatatttaatcacattttgagtggcag

SEQ ID NO: 79 - RNA, 151 nt, synthetic construct
  source 1..151: mol_type = other RNA
aatcaaaagcacttatatatgtacggagggctaacttcggaatttaagcctttggagagccatacaaatcgtagtagcttaggcaaggcgaggctttatgaagaaggaataatctccatatggatatatttgaccatattttgagtagcag

SEQ ID NO: 80 - RNA, 168 nt, synthetic construct
  source 1..168: mol_type = other RNA
cactgaaacgttaatgcaaatatgtacggatacgttagtccggaatttacgcctatggagagtacaagaacttgtgagtagatagatatttatatcatcaaaagcatactcgttgaagtaggaatgaaacatggaagtttataactttttataagttttcagtaacgg

SEQ ID NO: 81 - RNA, 168 nt, synthetic construct
  source 1..168: mol_type = other RNA
cactgaaacgttaatgcaaatatgtacggatacgttagtccggaatttacgcctatggagagtacaagaacttgtgagtagatagatatttatatcatcaaaagcatactcgttgaagtaggaatgaaacatagaagtttgtaactttttataagttttcagtaacgg

SEQ ID NO: 82 - RNA, 168 nt, synthetic construct
  source 1..168: mol_type = other RNA
cactttcaagttaatacagatatgtactgattcgttagtcaggaatttacgcctatggagagtacaagaacttgtaagtagtgattgacttaccaacacgaaagcatactcgttgaaataggaatgaaacataaaagtttataactttttataagttttcagtaacgg

SEQ ID NO: 83 - RNA, 168 nt, synthetic construct
  source 1..168: mol_type = other RNA
cactgaaacgttaatgcaaatatgtacggatacgttagtccggaatttacgcctatggagagtacaagaacttgcgagtagatagatatttatatcatcaaaagcatactcgttgaagtaggaatgaaacatgggagtttataactttttataagttttcagtaacgg

SEQ ID NO: 84 - RNA, 188 nt, synthetic construct
  source 1..188: mol_type = other RNA
ttaaaagcaaatgtaagtatgtaccgaaggctatttcgggaattaacgactgtggagtgtacaagaacctgtgagtagcgtattgcctgcaatcgtcaaaacatacacaatgaagcagtaagaagtattcgtgaggacttcaatttctcgatgtgtttgagtatatttaaacatatttggagtggcag

SEQ ID NO: 85 - RNA, 185 nt, synthetic construct
  source 1..185: mol_type = other RNA
ataaacgcaaacgtaggtatgtaccgaaggctatttcgggaatttacgactatggagtgtacaagaacttgtgagtagatatgatttcggtcaatccaaagcatacacgatgaaatagtaagtaatgttcgtgagaacttacattatctcgatgtgagtatatttaatcatattttgagtggcag

SEQ ID NO: 86 - RNA, 138 nt, synthetic construct
  source 1..138: mol_type = other RNA
tacgttagtcaggaatttatgcctatggagtgtacaagaacttgcgagtagattatgagaaatcatgtcaaaagcatatacgatgaagtaggaatgaaacataaaggtttataactttttataagttttcagtaacgg

SEQ ID NO: 87 - RNA, 168 nt, synthetic construct
  source 1..168: mol_type = other RNA
gattgaaacgttaatgcaaatatgtacggatacgttagtccggaatttacgcctatggagagtacaagaacttgtgagtagatagatatttatatcgtcaaaagcatactcgttgaagtaggaatgaaacatagaagtttataactttttataagttttcagtaacgg

SEQ ID NO: 88 - RNA, 168 nt, synthetic construct
  source 1..168: mol_type = other RNA
cacttaaatgttaatgcaaatatgtacggatgcgttaattcggaatttacgcctatggagagtacaagaacttgtgagtagataggtatttatgccatcaaaagcatactcgttgaagtaggaatgaaacataaaagtttataactttttataagttttcagtaacgg

SEQ ID NO: 89 - RNA, 168 nt, synthetic construct
  source 1..168: mol_type = other RNA
cactgaaacgttaatgcaaatatgtacggatacgttagtccggaatttacgcctacggagagtacaagaacttgtgagtagatagatatttatatcatcaaaagcatactcgttgaagtaggaatgaaacatagaagtttataactttttataagttttcagtaacgg

SEQ ID NO: 90 - RNA, 155 nt, synthetic construct
  source 1..155: mol_type = other RNA
tttcacaaacaagaaaatgctaatgtgtaggattcgttgtatccgaatttaagcctttggagagttatattaaacgaaagtagctacggcaaaatcggattctatgaagaaggaaataaacaaatttctatatttttatagatttttggcaacgg

SEQ ID NO: 91 - RNA, 155 nt, synthetic construct
  source 1..155: mol_type = other RNA
tttcacaagcaagaaaatgctaatatgtaggattcgttgtatccgaatttaagcctttggagaatcatatcaaacgaaagtagctacggcaaaatcggattcaataaagaaggaaataaacaaatttttatatttttatagatttttggcaacgg

SEQ ID NO: 92 - RNA, 196 nt, synthetic construct
  source 1..196: mol_type = other RNA
tagagaagttctgatatatgtaccgatggctagtcgggaacaggctcttatccttgcattgcaaggagcgtgaagccaacgccttcggagtgttatacaaacgtcagtagcctgtctttcggacggcgagggcgaacacgatgaacaaggaatatttctaagatacatacattgagggttatgttcttagtagcag

SEQ ID NO: 93 - RNA, 200 nt, synthetic construct
  source 1..200: mol_type = other RNA
ttcacgctctcacatttgataaatgtaccgatggctagtcggaaacaggctgttgccttgcctagcaaggaatacagccaacgcctttggagtgtacaagaacttgtgagtagtctcaggcttgcctgggcaaaagcatacacgatgaataaggaatttttctaagacatgcacatttttgtctatgtttttagtagcag

SEQ ID NO: 94 - RNA, 219 nt, synthetic construct
  source 1..219: mol_type = other RNA
aaagaagtccatgcagatatgtaccgattcgttagtcgggaacagacgtgccttcttgcgatggtaaggagtgtcatgtcaaagcctccggagcattatatcaaacgtgagtagcttgtccttcgggatggcaaaaacgaatgcgatgaacgaggaatgaaacgtaaaagtcagtttttcatcaatagactatggctttttataagttttcagtaacgg

SEQ ID NO: 95 - RNA, 172 nt, synthetic construct
  source 1..172: mol_type = other RNA
actgtaaactaaatgtgtaccgatacgttagtcgggaatttaagcctttggagaagtatacaaactatagtagctattctttgaatggtgaaataggctttggtgaattaggaaatagagtttgtagttttttataagtaatttatagaaatttatagattttatgtaacgg

SEQ ID NO: 96 - RNA, 168 nt, synthetic construct
  source 1..168: mol_type = other RNA
cactaaaacgttagtgcaaatatgtacgggtacgttaattcggaatttacgcctatggagagtacaagaacttgtgagtagatagatatttatatcatcaaaagcatactcgttgaagtaggaatgaaacatagaagtttataactttttataagttttcagtaacgg

SEQ ID NO: 97 - DNA, 187 bp, synthetic construct
  source 1..187: mol_type = other DNA
ctaaaacgcaaacgtaagtatgtaccgaaggctattttcgggaatttacgactgcggagtgtacaagaacttgtgagtagatatgatttcggtcaatcaaaaacatacacggtgaagcagtaagtagaactcgtgagagtctacgttatctcgatgtgagtatatttacacatattttgagtggcag

SEQ ID NO: 98 - RNA, 189 nt, synthetic construct
  source 1..189: mol_type = other RNA
ttcactataaagaattttataactatgtacctatcgttactggggaatttaagtctacagagtgttataacaaatgagagtagcttaggcaaaatcagacacgatgaagtagaaagtctaattcgtgagaatagacatggtgtagatatttataaattataatagatttgtacatatttatcgtaacgg

SEQ ID NO: 99 - RNA, 187 nt, synthetic construct
  source 1..187: mol_type = other RNA
tgaacgcaaacgtaagtatgtactgcgggctatcgcaggaatttacgactgtggagtgtacaagaacttgtgagtagcgtattgtttacaatcgccaaagcatacacaatgaagcagtaagaagtatccgcaaggacttcaatttctccatgtgtttgagtatatttaaacatattttgagtggcag

SEQ ID NO: 100 - RNA, 187 nt, synthetic construct; identical to SEQ ID NO: 65
  source 1..187: mol_type = other RNA

SEQ ID NO: 101 - RNA, 138 nt, synthetic construct; identical to SEQ ID NO: 86
  source 1..138: mol_type = other RNA

SEQ ID NO: 102 - RNA, 168 nt, synthetic construct
  source 1..168: mol_type = other RNA
gactgaaacgttaatgcaaatatgtacggatacgttagtccggaatttacgcctatggagagtacaagaacttgtgagtagatagatatttatatcatcaaaagcatactcgttgaagtaggaatgaaacataaaagtttataactttttataagttttcagtaacgg

SEQ ID NO: 103 - RNA, 185 nt, synthetic construct
  source 1..185: mol_type = other RNA
ataaacgcaaacgtaggtatgtaccgaaggctatttcgggaatttacgactatggagtgtacaagaacttgtgagtagatatgatttcggtcaatccaaagcatacacgatgaagtagtaagtagaactcgtgagagtctacgttatctcgatgtgagtatatttacacatattttgagtggcag

SEQ ID NO: 104 - RNA, 195 nt, synthetic construct
  source 1..195: mol_type = other RNA
ataaacgcaaacataagtatgtaccgaaggctatttcgggaattaacgactgtggagtgtacaagaacttgtgagtagcgtattttttatttcttgcaatcgccaaaacatacacaatgaagcagtaagaagaatccgtgaggacttcaatttctcgatgtgtttgtgtatatttaatcacattttgagtggcag

SEQ ID NO: 105 - RNA, 188 nt, synthetic construct
  source 1..188: mol_type = other RNA
tgaaagcaaacgtaagtatgtactgcgggctatcgcaggaatttacgactgtggagtgtacaagaacttgtgagtagcgaattgtttacaatcgccaaagcatacacattgaagcagtaagaagtatccgcaaggacttcaatttctcgatgcgtttgagtatatttcaacacattttgagtggcaga

SEQ ID NO: 106 - RNA, 188 nt, synthetic construct
  source 1..188: mol_type = other RNA
tcaacgcaaacgtaagtatgtactgcgggctatcgcaggaatttacgactgtggagtgtacaagaacttgtgagtagcgtattgtttacaatcgccaaagcatacacaatgaagcagtaagaagtatccgcaaggactttaatttctcgatgtgtttgagaatatttaaacacattttgagtggcaga

SEQ ID NO: 107 - RNA, 187 nt, synthetic construct
  source 1..187: mol_type = other RNA
acaagcaaatgtaagtatgtaccgatggcttagtcgggaatttacgactgtggagtgtacaagaacttgtgagtagtatgtcacttgtgactacaaaagcatacacgaagaagcagtaagtaatgttcgtgagaacttacatatctcgatgtgtttgagtatatttaaacacattttgagtagcaga

SEQ ID NO: 108 - RNA, 186 nt, synthetic construct
  source 1..186: mol_type = other RNA
ataaaacgcaaacgtaggtatgtaccgaaggctgtttcgggaatttacgactatggagtgtacaagaacttgtgagtagatatgatttcggtcaatccaaagcatacacgatgaagtagtaagtagaactcgtgagagtctacgttatctcgatgtgagtatatttaatcacattttgagtggcag

SEQ ID NO: 109 - RNA, 185 nt, synthetic construct
  source 1..185: mol_type = other RNA
ataaacgcaaacgtaggtatgtaccgaaggctatttcgggaatttacgactgtggagtgtacaagaacttgtgagtagatatgatttcggtcaatccaaagcatacacgataaagcagtaagtagaactcgtgagggtctacattatctcgatgtgagtatatttaatcacattttgagtggcag

SEQ ID NO: 110 - RNA, 185 nt, synthetic construct; identical to SEQ ID NO: 85
  source 1..185: mol_type = other RNA

SEQ ID NO: 111 - RNA, 185 nt, synthetic construct
  source 1..185: mol_type = other RNA
ataaacgcaaacgtaggtatgtaccgtgggcttacacgggaattaacgactgtggagtgtacaagaacttgtgagtagatatgattttggtcaatccaaagcatacacgatgaaacagtaagtaaaacttgtgagagtttacattatctcgatgtgagtatatttacacacattttgagtggcag

SEQ ID NO: 112 - RNA, 185 nt, synthetic construct
  source 1..185: mol_type = other RNA
ataaacgcaaacgtaggtatgtaccgtgggcttacacgggaattaacgactgtggagtgtacaagaacttgtgagtagatatgattttggtcaatccaaagcatacacgatgaaacagtaagtaaaacttgtgagagtttacattatctcgatgtgagtatatttacatacattttgagtggcag

SEQ ID NO: 113 - RNA, 186 nt, synthetic construct
  source 1..186: mol_type = other RNA
ggacgcaaatgtaagtatgtaccgaaggctatttcgggaattaacgactgtggagtgtacatggatctacgagtagatatgatttcggtcaatcaaaagtatacacaaggaagcagtaagtaagattcgtgagaacttacattatctcgatgtgataagtatatttaatcacattttgagtggcag

SEQ ID NO: 114 - RNA, 186 nt, synthetic construct; identical to SEQ ID NO: 113
  source 1..186: mol_type = other RNA

SEQ ID NO: 115 - DNA, 1464 bp, synthetic construct
  source 1..1464: mol_type = other DNA
atgaggaaattgctgaagagcttcaagacggaaataaatccaacagaagagcagaaagtcaggattcgtaaaacaataggtacttgcagatttatctataacttctatcttgctcataataaggagcttcatgaaagtggcaaaaagtttatgagcagcagccaattcagagtatggctcaacaatgaatatcttccgagccatccggaatattcctggatcaaggaagcttattcaaaatcggtaacacaggcagtaaatgatggacagaccgcatttacaagattctttaatcacaagagcgcctttccaaagtttaaaaagaaaggcaagtctgatgtgaaaatgtattttgtaaggaataatccaaaagattgcagctgcgaaagacacagaatcaagattccttcacttggctgggtccgtatcaaagagaaaggatatattcccactacaaaagatggatatgtaattaagagcggtcatgtctcaataaaagctgatagatactatgtttcagttcttatagaaatcccagacaaaacgtcagctagtaattccagtgaaggaattggcattgaccttggattaaaggactttgcgattgcttctaatggaaagacttataagaacatcaacaaatctgctaaattaaagaagcttgaaaaaaagcttattcgagaacagagaagtctttctcgaaagtatgaaaatataaagaaaggagggtccactcaaaaaagaaatatacaaaaacaaaggcttaagatacagaaacttcatcatagaatcgacaatattcgtactgattacatcaataagataatcgcagagatagtgaaaaccaagccatcttatataacgattgaagatctaaacatatcagggatgatgaagaacagacatctttcaaaggcagttgcttcgcagaagttttatgaatttaagacaaagcttcaagctaaatgcagggagaacggaatcgaacttcgagttgtagatagatggttcccatcatctaagacttgtcactgctgtaaaagtatcaagaaagatttaaagctttcagatagacttttcagatgtgattgtggatatatcgaagacagagatttcaatgctgcactaaatctgagagatgctacaacttacgaagttgcaagcagccatcaccatcatcaccactaaatgaacgcaaacgtaagtatgtaccgaaggctatttcgggaatttacgactatggagtgcactagaacttgtgagtagatatgatttcggtcaatccaaagcatgcacagtgacatagtaagtaatgttcgtgagaacttacattatctcgatgtgagtatatttaatcacattttgagtggcagctgatggtccatgtctgttaggccggcatggtcccagcctcctcgctggcgccggctgggcaacatgcttcggcatggcgaatgggacccg

SEQ ID NO: 116 - DNA, 20 bp, synthetic construct
  source 1..20: mol_type = other DNA
ctgatggtccatgtctgtta

SEQ ID NO: 117 - DNA, 1463 bp, synthetic construct
  source 1..1463: mol_type = other DNA
atgaggaaattgctgaagagcttcaagacggaaataaatccaacagaagagcagaaagtcaggattcgtaaaacaataggtacttgcagatttatctataacttctatcttgctcataataaggagcttcatgaaagtggcaaaaagtttatgagcagcagccaattcagagtatggctcaacaatgaatatcttccgagccatccggaatattcctggatcaaggaagcttattcaaaatcggtaacacaggcagtaaatgatggacagaccgcatttacaagattctttaatcacaagagcgcctttccaaagtttaaaaagaaaggcaagtctgatgtgaaaatgtattttgtaaggaataatccaaaagattgcagctgcgaaagacacagaatcaagattccttcacttggctgggtccgtatcaaagagaaaggatatattcccactacaaaagatggatatgtaattaagagcggtcatgtctcaataaaagctgatagatactatgtttcagttcttatagaaatcccagacaaaacgtcagctagtaattccagtgaaggaattggcattgaccttggattaaaggactttgcgattgcttctaatggaaagacttataagaacatcaacaaatctgctaaattaaagaagcttgaaaaaaagcttattcgagaacagagaagtctttctcgaaagtatgaaaatataaagaaaggagggtccactcaaaaaagaaatatacaaaaacaaaggcttaagatacagaaacttcatcatagaatcgacaatattcgtactgattacatcaataagataatcgcagagatagtgaaaaccaagccatcttatataacgattgaagatctaaacatatcagggatgatgaagaacagacatctttcaaaggcagttgcttcgcagaagttttatgaatttaagacaaagcttcaagctaaatgcagggagaacggaatcgaacttcgagttgtagatagatggttcccatcatctaagacttgtcactgctgtaaaagtatcaagaaagatttaaagctttcagatagacttttcagatgtgattgtggatatatcgaagacagagatttcaatgctgcactaaatctgagagatgctacaacttacgaagttgcaagcagccatcaccatcatcaccactaaatgaacgcaaacgtaagtatgtaccgaaggctatttcgggaatttacgactatggagtgcactagaacttgtgagtagatatgatttcggtcaatccaaagcatgcacagtgacatagtaagtaatgttcgtgagaacttacattatctcgatgtgagtatatttaatcacattttgagtggcagctgatggtccatgtctgttaggccggcatggtcccagcctcctcgctggcgccggctgggcaacatgcttcggcatggcgaatgggaccc

SEQ ID NO: 118 - DNA, 2754 bp, synthetic construct
  source 1..2754: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcctggacatccgcgagcacagctaaggccannnnnnnctgatggtccatgtctgttactcacaccgaccggcacagcatcaagaagcgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 119 - DNA, 2700 bp, synthetic construct
  source 1..2700: mol_type = other DNA
tgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcctggacatccgcgagcacagctaaggccagggaggagctgatggtccatgtctgttactcacaccgaccggcacagcatcaagaagcgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaa

SEQ ID NO: 120 - DNA, 60 bp, synthetic construct
  source 1..60: mol_type = other DNA
cctggacatccgcgagcacagctaaggccannnnnnnctgatggtccatgtctgttactc

SEQ ID NO: 121 - DNA, 49 bp, synthetic construct
  source 1..49: mol_type = other DNA
cttcttgatgctgtgccggtcggtgtgagtaacagacatggaccatcag

SEQ ID NO: 122 - DNA, 38 bp, synthetic construct
  source 1..38: mol_type = other DNA
ggcacagcatcaagaagcgggtaccgagctcgaattca

SEQ ID NO: 123 - DNA, 43 bp, synthetic construct
  source 1..43: mol_type = other DNA
agctgtgctcgcggatgtccaggcctgcaggcatgcaagcttg

SEQ ID NO: 124 - DNA, 73 bp, synthetic construct
  source 1..73: mol_type = other DNA
ctcctcgctggcgccggctgggcaacatgcttcggcatggcgaatgggacccggaattcccgggtcgactcga

SEQ ID NO: 125 - DNA, 83 bp, synthetic construct
  source 1..83: mol_type = other DNA
ccagccggcgccagcgaggaggctgggaccatgccggcctaacagacatggaccatcagctgccactcaaaatgtgattaaat

SEQ ID NO: 126 - DNA, 27 bp, synthetic construct
  source 1..27: mol_type = other DNA
tgagtgataacactgcggccaacttac

SEQ ID NO: 127 - DNA, 28 bp, synthetic construct
  source 1..28: mol_type = other DNA
ttctgtgactggtgagtactcaaccaag

SEQ ID NO: 128 - DNA, 55 bp, synthetic construct
  source 1..55: mol_type = other DNA
ggccaccattaggagctgatggtccatgtctgttactcacaccgaccggcacagc

SEQ ID NO: 129 - DNA, 55 bp, synthetic construct
  source 1..55: mol_type = other DNA
gctgtgccggtcggtgtgagtaacagacatggaccatcagctcctaatggtggcc

SEQ ID NO: 130 - DNA, 55 bp, synthetic construct
  source 1..55: mol_type = other DNA
ggccaccattttgatctgatggtccatgtctgttactcacaccgaccggcacagc

SEQ ID NO: 131 - DNA, 55 bp, synthetic construct
  source 1..55: mol_type = other DNA
gctgtgccggtcggtgtgagtaacagacatggaccatcagatcaaaatggtggcc

SEQ ID NO: 132 - DNA, 50 bp, synthetic construct
  source 1..50: mol_type = other DNA
cggtatttcacaccgcatatggtgcactctcagtacaatctgctctgatg

SEQ ID NO: 133 - DNA, 20 bp, synthetic construct
  source 1..20: mol_type = other DNA
taacagacatggaccatcag

SEQ ID NO: 134 - DNA, 81 bp, synthetic construct
  source 1..81: mol_type = other DNA
ataaggagctggagaacctggagatcaatctgcggaaggagatcgccaaggccttcaagggcaacgagggctacaagtcct

SEQ ID NO: 135 - DNA, 80 bp, synthetic construct
  source 1..80: mol_type = other DNA
ggacttgtagccctcgttgcccttgaaggccttggcgatctccttccgcagattgatctccaggttctccagctccttat

SEQ ID NO: 136 - DNA, 38 bp, synthetic construct
  source 1..38: mol_type = other DNA
ggagtgagtacggtgtgcataaggagctggagaacctg

SEQ ID NO: 137 - DNA, 42 bp, synthetic construct
  source 1..42: mol_type = other DNA
gagttggatgctggatgggagcggataacaatttcacacagg

SEQ ID NO: 138 - DNA, 3251 bp, synthetic construct
  source 1..3251: mol_type = other DNA
catgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgctggatccttgacagctagctcagtcctaggtataatactagtattgtcaccagactggcgtcgttttagagctagaaatagcaagttaaaataaggctagtccgttatcaacttgaaaaagtggcaccgagtcggtgctttttttgaattctctagagtcgacctgcagaagcttagatctattaccctgttatccctactcgagttcatgtgcagctccataagcaaaaggggatgataagtttatcaccaccggggaggagctgatggtccatgtctgttaactatttgcaacagtgccgttgatcgtgctatgatcgactgatgtcatcagcggtggagtgcaatgtcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagatgccgctcgccagtcgattggctgagctcataagttcctattccgaagttccgcgaacgcgtaaaggatctaggtgaagatcctttttgataatctcatgaccaaaatcccttaacgtgagttttcgttccactgagcgtcagaccccgtagaaaagatcaaaggatcttctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaacgccagcaacgcggcctttttacggttcctggccttttgctggccttttgctca

SEQ ID NO: 139 - DNA, 25 bp, synthetic construct
  source 1..25: mol_type = other DNA
aggagctgatggtccatgtctgtta

SEQ ID NO: 140 - DNA, 25 bp, synthetic construct
  source 1..25: mol_type = other DNA
gagggctgatggtccatgtctgtta

SEQ ID NO: 141 - DNA, 25 bp, synthetic construct
  source 1..25: mol_type = other DNA
aggagaggtagggcaaaccgcccct

SEQ ID NO: 142 - DNA, 25 bp, synthetic construct
  source 1..25: mol_type = other DNA
gagggcaaaggcctcccggaagttc

SEQ ID NO: 143 - DNA, 3480 bp, synthetic construct
  source 1..3480: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctaaaacgcaaacgtaagtatgtaccgaaggctattttcgggaatttacgactatggagtgtacaagaacttgtgagtagatatgatttcggtcaatccaaagcatacacgatgaaatagtaagtaatgttcgtgagaacttacattatctcgatgtgagcatatttaatcacattttgagtggcagctgatggtccatgtctgttaggccggcatggtcccagcctcctcgctggcgccggctgggcaacatgcttcggcatggcgaatgggacttttttgttttagagctagaaatagcaagttaaaataaggctagtccgtttttagcgcgtgcgccaattctgcagacaaatggctctagaggtacccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactataaggaccacgacggagactacaaggatcatgatattgattacaaagacgatgacgataagatggccccaaagaagaagcggaaggtcggtatccacggagtcccagcagccatgtggaaattgctaaagagcttcaagacggaaataaatccgtcagaggaacagaaagtcaagattcataagacgataggaacttgtagattcatttataatttctatcttgctcataacaaagaactttacgataaaggcgaaaagtttatgagtggtaaaagttttagtgtttggctgaacaatgagtatcttccacaaaatccagacaagttatggattaaggaagtcagttcaaaatctgtaaagcactcaattgaaaatggttgtatcgcatttaccagatttttcaaacatcaaagtgcttttccgaatttaaaaaagaaaggcaaatctgatgtaaaaatgtattttgtaaagaataatccaaaagattgtcgttgcgaaagacacagaatcaatattccgtcacttggttgggttcgcatcaaagaaaaaggatatattccaacaactaaagacggatatgtgattaaaagcggcacggtctcaatgaaagctgacagatactatgtttcagttcttgtagaaatttcaaataacaagatagccaataattccaatgcaggaattggcattgaccttggattaaaggattttgccattgtttctaatggtaaaacttataagaacattaacaaatctgcaagattaaagaaacatgaaaaacagcttattcgagaacagagaagtctttctcgaaagtatgaaaatttaaagaaaggagagtccactcaaaaagcaaatatacaaaaacaaaggcttaaggtacagaaacttcatcatagaatggataatattcgtactgattacatcaataaaacaatcgcagagatagtaaaaaccaaaccatcttatattacgattgaagatttgaatgtaaaaggtatgatgaaaaacagacatctttcaaaagctgttgcttcacagaagttttatgaatttagaacaaagcttcaagctaaatgcaatgaaaatggaatcgaacttcgagtagtagacagatggtatccatcatctaaaacctgtcactgctgtggcgctgtcaaaaaagatttaaagctttcagatagaatatttaaatgcagttgcggttatgtcgaagacagagatttcaatgctgcacttaatttgagagatgcgataacttacgaagttgcaaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactcgag

SEQ ID NO: 144 - DNA, 25 bp, synthetic construct
  source 1..25: mol_type = other DNA
aggagaaggccaagtggtcccaggc

SEQ ID NO: 145 - DNA, 25 bp, synthetic construct
  source 1..25: mol_type = other DNA
aggagttgtactctggtctgagact

SEQ ID NO: 146 - DNA, 25 bp, synthetic construct
  source 1..25: mol_type = other DNA
aggaggagggcagaatcatcacgaa

SEQ ID NO: 147 - DNA, 25 bp, synthetic construct
  source 1..25: mol_type = other DNA
aggagtctgatgtcgggttatacac

SEQ ID NO: 148 - DNA, 25 bp, synthetic construct
  source 1..25: mol_type = other DNA
aggagacaaagttcctgacattctc

SEQ ID NO: 149 - DNA, 25 bp, synthetic construct
  source 1..25: mol_type = other DNA
aggagaaggccaggtgacagagggt

SEQ ID NO: 150 - protein, 367 aa, unidentified
MVYKIEISPTQEQKSIIFRTFDATRLVYNYFIEVNKARHENGYPYMGNLEFSKTFNNYMSRLEWVRDKSLKISSKAVKEAMKDADKAYQRFFKGISRYPRFKSRREPRKSYYLCGSIHVKRHAVRLPNLGWVLLKEKNYIPAGSGVVSCRLIYEGGRFYVAILTQDNNSVNNQLSNDGLGVDVGIRFLATDSDKDVYPNINKSSRVKHLKKRLKREQRKLVRRNKKSLRREKTRQIINRIHYKISCICIDHKNKVVSALVKKKPSYITVETLNIKGMLKNRHLSRVIHEQGLHDFMTRLANKARQYGIELRRVSQYFASSKLCSTCGAKNDPKHKTFWVCTSCGTRHDRDINASLNLLNATQYTIDK

SEQ ID NO: 151 - protein, 382 aa, unidentified
MIRGYRIKIFPNEEQKKMIHKSFGCSRFIYNWCIDRISENYNETKKTLSSIELQKEIVILKKQIEYSWLNEVSANMLKQVTIDCSNAYKRWFKLIKQNNNIKGKPRYKSRKSKQNCPTRTDRMTFNGRYVHLEKIGVVKLSKVKIDLDGKLMNARLNFDGIDYWLSFSVEYKDVQLEEKPKAEPIGIDLGLKTLVYCSNGNIYKKPHTKMIDKKIKHLQRRISKIYQPMIDYCKETRTKFSTLKKSNNLTKLEKELRKYQIRKTNILDSNIHRITSDLIKINPEKIVIEDLNVKGMMSNHKLARSIQSSKFYEIRRQLMYKCKNNNIELIVADRFYPSSKTCSSCGKIKDDLKLKDRVYRCPQCGAVIDRDLNAAINLSRIS

SEQ ID NO: 152 - protein, 379 aa, unidentified
MNDVIRSYKIKLYPTKEQEQLMHKHIGCCRYIWNYMINLQCSNYESGNKYMSAFTMINLLKPLKNDGEHKWLCEVSNQSLATVCQDVDKAYQRLFKKQAKKPKFKSKKRSKQAFPVRGTIWFKDNVVTIEKLGKVKFKSDFEFPQGRGNKFTNTRISEINGKWMLSFGMECENQAPELNDYSMGIDLGVKDLAIAECNGQIVFHNINKSKKMRALNKKLKHLQRAVSRKYEANKQGKKYIKTKNIEREEQKVRNIHNRISNIRNNYIHQCTHALISMLPKRVVVEDLNVKGMMKNKYLSRAIQEQCFGEFLRQMQYKCEWNGIEFVMADRFYPSSKTCSCCGVVKTNLKLSDRIYRCESCGLVIDRDYNAAINLSRYVA

SEQ ID NO: 153 - protein, 397 aa, Clostridioides difficile
MIKAIKVRLYPTKKQEVLMFKSAGISRFSYNWGLAFLNKYYEENNKFLQIDKLRKEFTRLRNNNEFSWLKEVSSEIPQQALKDLGESFKKFFKKESLYPRFKKKGKCEVSFFHLNNKFVVKENYIKLEKIGYVKMKDEGRLPIGNYKKDKIKVLNPRVKFNGKYWYLSLAMEVEEKTNIKLTNESVGVDLGIKDLAIVSNIDEPFKNINKSKEVKRLNKKLRKLQRQVSRKYDMLKSGNTFQKGEKLVKTKNIIKVEKQIKLVYKKLSDIRLNHIHQTTNTIVKTKPCRIVVEDLNVRGLLKNKHLSKSISEQCFNKFINILEYKCKWNKIEFVKADRFYPSSKMCSCCGTIKKDLKLKDRTYVCSNEKCNLVIDRDKNASINLANYKSNYKCGYLN

SEQ ID NO: 154 - protein, 331 aa, Desulfofarcimen acetoxidans
MYAGTCVTSIFKLIKKGISKAYLLCLQMILTNGLTMSILNSKDLSGLRVYHLKHVRSLLSMRRGLLKDSLKNWVNSIPTIGWMRLKEFGYIPYGVTVKSGTVSYKAGRYYVSVIVDIQLVGVEKKCSEGIGVDLGVKVLATVSSGIKFKNINKTSKVKKLERRLKREQRKLSRKYHMRKQRGGTATALRCANIDKQRLKVQKLHQRLTNIQTDYVNKTVNELVKTKPEYITVENLNVSGMMKNRHLSKAIAQQKFYEFNIKLKSKTQWHNIELRVVDRFYPSSKICSCCGHIKWDLKLSDRIYICDNCGFVEDRDVNASINLRNAKQYKIS

SEQ ID NO: 155 - protein, 378 aa, Clostridium tetani
MIKALNIRLYPTQEQAALMYRHIGCMRFVYNWALARQIDSYKHSGKKLSIAELGKELTVLKNTEGYEWLYEVSNATLKEGIRDLDKAYGNFFKGRGFPKFKSKKKSEPKFYSRYEKAYFKDNLVNLEKIGKVKCKVDYDIDLTTIKKFKNPRVKFNGRVWVLSVGIEIPAKKVKLNDFSLGVDLGIKQLAITNIDNLDTLNINKTNKVKKLNKKLKKLQRQCSKKYIINKKGGSYQKTKNIGKLELKIKKLHSKLKNIRLNHTHQTTSKMVKAKPYRVVMEDLKVSNMIKNKHLSKAISEQGFNIFINQMKYKCQKYGIEFIQVSTFYPSSKTCSHCGTIKKDLKLSDRTYKCDCGFTCDRDKNASYNLANYGLEISI

SEQ ID NO: 156 - protein, 374 aa, Gracilibacillus timonensis
MMKAYKIKLNPNNKQRTLLERSAGVSRWAYNWALEQKKKRYEETGKTVSQADLRKRLTKLKQTDDYQWLFQFSNNITKQAIKDSDEAFKRFFKGQARYPRFKSKRKSKWSFYNDNHKVKRSDTHIRLEKIGWIRFAETGYLPIEFKPLSYRVSREGLHWFVSITVEIPDSSDYSTPVNEPIGIDLGIKTLATVSDGSVYVNINKKHEVRQMEKRLRRLQRQISRKYKMNKEGSRYRKTCNIIKTEKEIARLQTRLNHIRTNYIHQMTSEIVRTKPSHIVMEDLNVRGMMKNKHLADHIAKCHFREIRRQLTYKANQSGVSLVVADRWYPSSRICCECGVMDKTLKLSDRKMKCECGNRIDRDMNASINLSRLAV

SEQ ID NO: 157 - protein, 412 aa, Mitsuokella multacida
MFRGRKIRLLPTKEQELLFWKSAGTARWAYNYFLAENEAAYRAYLKRGKTGPRRISADDVRKYINNNLKKTTHQWLKEVSNNVMKMAVKKADYAVKKFLKGLAGKPKFKTKHRTKPSFYVNYASLARKNGGFHGERLGFIRTAEPLPRLPKGKRYSDPWISFDGKYWYLGLSYEVSASEAKELSGDTIGIDLGIKTLATCYVSGTKKKMTFQNINRTKKVRRLEKKLRREQRKQSRKLVANTARHKSIQREGKTIGRKPIWKRPLHECLNLKRQRRKIALIHRRLANIRQNHVHQTTATIVKTKPSRVVLENLNVKGLMKNKHLAKAIAGAKFYEFRRQMIYKCEEHGIQVIIADRFYPSSKTCSYCGYLKKNLKLNDRVYVCPSCGKAIDRDLNAAINLANYFQSDTACTA

SEQ ID NO: 158 - protein, 414 aa, Enterococcus mundtii
MSHLKIDKYNKEIEGDKDVSGVQNRNSSNRRTKKIINRTIGVARFVKNFYIYHNQEVYKSGGKFVSGIDFSKWLNNDYIPNHPDKLWIKAVYAKSTKHAIMETEKAFRNFFQGKANFPKFKKKKHQDVSMYFVKNDKKTIIRCERHRIKVPSLNWVKLNEKGYIPKHDDKHSIKSGTISQQANRYYLSVIVEQDCTAQVLNDTPTEGVGIDLGLKDFAILSTGKTYKNINKSGKIRKIEKQLKRQQRKLSRKYESEKKRVNEKQSKRGEATRKNIEKQVLKVQKLHKRLTDIRRDRVNKIVLELVKTKPAYITIEDLNVKGMMKNRHLSKAVREQSFYAFRKKLADKCKVSTIELRLADRFYPSSKLCHECGHKKVALKLSERTYRCENCGYVADRDYNASMNLKDVKEYNVIA

SEQ ID NO: 159 - protein, 391 aa, Anoxybacillus flavithermus
MIRTYKVMLLPNNKQKTKLFQCAGVARWAYNFALAQQQENDKQGGKFLSDGELRKRLTQLKQTKEYSWLNQFSNNITKQAIKDAYQAYKNFFEGRAKFPKFKTKKRTRPSFYQDTSKIKITDTHVKLEKLTPSKKKNRQKFNWIRLAERGRIPHGAHIKYVNPRIVFDGLHWFLTVGVEEAEVKHGTYNEGIGIDLGVKSFATVSNGMTFPNIHHMPQVKKLEKQMKRMQRKISRKYEMNKIQTERGEFRYRKTENIKKLEFLVLKKRRRLKNTQLNYTHHITTTLVKTKPAYVVMEDLNTSGMLKNRKLSKAIQQQTFHEFKRQMTYKCAWHGIELIVADRFYPSSKKCSRCGHVKEKLSLSERTYRCEACGIEMDRDLNASINLKNYAK

SEQ ID NO: 160 - protein, 387 aa, Fusobacterium pseudoperiodonticum
MYYKALKIEIKLTEEQKIQVNKTIGTERFIYNEYIKYNQEQYELGNKFVSANDFSKYINNIYLPNNPDKKWIKDVSSKSVKQAMIYGEKAFKNFFKGLSSFPVFKKKGKNELGAYFVKNNKTDFEFYRHKIKIPTLKFVRVKEYGYIPKNANIKSGTITKIADRYFLSLIIEIEDTVKATNTNSKGLGVDLGIKDTAICSNGKVFKNINKTKKVKKLKKKLKREQRKMSRSVEYSKSKKIKLKECKNFNKKKLKVQKLFYRLNCIRDDYNNKIVDEITRAKLKYIAIEDLKVSNMMKNKHLSKAIQEQNFYAIRTKLINKCKERNIELRVVNTFYPSSKTCSCCGSIKKNLKLNDRIYKCCNCGLEIDRDYNASINLEKAKIYKVIA

SEQ ID NO: 161 - protein, 407 aa, unidentified
MEFEEQPIILFKGGLKILKSYKTEIDPTEEQTKKIKKTIGTCRFIYNFYLAKNKEIYEKSNQSMSAFSFMKWLNNEYLKENPDKLWIKEVSTVSIQKATQNATDAFRRFFKGQNRFPKFKKKNKQDVKMYFCRQGKNDSIKCERHRIKIPTLGWVKLKEKGYIPTNPNTHIIRKGTVSYKAGRYYVSVLVEEMQPMKERLNDFGIGIDLGVKEFATLSNGTIYQNINKTNRVKDLEKKLNRERRKWSRKYKSYLRRKELDENCTKENLLKQQIKMQKVFHQLDNARTDYVNKCISEIVKTKPSYITIEDLDVMRMMKNKHLSMEVQKQKFFEFRTKLADKCREYGIELRVVDRWYPSSKTCHNCGCIKHNLKLSERVYKCPECGFKIDRDLNAALNLRDSKIYKIIP

SEQ ID NO: 162 - protein, 397 aa, Lachnoclostridium phytofermentans
MKRLQRAYKVELKPTNQQIQKINQCLGVCRWLYNAYLAKNQSLYQDFKEGKLNKKKAFMSANDFDKYINNDIKVLDDCKWINDCGSKARKKAICNAEFAYKKFFKGEAQFPRFKKKGKSKVGLYFPKNNKTDFKIERHKINIPTLKWVRLKEFGYIKANANVISGTVTEQAGKYYISIVCEVEEVKNSLPKTEPLGLDLGIKDFAIFSNEIVKKNINKTSRVKKLEKKLKREQVRLSRKYECLKKQNKIRKGETTRQNIRKQIVKVQRLQQRLANIRTDYINKCVSEIVGQKPSSITIEDLNVKGMMKNRHLSKAVAGQKFYEFRIKLTNKCKQNDIELRIVDRWYPSSKLCSCCGNVKKDLKLSDRIYVCKECGLVIDRDYQASINLREAKTYMIA

SEQ ID NO: 163 - protein, 396 aa, Petrotoga mobilis
MMRLLKSYKTEINPTLEQEQQIKKTLGVCRFIYNFYIAHNKEIYEKERRFVSGMEFSVWLNNEFIPNSQEFNWIKDVSSKAVKQSIMNGERAFKKFFKGKSKFPRFKKKKNNDVKAYFPRNNKTDWTIERHRVKIPTLGFVRLKEKGYIPTNAKVGSGSVSFEAGRYYVSVLVEEDSLTAVEKGIKQDNISFSEGIGIDVGIREFVVASNGIKKKNINKSKEIKKLEKKLKREQRRLSKKYENKKKGGTATNSAKNIDKQVLKVQKLHQKLKNKRHDYVNKVVCELVKTKPEYFTIEDLNVKGMMKNRHLSKAIAKQNFHYFKIRLENKCKHLGIQLRIVSRFYPSSKICSHCGNIKKDLRLSDRVYICEECGSKIDRDLNAAINLKNAKEYAIAC

SEQ ID NO: 164 - protein, 404 aa, Lachnospira sp.
MVWSLVKYKLEREVRKMIKTIRVMLVPNNKQKAKLFQYANTARFAYNWALGREKENHENRGKFICEGDLRKEFTQLKKKEEYLWLKKISNNVPKQAIRDACTAYQNFFNGVSKAPRFKSKKHSIPKFYQDTKKIKFTDTHVKVEGFATSKRHNKQKLNWIRLAEHCRIPTDCKHFNPRIKYDGLNWYMTVGIEYKDKESIPSNEGIGIDLGIKDLAICSDKNKYKNINKIQKVKKLEKRKRRLQRCISRRYEKNKNGECYCKTSNIKRKEKELLKVSRKLANIRLNYLHQITTEIIKRKPSFICIEDLNVKGIMKNKHLSKAIQQQCFGEFRRQIEYKSKWNNTKVIIADRFFPSSRLCSCCNKIKKDLNLSDRIYRCECGNVIDRDYQAAINLKNYGEMKISV

SEQ ID NO: 165 - protein, 313 aa, unidentified
MRYYLIGEFAEKIGKAIQTLRNWHKNNTFKPAYVTAAGTRYYSQEQLNNFLGLKIKTKIKDKKALIEKVGWIKINEQIPSDIKYSNPRITYDNKYWYISVGIEVNKKQENLTDISLGIDLAICSNGQVFKNINKTIKVRKLEKRLKQKQRQISRKYEMNKIKKEGGVRCQFIKTKNIEKLENTTKLIHRKLANIRNNYLHQVTTSIVKNKPYRIVIEDLNVSGMMKNKHLSDSVRKQCFYKFRQYLTYKTELYGIKLVIADRFYPSSKTCSQYSSIKKDLKLKDIVYRCPHCGAVIDRDYNAALNLSMYKSNM

SEQ ID NO: 166 - protein, 353 aa, unidentified
MYNYMLDLQKKRYEAGEKHLSAFSMIKLITPLKQQEGYEWLCRVSTTSMQRVCQDLANAYENFFSKRAKFPRFKSRKRSRASYPVRETVYFIDGKTQIEKIGKVVYQTNYDLPQGRGHKFSNPRISNNNGKWILTVGIECESQAPELTDKPMGIDVGVKDLAIVAFGEDQIVFHNINKSKRVRNLSRKKRKVQRTISRKYRTNGSYSKTKGIEKYERILKTIHYKLANIRSNYIHQSTHKLVSMLPKVVCVEDLNVTGMMKNRHLSRAIQEQCLGEFLRQMEYKCAWNGIELVKVDRFYPSSRTCSCCGEIKEDLKLSDRTYVCPHCGLVIDRDYNAALNLMKYAASQTRAAA

SEQ ID NO: 167 - protein, 378 aa, Clostridium argentinense
MINLIKALNIRLYPTKEQIILMYKHIGCMRFVYNWALSKQIENYKINNKKLSVSELAKELTILKNTKGYEWMYEISNATLKESIRDLDKAYKNFFNGSSFPKFKSKKKSNHVFYTRYDRIYFKEDFVNLEKIGKVKCRVDYDIDLATITKFQNPRIHFNGRVWVLSVGIETSVEKAKLNDFSLGIDLGITQLAITNIDNLDTKNINKTNSVKKLNKRLKRLQRQCSRKYEKNKEGGSYQKTKNIVRLEKKIKKLHSKLKNIRLNHIHQSTSKMVKAKPFRVVMEDLKVSNMMKNKHLSKAIAEQGFNKFINQMKYKCEKYGIEFIQVPILYPSSKTCSHCGYIKKDLKLSDRIYKCSCGFSCDRDKNASYNLANYGLV

SEQ ID NO: 168 - protein, 376 aa, Acetoanaerobium sticklandii
MIKSIKIRLKPTKEQEILMFKSVGVARFAYNWGLAKWEEMYKKGLKPSKAKIRKEFNNSVKKSDDFKWLYEVSGQITSQAFTDLEYAYKNFFNKTSDKPKFKTKKKSKKSFYVRYDALKFKDAKVNIEKIGKVKYSSNYNIPQLDNYVNPRCHFDGKYWYLTFGFEHSENKAKLDYNLSIGIDLGIKELAYVSCFNKPIKNINKSVKVKKLKKRLIRLQKQVSRKYEANKSGDRYNKTSNIIKLEKQIKLLHRKLNNIRNNHLHQTTKMIVKLNPYRIVMEDLNVTGMMKNKHLSKAIAEQKFYEFIRQIKYKCEFNNIEFLQVDRFYPSSKTCSSCSGIKQDLKLKDRIYHCDKCGLTMDRDKNASINLANYKIV

SEQ ID NO: 169 - protein, 409 aa, Mitsuokella jalaludinii
MLRGKKIRLLPTPEQEILFWKSAGTSRWAYNYFLARNEEQYKTYLANGRKGKKSISEGVIRKEINNQLKPITHQWLKEVGSNVMKQAVKEAGRAMKKFLSGAAGRPKFKTKRRTTPRFYVNYESLTRKNGGFHGEKIGFVRTAEPLPKIPKGCHYSNPWISFDGKYWYIGFGYEVPDRLNVELTGDSIGIDLGVKELAVCYAFGERKKTVFKNINKTKEVHRLEKKLRREQRRQSRKFTANIDHYETVQRNEKKTGRRPIWKRPIRECRNIERQRQRIILLHRRLKNIRENHIHQTTAAIVKTKPSRIVMEDLNVKGMMKNRYLAKEIASEKFYEFRRQMEYKCEPYGIQLVFADRFYPSSKTCSHCGHIKKDLKMNNRTYICPACGHVIDRDLNAAINLAKYFKSEIA

SEQ ID NO: 170 - protein, 409 aa, Mitsuokella multacida
MIVGKKIRLHPTSEQETLFWKSAGTARWAYNYFLIRQEEVYQEYLVNGQKGRRGISEGTVRKEINRVLKPTTHAWLKDIGSNVMKQAVKTADKAWKKYLKGLAWKPKFKSKKMTKPNFYVNYESLSRKNGGFHGEKIGFVRTAEPLPKLPHKKHYLNPWISFDGKYWYLGFSYEVEAHRESLTGDHIGIDLGVKSLAVLYSVDTGQSKVIKNINKSRKVLRLEKKLKRAERKRTRQLKSNVLRYEPIQRDGKRPGRKPVWKRPVPECKNIEKQRRKIKLIYRKLKNIRVNHLHQATTAIVKTKPSRVVLETLNIQWMMKNKHLAKAISKAKLYEFRRQIEYKCQKCGIEVVFADKFFPSSKQCSSCGEIHHELTLSDRMFSCSCGLHIDRDLNAAINLARYTPSKIAHN

SEQ ID NO: 171 - protein, 409 aa, Mitsuokella sp.
MLRGKKIRLLPTPKQEVLFWKSAGTARWAYNYFLGENQAVYKAYLEHGKVGPKRISEGAIRKHINNELKKTTHQWLKEVGSNVMKMAVKEADRALKRFLKGIAGKPRFKTKHKTKPSFYVNYESLVRKNGGFHGEKIGFVRTRETLPKLPKGNHYCNPWISFDGKYWYLSFSYEVPEQETEKLTGDSMGIDLGIKVLATCYASGERKATVFKSVNKTKEVRRLEKKLRREQRKQSRKILANTARYETVQRKGKKPGRKPIWKRPLRECQNIEKGRRKIALIHRRLANIRQNHIHQTTAAIVKTKPSRIVMEDLNVKGMMKNKHLAKGIAGEKFYEFRRQMTYKCENNGIQLVFADRFYPSSKTCSCCGHVKENLKLNDRKYVCQACGTTIDRDLNAAINLANYFQSETA

SEQ ID NO: 172 - protein, 301 aa, Eisenbergiella sp.
MPAQPIKTFSRVYNGTQGLRRKRNQCLNSIKIQFRDTHVKFEGFSFSRKANKQKLNWVKLAEHGRIPTDAKYRNPRVSFDGLNWWISVCVEFPDCRDRSNHDGIGIDLGIKNLAICSDGNTYKNINKSQTVKKLEKCKRRLQRSVSRKYEHNKKGGSYCKTNNIVKKEKLLLKVNHRLANIRKDYLNQTTSEIVNRKPRFICIEDLNVSGMIKNRHLSKAVQNQGFFEFRKQLEYKCNDRGIQLIVADRFYPSSKRCSCCGKIKKDLRLSDRTYKCECGNVIDRDFQASLNLKAYGEQFAS

SEQ ID NO: 173 - protein, 387 aa, Finegoldia magna
MLITYKTEIKPNPEQVIKIKQTMGVCRFIYNFYIVENQKAYKNNMPFITAFNFSKWLNNVFIPNNLNYSWIRDVSSKSVKQSIVCAERAFKRFFKKESKFPRFKKKNKSNVKMYFVRNNKGDCLCERHRIKIPTLGWIKIKEKGYIPTSKDDYVVKSGAISYKAGRYYISCLVDIEEPPKQILKPFGLGIDLGLEKFAVISNGNVYKNINKSKKIIKLEKKLRHEQKCLSRKYEQLKNNKKGESTHKNILKQIAKVQKTHLKLSNIRTDYINKIINELVKTKPAYIVIENLNISGMIKNRYLSKSISNQRFYEFRQKLFNKCNFHGIELRIADRFYPSSKTCHSCGSIKKDLKLSDRIYKCPTCGEIIDRDLNASLNLRDTTSYKIA

SEQ ID NO: 174 - protein, 392 aa, Clostridium aceticum
MKKMIKGIKVMLIPNNKQNTKLFQYAGASRFAYNWGLAKQQENYKNSGKFINDGELRKEFTQLKKQEGYEWLNNISNNVTKQAIKDACNAYKRFFKGESKFPRFKSRKKSAPKFYQDVEKIQFNGTHVKFEGFSTSKKKNKQKLNWVRLAEYNRIPFGENEKYINPRVSFDGLNWWVSVGIEHEDEVKQPTNEGIGIDLGIKDLAICSDIDKPYKNINKTSKVKKIKKKLRRLQRKISKKYEMNKEGSRYKKTCNIIKLENKLKKVHHQLKSIRHNYIHQTTCEIVNREPMFIVLEDLAVSNMMKNKHLAKAIGEQCFYEFYRQIEYKALWNNVKFIIADRFYPSSKMCSVCGAIDKDLKLSDRTYICECGNKIDRDKNASVNLKNFGELAI

SEQ ID NO: 175 - protein, 391 aa, Eubacterium sp.
MLKSYKTEIDPTPEQKQHISRTIGVCRYVYNFYLAHNQEIYEVEKRFVSGMDFSKWLNNEFIPNNPDFHWIKEVSSKSVKQSIMDAERAFKNFFKGKSRFPRFKKKARSDVKMYFVKTDAKVIIKCERHRIKIPTLGWVRLKEKGYIPTNPKTHIIKSGAVSCKAERYYVSVLVEEQEPQKPVLNDFGIGIDLGLKDFAVCSNGKTYKNINKSSQIRKLEKKLKREQRSLSRKYESYKKLNKNMKGVATRQNIQKQKLKVQELHQRLDNIRTDHIKKVVSELAKTKPMWITIEDLNISGMMKNRHLSKAIAQQKLFEFRTKLLAKCNSYGIELRIVDRFYPSSKTCHNCGSVKSDLKLSDRTYHCCECGYAEDRDYNASLNLRDCRTYKIA

SEQ ID NO: 176 - protein, 392 aa, Monoglobales sp.
MLITKKIRLKPTTEQEILFRKSAGVSRWAYNFFLGENKRIYREYIDNGKVGKKSISEGDVRKYINNVLKPTTHSWLKEVGSNVMKQGVKDANLALQRYFKDLSGKPKFKSKHKDRPSFYVNYESLSRKQGGFQGEKIGFVKTSESLPKLNKNEKYSNPRITFDGKYWYLSVGYEAKENTVELTDESLGIDLGVKDLAICSNGKVYKNINKTQRVKNLEKKLKREQRKLSRKIENNIQSYKSNRSPVYKRPLKECQNVQKQNRLIRNIHRKLANIRQNYLHQTTTEIVKTKPSRIVMESLNVSEMMKNKHLSKAIQQQKFYEFKRQIEYKSKLNGIEFIQVDKFYPSSKTCSCCGNIKKDLKLSDRMYKCDVCGLVIDRDYNASINLANYEVI

SEQ ID NO: 177 - protein, 371 aa, Intestinibacter bartlettii
MILAKKVRLCPSELQEEKLWQSVGTARFIYNWALNRQEENYKGGGKFIPDSVLRKEITQLKKTDLSWLNEVSNNVAKQAVKDACDSYKRFFKGLSGKPRFKSRKRSKKSFYHDNVKLKVKDNKLVKIEKIGWIKTNEQLSIGAKYSNPRISYDNKYWYLSVGIEQEEIQEELTDASLGIDLGLKNLAICSDGTVYKNINKTCTVKKIEKKLKRLQRQVSRKYEKNKKGKEYVKTKNIIKLEKQIQQTHRRLANIRNNYLHQTTTSIVKTKPYRVVIEDLAVSNMMKNKHLSDAIRKQGFYEFRRQLEYKCNFRGIKLVVADRFYPSSKICSQCGEIKKDLKLSDRVYKCNCGLVIDRDLNASINLSKYKLA

SEQ ID NO: 178 - protein, 399 aa, unidentified
MNEEDKGDQERMLKSYKTEINPTMEQIHKINKTIGTCRFVYNFYLARNKEVYEKEKKFVSGMEFSKWLNNVYLKENPEYVWIKEVSTKSVKQSIMNSERSFKRFFKGQSGFPKFKKKGKSDVKMYFVKTNSQTIITCERHRIKISTLGWVRLKEKGYIPWDSNTYIIKSGTVSCRAGRYYVSVLVEQPERERLQLQDFGFGIDLGVKNFAVISNGLVKKNINKTNKVKKLEKQLKREQRCLSRKYESLKKNNKKGESSRENIQKQILKVKRLYCRIDNIRTDYINKTIAEIVKTKPSYIAVEDLNVSGMMKNRHLSKAVASQKFYEFRVKLEAKCKEYGIELRIVDRWYPSSKLCHECGCIRKDLKLSDREYVCECGYHADRDYNASLNLRDATSYKVA

SEQ ID NO: 179 - protein, 371 aa, Clostridium sporogenes
MILAKKIRIIPNMEQEQQLWKSVGTARFIYNWTLARQEGNYKNGGKFIKDGDLRKEITIMKQTEEYKWLKEVSNNVAKQAVKDGCNAYKRFFKGFADKPRFKSRKKSRPSFYNDNLKLKVKPKMALIEKVGWIKTAEQVPMNIKYTNPRVSFDGKYWYLSVGVEKENSKVELTGESIGIDVGIKDLAICSNGMTFKNINKTRLVKKLEKRLRRLQRKISKKYELNKEGRKFVKTSNIIKLEKQIRLLHRRLSNIRNNYLHQATTKIVKNKPSRVVMETLNIKGMMKNRHLSKAIAQQGLYEFKRQLQYKCEFYRIEFVEADKWYPSSKICSECGHKKTKLSLSERTYICEECGVVIDRDFNASINLSRYSA

SEQ ID NO: 180 - protein, 379 aa, Clostridium novyi
MKMILLKKVRLYPTGIQEQKLWQSVGTARFIYNWTLNKQQENYKNGGKFIKDSDLRKEITLMKKTDEYKWLSEVSNNVAKQAVKDCCNAYKNFFKGLSDKPRFKSRKKSKPSFYNDNVKLKVKTKLVNIEKVGWIKTKEQIPINVKYTNPRISFDGKYWYITVGVEQEQSTIELTNKSIGIDVGIKDLAICSNGMTFKNINKSKEVKRLKKVLKRKQRKVSRKYDMNKKIEGGENRCQYKKTNNIIKLEKEIRLLHRRLRNIRNNHIHQATNKIVKTKPSRVVMETLNIKGMLKNKHLSKAISEQCLYDFKTKMQYKCKLYGIEFIEADKWYPSSKTCSCCGAIKKDLKLSDRVYKCKCGLVIDRDLNASINLSRYKLA

SEQ ID NO: 181 - protein, 373 aa, Clostridium sporogenes
MILGKKVRLYPTKDHEQKLWQSVGTARFIYNWTLARQEESYKNGGKFISDNDLRKELTILKKTELNWLNEVSNNVAKQAVKDGCEAYKKFFKGLADRPRFKSRRKSKPSFYNDTSKLKVKENSVLIEKVGWINIKKNSIPMNCKYSNPRISFDGKYWFIAVGIEKEKPIVELTTESIGIDVGVKDLAICSNGMTFKNINKARVVKQLEKRLRRLQRRVSRKYLKNKEGSKFVKTSNIIKIEKQIKLLHRKLANIRSNYNHQATNKIVKTKPSRVVMETLNIKGMMKNKYLSKAIAKQCLYEFKKQMQYKCEFNGIKFVEADKWYPSSKTCSECGHIKPKLSLSERTYICEECGCVIDRDYNASINLSRYELVG

SEQ ID NO: 182 - protein, 388 aa, unidentified
MIILLKSFKTEINPTAEQIIKINKTIGVCRFIYNFYIAHNKELYENGEKFISGRRFNVWLNNEYLPNNPDKMWIKDVYTKAVAKSMDNACVAFTRFFMCKSKFPRFKKKNKSDVKMYFVKNNKTDCLSERYRIKVPTLGWVRLKEKGYIPTSKDGFIIRSGTISHKAGRYYISVLVDIEKQDTEMSNNFGIGIDLGLKSLAVCSDGSTYCNINKTYNIRKVEKILKREQRKLSRKVISIKKGESTRKNFVKQRLKVQKLYQRLDNIKTDYINKTISKVVKTKPSYIVIEDLNVSGMMKNRHLSKAVAQQKFFEFKTKLISKCKENNIELRVVDRFYPSSKLCHNCDNVKKDLKLSDRIYKCSECGYVEDRDINASLNLRDAKTYTIIQ

SEQ ID NO: 183 - protein, 372 aa, Bacillus thuringiensis
MMLAKKVRIKPNKGQELQLWKSVGTARWAYNWTLAKQEENYKNGGKFISDVFLRKELTVLKQTEEYAWLYDVSNNIAKQAIKDACEAYKRFFKGLADKPRFKSRRKAKLSFYNDNVKLKVKEKQVLIEKVGWVKTSEKLPVGVKYSNPRVSFDGKYWYLSVGIEQEFTKEKLTAVSLGIDIGIKELAYCSDGQRKNNINKTKMVRQVEKRLRRLQRQVSCKYEMNKEGNRFVKTSNIIKIEKEIRQLHRRLSNIRKNHLHQSTSEIVKTKPFRIVMETLHMKGLMKNRYLAKAIAKQCLYEFKRQIQYKCEKYGIEFVEADTWYPSSKMCSSCGSINKELKLSDRVYKCTCGHEMDRDLNAAINLSRYELAS

SEQ ID NO: 184 - protein, 372 aa, Bacillus thuringiensis
MILVKKVRLKPNREQEMQLWKSTGIARWAYNWALSRQEENYKQGGKFISDGTLRKELTQLKQTEEYAWLYDVSNNVTKQAVKDLCEAYKRFFKGVVDKPRFKSRRHSKPSFYNDNVKLKVMKNMVLIGKVGWVKTSECIPVVNKYSNPRISFDGKYWYLAVGIEQEIIHQELTNASIGIDVGIKELAFCSNGMKFKNINKTKVVKQVEKRLRRLQRQVSRKYEINKEGSCFVKTSNIIKLEKKIQHLHRRLTNIRDNHIHQVTCAIVKTKPCRVVMETLNIKGMMKNKHLSKAIAQQKLYDFKMKMKYKCEKYGIEFVEADKWFPSSKQCSNCGEVNKHLTLSDRTYHCKCGHHIDRDLNASINLSRYQLVN

SEQ ID NO: 185 - protein, 397 aa, Anaerostipes hadrus
MMVVKKMIKTIRVMLIPNNKQNTKLFRYANTARFAYNWALGREKENYKNGGKFLSDSDLRKEFTQLKKTEEYSWLKEVSNNVTKQAIKDACHAYKRFFKGCSKFPKFKSRKFSIPSFYQDNVKIQFSDTHVKIEGFAASKKKNKQKINWIRLAEKNRIPTDCNYSNPPIRYDGINWWITVGIEYEDSVTVPSNDGIGIDLGIKDLAICSDGNKYKNINKTKKVKKLEKQKRRLQRSISRSYENNKQGKEYCKTKNVIKKEKLLLILNHRLTNIRHDHLHHITSEIVKREPSFICIEDLNVKGMMKNRHLSKAVQQQGFYEFRRQMEYKSKWNNIQAIIADQFFPSSKLCSCCGKIKKDLKLSERIYKCECGNVIDRDLQAALNLKKYGEDVLKRSVA

SEQ ID NO: 186 - protein, 385 aa, Intestinibaculum porci
MYKSFKTEINPTPTQKAKIDKTIGTCRFVYNFYLAHNIELFNAGEDLMTGKMFSVWLNNEYLPNNPEYNWIKDTYSKAIKKSIEDGFTSFKRFFKHESDFPRFKKKGKSNVKMYFVKNNKTDCKCERHRIKIPTLGWIRIKEKGYIPTEKQGWNIKSGTVSYKAGRYYVSVLAEIPDIEYEKTSNEGVGIDLGIKDFSVISNGNVHENINKSHKIRKLEKKLRREQRCFSRKYENLKKEGEPTQKKNIEKQKLKVQKIYHKMDNIRTDYINKTIAEIVKTKPSYITIEDLNVKGMMKNKHLSKAIASQKFCEFRRKLKIKCDNQNIELRIVDKFYPSSKLCHHCDSIKKDLKLSDRVYRCKCGYIEDRDRNAALNLKDAQSYVVA

SEQ ID NO: 187 - protein, 370 aa, Paenibacillus durus
MILTKKVRIRPALEQEQQLWRSVGTARWVYNWTLSRQEENHKHGGKFLSDGDLRKELTQLKHTEEYAWLCNVSNNVAKQAVKDACDAYKKMFKGLAKKPKFKSRKKSRPSFYNDTAKLKVKDQVVLLEKVGWMKTVEQLPIGVSYSNPRVTFDGKYWYLSVGINQEQVQQKLTDEVVGIDVGVKELAVCSNGMVFKNINKSAAVRKTEKRLRRLQRKVSRKYEMNKEGSRFVKTSNIVKVEKSIRLLHRRLTNIRTNHIHQATSAVAKTKPRAVVMETLNVSGMMKNRHLSQAIANQKLNEFIRQMKYKCEKRGATFMQADRWFPSSKCCSGCGQVKTHLKLSDRIYECDCGLKIDRDLNAAINLAQLAV

SEQ ID NO: 188 - protein, 390 aa, Clostridium sp.
MKKLKKAYKTEIKPTKEQIIKIRRTIGVSRFIYNFYIAHNKEVYEKEKKFVSGMDFSRWLNNEYIPNNQDKVWIKKVSSKAVKQAIMNGEKAFKKFFKGEAGFPKFKKKKNQDIKAYFPKNNETDWTIERHRIKIPTLGWIGLKEYSYIPANGRITSGTVSQKADRYYVSVLVDEDIKINNKRCSEGIGVDLGLKDFAICSNGITKKNINKTKTVKKAEKKLKREQRKLSRKYESLKLRNKKEKGEATRQNIQKQIVKVQKLHQRLTNIRTDYINKTVSELIKQKPRFITIEDLNVRGMMKNRHLAKAVAQQKFYEFRIKLISKSKQDGIEVRIVDRFYPSSKTCSCCGRIKKDLKLSDRVYKCDNCNTSIDRDLNASINLANAKEYKIA

SEQ ID NO: 189 - protein, 433 aa, Selenomonas ruminantium
MIRSVRIRLLPNNKQRTKLFQFAGASRFAYNWALDKQMENFREGRKLQSDYDLRKEFTVLRNSEEYLWLLGISNNVTKQAIKDLCIAYKNFFHKQRQKGYVKYSPKKLAHFARIARTPTIYDMNGHPKFRSKKNGDFRFYQDNVKIQFTATHVKLENIAGSRKKNRQRLNWIRLAEKGRIPTKGRYTQPRITFDGENWWLSVGIRTSYTLKPMRGLNFSRKQPGRFCTEGVGIDLGIKDLAIISDSTVVKNINKSHTIRNIKKKRRRLQRQVSRKYQMNKKGERYCKTSNLIKSEKRLLRINHRLADIRANHVHQATRIIINRKPRFICLEDLNVQGMMKNKHLSEKVQEQNFYEFRRQIEYKAHWARIPVVIADRWFPSSKTCVECGYINKDLKLSDRTYVCPVCGNVIDRDFQASLNLKRYGETELSKSAS

SEQ ID NO: 190 - protein, 393 aa, Blautia wexlerae
MMIKTIRVMLVPNNKQKTKLFQYANTARFAYNWALGREQENYKNGGKFISDGDLRKEFTQLKKKEEYAWLNYVSNNVTKQAIKDACEAYRDFFKGYTKFPRFKSKKHSVPKFYQDNVKIQFTDTHVKVEGFATSRKKNKQKLNWIRLAEHGRIPTEKVKYMNPRIKYDGLNWYITVGIEYEDSTTLSTNEGIGIDLGIKDLTICSDGNTYKNINKTHKIKKLEKRKRRLQRSISRRYEKNKKGGSYCKTSNIIKSEKELLKLNHRLTNIRQNYLHQTTTEIVKTRPSFIVLEDLNVKGMMKNKHLSKSVQQQCFGEFRRQIEYKSAWNNIQVIIADRFFPSSKLCSYCGNIKKDLKLSDRIYRCECGNVIDRDYQAALNLKKYGEMALEQSVA

SEQ ID NO: 191 - protein, 370 aa, Brevibacillus brevis
MILAKKVRLKPTPEQERQLWKSVGTARWAYNWTLAKQEENYRNGGKFLLDGDLRKELTQLKQSEEYSWLYEVSNNITKQAVKDACDAYKKFFKKLAEKPTFKSRRRSKPSFYNDNVKLKVKQNHVLIEKVGWVTTSEQLPTDVNYTMPRISFDGKYWYLSVGVEQEFIQPELSGESLGIDVGVKELAVCSNGMTFKNINKTKVMKKAEKRLRRLQRKVSRKYEMNKEGSRFVKTCNIVKVEKHIRLLHRRLTNIRTNHSHQATSAIVKTKPSTVVMETLNIKGMMRNRHLSKAIAQQKLYEFKRQLQYKCEKYGIRFMEADKWYPSSKTCSDCGEVKKDLKISDRIFKCVCGLVLDRDVNASINLAKLAS

SEQ ID NO: 192 - RNA, 256 nt, synthetic construct
  source 1..256: mol_type = other RNA
taaaaccatagttataaggacttcacatgacctaaaacattatagtacaatgaataatgagaccaattaacccgttatgtctccaaaaactaccgttggttctaccgtgggttggcaggtaagttacgcacgtggactgttacatcaaaccgtagtagacactccttgcagtttcgagacgggacaggaagaagcgtgaaaaagctcaaccagataatgataatatgcgtatggttgagttactggcatggaacag

SEQ ID NO: 193 - RNA, 139 nt, synthetic construct
  source 1..139: mol_type = other RNA
tttatataaaatagtgagactgctctcacgaatttaagcctatggagagttaaaaccacttgagtagttgcgacgaaaaagaattcgaagaagtaggaaatattagaatatacaactttgtataagtttaatatagcag

SEQ ID NO: 194 - RNA, 207 nt, synthetic construct
  source 1..207: mol_type = other RNA
atagagaggctataacctcaaggtgtcgttgcacctttaagctgtggagtgttatataaacttgagtaggattacccaaaagaggacgctgagaagcagtaagttagattggctaacacaacggaaaaggaatgatttatatggttaaaatctcgcaggaggaagcttttaagattagaaagaagtttccgggaacgcatataacgg

SEQ ID NO: 195 - RNA, 184 nt, synthetic construct
  source 1..184: mol_type = other RNA
catagaaaatcaaataaaaagattgtgctaatatgtagacattgacgaggtcgaatttaagcctttggactgctacattaaactagagtagaaattgaatttcaaaatagagcaggaggaaaaaggaaataaaactataaaaaatataaagtatttatataaatttatagagtttatacgtcag

SEQ ID NO: 196 - RNA, 153 nt, synthetic construct
  source 1..153: mol_type = other RNA
ttaacaaagttcttgtatatgtaccgatggctagtcgggaatttaagactgtggagtgttacatcaaatgcgagtagctttgtggcaaaagcagacattatgaagcagtaaaactctcttgatatagatttttgtctatatttaaagtagcag

SEQ ID NO: 197 - RNA, 167 nt, synthetic construct
  source 1..167: mol_type = other RNA
aaagatattctaaattgggtaatcgttacaacccattaccttgctaggtaacaaagcctatggagtgttatagcaaacgaaagtagcttaggcaaaatcggatattatgaagtaggaaagaaacaaaaatctttaatttatagatttttataggtttttggtaacgg

SEQ ID NO: 198 - RNA, 170 nt, synthetic construct
  source 1..170: mol_type = other RNA
tcacgaacaagacacggctaataggtaccgtgagttacacgggaatgaacgccctcggagtgttacagcaaacgagagtagtatctgagacagatacaaaatcggacacggtgaacagggaagaaaacaaaaaatctcttttacaggtttttataagtttttcgtaacgg

SEQ ID NO: 199 - RNA, 212 nt, synthetic construct
  source 1..212: mol_type = other RNA
aacttagagaaattctcagacaggtacctatcgctactagggaaccggctttcctaaaaagattgtctaagcctgcggagcgttatatcaaacgtgagtagccatcgagcttgcttgaggccaaaacggacgcgatgaaccaggaagaaaacatcgcaagagttttcgaagtataactctgtatgataatttatacagatttatcgtagcga

SEQ ID NO: 200 - RNA, 177 nt, synthetic construct
  source 1..177: mol_type = other RNA
gcacgcaactttgtatatgtaccgatggcttagtcgggaatcaacgactgtggagaacgagacaaaccgtagtagtgtcgaagtgacgaggcggcgttcggtgaaacagtaattctacctacgtgagtggtagagaaatctcgatataggtatttttgtctatattttgtgtagcag

SEQ ID NO: 201 - RNA, 154 nt, synthetic construct
  source 1..154: mol_type = other RNA
agcacaacatgtacccatccgttagtggggaagtgaagccttcggagcgccaaggaaacgagagtagcttcggcaaaatcgggcgcgatgaacaaggaaggaaacatagacttttataggtttcttataaagtcttgtaagttttctgtaacgg

SEQ ID NO: 202 - RNA, 157 nt, synthetic construct
  source 1..157: mol_type = other RNA
caagttaaaatatctatatgtaccgatggctagtcgggaatttacgactatggagagtacaaggaactatgagtagatactaagtattgaaaatatactcgttgaagtagtaacattctagtgtattaaatatgttttaatacatttatagtagcag

SEQ ID NO: 203 - RNA, 168 nt, synthetic construct
  source 1..168: mol_type = other RNA
ctaatctgtaaatatgtaccgatggtttgtcgggaatttacgactgtggagtatactagaacttgtgagtagcgtgttattcgtaaccgcaaaagcatatacgaagaagcagtaagaggtatctgtaaaaccctcaattctcaatacttatgtattttagagaaacag

SEQ ID NO: 204 - RNA, 160 nt, synthetic construct
  source 1..160: mol_type = other RNA
ccacgttactgtaagtatgtacccaaggctatttggggaatttacgccctcggagtgttatagcaaatgagagtagtgtattataacaatcaccaaatcagacacgttgaacagggaataatctcgatatgggtagatttgtccatattttgagtggcag

SEQ ID NO: 205 - RNA, 149 nt, synthetic construct
  source 1..149: mol_type = other RNA
aattgctttgcatatgtgtaccgatggccagtcgggaattcacgcctgtggagtgtcaaacaaaccgtagtagatatatatcgaggcgggacacgttgaagcaggaaacttctggatataggcatgtttgtctatgttttcagtggcag

SEQ ID NO: 206 - RNA, 172 nt, synthetic construct
  source 1..172: mol_type = other RNA
aacacgaaaaagttaatacagatgtgtacgaatacgttagttcggaatttacgcctatggagtgtacaaggaactgtaagtagtaactaaatttatttagtacaaaagcatacacgatgaaataggaatgaaacataaaagtttataacttgttataagttttcagtaacgg

SEQ ID NO: 207 - RNA, 125 nt, synthetic construct
  source 1..125: mol_type = other RNA
gacacgttgtatccaaatttaagcctttggagaatcatatcaaacgaaagtagctatggcaaaatcggattctatgaagaaggaagtaaacaaatttatatattgttatagattgttggcaacgg

SEQ ID NO: 208 - RNA, 212 nt, synthetic construct
  source 1..212: mol_type = other RNA
gggaatcgttgcacccctgaatgctgtggagtgtcatacaaacccgagtagctaaggccaaaggggacacaatgaagcagtgattggaattaccaaacgcaacggtacaacgtgctggatgtagaggtcatggctaagattctctatcgggaagctcgtggcatctccagcaagacagaacaggcctgcgtcggctgggtgatctgtaacag

SEQ ID NO: 209 - RNA, 188 nt, synthetic construct
  source 1..188: mol_type = other RNA
tcaattaaaagattaactaaattggggaatcgttataacccactaccttattattaggtagcaaagtctatggagtgttatatcaaacgaaagtagcttaggcgaaatcggacattatgaagtaggaaataaacaaagctataaacttatataatctttttatagagtttttagtattttggtaacgg

SEQ ID NO: 210 - RNA, 178 nt, synthetic construct
  source 1..178: mol_type = other RNA
ttttgaaaaattatactatatgtatcctgcgttgtgggagaatttaagtccttggagagttacaccaaactaaagtagagtgagaaatcattcaaaatagaactcgatgaataggaaatgttatttggtaacaaataatgtaagtaaaatttatatatttataagttttatgcaacgg

SEQ ID NO: 211 - RNA, 197 nt, synthetic construct
  source 1..197: mol_type = other RNA
catttcgagaagcacttagatatgtacctatcgctactggggaatttaagcctgtggagcgttatatcaaacgaaagtagctgcctgcttcggcagggcaaaatcgaacgcaatgaaacaggaaggatgtttgtgaaaacattcaaagtgtaaatttgtatagtataaaatagatatttacagatttatcgtagcgg

SEQ ID NO: 212 - RNA, 179 nt, synthetic construct
  source 1..179: mol_type = other RNA
gagcagtttcgatatgtacctatcgctgctggggaatttaagcctgcagagcattatatcaaacgagagtagcttgcaatgcgttgtggcaaaatcggatgcgatgaagcaggaagagtgctcgtgagagcagtcaacgtattgatttatgatatatatcggtaaatcaatcgcagcgg

SEQ ID NO: 213 - RNA, 200 nt, synthetic construct
  source 1..200: mol_type = other RNA
aatacagagaagcactcagatatgtacctatcgctactggggaattcaagcctgtggagcgttacatcaaacgcgagtagccatcgagcttgctcgggggcaaaaacggacgcaatgaaacaggaagaaaatatcgtgagaattttcatcgtataaatttgtgcgaattatattatatttatacagatttatcgtagcgg

SEQ ID NO: 214 - RNA, 168 nt, synthetic construct; identical to SEQ ID NO: 88
  source 1..168: mol_type = other RNA

SEQ ID NO: 215 - RNA, 159 nt, synthetic construct
  source 1..159: mol_type = other RNA
taaaaaaagctatataagatatgtaccgatggctagtcgggaattgacgactgtggagtgtacaagaacttgttagtagcaaaatctttgcaaaagcatacacaatgaagcagtaatttatctcgatgtagacttatttgtctatgttttgagtagcag

SEQ ID NO: 216 - RNA, 163 nt, synthetic construct
  source 1..163: mol_type = other RNA
tcacttaaaagatattgctaatatgtactcatacgttaatgaggaatttatgcctttggagtgttatatcaaacgaaagtagcataggcaaaatcggacacgatgaacaaggaaagaaacacaaaagtttattttataactttttataagttttctgtaacgg

SEQ ID NO: 217 - RNA, 151 nt, synthetic construct
  source 1..151: mol_type = other RNA
acaagctaatgtaggtatgtaccgtaggctatacgggaatttacgcctgtggactatacaagaacttgtgagtagtcttaggacaaaagcatataggttgaagcaggaattttctcaatatgggtgtatttgtccatattttgagtagcag

SEQ ID NO: 218 - RNA, 190 nt, synthetic construct
  source 1..190: mol_type = other RNA
attcacttaaaagaattatataactatgtacctatcgctattagggaatttacgtctatggagtgtcataacaaatgaaagtagctacggcaaaatcagacacgttgaagtagaaagtcttaatcgtgagataagacacagtatagatttatatagattagttgtaatttatatatatttatcatagcgg

SEQ ID NO: 219 - RNA, 168 nt, synthetic construct
  source 1..168: mol_type = other RNA
tatcattaataagataatgctaatatgtaggatgcgttgtatccgaatttacgcccttggagagttatatcaaacgaaagtaggttgttatttataatttccaaaatcgaattcgtagaacagggaattaaacaagatttatagatatttatagatttttggcaacgg

SEQ ID NO: 220 - RNA, 184 nt, synthetic construct
  source 1..184: mol_type = other RNA
acaagcatttgcaggtatgtaccgatggctagtcgggaattgacgactgtggagtgtacaagaacttgtgagtagtatctgtatttactcagggcgaaagcatatacagagaagcagtaagaaatgttcgcaagaactttcaatatctcgatatggatatctttgttcatattttgagtagcag

SEQ ID NO: 221 - RNA, 154 nt, synthetic construct
  source 1..154: mol_type = other RNA
ctacttaaacagttatgtttgaggtgtaggattcgttgtatccgaagttaagccctcggagtgctatacaaaccaaagtagctacggcaaaatggagtacgttgaacagggaattaaacaagctttatagagttttataaggttttggcaacgg

SEQ ID NO: 222 - RNA, 156 nt, synthetic construct
  source 1..156: mol_type = other RNA
tatcacttaaaaagataatgctaatatgtaccattcgttgtatgggaatttaagcccttggactgttatacaaaccaaagtagcctcggcaaaatggaacaggtggaacagggaattaaacaaggtttatagatatttatagatttttggcaacgg

SEQ ID NO: 223 - RNA, 156 nt, synthetic construct
  source 1..156: mol_type = other RNA
tcacttaaaagatactactaatatgtaccatgtgttgtatgggaatttaagccctcggagtgttatagcaaacgaaagtagctttatagcaaaatcggacacgttgaacagggaattaaacaagctttatagagttctataatgttttggcaacgg

SEQ ID NO: 224 - RNA, 184 nt, synthetic construct
  source 1..184: mol_type = other RNA
atatagtgtataagtatgtaccaatggctagttgggaatttacgactgtggagtatagaagaacttgtgagtaatatattcaactttgttgaattatgaaagcatatacgttgaagcagtaagtaaaaatcgtgaggttttacaattctcattatagacattaggtctataatttgagtagcag

SEQ ID NO: 225 - RNA, 155 nt, synthetic construct
  source 1..155: mol_type = other RNA
catctaaaaaagttacggctaatatgtaggattcgttgtatccgaatttacgccctcggagtgttacatcaaacgaaagtagcttctgtgaaatcggacacgtagaacagggaaataaacaaaatttatagatttttataagtttatggcaacgg

SEQ ID NO: 226 - RNA, 153 nt, synthetic construct
  source 1..153: mol_type = other RNA
cactaaaacgttattgctaatatgtaggattcgttgtatccgaatttacgcccttggagtgttatgtcaaacgaaagtagcttcggcaaaatcggacacgtagagtagggaagtaaacagactttataaagttttataaggttttggcaacgg

SEQ ID NO: 227 - RNA, 170 nt, synthetic construct
  source 1..170: mol_type = other RNA
tatattcaagtgattacagatatgtactgatacgttagtcaggaattaacgcctatggagagtacaggaacttgtgagtagtacatgaattgattcgttacaaaagcatactcgttgaagtaggaatgaaacataaaggtttataactttttataagttttcggtaacgg

SEQ ID NO: 228 - RNA, 187 nt, synthetic construct
  source 1..187: mol_type = other RNA
aagatagaaaagaattttaagcatgtaccaaaggctaatttgggaattaacgactgcggagtgtacaagaacttgttattaatgccttattaaagacatgaaagcatacacgttgaaacagtaagaagtatctgtgaagacttcaatttctcaatatgttaagtatattaatatgattgagtggcag

SEQ ID NO: 229 - RNA, 155 nt, synthetic construct
  source 1..155: mol_type = other RNA
catcttaaagcttacgccaatgtgtaggattcgttgcatccgaatttacgcctgtagagtgttataccaaacgtaagtagcttcggcaaaagcgggcacgttgaagcaggaagcaaacaagagttgtgtagacttgtacaatctcttggcaacgg

SEQ ID NO: 230 - RNA, 151 nt, synthetic construct
  source 1..151: mol_type = other RNA
ttaaacaagcgcttatatgtgtaccgaaggctatttcgggaatttacgcctgtggagtgctatacaaactgtagtagcttaggcaaggcagagtacaatgaaacaggaatcttctcagtatggatacatttgaccatattttgagtagcag

SEQ ID NO: 231 - RNA, 186 nt, synthetic construct
  source 1..186: mol_type = other RNA
caccgacaagtcaatgcagatatgtaccgattcgttagtcgggaatttaagcctctggagcgttatatcaaacgtgagtagtctgttcttcgggacggcaaaagcggacgcagtgaatgaggaatggaacgtaaaagttaactttctacggaaggtttatgactttttataagttttcagtaacgg

SEQ ID NO: 232 - RNA, 170 nt, synthetic construct
  source 1..170: mol_type = other RNA
cacattcaagttaatacagatatgtacggattcgttagttcggaatttaagcctatgaagtgtacaagaacttgtgagtagtatctgggtttattcagtacaaaagcatacacgacgaagtaggaatgaaacataaaagtttataactttttataagttttcagtaacgg

SEQ ID NO: 233 - RNA, 155 nt, synthetic construct
  source 1..155: mol_type = other RNA
ccccaataagttaccgctaatctgtaggattcgttgtatccgaatttacgcctgtggagtgtccgatcaaatgcgagtagcttcggccaaagcagacacagagaagcaggaagcaaacaaaagatgtgtagacttttacaatcttttggcaacgg

(19) United States
(12) Patent Application Publication
(10) Pub. No.: US 2026/0209761 A1
(43) Pub. Date: Jul. 23, 2026

(54) RNA-GUIDED ENDONUCLEASE SYSTEM AND USE THEREOF IN GENE EDITING

(71) Applicants: HARBIN INSTITUTE OF TECHNOLOGY, Harbin (CN); WESTLAKE UNIVERSITY, Hangzhou (CN)

(72) Inventors: Zhiwei Huang, Harbin (CN); Fan Zhang, Harbin (CN); Kuan Ren, Harbin (CN); Fengxia Zhou, Harbin (CN)

(21) Appl. No.: 19/144,565

(22) PCT Filed: Jan. 2, 2024
(86) PCT No.: PCT/CN2024/070149

§ 371 Date: Jun. 30, 2025

(30) Foreign Application Priority Data
Jan. 3, 2023 (WO) PCT/CN2023/070108

(51) Int. Cl. C12N 15/113; A61K 38/46; A61K 48/00; A61P 31/12; C12N 7/04; C12N 9/22; C12N 15/10; C12N 15/52; C12N 15/86

(52) U.S. Cl. CPC C12N 15/113; A61K 38/465; A61K 48/005; A61P 31/12; C12N 7/045; C12N 9/22; C12N 15/102; C12N 15/52; C12N 15/86; C12Y 301/21

(57) ABSTRACT

Provided in the present application are an RNA-guided endonuclease system and the use thereof in gene editing. The endonuclease complex includes an RNA-guided endonuclease and RNA, wherein the RNA-guided endonuclease includes an amino acid sequence selected from SEQ ID NOs: 1-57 and SEQ ID NOs: 150-191, guide RNA includes RNA associated with the RNA-guided endonuclease and targeting RNA, the RNA associated with the RNA-guided endonuclease includes the sequence of any one of SEQ ID NOs: 58-114 and SEQ ID NOs: 192-233, and the targeting RNA is at the 3′-end of the RNA associated with the RNA-guided endonuclease and is identical or complementary to the targeting sequence of a genome of interest. The endonuclease complex of the present application can be used for efficient gene cleavage and gene editing.

### CROSS REFERENCE TO RELATED APPLICATIONS

The present application is a national phase entry of International Application No. PCT/CN2024/070149, filed on Jan. 2, 2024 which claims priority International Application No. PCT/CN2023/070108, filed on Jan. 3, 2023, the contents of which are incorporated by reference in their entirety.

### SEQUENCE LISTING

A sequence listing contained in the file named “P24H05944A-US01-seq” which is 273,788 bytes and created on Mar. 29, 2025, is filed electronically herewith and incorporated by reference in its entirety.

### TECHNICAL FIELD

The present disclosure relates to the technical field of gene editing, in particular to an endonuclease complex and an application thereof.

### BACKGROUND OF THE DISCLOSURE

Genes are the most important genetic material in living individuals. They not only determine the phenotype of various lives, but also are highly associated with various diseases. With the progress of science and technology in recent years, the comprehensive development of sequencing technology and the completion of various genome projects, one has been able to analyze and study huge genetic data. What is hoped is not only to understand genes, but also to modify genes by means of technical methods to solve the problems of major genetic diseases and to optimize and improve the genes of animals and plants, etc. Against this background, gene editing technology is developing rapidly and innovating.

Gene editing technology refers to the technology of targeted modification of a gene achieved by changing the biological genome sequence by artificial means. In this technology, breaks in double-stranded DNA are produced at specific sites in the genome by using genetically engineered nucleases. Then, the DNA repair systems of the cell itself are used to repair the breaks in double-stranded DNA, including by means of non-homologous end joining or homologous recombination. Insertion and deletion of random bases may be induced during the DNA repair, leading to mutations in the genome at specific sites. The final functions of genes are changed due to sequence changes of the genome after the DNA repair.

The development of gene editing technology can be traced back to the 20th century. Prior to the 1990s, genetically mutated model organisms were predominantly procured through random mutagenesis. This approach was not only extremely time-consuming but also introduced random mutations, thereby rendering it arduous to achieve the desired gene modifications. After the 1990s, Chandrasegaran et al. developed the first generation of gene editing tools, zinc finger nucleases (abbreviated as ZFNs). In gene editing technology, ZFNs are the first new endonucleases formed by artificially fusing zinc finger proteins with Fok I endonuclease. In recent years, ZFNs have been used for gene editing in crops, fruit flies, zebrafish, mice, human cells, etc. However, zinc finger protein modules, which recognize DNA sequences, operate on the principle that each zinc finger recognizes three consecutive bases. This characteristic makes the design process for targeting extremely time-consuming and labor-intensive. Moreover, zinc finger nuclease technology faces significant limitations in large-scale applications. This is due to issues like non-specific cleavage by Fok I enzyme, a shortcoming that restricts its broader use in various fields. In 2009, the Ulla Bonas team discovered that the transcription activator-like effector (abbreviated as TALE) is closely associated with the base correspondence to DNA. Transcription activator-like effector nucleases (abbreviated as TALENs) were developed by fusing TALE and Fok I. TALENs are similar to ZFNs in principle, but the preparation steps of TALENs are relatively simple and their specificities are higher than those of ZFNs. In 2010, TALEN protein was firstly used in yeast. Since then, TALEN technology has been widely used in crops, mice, zebrafish, human cells and other areas. In 2012, TALEN technology was named one of the top ten scientific breakthroughs by the top scientific journal Science. In the same year, the Clustered Regularly Interspaced Short Palindromic Repeat sequences (CRISPR) and CRISPR associated proteins (Cas) (CRISPR/Cas) system began to attract widespread attention from worldwide researchers. Emmanuelle Charpentier and Jennifer A. Doudna established a system for CRISPR/Cas9 to cut double-stranded DNA in vitro for the first time. As reported by Feng Zhang in the journal Science, CRISPR/Cas9 has been used for gene editing in human cells. The gene editing technology of CRISPR/Cas9 has been applied to the researches in eukaryotes. Thus, the third generation of gene editing technology is unveiled. CRISPR/Cas, like ZFNs and TALENs, can produce breaks in double-stranded DNA at specific sites in genes, and thus can be applied in gene editing. Compared with the other two technologies, CRISPR/Cas technology shows obvious advantages with its simpler operation, lower cost, higher efficiency and stronger specificity. CRISPR/Cas is considered to be a gene editing tool with broad application prospects and is an alternative to the first two gene editing technologies.

The CRISPR/Cas gene editing technology comprises two important components: RNA acting as a guide and a Cas effector protein that acts to cleave DNA. Guided by the RNA, the effector protein recognizes the protospacer adjacent motif (PAM), binds to a specific location on the genome in a targeting manner, and exerts its endonuclease function, thereby causing a break in double-stranded DNA and enabling gene editing. Currently, the most widely used CRISPR/Cas system is the well-studied CRISPR/Cas9 system. In the CRISPR/Cas system, systems involving for example Cas12a also continue to receive attention.

Gene editing technology has extensive applications. It has important applications in research on gene function, plant breeding, modification and improvement of animal genes. But what is of most concern is undoubtedly the treatment of genetic diseases. Since the emergence of gene editing technologies, these technologies have played an important role in the treatment of genetic diseases in animal disease models and even in clinical settings, becoming a key component of the gene therapy field. Currently, gene therapy mainly involves two aspects: one is in vivo gene therapy, which involves delivering therapeutic genes to target organs or the whole body through viruses or materials; the other is ex vivo cell therapy, which uses cells from a patient or a healthy subject as carriers to deliver cells successfully repaired in vitro or cells capable of expressing therapeutic proteins to patients.

In vivo gene therapy methods have been developed for many years. Before the advent of gene editing technology, researchers had already begun using gene overexpression for gene therapy. After the discovery of adeno-associated virus (AAV), AAV-mediated gene therapy achieved success in animal models of various diseases, particularly demonstrating favorable effects in the clinical treatment of genetic diseases such as hemophilia. However, due to the inability of AAV to completely correct pathogenic genes at the genetic level, patients require multiple viral injections to alleviate symptoms, which may trigger systemic immune responses and pose life-threatening risks. The emergence of gene editing technologies, particularly CRISPR/Cas9, has significantly accelerated the progress of gene therapy. Using this technology, long-term disease treatment can be achieved by destroying pathogenic genes or repairing mutant genes in situ. This approach has been successfully applied to treat muscle diseases such as Duchenne muscular dystrophy and liver metabolic disorders such as type I tyrosinemia, hemophilia, and phenylketonuria.

Cell therapy has developed fast in recent years, mainly focusing on hematopoietic stem cells and immune cells. Infusing the in vitro edited and functionally restored cells back into patients can successfully treat anemic diseases and eliminate tumor cells. At present, many studies have been successfully applied to the clinic. Hematopoietic stem cell transplantation is currently the most promising method for treating thalassemia and sickle anemia. The combination of immune cells, especially T-cell, with gene editing technology is a hot spot in the field of tumor treatment currently. Chimeric antigen receptor T-cell (abbreviated as CAR-T) therapy has saved the lives of many cancer patients in clinical practice.

Gene editing technology has greatly promoted the development of gene therapy, but it also faces various problems, mainly focusing on the efficiency of gene therapy and the cellular safety in vivo. The efficiency of gene therapy depends on the efficacy of gene editing, which is constrained by two factors: the delivery vector and the rate of gene integration. How to efficiently deliver gene editing tools to cells or the body has always been challenge for researchers. Taking the current AAV delivery method suitable for clinical treatment as an example, since the maximum packaging capacity of AAV vectors is less than 5 kb, it is currently difficult to integrate both a CRISPR/Cas system and a repair template into a single AAV vector. The CRISPR/Cas system and repair template are mainly delivered separately, which greatly affect the effect of gene therapy. The smaller the gene editing tool system, the more efficient it becomes, making the construction of delivery vectors considerably easier. Currently, mainstream systems such as CRISPR/Cas9 and Cas12a exceed 5 kilobases (kb) in size, posing significant challenges for vector packaging and delivery. Consequently, the development of more compact gene editing tool systems has been a major focus for researchers in the field. Gene integration efficiency refers to the efficiency of the integration of the repair template into the cell. Efficient and targeted integration is beneficial to treat various genetic diseases, but integration efficiency is closely tied to the inherent DNA repair mechanisms in cells. Researchers have been optimizing strategies to enhance DNA damage repair efficiency, but there is still no effective technology to fully address this issue. The safety of gene editing technology is the most important issue. Since the emergence of gene editing technology, its off-target problem has been constantly appearing in the research process. The off-target problem has been solved neither in the ZFN nor in the CRISPR/Cas9 system. In addition to predictable off-target cleavage, deletions of genomic fragments can also occur in vivo, leading to unpredictable impacts on cellular functions and posing safety risks. Furthermore, the introduction of exogenous gene editing tool proteins can affect cells due to the intrinsic properties of these proteins. These challenges have driven researchers to develop more precise and safer gene editing tools.

In the past decade, the CRISPR/Cas system, as the mainstream gene editing technology, has developed a variety of different gene editing systems, including single-base editing technology based on the CRISPR/Cas system, which may induce specific base conversions to modify genes without introducing breaks in double-stranded DNA. Although gene editing technologies are becoming increasingly sophisticated, the challenges they face in practical applications remain unresolved. Researchers continue to explore novel gene editing tools and to develop innovative technologies, aiming to address these issues through the advancement of new gene editing systems. In 2021, Zhang Feng et al. speculated that TnpB of the IS605/IS200 family has RNA-guided nuclease activity. Subsequently, through a series of biochemical experiments, Virginijus et al. confirmed that TnpB is a reprogrammable RNA-guided functional nuclease. TnpB does not have clustered regularly interspaced short palindromic sequences (abbreviated as CRISPR), and it is guided by a new category of reRNAs to cleave human genomic DNA.

Furthermore, in the development of gene editing technology, it is worth noting that whether it is the zinc finger nuclease (ZFN), the transcription activator-like effector nuclease (TALEN) or the CRISPR/Cas system, they are basically optimized by Chinese researchers based on foreign developments.

There is a requirement for new gene editing technologies in this field.

### SUMMARY OF THE INVENTION

In view of the lack of gene editing tools with improved performance in this field, the inventors provide the use of an endonuclease complex (also referred to herein as RAD or IS607 TnpB system) and an endonuclease (also referred to herein as RAD or IS607 TnpB protein) having a smaller molecular weight and enabling a wider range of applications as compared to the products in the gene editing field. The RAD (IS607 TnpB) system of this present disclosure is an RNA-guided endonuclease system.

The novel gene editing system provided by this disclosure, derived from the IS607 transposase system, consists of two coding sequences ORFA and ORFB encoding proteins, upstream and downstream transposition-related motifs. It has been proven that ORFB can be used in the IS605 system for gene editing. Although the names of IS605 and IS607 are similar, the mechanism of transposition between them is different. Similarly, ORFA plays transposase activity in the IS607 system, but there has been no research showing the specific function of ORFB. Thus, whether ORFB in the IS607 system has the characteristics of gene editing function has not been concerned and proved through biochemical experiments by those skilled in the field of gene editing.

In one aspect, the present disclosure provides an endonuclease complex comprising an RNA-guided endonuclease and an RNA.

In one embodiment, the RNA-guided endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191.

In one embodiment, the guide RNA comprises an RNA-guided endonuclease-associated RNA and a targeting RNA. The RNA-guided endonuclease-associated RNA is located at the 5′ end of the guide RNA and the targeting RNA is located at the 3′ end of the guide RNA, both of which may be directly linked or be adjacent to each other.

In one embodiment, the RNA-guided endonuclease-associated RNA comprises the sequence of any one of SEQ ID NOs: 58-114, and SEQ ID NOs: 192-233 or a sequence having at least 30%, 40%, 50%, 60%, 70%, 80%, 85%, 90%, 95%, 96%, 97%, 98%, 99% or 100% identity to the sequence of any one of SEQ ID NOs: 58-114, and SEQ ID NOs: 192-233. The RNA-guided endonuclease may be derived from IS607 TnpB.

In one embodiment, the targeting RNA is at the 3′ end of the RNA-guided endonuclease-associated RNA. In one embodiment, the targeting RNA is at least 70%, 80%, 85%, 90%, 95%, 96%, 97%, 98%, 99%, or 100% identical or complementary to a targeted sequence in a gene of interest, such as the genome.

In one embodiment, the targeting RNA has a length of 12-40 bp. In one embodiment, the targeting RNA has a suitable length, such as 18-40 bp in length, and the first 14, 15, 16, 17 or 18 nucleotides of the targeting RNA are completely complementary to the targeted sequence in the gene of interest, such as the genome.

In one embodiment, the RNA-guided endonuclease protein comprises a RuvC domain. In one embodiment, the RNA-guided endonuclease protein comprises an amino acid selected from the group consisting of Q18, G27, R30, N34, F50, W73, A92, F97, P104, K107, Y117, E145, P150, S173, G192, D194, G196, K198, A201, S204, N211, 1212, N213, R227, Q229, S233, R234, E237, G242, N249, K252, N266, V280, K283, P284, E290, L292, N293, G296, M297, M298, K299, L303, S304, K305, Q310, K322, P338, S339, S340, C343, C346, L353, L355, R358, C362, C364, D369, R370, D371, A374, N377 and L378 at a position corresponding to the amino acid sequence of SEQ ID NO: 6.

In one embodiment, the gene of interest (such as the genome of interest) is a single-stranded or double-stranded DNA. In one embodiment, the gene of interest (such as the genome of interest) comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, for systems numbered 1-57, the gene of interest (such as the genome of interest) comprises a motif selected from the group consisting of AGGAG or GAGGG.

In another aspect, the present disclosure provides a polynucleotide comprising a polynucleotide of an RNA-guided endonuclease described herein and a polynucleotide for an RNA molecule. The polynucleotide for the RNA molecule may be a polynucleotide that transcribes the RNA molecule.

In another aspect, the present disclosure provides a vector comprising a polynucleotide molecule described herein. The vector comprises an expression control sequence, such as a promoter or terminator. In one embodiment, the vector is an expression vector.

In another aspect, the present disclosure provides a viral particle such as an adeno-associated viral particle, comprising an endonuclease complex, a polynucleotide or a vector described herein.

In another aspect, the present disclosure provides a method for specifically cleaving genomic DNA, comprising the step of contacting an endonuclease complex described herein with the genomic DNA.

In one embodiment, the DNA is genetic DNA of a eukaryotic cell, a prokaryotic cell, or a virus, such as the genome of interest.

In one embodiment, the DNA is a single-stranded or double-stranded DNA.

In one embodiment, the method comprises the step of introducing a polynucleotide or vector described herein into a cell.

In one embodiment, the targeted sequence of DNA, such as genomic DNA, comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, for systems numbered 1-57, the gene of interest (such as the genome of interest) comprises a motif selected from the group consisting of AGGAG or GAGGG. In one embodiment, the 3′ end of the motif is DNA that is at least 70%, 80%, 85%, 90%, 95%, 96%, 97%, 98%, 99%, or 100% identical or complementary to the targeting RNA.

In one embodiment, the method is performed in vivo or in vitro.

In one embodiment, the method is performed under conditions selected from the group consisting of 0.5 to 10 mM Mg²⁺, 25 to 250 mM Na⁺, the reaction temperature between 27 and 57° C. and the reaction time between 3 and 60 minutes.

In another aspect, provided is a method for gene editing comprising the step of contacting the endonuclease complex described herein with genomic DNA of a cell for gene editing.

In one embodiment, gene editing results in an insertion and/or a deletion in genomic DNA.

In one embodiment, the gene editing is performed in a mammalian cell to treat or prevent a disease, or to introduce a desired trait. In one embodiment, the gene of interest (such as the genome of interest) of the mammalian cell comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, for systems numbered 1-57, the gene of interest (such as the genome of interest) comprises a motif selected from the group consisting of AGGAG or GAGGG.

In another aspect, the present disclosure provides a kit comprising one or more of following: an endonuclease complex described herein, a viral particle described herein, a polynucleotide described herein, and a vector described herein.

In another aspect, the present disclosure provides a method for inactivating or identifying a virus, comprising contacting the virus with an endonuclease complex or an endonuclease described herein. In one embodiment, the endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN and GGGGN. In one embodiment, the gene of interest (such as the genome of interest) of the virus comprises a motif selected from the group consisting of AGGAG or GAGGG, for example, for systems numbered 1-57.

In another aspect, the present disclosure provides an endonuclease complex or an endonuclease as described herein for use in inactivating or identifying a virus, comprising contacting the virus with an endonuclease complex or an endonuclease as described herein. In one embodiment, the endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, the gene of interest (such as the genome of interest) of the virus comprises a motif selected from the group consisting of AGGAG or GAGGG, for example, for systems numbered 1-57.

In another aspect, the present disclosure provides use of an endonuclease complex or an endonuclease described herein in the manufacture of a medicament for inactivating or identifying a virus. In one embodiment, the endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, the gene of interest (such as the genome of interest) of the virus comprises a motif selected from the group consisting of AGGAG or GAGGG, for example, for systems numbered 1-57.

In another aspect, the present disclosure provides a method for treating a viral infection, comprising administering an endonuclease complex described herein to a patient infected with a virus. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, the gene of interest (such as the genome of interest) of the virus comprises a motif selected from the group consisting of AGGAG or GAGGG, for example, for systems numbered 1-57.

In another aspect, the present disclosure provides an endonuclease complex as described herein for use in treating a viral infection, the use comprising administering an endonuclease complex as described herein to a patient infected with a virus. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, the gene of interest (such as the genome of interest) of the virus comprises a motif selected from the group consisting of AGGAG or GAGGG, for example, for systems numbered 1-57.

In another aspect, the present disclosure provides use of an endonuclease complex as described herein in the manufacture of a medicament for the treatment of a viral infection. In one embodiment, the gene of interest (such as the genome of interest) of the virus comprises a motif selected from the group consisting of NGGAG, NGGNN, AGGAG, GAGGG, GGGGG, NNGGG, NNAGG, AGGNN, NTAAA, NGAGG, NNGGN or GGGGN. In one embodiment, the gene of interest (such as the genome of interest) of the virus comprises a motif selected from the group consisting of AGGAG or GAGGG, for example, for systems numbered 1-57.

In another aspect, the present disclosure provides a viral vector comprising a polynucleotide or vector described herein. In one embodiment, the viral vector is an adeno-associated virus.

In another aspect, the present disclosure provides a gene therapy method for treating a disease comprising using a viral vector or a viral particle or an adeno-associated viral particle described herein.

In another aspect, the present disclosure provides a viral vector or a viral particle or an adeno-associated viral particle described herein for use in gene therapy for the treatment of a disease.

In another aspect, the present disclosure provides the use of a viral vector or a viral particle or an adeno-associated viral particle described herein in the manufacture of a medicament for use in a gene therapy for the treatment of a disease.

In another aspect, the present disclosure provides a cell comprising one or more of the following: an endonuclease complex, a polynucleotide, a vector, or a viral particle described herein. In one embodiment, the cell is a eukaryotic cell or a prokaryotic cell. In one embodiment, the eukaryotic cell is a fungal cell, a plant cell, or an animal cell.

The disclosure offers the following advantageous effects:
1. The applicant independently found and verified the RNA sequences in the endonuclease complexes described herein (i.e., RAD or IS607 TnpB systems) that are responsible for guiding a cleavage, which are discovered for the first time worldwide. Additionally, the functions of the RAD (IS607 TnpB) proteins within the endonuclease complexes are also discovered for the first time worldwide. (The sequence information of each component of the system is presented in Table 2 in detail);
2. The RAD proteins in the RAD (IS607 TnpB) systems are relatively smaller in size. For instance, they are made up of only 387 amino acids, which are less than one-third the size of the Cas9 protein in the currently most widely used CRISPR/Cas9 system. This small size gives the proteins greater advantages when it comes to the delivery of gene therapy vectors.
3. The RAD (IS607 TnpB) systems have an ability to specifically cleave double-stranded DNA, which is an essential prerequisite for their development into a gene editing tool. The systems recognize 5′ terminal motif sequences such as NGGAG, NGGNN, AGGAG, GAGGG, GGGGG, NNGGG, NNAGG, AGGNN, NTAAA, NGAGG, NNGGN, or GGGGN (for systems numbered 1-57, the motifs are AGGAG or GAGGG). Currently, there are no gene editing tools that can recognize this type of motif sequence. As a result, the RAD (IS607 TnpB) systems broaden the range of recognition for gene editing tools. Moreover, they have a higher specificity compared to the most commonly used CRISPR/Cas9 system.
4. The RAD (IS607 TnpB) systems have a non-specific activity for cleaving single-stranded DNA and an activity for trans-cleaving single-stranded DNA. This property allows the RAD systems to cleave and eliminate single-stranded DNA viruses present in nature. They can also be used as a tool for identifying single-stranded DNA viruses.
5. The RAD (IS607 TnpB) systems have successfully induced DNA interference in both prokaryotic cells (Escherichia coli) and eukaryotic cells (human cells). This property indicates that the RAD systems can serve as a new type of gene editing tool, laying the groundwork for their development into an efficient and precise gene editing tool.
6. The RAD (IS607 TnpB) systems from 65 strains belonging to 55 different genera have in vitro DNA cleavage activity. The RAD systems from 16 strains belonging to 11 different genera have intracellular DNA cleavage activity. These diverse RAD systems are representative, demonstrating that the DNA cleavage activity in RAD systems is ubiquitous. This also shows that more RAD systems can be developed as gene editing tools.

### DESCRIPTION OF THE DRAWINGS

FIG. 1 shows the acquisition process of the RAD (IS607 TnpB) systems.

FIG. 2 shows the results of matching RNA sequencing to the locus.

FIG. 3 shows the results after treatment of RAD complexes with DNase and RNase, respectively.

FIG. 4 shows the results of the analysis of transposon-associated motif (abbreviated as TAM) sequences required by RAD systems from different species.

FIG. 5 shows the TAM sequence of FbRAD1 protein and the results of the cleavage of double-stranded DNA.

FIG. 6 shows the results of the cleavage of DNA by FbRAD1 protein with a mutation in the active center of the RAD protein.

FIG. 7 shows a schematic diagram of the predicted secondary structure of RAD-RNA.

FIG. 8 shows the analysis of the alignment of RNA sequences in RAD systems with DNase activity.

FIG. 9 shows the analysis of the alignment of protein sequences in RAD systems with DNase activity.

FIG. 10 shows the results of the analysis of cleavage-occurring positions.

FIG. 11 shows the results of the cleavage of single-stranded DNA.

FIG. 12A shows cleavage results in presence of different motif sequences and mismatched bases.

FIG. 12B shows the results of the conditions of double-stranded DNA cleavage by RAD proteins.

FIG. 13 shows the results of an intracellular DNA interference experiment in E. coli.

FIG. 14 shows the DNA interference by different RAD systems in E. coli (with a TAM sequence of AGGAG).

FIG. 15 shows the DNA interference by different RAD systems in E. coli (with a TAM sequence of GAGGG).

FIG. 16 shows the results of DNA cleavage by RAD systems in human 293F cells.

FIG. 17 shows the results of DNA cleavage by different RAD systems in 293F cells.

FIG. 18 shows a process of the expression and purification of RAD protein nucleic acid complexes and RNA sequencing.

FIG. 19 shows a process of the digestion of a plasmid library by a RAD protein and high-throughput sequencing.

FIG. 20 shows a process of the cleavage of DNA by RAD protein in E. coli and screening.

FIG. 21 shows a process of the DNA editing by RAD protein in 293F cells and detection.

### DETAILED DESCRIPTION OF THE DISCLOSURE

Unless specifically indicated otherwise, all technical and scientific terms used herein have the same meaning as commonly understood by one of ordinary skill in the art. Furthermore, any methods or materials similar or equivalent to those described herein can be used in the practice of the present disclosure. All publications cited herein are incorporated by reference in their entirety.

Unless specifically defined herein, all terms used herein have the same meaning as they would have to those skilled in the art of the present disclosure. For definitions and terminology in the art, practitioners pay particular attention to Sambrook J. et al. (eds.), Molecular Cloning: A Laboratory Manual, 3rd Edition, Cold Spring Harbor Press, Plainsview, New York (2001); Ausubel, F. M., et al. (eds.), Current Protocols in Molecular Biology, John Wiley & Sons, New York (2010); and Coligan, J. E. et al., (eds.), Current Protocols in Immunology, John Wiley & Sons, New York (2010). In addition, definitions of common terms in molecular biology can be found in Benjamin Lewin, Genes IX, 2008, published by Jones and Bartlet (ISBN 0763752223); Kendrew et al. (eds.), The Encyclopedia of Molecular Biology, 1994, published by Blackwell Science Ltd. (ISBN 0632021829); and Robert A. Meyers (ed.), Molecular Biology and Biotechnology: A Comprehensive Desk Reference, 1995 (ISBN 9780471185710), published by VCH Publishers, Inc. In case of conflict, the terminology in the present disclosure shall prevail.

The term “gene editing” refers to a type of genetic engineering in which DNA is inserted, replaced, or removed from a target DNA (such as the genome of a cell) using one or more nucleases. Nucleases produce specific double-strand breaks (DSBs) at desired locations in the genome. Subsequently, using the endogenous machinery of the cell, the induced breaks are then repaired by homology-directed repair (HDR) (such as homologous recombination) or non-homologous end joining (NHEJ). Any suitable nuclease including, but not limited to, a CRISPR-associated protein (Cas) nuclease, a zinc finger nuclease (ZFN), a transcription activator-like effector nuclease (TALEN), a meganuclease, other endo- or exo-nucleases, variants thereof, fragments thereof, and combinations thereof, may be introduced into a cell to induce gene editing of a target DNA sequence. Nuclease-mediated gene editing can be performed by using the RNA with a stem-loop structure in combination with the RAD protein as described herein.

The term “nucleic acid”, “nucleotide” or “polynucleotide” refers to deoxyribonucleic acid (DNA), ribonucleic acid (RNA), and their polymers in single-stranded, double-stranded or multi-stranded form. These terms include, but are not limited to, single-stranded, double-stranded, or multi-stranded DNA or RNA, genomic DNA, cDNA, DNA-RNA hybrids, or polymers comprising purine and/or pyrimidine bases or other natural, chemically modified, biochemically modified, non-natural, synthetic, or derivatized nucleotide bases. The term nucleic acid may be used interchangeably with gene, cDNA, and the mRNA encoded by the gene.

The term “gene” or “nucleotide sequence encoding a polypeptide” refers to a segment of DNA involved in the production of a polypeptide chain. DNA segments may include regions involved in transcription/translation of gene products and regulation of transcription/translation upstream and downstream of coding regions (leader and tail sequences), as well as intermediate sequences (introns) between individual coding segments (exons).

The term “complementary” refers to the ability of one nucleic acid to form hydrogen bonds with another nucleic acid sequence by conventional Watson-Crick or other non-conventional types. Complementarity percentage represents the percentage of residues in a nucleic acid molecule that are capable of forming hydrogen bonds (such as Watson-Crick base pairing) with a second nucleic acid sequence such as 50%, 60%, 70%, 80%, 90%, and 100% complementarity. “Completely complementary” means that all contiguous residues of a nucleic acid sequence will be hydrogen bonded to the same number of contiguous residues in a second nucleic acid sequence.

The term “expression vector” is a recombinantly or synthetically produced nucleic acid construct having a series of specific nucleic acid elements that allows transcription of the specific polynucleotide sequence in a host cell. The expression vector may be part of a plasmid, a viral genome, or a nucleic acid fragment. Typically, an expression vector includes a polynucleotide to be transcribed operably linked to a promoter.

In this disclosure, a RAD (IS607 TnpB) system refers to the RAGATH-18 containing RNA Associated DNase system (RAD), which is an abbreviation for RAGATH18-Associated DNA endonuclease. Usually, a system newly discovered by the inventors is named according to its characteristics. In the present disclosure, a RAD (IS607 TnpB) system is used interchangeably with an endonuclease complex or an RNA-guided endonuclease system. In the disclosure, a RAD (IS607 TnpB) protein is a DNase in the RAD (IS607 TnpB) system and can also be used interchangeably with an endonuclease herein.

A “guide RNA” refers to a polynucleotide comprising: 1) a guide sequence (also referred to as a “targeting RNA”) capable of hybridizing with or being complementary to a target sequence (also referred to herein as a “targeted sequence”); and 2) a scaffold sequence capable of interacting with an endonuclease (referred to herein as “RNA-guided endonuclease-associated RNA sequence”). The guide nucleic acid may be RNA. The guide RNA may be encoded by a DNA sequence on a polynucleotide molecule.

As used herein, the term “RuvC domain” refers to a conserved domain or motif of amino acids having a nuclease activity (such as endonuclease activity). As used herein, a protein having a split RuvC domain refers to a protein having two or more RuvC motifs at sequentially different sites within the sequence, wherein the RuvC motifs interact with each other in a tertiary structure to form a RuvC domain.

The term “subject” includes a human or an animal. For example, an animal subject may be a mammal, a primate (such as a monkey), a livestock animal (such as a horse, a cow, a sheep, a pig, or a goat), a companion animal (such as a dog or a cat), a laboratory test animal (such as a mouse, a rat, a guinea pig, a bird), an animal of veterinary significance, or an animal of economic significance.

The term “sequence identity” or “identity” is used to describe the correlation between two amino acid sequences or between two nucleotide sequences. The sequence identity between two amino acid sequences can be determined as the output of the “longest identity” using the Needleman-Wunsch algorithm (Needleman and Wunsch, 1970, J. Mol. Biol. 48: 443-453), as implemented in the Needle program of EMBOSS software package (EMBOSS: The European Molecular Biology Open Software Suite, Rice et al., 2000, Trends Genet. 16: 276-277, preferably version 6.6. 0 or later). The parameters used are a gap open penalty of 10, a gap extension penalty of 0.5, and an EBLOSUM62 (EMBOSS version of BLOSUM62) substitution matrix. In order to make the Needle program report the longest identity, the nobrief option must be specified on a command line. The output of the “longest identity” marked by Needle is calculated as follows:

(the number of identical residues×100)/(the length of the alignment−the total number of gaps in the alignment)

The sequence identity between two polynucleotide sequences can also be determined as the output of the “longest identity” using the Needleman-Wunsch algorithm (Needleman and Wunsch, 1970, supra), as implemented in the Needle program of EMBOSS software package (EMBOSS: The European Molecular Biology Open Software Suite, Rice et al., 2000, supra), preferably version 6.6. 0 or newer. The parameters used are a gap open penalty of 10, a gap extension penalty of 0.5, and an EDNAFULL (EMBOSS version of NCBI NUC4.4) substitution matrix. In order to make the Needle program report the longest identity, the nobrief option must be specified on a command line. The output of the “longest identity” marked by Needle is calculated as follows:

(the number of identical deoxyribonucleotides×100)/(the length of the alignment−the total number of gaps in the alignment)

Provided herein is an endonuclease complex comprising an RNA-guided endonuclease and an RNA. The RNA-guided endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191. The guide RNA may comprise an RNA-guided endonuclease-associated RNA and a targeting RNA. The RNA-guided endonuclease-associated RNA may comprise the sequence of any one of SEQ ID NOs: 58-114 and SEQ ID NOs: 192-233, or a sequence having at least 30%, 40%, 50%, 60%, 70%, 80%, 85%, 90%, 95%, 96%, 97%, 98%, 99% or 100% identity to the sequence of any one of SEQ ID NOs: 58-114 and SEQ ID NOs: 192-233. The endonuclease system of the present disclosure may comprise: an endonuclease of SEQ ID NO: 1 and an RNA guide sequence comprising SEQ ID NO: 58; an endonuclease of SEQ ID NO: 2 and an RNA guide sequence comprising SEQ ID NO: 59; an endonuclease of SEQ ID NO: 3 and an RNA guide sequence comprising SEQ ID NO: 60; an endonuclease of SEQ ID NO: 4 and an RNA guide sequence comprising SEQ ID NO: 61; an endonuclease of SEQ ID NO: 5 and an RNA guide sequence comprising SEQ ID NO: 62; an endonuclease of SEQ ID NO: 6 and an RNA guide sequence comprising SEQ ID NO: 63; an endonuclease of SEQ ID NO: 7 and an RNA guide sequence comprising SEQ ID NO: 64; etc. In other words, the endonuclease system of the present disclosure may comprise an endonuclease comprising SEQ ID NO: N paired with an RNA guide sequence comprising SEQ ID NO: N+57, wherein the N is an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57. The endonuclease system of the present disclosure may comprise an endonuclease comprising SEQ ID NO: M paired with an RNA guide sequence comprising SEQ ID NO: M+42, wherein the M is an integer of 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, or 191. The endonuclease system of the present disclosure may comprise 99 types of RNA-guided endonucleases paired with RNAs listed herein.

The RNA guide sequence may comprise a RAGATH18-associated RNA sequence or an endonuclease-associated RNA sequence and a targeting RNA sequence. There may be sequence changes in the endonuclease and RNA guide sequence.

In one embodiment, the endonuclease complex comprises an RNA-guided endonuclease of SEQ ID NO: 6 and a guide RNA of SEQ ID NO: 63; an RNA-guided endonuclease of SEQ ID NO: 11 and a guide RNA of SEQ ID NO: 68; an RNA-guided endonuclease of SEQ ID NO: 13 and a guide RNA of SEQ ID NO: 70; an RNA-guided endonuclease of SEQ ID NO: 21 and a guide RNA of SEQ ID NO: 78; an RNA-guided endonuclease of SEQ ID NO: 43 and a guide RNA of SEQ ID NO: 100; an RNA-guided endonuclease of SEQ ID NO: 51 and a guide RNA of SEQ ID NO: 108; an RNA-guided endonuclease of SEQ ID NO: 52 and a guide RNA of SEQ ID NO: 109; an RNA-guided endonuclease of SEQ ID NO: 54 and a guide RNA of SEQ ID NO: 111; or an RNA-guided endonuclease of SEQ ID NO: 55 and a guide RNA of SEQ ID NO: 112.

The guide RNA may exhibit a stem-loop structure. The targeting RNA may be at the 3′ end of the RNA-guided endonuclease-associated RNA. The targeting RNA may be at least 70%, 80%, 85%, 90%, 95%, 96%, 97%, 98%, 99%, or 100% identical or complementary to a targeted sequence in the genome. The targeting RNA has a length of 12-40 bp. For example, the targeting RNA has a length of 13, 14, 15, 16, 17, 18, 19, 20, 21, 22, 23, 24, 25, 26, 27, 28, 29, 30, 31, 32, 33, 34, 35, 36, 37, 38, 39, or 40 bp.

The RNA-guided endonuclease protein may contain a functional RuvC domain. The RNA-guided endonuclease protein may comprise an amino acid selected from the group consisting of Q18, G27, R30, N34, F50, W73, A92, F97, P104, K107, Y117, E145, P150, S173, G192, D194, G196, K198, A201, S204, N211, 1212, N213, R227, Q229, S233, R234, E237, G242, N249, K252, N266, V280, K283, P284, E290, L292, N293, G296, M297, M298, K299, L303, S304, K305, Q310, K322, P338, S339, S340, C343, C346, L353, L355, R358, C362, C364, D369, R370, D371, A374, N377 and L378 at a position corresponding to the amino acid sequence of SEQ ID NO: 6.

The genome of interest may be single-stranded or double-stranded DNA. The genome of interest may comprise a motif selected from the group consisting of NGGAG, NGGNN, AGGAG, GAGGG, GGGGG, NNGGG, NNAGG, AGGNN, NTAAA, NGAGG, NNGGN, or GGGGN.

The present disclosure provides a method for specifically cleaving genomic DNA, comprising the step of contacting an endonuclease complex described herein with the genomic DNA. The genomic DNA may be the genomic DNA of a eukaryotic cell, a prokaryotic cell or a virus. The eukaryotic cell is not particularly limited and includes, but is not limited to, a mammalian (such as human) cell, or a yeast cell. The prokaryotic cell is not particularly limited and includes, but is not limited to, an E. coli cell. The genomic DNA may be a single-stranded or double-stranded DNA. The method may comprise the step of introducing a polynucleotide or vector described herein into a cell. The targeted sequence of genomic DNA may comprise a motif selected from the group consisting of NGGAG, NGGNN, AGGAG, GAGGG, GGGGG, NNGGG, NNAGG, AGGNN, NTAAA, NGAGG, NNGGN, or GGGGN. The 3′ end of the motif may be DNA that is at least 70%, 80%, 85%, 90%, 95%, 96%, 97%, 98%, 99%, or 100% identical or complementary to the targeting RNA. The method may be performed in vivo or in vitro. The method is carried out under conditions selected from the group consisting of 0.5 to 10 mM of a divalent metal ion selected from Mg²⁺, Mn²⁺ and Ca²⁺, 25 to 250 mM Na⁺, a reaction temperature between 27 and 57° C., and a reaction time between 3 and 60 minutes.

The concentration of the divalent metal ion may be 0.5 to 10 mM, for example 1, 2, 2.5, 3, 4, 5, 6, 7, 8, 9, 10 mM. The type of salts providing the divalent metal ion is not particularly limited, and includes, but is not limited to, a chloride salt.

The concentration of the Na⁺ may be 25 to 250 mM, such as 50, 100, 150 or 200 mM. The type of sodium salts is not particularly limited, and include, but are not limited to, chloride salts.

Provided is a method for gene editing comprising the step of contacting the endonuclease complex described herein with genomic DNA of a cell for gene editing. The gene editing may result in an insertion and/or a deletion in genomic DNA. The gene editing may be performed in mammalian cells to treat or prevent a disease, or to introduce a desired trait.

Provided is a kit comprising one or more of the following: an endonuclease complex described herein, a polynucleotide described herein, and a vector described herein. Alternatively, a kit may comprise any of the components used herein.

Provided is a method for inactivating or identifying a virus, comprising contacting the virus with an endonuclease complex or an endonuclease described herein. In one embodiment, the endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191.

The RAD (IS607 TnpB) system of the present disclosure comprises an effector protein of Table 1. However, an effector protein with a similar function can also be used as endonuclease in the system of the present disclosure.

TABLE 1
Source strains and effector proteins of 57 RAD (IS607 TnpB) systems

| Number | Name of strain | Strain ID | Protein ID |
|---|---|---|---|
| 1 | Catenibacterium sp. AM22-6LB | QTUM01000055.1 | RGE94338.1 |
| 2 | Mitsuokella sp. AF33-22 | QRQA01000020.1 | RHM53169.1 |
| 3 | Blautia sp. AF14-40 | QUHK01000008.1 | RHS04595.1 |
| 4 | Ruminococcus sp. AF17-12 | QUGY01000003.1 | RHR70509.1 |
| 5 | Clostridium sp. AF27-5AA | QUFH01000002.1 | RHQ35082.1 |
| 6 | Firmicutes bacterium AM43-11BH | QUII01000032.1 | RHS75625.1 |
| 7 | Lachnospiraceae bacterium AM26-1LB | QUJW01000020.1 | RHU00256.1 |
| 8 | Dorea formicigenerans strain AF19-4AC | QRWH01000001.1 | RGT12103.1 |
| 9 | [Eubacterium] hallii strain AF31-17AC | QRQO01000101.1 | RHN04853.1 |
| 10 | Clostridiaceae bacterium AF18-31LB | QUGS01000002.1 | RHR46218.1 |
| 11 | Roseburia inulinivorans strain AF24-4 | QRUN01000005.1 | RGR69673.1 |
| 12 | Anaerobutyricum hallii strain AM34-3LB | QSID01000021.1 | RHC60416.1 |
| 13 | [Eubacterium] rectale strain AM42-1 | QSGF01000003.1 | RHB06265.1 |
| 14 | Bacillus cereus strain OM04-20 | QSUV01000008.1 | RGN78093.1 |
| 15 | Weizmannia coagulans strain AF24-21 | QRUS01000007.1 | RGR87203.1 |
| 16 | Blautia sp. TF11-31AT | QUKH01000003.1 | RHU47384.1 |
| 17 | Blautia sp. TM10-2 | QUJZ01000004.1 | RHU18041.1 |
| 18 | Blautia sp. OM07-19 | QULA01000010.1 | RHV03091.1 |
| 19 | Butyricicoccus sp. AM28-25 | QUJL01000001.1 | RHT78732.1 |
| 20 | Clostridiaceae bacterium AF18-31LB | QUGS01000010.1 | RHR42937.1 |
| 21 | Clostridiales bacterium AM23-16LB | QWVV01000023.1 | RGD88909.1 |
| 22 | Clostridium perfringens strain AF30-3 | QRQT01000001. | RHN30353.1 |
| 23 | Coprococcus catus strain AM28-39 | QVFD01000036.1 | RGC42877.1 |
| 24 | Coprococcus comes strain AF18-12LB | QRXJ01000026.1 | RGT87174.1 |
| 25 | Coprococcus sp. AM25-15LB | QVGH01000002.1 | RGC76021.1 |
| 26 | Dorea formicigenerans strain AF25-11 | QRUK01000025.1 | RGR57390.1 |
| 27 | Dorea formicigenerans strain AF12-11 | QSAJ01000004.1 | RGW55202.1 |
| 28 | Dorea sp. AF36-15AT | QUDV01000004. | RHP08869.1 |
| 29 | Firmicutes bacterium AM55-24TS | QTVU01000021.1 | RGF95087.1 |
| 30 | Firmicutes bacterium AF16-15 | QTXI01000030.1 | RGH01875.1 |
| 31 | Firmicutes bacterium TM09-10 | QUKC01000001.1 | RHU31723.1 |
| 32 | Lachnospiraceae bacterium AM48-27BH | QUFC01000010.1 | RHQ17086.1 |
| 33 | Megamonas funiformis strain AF12-42 | QSAH01000092.1 | RGW42851.1 |
| 34 | Megamonas rupellensis strain AF29-2 | QRST01000001.1 | RGQ08395.1 |
| 35 | Mitsuokella multacida strain AM25-21AC | QRHE01000009.1 | RHF50970.1 |
| 36 | Mitsuokella multacida strain AM25-21AC | QRHE01000003.1 | RHF52474.1 |
| 37 | Mitsuokella sp. AF33-22 | QRQA01000008.1 | RHM55346.1 |
| 38 | Peptoclostridium sp. AF21-18 | QUGE01000002.1 | RHQ99195. |
| 39 | Ruminococcus sp. AF17-12 | QUGY01000008.1 | RHR68682.1 |
| 40 | Ruminococcus sp. AF17-12 | QUGY01000002.1 | RHR71212.1 |
| 41 | Ruminococcus sp. AF18-29 | QTWT01000012.1 | RGG64615.1 |
| 42 | Firmicutes bacterium TM09-10 | QUKC01000010.1 | RHU26542.1 |
| 43 | Firmicutes bacterium AM41-5BH | QTXW01000019.1 | RGH37793.1 |
| 44 | Monoglobales | GUT_GENOME084711_{—} | GUT_GENOME084711_{—} |
| | | 28 | 28_1717_2925_+ |
| 45 | Eisenbergiella sp | GUT_GENOME112273_{—} | GUT_GENOME112273_{—} |
| | | 39 | 39_14283_15425_+ |
| 46 | Anaerostipes hadrus | SRR7352028_380524_{—} | SRR7352028_380524_{—} |
| | | 48115 | 48115_816_1829_− |
| 47 | Eisenbergiella sp | GUT_GENOME129951_{—} | GUT_GENOME129951_{—} |
| | | 100 | 100_5873_7024_+ |
| 48 | Mageeibacillus indolicus UPII9-5 | NC_013895.2 | WP_005609868.1 |
| 49 | [Eubacterium] rectale ATCC 33656 | NC_012781.1 | WP_012741946.1 |
| 50 | Erysipelotrichaceae bacterium SG0102 | AP019309.1 | BBH26708.1 |
| 51 | Dorea formicigenerans | GUT_GENOME239804_{—} | GUT_GENOME239804_2_{—} |
| | | 2 | 85784_86938_+ |
| 52 | Agathobacter rectalis | GUT_GENOME047820_{—} | GUT_GENOME047820_36_{—} |
| | | 36 | 478_1632_− |
| 54 | Dorea sp | GUT_GENOME001213_4 | GUT_GENOME001213_4_{—} |
| | | | 160075_161238_+ |
| 54 | Hungatella | GUT_GENOME022845_45 | GUT_GENOME022845_45_{—} |
| | | | 3955_5109_+ |
| 55 | Agathobacter faecis | GUT_GENOME112567_131 | GUT_GENOME112567_131_{—} |
| | | | 1855_3009_− |
| 56 | Clostridium sp | GUT_GENOME034484_15 | GUT_GENOME034484_15_{—} |
| | | | 4023_5177_− |
| 57 | Fusicatenibacter saccharivorans | GUT_GENOME040537_31 | GUT_GENOME040537_31_{—} |
| | | | 7690_8853_+ |
| New Number | Strain Name | Strain ID | Protein ID |
| 58 | NA | ERR3307045_63958_{—} | ERR3307045_63958_{—} |
| | | 30277 | 30277_4897_6000_− |
| 59 | Fusobacterium | GUT_GENOME151180_45 | GUT_GENOME151180_45_{—} |
| | | | 9178_10326_+ |
| 60 | NA | ERR2017626_1022590_{—} | ERR2017626_1022590_{—} |
| | | 12863 | 12863_8034_9173_+ |
| 61 | Clostridioides difficile | GUT_GENOWE142162_18 | GUT_GENOWE142162_18_{—} |
| | | | 64466_65659_− |
| 62 | Desulfofarcimen acetoxidans DSM 771 | NC_013216.1 | NC 013216_1_{—} |
| | | | 421908_422903_+ |
| 63 | Clostridium tetani E88 | NC_004557.1 | WP_035125047.1 |
| 64 | Gracilibacillus timonensis | GUT_GENOME096560_11 | GUT_GENOME096560_11_{—} |
| | | | 244135_245259_− |
| 65 | Mitsuokella multacida | GUT_GENOME083533_37 | GUT_GENOME083533_37_{—} |
| | | | 11359_12597_− |
| 66 | Enterococcus mundtii QU 25 | NC_022878.1 | WP_023518930.1 |
| 67 | Anoxybacillus flavithermus strain DSM | NZ_CP020815.1 | WP_012574395.1 |
| | 2641T | | |
| 68 | Fusobacterium pseudoperiodonticum strain | NZ_CP024701.1 | WP_099988772.1 |
| | KCOM 1277 | | |
| 69 | Clostridia | GUT_GENOME228548_94 | GUT_GENOME228548_94_{—} |
| | | | 18418_19641_+ |
| 70 | Lachnoclostridium phytofermentans ISDg | NC_010001.1 | WP_012201916.1 |
| 71 | Petrotoga mobilis SJ95 | NC_010003.1 | WP_081429131.1 |
| 72 | Lachnospira | GUT_GENOME249453_33 | GUT_GENOME249453_33_{—} |
| | | | 14687_15901_− |
| 73 | NA | GUT_GENOME046003_7 | GUT_GENOME046003_7_{—} |
| | | | 13445_14386_− |
| 74 | NA | GUT_GENOME043660_1 | GUT_GENOME043660_1_{—} |
| | | | 131655_132716_− |
| 75 | Clostridium argentinense strain 89G | NZ_CP014176.1 | WP_039636020.1 |
| 76 | Acetoanaerobium sticklandii strain | NC_014614.1 | WP_013360293.1 |
| | DSM 519 | | |
| 77 | Mitsuokella jalaludinii | GUT_GENOME243333_13 | GUT_GENOME243333_13_{—} |
| | | | 9207_10436_+ |
| 78 | Mitsuokella multacida | GUT_GENOME228909_131 | GUT_GENOME228909_131_{—} |
| | | | 318_1547_− |
| 79 | Mitsuokella sp. AF21-1AC AF21-1AC.Scaf4 | QRVW01000004.1 | RGS74140.1 |
| 80 | Eisenbergiella sp | GUT_GENOME029159_118 | GUT_GENOME029159_118_{—} |
| | | | 4969_5874_− |
| 81 | Finegoldia magna ATCC 29328 plasmid | NC_010371.1 | WP_012289953.1 |
| | pFMC | | |
| 82 | Clostridium aceticum strain DSM 1496 | NZ_CP009687.1 | WP_044824965.1 |
| 83 | Eubacterium sp | GUT_GENOME100288_478 | GUT_GENOME100288_478_{—} |
| | | | 819_1994_− |
| 84 | Monoglobales | GUT_GENOME089678_61 | GUT_GENOME089678_61_{—} |
| | | | 13091_14269_− |
| 85 | Intestinibacter bartlettii | GUT_GENOME190643_17 | GUT_GENOME190643_17_{—} |
| | | | 8068_9183_− |
| 86 | NA | ERR1539620_45264_{—} | ERR1539620_45264_{—} |
| | | 57524 | 57524_39821_41020_− |
| 87 | Clostridium sporogenes strain CDC_1632 | NZ_CP013243.1 | WP_072587064.1 |
| 88 | Clostridium novyi strain 150557 | CP029458.1 | AYF55140.1 |
| 89 | Clostridium sporogenes strain AM1195 | CP013700.1 | AUM93701.1 |
| | plasmid pRSJ11_1 | | |
| 90 | NA | SRR5892202_233507_{—} | SRR5892202_233507_{—} |
| | | 16022 | 16022_11195_12361_+ |
| 91 | Bacillus thuringiensis BMB171 | NC_014171.1 | WP_000978855.1 |
| 92 | Bacillus thuringiensis serovar | NC_017209.1 | WP_000604588.1 |
| | chinensis CT-43 plasmid pCT14 | | |
| 93 | Anaerostipes hadrus strain BPB5 | NZ_CP012098.1 | WP_077326813.1 |
| 94 | Erysipelotrichaceae | GUT_GENOME170581_2 | GUT_GENOME170581_2_{—} |
| | | | 14431_15588_− |
| 95 | Paenibacillus durus ATCC 35681 | NZ_CP011114.1 | WP_025697990.1 |
| 96 | Clostridium sp. JN-9 | CP035280.1 | QAT40560.1 |
| 97 | Selenomonas ruminantium subsp. | NC_017078.1 | WP_014431089.1 |
| | lactilytica TAM6421 plasmid pSRC1 | | |
| 98 | Blautia wexlerae | GUT_GENOME088555_122 | GUT_GENOME088555_122_{—} |
| | | | 512_1693_+ |
| 99 | Brevibacillus brevis strain DZQ7 | CP030117.1 | AWX54000.1 |

Exemplary endonucleases of the present disclosure and their associated RNA sequences are listed in Table 2. The endonuclease system of the present disclosure may comprise: an endonuclease of SEQ ID NO: 1 and an RNA guide sequence comprising SEQ ID NO: 58; an endonuclease of SEQ ID NO: 2 and an RNA guide sequence comprising SEQ ID NO: 59; an endonuclease of SEQ ID NO: 3 and an RNA guide sequence comprising SEQ ID NO: 60; an endonuclease of SEQ ID NO: 4 and an RNA guide sequence comprising SEQ ID NO: 61; an endonuclease of SEQ ID NO: 5 and an RNA guide sequence comprising SEQ ID NO: 62; an endonuclease of SEQ ID NO: 6 and an RNA guide sequence comprising SEQ ID NO: 63; an endonuclease of SEQ ID NO: 7 and an RNA guide sequence comprising SEQ ID NO: 64; etc. That is, the endonuclease system of the present disclosure may comprise an endonuclease comprising SEQ ID NO: N and an RNA guide sequence comprising SEQ ID NO: 57+N, wherein the N is an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57. The RNA guide sequence may comprise a RAGATH18-associated RNA sequence or an endonuclease-associated RNA sequence and a targeting RNA sequence. There may be sequence changes in the endonuclease and RNA guide sequence. The endonuclease may comprise an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191. A guide RNA may comprise an RNA-guided endonuclease-associated RNA and a targeting RNA, wherein the RNA-guided endonuclease-associated RNA may comprise the sequence of any one of SEQ ID NOs: 58-114 and SEQ ID NOs: 192-233, or a sequence having at least 30%, 40%, 50%, 60%, 70%, 80%, 85%, 90%, 95%, 96%, 97%, 98%, 99% or 100% identity to the sequence of any one of SEQ ID NOs: 58-114 and SEQ ID NOs: 192-233. The targeting RNA is at the 3′ end of the RNA-guided endonuclease-associated RNA and is at least 70%, 80%, 85%, 90%, 95%, 96%, 97%, 98%, 99% or 100% identical or complementary to the targeted sequence in the gene of interest.

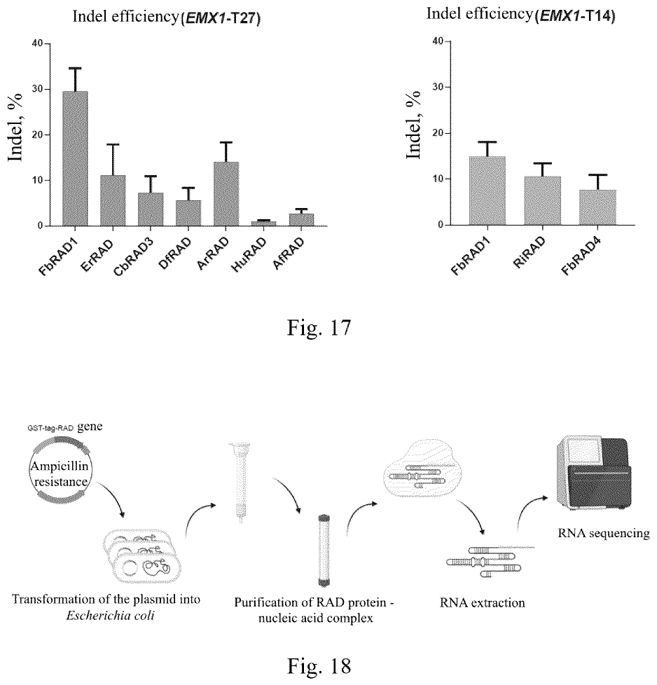

Under the conditions where the TAM sequence is AGGAG or GAGGG, a total of 12 RAD (IS607 TnpB) systems are found to successfully achieve a cleavage in prokaryotes. Those 12 RAD (IS607 TnpB) systems are from 7 different species. They are (6) FbRAD1/ISFba1 (RHS75625.1), (10) CbRAD1/ISCba1 (RHR46218.1), (11) RiRAD/ISRin (RGR69673.1), (13) ErRAD/ISEre1 (RHB06265.1), (15) WcRAD (RGR87203.1), (18) BsRAD/ISBsp4 (RHV03091.1), (20) CbRAD2/ISClsp2 (RHR42937.1), (21) CbRAD3/ISClsp3 (RGD88909.1), (24) CcRAD/ISCco (RGT87174.1), (30) FbRAD2/ISFba2 (RGH01875.1), (42) FbRAD3/ISFba3(RHU26542.1), or (43) FbRAD4/ISFba4 (RGH37793.1), respectively. Different RAD systems are expressed in 293F cells to verify whether they all have an in vivo editing function in mammalian cells. It is found that 9 RAD (IS607 TnpB) systems are able to edit in vivo, and they are from 8 different species. They are: (6) FbRAD1/ISFba1 (RHS75625.1), (11) RiRAD/ISRin (RGR69673.1), (13) ErRAD/ISEre1 (RHB06265.1), (21) CbRAD3/ISClsp3 (RGD88909.1), (43) FbRAD4 (RGH37793.1), (51) DfRAD/ISDfo4 (GUT_GENOME239804_2_85784_86938_+), (52) ArRAD/ISAre (GUT_GENOME047820_36_478_1632_−), (54) HuRAD/ISHun (GUT_GENOME022845_45_3955_5109_+) and (55) AfRAD/ISAfa (GUT_GENOME112567_131_1855_3009_-) (FIG. 17).

TABLE 2
Sequences of the proteins and RNAs of 57 RAD (IS607 TnpB) systems

| SEQ | | | SEQ | |
|---|---|---|---|---|
| ID | Protein | | ID | RAGATH18-associated |
| NO | ID | The amino acid sequence of the protein | NO | RNA sequence |
| 1 | RGE94338.1 | MRKLLKSFKTEINPTEEQKVRIRKTIGTCRFIYNFYLAHNKELHESGK | 58 | AUGAACGCAAACGUAAGUAUGUA |
| | | KFMSSSQFRVWLNNEYLPSHPEYSWIKEAYSKSVTQAVNDGQTAFT | | CCGAAGGCUAUUUCGGGAAUUUA |
| | | RFFNHKSAFPKFKKKGKSDVKMYFVRNNPKDCSCERHRIKIPSLGW | | CGACUAUGGAGUGCACUAGAACU |
| | | VRIKEKGYIPTTKDGYVIKSGHVSIKADRYYVSVLIEIPDKTSASNSSE | | UGUGAGUAGAUAUGAUUUCGGUC |
| | | GIGIDLGLKDFAIASNGKTYKNINKSAKLKKLEKKLIREQRSLSRKYE | | AAUCCAAAGCAUGCACAGUGACA |
| | | NIKKGGSTQKRNIQKQRLKIQKLHHRIDNIRTDYINKIIAEIVKTKPSY | | UAGUAAGUAAUGUUCGUGAGAAC |
| | | ITIEDLNISGMMKNRHLSKAVASQKFYEFKTKLQAKCRENGIELRVVD | | UUACAUUAUCUCGAUGUGAGUAU |
| | | RWFPSSKTCHCCKSIKKDLKLSDRLFRCDCGYIEDRDFNAALNLRDA | | AUUUAAUCACAUUUUGAGUGGCA |
| | | TTYEVA | | G |
| 2 | RHM53169.1 | MKRLLKSFKTELRLTESQTDKVRRSLGICRFLANQYIARNIWLCRLY | 59 | UUCACGUUUUCACGUCUUGAUAC |
| | | QRGVLDEKQPHFITANDFDKYVNHVLKKKFPWIDQCGSKARKKVLV | | AUGUACCGAUGGCUAGUCGGGAA |
| | | NAEQAFRRFFKGPARFPRFKRKNQQNVKLYFPKNNKGDWTIWRHKI | | CAGGCCGUUGCCUUGUGAGGCAA |
| | | QIPTIGRVRLKEFGYLPQGVTIKNGHVSCEAGRYYVSVTAEIEETSRY | | GGAAUACGGCCAACGCCUUUGGA |
| | | NNDLEASYHPQSDGIGVDLGVKSLAIVSDGRIFENINTSSRARRLEKR | | GUGUACAAGAACUUGUGAGUAGU |
| | | LRREQRRLSRKYEIRKKKGGSTATASANIEKKRLSVQKLHQRLTNLR | | CUCAGGCUUGCCUGGACAAAAGC |
| | | RNYENQVIHALVEQKPRFITMEDLNVTGMMKNRHLSKSVAQQRFYS | | AUACACGAUGAAUAAGGAAUUUU |
| | | LREKLRRKAEIIGIEFRIADRFYPSSKTCHACGHVHGGLKLKDRIYVC | | UCUAGGCAUAGAUAAUUUCUAUG |
| | | PVCGYTEDRDLNAALNLRDTKNYRIA | | UUAUUCUAUGUUUUUAGUAGCAG |
| 3 | RHS04595.1 | MRKLQKSFKTEINPTDEQKLKIRRTIGTCRYVYNFYLDQNRKRYEKG | 60 | GAAACGCAGAGAUACAUAUGUAC |
| | | EKFMSGKSFSVWLNNEYLPQNPEYAWIKEVSSKSVKRSLENACTAFS | | CGAAGGCCAUUUCGGGAAUUAAC |
| | | RFFKGQSHFPRYKKKNKSNVKMYFVKNNRTDCFCERHRVNIPTLGW | | GACUGUGGAGUGUACCAGAACUU |
| | | VRIKEKGYLPTTKDGYVIKSGTVSMRAGRYYVSVLVEVPDPPKEENR | | GUGAGUAGAUACAGCUCCGGCUG |
| | | NEGIGIDLGIKSFAVLSDGTVYQNINKSAKIKKLEKQLRREQRSLSRK | | GUCAAAAGCAUACACGUUUGAAA |
| | | YENLKKGVSTQRANIQKQKLKVQRLYQRIDNIRTDYIKKTIAGIVKT | | CAGUAAGUAGGAUUCGUGAGAAC |
| | | KPSFVTIEDLNVKGMMKNRHLSKATASQKFYEFRTRLQGKCAEKGI | | UACAAUAUCUCGAUAUGAGUAUA |
| | | ELRIADRWYPSSRRCHNCGQIRKDLKLSNRVYQCSCGYCEDRDLNA | | UUUGAUCAUAUUUUGAGUGGCAG |
| | | ALNLRDVKTYQSA | | |
| 4 | RHR70509.1 | MRRLLKSFKTEIDPTEEQKIKIHKTIGTCRYIYNFYLFHNKERYDAGE | 61 | UCCCGCAAACGUAAGUAUGUACC |
| | | RFMSGKSFSVWLNNEYLPGHPEYFWIREVSSKSVKNTIENACTAFSR | | GAAGGCUAUUUCGGGAAUUAACG |
| | | FFHHQSSFPRYKKKGRSDVKMYFVKNNPKDCLCERHRIKIPTLGWV | | ACUGUGGAGUGUACUUAGGAUCU |
| | | RLKEKGYIPASKDGFVIRSGAVSMKAGRYYVSALVDIPAPEADGNFT | | GUGAGUAGAGAUAACUCCGGUUA |
| | | DGIGIDLGLKDFAVVSNGTVYRNINKTARIRKLEKQLRRAQRKLSRK | | CUCAAAAGCAUACACAAAGAAAC |
| | | YENLKKGESTQRANICKQKLKVQKLHHRIENIRTDHINKVISEIVKTK | | AGUAAGCGGUAUUCGUGAGAAUC |
| | | PSHITIEDLNISGMMKNRHLSKAVASQKFYEFRTKLKIKCAEYGIELR | | CGCAUUAUCUCGAUGUGAGUAUG |
| | | VVDRWYPSSRICHCCSCIKKDLKLSDRIYRCTCGYIEDRDLNAARNL | | UUUAAUCACAUUUUGAGUGGCAG |
| | | RDAETYEVA | | |
| 5 | RHQ35082.1 | MWKLLKSFKTEINPTEEQKVKIRKTIGTCRYIYNFYLAHNKELHDNG | 62 | UGAAAAGCAAAUGUAAGUAUGUA |
| | | KKFMSGKSFSVWLNNEYLPQNPDKLWIKEVSSKSVKRSIENGCVAFT | | CCGAAGGCUAUUUCGGGAAUUAA |
| | | RFFKHQSAFPNFKKKGKSDVKMYFVKNNPKDCRCERHRINIPSLGW | | CGACUGUGGAGUAUACAAGAACU |
| | | VRIKEKGYIPTTKDGYVIKSGTVSIKAGRYYVSVLVEIPDMLIDSSNN | | UGUGAGUAGCAUAUCUGUUUACA |
| | | DGIGIDLGLKNFAIVSNGKTYKNINKSARLKKLEKQLRREQKCLSRK | | GAUUGUAAAAGCAUAUACGAUGA |
| | | YEYLKKGESTQRANIQKQKLKVQKLYHRLDNIHTDYINKIIAEIVKTK | | AGCAGUAAGUAAUGUUCGUGAGA |
| | | PSYITIEDLNVSGMMKNRHLSKAVASQKFYEFRTTLKSKCDDNNIEL | | ACUUACAACAUCUCGAUGUGAGU |
| | | RVVDRFYPSSKLCHCCGSIKKDLKLSDRIYRCDCSYVADRDLNAALN | | AUAUUUACACACAUUUUGAGUGG |
| | | LKDAKTYKIA | | CAG |
| 6 | RHS75625.1 | MWKLLKSFKTEINPSEEQKVKIHKTIGTCRFIYNFYLAHNKELYDKG | 63 | CUAAAACGCAAACGUAAGUAUGU |
| | | EKFMSGKSFSVWLNNEYLPQNPDKLWIKEVSSKSVKHSIENGCIAFT | | ACCGAAGGCUAUUUUCGGGAAUU |
| | | RFFKHQSAFPNLKKKGKSDVKMYFVKNNPKDCRCERHRINIPSLGW | | UACGACUAUGGAGUGUACAAGAA |
| | | VRIKEKGYIPTTKDGYVIKSGTVSMKADRYYVSVLVEISNNKIANNS | | CUUGUGAGUAGAUAUGAUUUCGG |
| | | NAGIGIDLGLKDFAIVSNGKTYKNINKSARLKKHEKQLIREQRSLSRK | | UCAAUCCAAAGCAUACACGAUGA |
| | | YENLKKGESTQKANIQKQRLKVQKLHHRMDNIRTDYINKTIAEIVKT | | AAUAGUAAGUAAUGUUCGUGAGA |
| | | KPSYITIEDLNVKGMMKNRHLSKAVASQKFYEFRTKLQAKCNENGIE | | ACUUACAUUAUCUCGAUGUGAGC |
| | | LRVVDRWYPSSKTCHCCGAVKKDLKLSDRIFKCSCGYVEDRDFNAA | | AUAUUUAAUCACAUUUUGAGUGG |
| | | LNLRDAITYEVA | | CAG |
| 7 | RHU00256.1 | MRKLLKSFKTEINPTEEQKAKIRKTIGTCRYIYNFYLAHNKELYNKGE | 64 | AUAAAUGCAAACGUAGGUAUGUA |
| | | KFMSSNQFRVWLNNEYLPKHPECFWIKEAYSKAVTQAVNNGQAAFI | | CCGAAGGCUAUUUCGGGAAUUUA |
| | | RFFKHQSSFPRFKKKNRSDVKMYFVKNNPKDCRCQRHRINIPSLGW | | CGACUAUGGAGUGUACAAGAACU |
| | | VRIKEKGYIPTTKDGYVIKSGHVSIKAGRYYVSALVEIPNNKIANSFN | | UGUGAGUAGAUAUGAUUUCGGUC |
| | | EGIGIDLGIKDFAIVSNGKIYKNINRSVRLKKLGKQLIREQKNPSRKYE | | AAUCCAAAGCAUACACGAUGAAG |
| | | NLKKGESTQKANIQKQRLKVQKLHHKIDNIRTDHINKTIAEIVKTKPS | | UAGUAAGUAGAACUCGUGAGAGU |
| | | YITIEDLNISGMMKNKHLSKAVASQKFYEFRIKLQAKCNENGIELRV | | CUACAUUAUCUCGAUGUGAGUAU |
| | | VDRWYPSSKTCHCCGAIKKDLKLSDRIFKCDCGYSEDRDFNAALNL | | GUUUAAUCACAUUUUGAGUAGCA |
| | | RDATTYEIA | | G |
| 8 | RGT12103.1 | MWKLLKSFKTEINPSEEQKVKIHKTIGTCRFIYNFYLAHNKELYDNG | 65 | CUAAAACGCAAACGUAAGUAUGU |
| | | EKFMSSNKFRVWLNHEYLSEHPEYSWIKEVYSKAVTQAVNNGQTAF | | ACCGAAGGCUAUUUUCGGGAAUU |
| | | ARFFNHESAFPNFKKKGKSDVKMYFVKNNPKDCRCERHRINIPSLG | | UACGACUAUGGAGUGUACAAGAA |
| | | WVRIKEKGYIPTTKDGYVIKSGTVSMKADRYYVSVLVEILNNKIANN | | CUUGCGAGUAGAUAUGAUUUCGG |
| | | SNAGIGIDLGLKEFAIVSNGKTYKNINKSARLKKLEKQLIREQRSLSR | | UCAAUCCAAAGCAUACACGAUGA |
| | | KYENLKKGESTQKTNIQKQRLKVQKLHHRIDNIRTDYINKTIVEIVKT | | AAUAGUAAGUAAUGUUCGUGAGA |
| | | KPSYITIEDLNVKGMMKNRHLSKAVASQKFYEFRTKLQAKCNENGIE | | ACUUACAUUAUCUCGAUGUGAGC |
| | | LRVVDRWYPSSKTCHCCGAVKKDLKLSDRIFKCSCGYVEDRDFNAA | | AUAUUUAAUCACAUUUUGAGUGG |
| | | LNLRDAITYEVA | | CAG |
| 9 | RHN04853.1 | MARLLKSFKTEINPSEEQKVRIYKTIGTCRFIYNFYLSHNKELYDKGE | 66 | AAAAGCAGAUGUAAGUAUGUACC |
| | | KFMSSNKFRLWLNNEFLPDNPDKMWIKEAYSKAVTQAVNHAQTAF | | GAAGGCUAUUUCGGGAAUUUACG |
| | | KKFFDHKTAFPRFKKKGKSDVKMYFVRNNPKDCRCERHRIHIPSLG | | ACUGUGGAGUGUACAAGAACCUG |
| | | WIRIKEKGYIPTTKDGYIIKSGTVSIKAGRFYVSALVEVPDRKITEPFN | | UGAGUAGAUAUGAUUUUGGUCAA |
| | | EGIGIDLGLKEFAVVSNGRIYKNINRSARIRKLEKQLIREQRRLSRKYE | | UCAAAAGCAUACACAAAGAAGCA |
| | | DLKKGETTQKANIQKQKRKVQKLHQRIQNIRTDYMNKIIAEIVKTKP | | GUAAGUAGAACUCGUGAGAGUUU |
| | | SYITIEDLNVKGMMKNRHLSKAVASQKFYEFRRKLQAKCTEKGMEL | | ACAUUAUCUCGAUGUGAGUAUAA |
| | | RIADRWYPSSKKCHCCGCINKELKLSDRIYRCGCGYVEDRDFNAAL | | UUACACACAUUUUGAGUGGCAG |
| | | NLRDARTYKTA | | |
| 10 | RHR46218.1 | MRKLLKSFKTEINPTEEQKTKIRKTIGTCRFIYNFYLAHNKKLHEDGK | 67 | UUAAAACGCAAAUGUAAGUAUGU |
| | | KFMSSNKFRIWLNNEYLPQHPEYLWIKEAYSKAVTQSVNNGQTAFT | | ACCGAAGGCUAUUUCGGGAAUUU |
| | | RFFNHESAFPNFKKKGKSDVKMYFVKNNPKDCHCERHRIKIPSLGW | | ACGACUGUGGAGUGUACAUGAAC |
| | | VRIKEKGYIPTTKDGYVIKSGSVSIKADRYYVSVLVEVSDNKTINHFD | | UUGUGAGUAGAUAUGACUACGGU |
| | | EGIGIDLGLKDFAIVSNGRTYQNINKSAKLKKLERQLIREQRCLSRKY | | CAAUCAAAAACAUACACAAUGAA |
| | | ENLKKGDATQRANIQKQKLKVQKLHHRIDNIRTDYINKTIAEMVKIK | | GCAGUAAGUAAUGUUCGUGAGAA |
| | | PSYITIEDLNVKGMMKNRHLSKAVASQKFYEFRAKLLTKCNKSGIEL | | CUUACAUUAUCUCGAUGUGGGUA |
| | | RVVDRWYPSSKICHCCGSIKKDLKLSDRIFKCRCGYIEDRDFNASLNL | | UAUUUAAUCACAUUUUGAGUGGC |
| | | RDAETYSIA | | AG |
| 11 | RGR69673.1 | MRKLLKSFKTEINPTVEQKIKINKTIGTCRYVYNFYLGHNKALYDKG | 68 | AACGCAAACGUAAGUAUGUACUG |
| | | EKFMTGKSFSVWLNNEYIPNNPDKIWIKEAYSKAVKKSIEDGCTAFT | | CGGGCUAUCGCAGGAAUUUACGA |
| | | RYFKHQSAFPNFKKKGKSDVKMYFVKNNPKDCRCERHKLNIPTLG | | CUGUGGAGUGUACAAGAACUUGU |
| | | WVRMKEKGYIPTTKDGWKIKSGTVSIKADRYYVSVLVEIPDAKITDN | | GAGUAGCGUAUUGUUUACAAUCG |
| | | SNDGMGIDLGLKDLAIVSNGKTYKNINKSARVKKLEKKLRREQRCL | | CCAAAGCAUACACAAUGAAGCAG |
| | | SRKYENLKKGESTQKNIQKQKLKVQRLHHKIDNIRTDYINKSIAEIVK | | UAAGAAGUAUCCGCAAGGACUUC |
| | | AKPSYITIEDLNVSGMMKNRHLSKAVASQKFYEFRTKLKAKCDENGI | | AAUUUCUCGAUGUGUUUGAGUAU |
| | | ELRVVDRWYPSSKICHCCGAVKKDLKLSDRIYRCDCGYIEDRDFNA | | AUUUCAACACAUUUUGAGUGGCA |
| | | ALNLRDALTYEVA | | G |
| 12 | RHC60416.1 | MRKLLKSFKTEIDPTEEQKVRIHKTIGTCRFIYNFYLAHNKELHKNGE | 69 | AUAAACGCAAACGUAGGUAUGUA |
| | | RFMSSSKFRVWLNNEYLPNHPEYSWIKEAYSKAVTQAVNNGQTAFT | | CCGAAGGCUAUUUCGGGAAUUUA |
| | | RFFNHESNFPNFKKKGRSDVKMYFVKNNPKDCCCERHRIKIPSLGW | | CGACUAUGGAGUGUACAAGAACU |
| | | VRIKEKGYIPTTKDEYVIKSGHVSIKADRYYVSVLVEIPDDKMVNNS | | UGUGAGUAGAUAUGAUUUCGGUC |
| | | NEGIGIDLGLKDLAIISNGKTYKNINKSAKLKKLEKQLIRGQRSLSRK | | AAUCCAAAGCAUACACGAUGAAA |
| | | YENSKKGESTQRANIQKQKIKVQKLYQKIDNIRTDYINKTIAEIVKTK | | UAGUAAGUAAAACUCGUGAGAGU |
| | | PSFITIEDLNVKGMMKNRHLSKAVASQKFYEFRIKLQAKCNENGIEL | | UUACAUUAUCUCGAUGUGAGUAU |
| | | RVVDRWYPSSKTCHCCKNIKKDLRLSDRIFKCDCGYIEDRDLNAALN | | AUUUAAUCACAUUUUGAGUGGCA |
| | | LRDAMTYEVA | | G |
| 13 | RHB06265.1 | MRKLLKSFKTEINPTIEQKIKINKTIGTCRYVYNFYLGHNKTLYDNGE | 70 | UAAACGCAAACGUAAGUAUGUAC |
| | | KFMTGKSFSVWLNNEYIPNNPDKIWIKEAYSKAVKKSIEDGCTAFTR | | UGCGGGCUAUCGCAGGAAUUUAC |
| | | FFKHQSAFPNFKKKGKSDVKMYFVKNNTKDCRCERHRLNIPTLGWV | | GACUGUGGAGUGUACACGAACUU |
| | | RIKEKGYIPTTKDGWKIKSGTVSVKADRYYVSVLVEIPNVKIANNSN | | GUGAGUAGCGUAUUGUUUACAAU |
| | | GGIGIDLGLKDLAIVSNGKTYKNINKSTRIKKLEKKLRREQRCLSRKY | | CGCCAAAGCAUACACAUCGAAGC |
| | | ENLKKGESTQKNIQKQKLKVQRLHHKIDNIRTDYINKSIAEIVKTKPS | | AGUAAGAAGUAUCCGCAAGGACU |
| | | YITIENLNVSGMMKNRHLSKAVASQKFYEFRTKLKAKCDENGIELRV | | UUAAUUUCUCGAUGUGUUUGAGU |
| | | VDRWYPSSKICHCCGAIKKDLKLSDRIYRCDCGYVEDRDFNAALNL | | AUAUUUGAACACAUUUUGAGUGG |
| | | RDALTYEVA | | CAG |
| 14 | RGN78093.1 | MKRAYLIEIKPTTEQVSKIHQTIGVCRYVYNLYISKNKEAYESEGKFL | 71 | AUAGAUUGUAUAUAUGUACGGUG |
| | | SGYDFSKWLNNIHTKECDQWIKEVSSKAVKQAIMNGDKAFKRFFKG | | GGCUACAUCGGAAUUUACGCCUG |
| | | LSKFPRYKKKRKQDVKCYFPKNNKTDWTVERHRVKVPTIGWVRLK | | UGGAGUGUCACAGCAAACUGUAG |
| | | EYGYIPKNATVKSGTVYQRAGRYFVSVLCERAEIRVKPMLNKVGLGI | | UAGCAAGUGAUUAGCGAGGCAGG |
| | | DLGLKDFAVRSDGKTHKNINKTVQVKKIEKRLKREQRSLSRKFENR | | GCACGUAGAAGCAGGAAUACUCU |
| | | KKRGEQSVTNKRANIDKNILRVQKLHARLANIRLAYVKSIVNDVVK | | AAGAAUAUACAUUUGUCUAUAUU |
| | | TKPTFITVEDLNVKGMMKSKHLSKTIAKQSFFAFKTWLLTKCEEYGI | | UUUAGUAGCAG |
| | | ELRQVDRFYPSSKLCSCCSQKKVDLKLSDRVYTCECGNVMDRDLNA | | |
| | | AINLLQAKEYTILT | | |
| 15 | RGR87203.1 | MILAKKVRLNPTEEQERQLWKSVGTARWVYNWTLSKQEESYKNDG | 72 | CACCUAAAACGUUAACGCUAAUA |
| | | KFISDGELRKELTQLKKTEEYSWLLDVSNNVAKQAVKDACDAYKKF | | UGUACGAUUCGUUGCAUCGGAAU |
| | | FKKLANKPRFKSRKRSKPSFYNDNVKLKVKEMLVLIEKVGWVKTSE | | UUACGCCUGUGGAGUGUUACACC |
| | | QLPINTKYVNPRVSFDGKYWYLSVGIQQKHVHNDLTDNIIGIDVGVK | | AAACGAAAGUAGCUUCGGCAAAA |
| | | ELAVCSNGMVFKNINKTKTVKKIEKRLRRLQRKVSRKYEMNKEGNR | | UCGGACACGAUGAAUCAGGAAGC |
| | | FVKTCNIIKTEKKIRLLHRRLMNIRTNHIHQTTNAIVKTKPSTVVME | | AAACAAGAAUUUAUAAGUUUUUA |
| | | KLNIKGMMKNKYLSKAVQNQKLYEFKRQMKYKCEFNGIKFIEADKW | | UAAUUUCUUGGCAACGG |
| | | YPSSKTCSNCGQIKKDLKLSERIFRCDCGLEIDRDLNASINLAKLAN | | |
| 16 | RHU47384.1 | MVKAIKVMLIPNNVQKTKMFQYAGASRFAYNWALAREKENYEKG | 73 | CACUGAAACGUUAAUGCAAAUAU |
| | | GRFIPDAELRKEFTKLRHSDEYAWLLSISNNVTKQAIKDACTAYKNF | | GUACGGAUACGUUAGUCCGGAAU |
| | | FKGLQKFPRFKYRKRSMPKFYQDNVKIRFSNTHVKFEGFSSGRKANK | | UUACGCCUGUGGAGAGUACAAGA |
| | | QKMNWVRLAEHGRIPTDAKYMNPRISFDGLNWWISVCVELPDCRET | | ACUUGUGAGUAGAUUGAUAUUUA |
| | | PNDDGVGIDLGIKNLAVCSDAVKYKNINKSQKVKKLEKQKRGLQRS | | UAUCAUCAAAAGCAUACUCGUUG |
| | | ISRSYEKNKRGESYCKTNNVIKKKKLLLKRNHRLTNIRKNYLNQTISE | | AAGCAGGAAUGAAACAUGGAAGU |
| | | IVNRKPRFICIEDLNVSGMMKNRHLSKAVQEQGFFRFRKQLEYKCSD | | UUAUAACUUUUUAUAAGUUUUCA |
| | | KGIQLIVADRFYPSSKLCSCCGNIKKDLKLSDRIYRCECGNRIDRDFQ | | GUAACGG |
| | | ASINLKIYGERFAS | | |
| 17 | RHU18041.1 | MLKSFKTEIHPTEEQKVRIHKTIGTCRFIYNFYLAHNKELYQNGEKFM | 74 | GGAAGCAAAUGUAAGUAUGUACC |
| | | SSSKFRVWLNHEYLPQHPEYLWIKEAYSKAVTQAVNHGQTAFKKFF | | GAAGGCUAUUUCGGGAAUUAACG |
| | | NHESAFPKFKKKGRSDVKMYFVKNNPKDCRCERHRVNIPSLGWVRI | | ACUGUGGAGUGUACAUGGACCUA |
| | | KEKGYIPTTKEGYIIKSGHVSMKAGRYYVSALIEIPDNKTADLSNEGI | | CGAGUAGAUAUGAUUUCGGUCAA |
| | | GIDLGLKDFAIVSNGKTYKNINKSARLKKLEKRLVREQRCLSRKYEN | | UCAAAAGUAUACACACUGAAGCA |
| | | LKKGESTQRANIQKQKLKVQKLHHKIDNIRTDYINKTIAEIVKTKPSY | | GUAAGUAAGAUUCGUGAGAACUU |
| | | ITIEDLNVSGMMKNKHLSRAVASQKFYEFRIKLKAKSRENGIELRVA | | ACAUUAUCUCGAUGUGAUAAGUA |
| | | DRWYPSSKICHCCGTIKKDLKLSDRTFRCHCGYVEDRDFNAALNLR | | UAUUUAAUCACAUUUUGAGUGGC |
| | | DVKTYKIA | | AG |
| 18 | RHV03091.1 | MRRLLKSFKTEINPTEEQKIKIHKTIGTCRYIYNFYLFHNKERYDIGEK | 75 | UCCUGCAAACGUAAGUAUGUACC |
| | | FMSGRAFSVWLNNEYLPKHPEYSWIREVSSKSVKNAIENACTAFSRF | | GAAGGCUAUUUCGGGAAUUAACG |
| | | FHHQSGFPRYKKKGRSDVKMYFVRNNPKDCFCERHRIKIPTLGWVR | | ACUGUGGAGUGUACUUAGGAUCU |
| | | LKEKGYIPTSRDGYVVRSGAVSMKAGRYYVSALIEVPTPKVDGNFS | | GUGAGUAGAGAUAACGCAGGUUA |
| | | DGIGIDLGLKNFAVVSNGTVYRNINKTVQIRKLEKQLTRAQRKLSRK | | CUCAAAAACAUACACGAAGAAAC |
| | | YENLRKGEFTQKANIQKQKLKVQKLYDRIENIRTDHINKTISEIVKTK | | AGUAAGCGGUAUUCGCGAGAAGC |
| | | PSHITIEDLNVSGMMKNRHLSKAVSSQKFYEFRTKLKAKCAEYGIEL | | CGCAUUAUCUCAAUGUGAGUAUG |
| | | RVVDRWYPSSKTCHCCGCIKKDLKLSDRIYKCTCGYVEDRDFNAAL | | UUUAAUCACAUUUUGAGUGGCAG |
| | | NLRDARTYKVA | | |
| 19 | RHT78732.1 | MRELLKSFKTEINPTAEQKVKINKTIGTCRFVYNFYLDHNKALHNKG | 76 | UUAACGCAACGUAAGUAUGUACC |
| | | EKFMTGKSFSVWLNNEYIPNNPDKAWIKEAYSKAVKKSIENGCIAFT | | GAAGGCUAUUUCGGGAAUUAACG |
| | | RFFKHQSAFPNFKKKGKSDVKMYFVKNNPKDCRCERHRLNIPTLGW | | ACUGUGGAGUGUACAAGAACUUG |
| | | VRIKEKGYIPTTKDGWRIRSGTISIKAGRYYVSVLIEVPDVKNTNNNS | | UGAGUAGCGUAUUGCUUGCAAUC |
| | | KGIGIDLGLKDFAIISNGKIYKNINKSARVKKLENKLRREQRCFSRKY | | GCCAAAGCAUACACAUUGAAGCA |
| | | ENLKKGESTQKNIQKQELKVQRLHHKIVNIRTDYINKTIAEIVKTKPS | | GUAAGAAGUAUCCGUGAGGACUU |
| | | YITIEDLNVSGMMKNKHLSKAVASQKFYEFRTKLKAKCDENGIELRI | | CAGUUUCUCGAUGUGUUUGAGUA |
| | | VNRWYPSSKICHCCGAIKKDLKLSDRIYRCGCGYVEDRDFNAALNL | | UAUUUAAACGCAUUUUGAGUGGC |
| | | RDALTYEIA | | AG |
| 20 | RHR42937.1 | MKKLLKSFKTEINPTVEQKIKINKTIGTCRYVYNFYLVHNRNLHDQC | 77 | UGAACGCAAAUGUAAGUAUGUAC |
| | | EKFMTGKSFSVWLNNEYIPNHPDKAWIKEAYSKAVKKSIEDGCTAFT | | CGAAGGCUAUUUCGGGAAUUAAC |
| | | RFFKHQSDFPNFKKKGKSDVKMYFVKNNPKDCRCERHRLNIPTLGW | | GACUGUGGAGUGUACCAGAACUU |
| | | VRIKEKGYIPTTKDGWKIKSGTVSIRAGRYYVSALVEVPDATIYNNN | | GUGAGUAGUGAGUUACUUGUAAC |
| | | NDGVGIDLGLKDLAIVSNGKTYKNINKSARVKKLEKQLRREQRCLSR | | CACAAAAGCAUACAUGGUGAAGC |
| | | KYESLKKGESTQKNIQKQKRKVQKLHHKIDNIRTDYINKTVAEIVKT | | AGUAAGAAGUAUCCGUGAGGACU |
| | | KPSYITIEDLNISGMMKNRHLSKAIASQKFYEFRIKLKAKCDENGVEL | | UCAAUUUCUCGAUGUGAUUGAGU |
| | | RVADRWYPSSKICHCCGTIKRDLKLSDRIYRCSCGYIEDRDFNASLNL | | AUGUUCAAACACAUUUUGAGUGG |
| | | RDAITYKIA | | CAG |
| 21 | RGD88909.1 | MRKLLKSFKTEINPTEEQKTKIRKTIGTCRFIYNFYLAHNKKLHEDGK | 78 | UUAAAACGCAAAUGUAAGUAUGU |
| | | KFMSSNKFRIWLNNEYLPQHPEYSWIKEAYSKAVTQSVNNGQTAFT | | ACCGAAGGCUAUUUCGGGAAUUU |
| | | RFFNHESAFPNFKKKGKSDVKMYFVKNNPKDCHCERHRIKIPSLGW | | ACGACUGUGGAGUGUACAUGAAC |
| | | VRIKEKGYIPTTKDGYVIKSGSVSIKADRYYVSVLVEVSDNKTINHFD | | UUGUGAGUAGAUAUGACUACGGU |
| | | EGIGIDLGLKDFAIVSNGRTYQNINKSAKLKKLERQLIREQRCLSRKY | | CAAUCAAAAACAUACACAAUGAA |
| | | ENLKKGDATQRANIQKQKLKVQKLHHRIDNIRTDYINKTIAEMVKIK | | GCAGUAAGUAAUGUUCGCGAGAA |
| | | PSYITIEDLNVKGMMKNRHLSKAVASQKFYEFRAKLLAKCNESGIEL | | CUUACAUUAUCUCGAUGUGGGUA |
| | | RVVDRWYPSSKTCHCCGSIKKDLKLSDRIFKCRCGYIEDRDFNAALN | | UAUUUAAUCACAUUUUGAGUGGC |
| | | LRDAETYSIA | | AG |
| 22 | RHN30353.1 | MKKLKRAYKIEINPTAEQKSKIHQTIGVSRFIYNFYIAHNKEIYAREG | 79 | AAUCAAAAGCACUUAUAUAUGUA |
| | | KFVSGMDFSKWLNNKYIPNNKDMKWIKEVSSKATKQAIMNGDKAF | | CGGAGGGCUAACUUCGGAAUUUA |
| | | RDFFKKAKGFPKFKKKKNQDVKAYFPKNNKTDWTLERHKVKIPTLG | | AGCCUUUGGAGAGCCAUACAAAU |
| | | WVRLKEFGYIPVNSIVKSGTVSQKADKYYVSILVEEDDKKVYKSTNE | | CGUAGUAGCUUAGGCAAGGCGAG |
| | | GLGIDLGVKEFVVCSNGIKFKNIN | | GCUUUAUGAAGAAGGAAUAAUCU |
| | | KTSTVKKVEKKLKREQRKLSRKYESLKIRNKNLKEGRATSQNIQKQL | | CCAUAUGGAUAUAUUUGACCAUA |
| | | VKVQKLHQRLTNIRTDYINKIVSSIIKQKPSYITIEDLAVSNLMKNKHL | | UUUUGAGUAGCAG |
| | | SKAIASQKFFEFKTKLMFKCKENHIELRIVDRFYPSSKTCSSCGKVKK | | |
| | | DLKLSDRVYKCDCGLTIDRDLNASINLKNAKEYKIA | | |
| 23 | RGC42877.1 | MVKAIKVMLIPNNVQKTKMFQYAGASRFAYNWALAREKESYEKGG | 80 | CACUGAAACGUUAAUGCAAAUAU |
| | | RFISDSELRKEFTKLRHSDEYAWLLNISNNVTKQAIKDACTAYKNFF | | GUACGGAUACGUUAGUCCGGAAU |
| | | KGLQKFPRFKSRKRSMPKFYQDNVKIQFSNTHVKLEGFSSSRKANKQ | | UUACGCCUAUGGAGAGUACAAGA |
| | | KKNWVRLAEHGRIPTDVKYMNPRISFDGLNWWISVCVEFPDCKETL | | ACUUGUGAGUAGAUAGAUAUUUA |
| | | NDDGVGIDLGIKDLAVCSDAVKYKNINKSQKVKKLEKQKRRLQRSIS | | UAUCAUCAAAAGCAUACUCGUUG |
| | | RSYEKNKKGESYCKTNNVIKKEKLLLKRNHRLTNIRKNYLNQTISEI | | AAGUAGGAAUGAAACAUGGAAGU |
| | | VNRKPRFICIEDLNVSGMMKNRHLSKAVQEQGFFWFRKQLECKCND | | UUAUAACUUUUUAUAAGUUUUCA |
| | | KGIQLIVADRFYPSSKLCSCCGNIKEDLKLSDRVYRCECGNMIDRDFQ | | GUAACGG |
| | | ASINLKAYGERFAS | | |
| 24 | RGT87174.1 | MVKAIKVMLIPNNVQKTKMFQYAGASRFAYNWALAREKENYEKG | 81 | CACUGAAACGUUAAUGCAAAUAU |
| | | GKFISDSELRKEFTKLRHSDEYAWLLNISNNVTKQAIKDACTAYKNF | | GUACGGAUACGUUAGUCCGGAAU |
| | | FKGLQKFPRFKSKKRSMPKFYQDNVKIQFSNTHVKFEGFSSSRKANK | | UUACGCCUAUGGAGAGUACAAGA |
| | | QKMNWVRLAEHGRIPTDVKYMNPRISFDGLNWWISVCVEFPDCKET | | ACUUGUGAGUAGAUAGAUAUUUA |
| | | LNDDGVGIDLGIKDLAVCSDAVKYKNINKSQKVKKLEKQKRRLQRS | | UAUCAUCAAAAGCAUACUCGUUG |
| | | ISRSYEKNKKGESYCKTNNVIKKEKLLLKRNHRLTNIRKNYLNQTIS | | AAGUAGGAAUGAAACAUAGAAGU |
| | | EIVNRKPRFICIEDLNVSGMMKNRHLSKAVQEQGFFWLRKQLEYKCS | | UUGUAACUUUUUAUAAGUUUUCA |
| | | DKGIQLIVADRFYPSSKLCSCCGNIKKDLKLSDREYRCECGNIIDRDF | | GUAACGG |
| | | QASINLKGYGERFAS | | |
| 25 | RGC76021.1 | MIKSIKVRLCPNNKQLTKLFQYAGCARFAYNWAIAREQENYKQGDK | 82 | CACUUUCAAGUUAAUACAGAUAU |
| | | FLSDNELRKEFTQLKKQSEYQWLSEVSNNVTKQAIKDACNTYKKFF | | GUACUGAUUCGUUAGUCAGGAAU |
| | | KGQCKYPKFKSRKHSSPSFYQDNIKIQFSDTHVKVEGFSMSKKKNKQ | | UUACGCCUAUGGAGAGUACAAGA |
| | | KLNWIRLCEKGRIPTNCKYMNPRFTYDELHWYISVGIEVEDSTTIPLN | | ACUUGUAAGUAGUGAUUGACUUA |
| | | NGIGIDLGIKDLAVCSDGNIYKNINKTTTVNKLEKRKRRLQRSISRRY | | CCAACACGAAAGCAUACUCGUUG |
| | | EKNKKGGIYCKTCNIIKKEKELLKVTKRLTNIRQNYLHQTTSEIIKRK | | AAAUAGGAAUGAAACAUAAAAGU |
| | | PSFICIEDLNVSGMMKNKHLSKAVQQQCFYEFRKQMEYKSAWNNIPV | | UUAUAACUUUUUAUAAGUUUUCA |
| | | IIADRFFPSSKLCSCCESIKKDLKLSDRIYKCECGNVIDRDYQASLNL | | GUAACGG |
| | | KMYGENVLKQQSVA | | |
| 26 | RGR57390.1 | MVKAIKVMLLPNNVQKTKMFQYAGASRFAYNWALAREKESYEKG | 83 | CACUGAAACGUUAAUGCAAAUAU |
| | | GKFISDSELRKEFTKLRHSDEYAWLLNISNNVTKQAIKDACTAYRNF | | GUACGGAUACGUUAGUCCGGAAU |
| | | FKGLQKFPRFKSRKRSMPKFYQDNVKIQFSNTHVKLEGFSSSRKANK | | UUACGCCUAUGGAGAGUACAAGA |
| | | QKKNWVRLAEHGRIPTDVKYMNPRISFDGLNWWISVCVEFPDCKET | | ACUUGCGAGUAGAUAGAUAUUUA |
| | | LNDDGVGIDLGIKDLAVCSDAVKYKNINKSQKVKKLEKQKRRLQRS | | UAUCAUCAAAAGCAUACUCGUUG |
| | | ISRSYEKNKKGESYCKTNNVIKKEKLLLKRNHRLTNIRKNYLNQTISE | | AAGUAGGAAUGAAACAUGGGAGU |
| | | IVNRKPRFICIEDLNVSGMMKNRHLSKAVQEQGFFGFRKQLEYKCND | | UUAUAACUUUUUAUAAGUUUUCA |
| | | KGIQLIVADRFYPSSKLCSCCGNIKKDLKLSDRVYRCECGNMINRDF | | GUAACGG |
| | | QASINLKTYGERFAS | | |
| 27 | RGW55202.1 | MYSKAVKKSIEDGCAAFTRFFKHQSDFPKFKKKGKSDVKMYFVKN | 84 | UUAAAAGCAAAUGUAAGUAUGUA |
| | | NPKDCQCERHRLKIPTLGWVRIKEKGYIPTTKAGYMIRSGTVSVKAG | | CCGAAGGCUAUUUCGGGAAUUAA |
| | | RFYVSVLVEIPDVNINNNLNEGIGIDLGLKDFAIISNGKTYRNINKSAG | | CGACUGUGGAGUGUACAAGAACC |
| | | LKKLEKQLIREQRSLSRKYENLKKGESTQRANIQKQKLKVQKLHHK | | UGUGAGUAGCGUAUUGCCUGCAA |
| | | MDNIRTDYINKTIAEIVKTKPSYITIEDLNVKGMMKNRCLSKAVASQ | | UCGUCAAAACAUACACAAUGAAG |
| | | KFYEFRKRLKAKCDEKGIELRIADRFYPSSKTCHHCGSIRKNLKLSDR | | CAGUAAGAAGUAUUCGUGAGGAC |
| | | IYRCECGYVADRDLNAALNLKDAKTYKIA | | UUCAAUUUCUCGAUGUGUUUGAG |
| | | | | UAUAUUUAAACAUAUUUGGAGUG |
| | | | | GCAG |
| 28 | RHP08869.1 | MRKLLKSFKTEINPTEEQKVKIRKTIGTCRFIYNFYLAHNKELHKNGE | 85 | AUAAACGCAAACGUAGGUAUGUA |
| | | KFMSSNKFRVWLNNEYLPNHPEYSWIKEAYSKAVTQAVNNGQTAF | | CCGAAGGCUAUUUCGGGAAUUUA |
| | | TRFFNHKSSFPNFKKKGKSDVKMYFVKNNPKDCRCERHRINIPSLGW | | CGACUAUGGAGUGUACAAGAACU |
| | | VRIKEKGYIPTTKDGYVIKSGHVSIKADRYYVSVLIEIPNVKIANNSN | | UGUGAGUAGAUAUGAUUUCGGUC |
| | | AGIGIDLGLKDFAIVSNGKTYKNINKSAKLKKLEKQLIREQRSLSRKY | | AAUCCAAAGCAUACACGAUGAAA |
| | | ENLKKGESAQKPNIQKQRLKVQKLHHRIDNIRTDYINKTIAEIVKTKP | | UAGUAAGUAAUGUUCGUGAGAAC |
| | | SYITIEDLNISGMMKNKHLSKAVASQKFNEFRTKLQAKCNENGIELR | | UUACAUUAUCUCGAUGUGAGUAU |
| | | VVDRWFPSSKTCHCCKNIKKDLKLSDRIFKCDCGYIEDRDFNAALNL | | AUUUAAUCAUAUUUUGAGUGGCA |
| | | RDATTYEVA | | G |
| 29 | RGF95087.1 | MIKSIKVRLNPNNKQSTKLFQYAGCARFAYNWAISREQDNYKQGNK | 86 | UACGUUAGUCAGGAAUUUAUGCC |
| | | FLSDSELRKEFTQLKKQSEYQWLNEVSNNVTKQAIKDACNTYKRFF | | UAUGGAGUGUACAAGAACUUGCG |
| | | KGQCKYPKFKSKKHSTPSFYQDNIKIQFTDTHVKVESFSMSKKQNKQ | | AGUAGAUUAUGAGAAAUCAUGUC |
| | | KLNWIKLCEKGRIPTGCKYMNPRFTYDGLYWYVSIGIEVNDNTTLPS | | AAAAGCAUAUACGAUGAAGUAGG |
| | | NEGIGIDLGIKHLAICSDGNTYKNINKTQTVKKLEKKKRRLQRSISRK | | AAUGAAACAUAAAGGUUUAUAAC |
| | | YEKNKKGANYCKTSNIIKREKELLKLNHRLTNVRQNYLHQTTSEIVK | | UUUUUAUAAGUUUUCAGUAACGG |
| | | REPSFICIEDLNVSGMMKNKHLSKAVQQQGFYEFRRQIEYKAMWNN | | |
| | | IPVVIADRFFPSSKLCSCCGSIKKDLKLSDRIYKCECGNVIDRDYQAAL | | |
| | | NLKQYGENVLKQQSVAYHFQVITDMY | | |
| 30 | RGH01875.1 | MVKAIKVMLIPNNVQKTKLFQYAGASRFAYNWALAREKENYEKGG | 87 | GAUUGAAACGUUAAUGCAAAUAU |
| | | KFISDSELRKEFTRLRHSDEYAWLLNISNNVTKQAIKDACTAYQNFF | | GUACGGAUACGUUAGUCCGGAAU |
| | | KGLQKFPRFKSRKRSMPKFYQDNVKIQFSNTHVKFEGFSSSRKANKQ | | UUACGCCUAUGGAGAGUACAAGA |
| | | KRNWVRLAEHGRIPTDAKYRNPRISFDGLNWWISVCVEFPDSKETLN | | ACUUGUGAGUAGAUAGAUAUUUA |
| | | EDGVGIDLGIKDLAVCSDAVKYKNINKSQKVKKLEKQKRRLQRSISR | | UAUCGUCAAAAGCAUACUCGUUG |
| | | SYEKNKKGESYCKTNNVIKKEKFLLKRNHRLTNIRKNYLNQTISEIV | | AAGUAGGAAUGAAACAUAGAAGU |
| | | NRKPRFICIEDLNVRGMMKNRHLSKAVQEQGFFGFRKQLEYKCSDK | | UUAUAACUUUUUAUAAGUUUUCA |
| | | GIQFIVADRFYPSSKLCSCCGNIKKDLKLSDRIYRCECGNRIDRDFQ | | GUAACGG |
| | | ASINLKAYGEQFAS | | |
| 31 | RHU31723.1 | MVKAIKVMLIPNNVQQTKLFQYAGASRFAYNWALAREKENYEKVG | 88 | CACUUAAAUGUUAAUGCAAAUAU |
| | | RFIPDTELRKEFTRPRNSDEYAWLLNVSNNVTKQAIKDACSAYKNFF | | GUACGGAUGCGUUAAUUCGGAAU |
| | | KGLQRYPRFKAKKKSMPKFYQDNIKIQFRDTNVKFEGFSSSRKANKQ | | UUACGCCUAUGGAGAGUACAAGA |
| | | KLNWVKLAEHGRIPTDAKYRNPRVSFDGLNWWISVCVEFPDCRDRS | | ACUUGUGAGUAGAUAGGUAUUUA |
| | | NHDGIGIDLGIKDLAICSDGNTYKNINKSQTAKKLEKCKRRLQRSVSR | | UGCCAUCAAAAGCAUACUCGUUG |
| | | KYEHNKKGGSYCKTNNIVKKEKLLLKVNHRLANIRKDYLNQTTSEI | | AAGUAGGAAUGAAACAUAAAAGU |
| | | ANRKPRFICIEDLNVSGMIKNRHLSKAVQNQGFFEFRKQLEYKCNDR | | UUAUAACUUUUUAUAAGUUUUCA |
| | | DFQASLNLKAYGEQFAS | | GUAACGG |
| 32 | RHQ17086.1 | MVKAIKVMLIPNNVQKTKMFQYAGASRFAYNWALAREKENYEKG | 89 | CACUGAAACGUUAAUGCAAAUAU |
| | | GRFISDSELRKEFTKLRHSDEYAWLLSISNNVTKQAIKDACIAYKNFF | | GUACGGAUACGUUAGUCCGGAAU |
| | | RGLQKFPRFKSRKRSMPKFYQDNVKIQFSNTHVKFEGFSSSRKANKQ | | UUACGCCUACGGAGAGUACAAGA |
| | | KMNWVRLAEHGRIPIDANYMNPRISFDGLNWWISVCVEFPDCRETL | | ACUUGUGAGUAGAUAGAUAUUUA |
| | | NDDGVGIDLGMKDFAVCSDAVKYKNINKSQKVKKLEKQKRRLQRSI | | UAUCAUCAAAAGCAUACUCGUUG |
| | | SRSYEKNKKGESYCKTNHVIKKEKLLLKRNHRLTNIRKNYLNQTVSE | | AAGUAGGAAUGAAACAUAGAAGU |
| | | IVNRKPRFICIEDLNVSGMMKNRHLSKAVQEQGFFLFRKQLEYKCSD | | UUAUAACUUUUUAUAAGUUUUCA |
| | | KGIQLIVADRFYPSSKLCSCCGNIKKDLKLSDRVYRCECGNMIDRDF | | GUAACGG |
| | | QASINLKAYGERFAS | | |
| 33 | RGW42851.1 | MILAKKVRIYPTKEQEQKLWQSVGTARFIYNYTLAKQEENYKNGGK | 90 | UUUCACAAACAAGAAAAUGCUAA |
| | | FISDGIIRKELTQLKKSELIWLNEVSNNVTKQAVKDACNAYKRFFKG | | UGUGUAGGAUUCGUUGUAUCCGA |
| | | LAKKPKFKIRKKSKPSFYNDTSKLKVKEKKVLIEKVGWIKTNEQIPID | | AUUUAAGCCUUUGGAGAGUUAUA |
| | | VKYNNPRVTYDNKYWYISVGVEVDKKQEELTDISLGIDLGLKDLAV | | UUAAACGAAAGUAGCUACGGCAA |
| | | CSDGKVFKNINKTKEVKNLEKRLKQKQRQISRKYEINKIKKGGSCQFI | | AAUCGGAUUCUAUGAAGAAGGAA |
| | | KTKNIEKLENTTKLMHRKLTNIRDNYIHQVTTSIVKTKPYRIVIEDLN | | AUAAACAAAUUUCUAUAUUUUUA |
| | | VSGMIKNKHLSDSVRKQCFNKFRQYITYKAELYGIGLVIADRFYPSS | | UAGAUUUUUGGCAACGG |
| | | KTCSECGSIKKDLKLKDRVYRCPHCGVVIDRDYNASLNLSMYKLA | | |
| 34 | RGQ08395.1 | MILAKKVRIYPTKEQEQKLWQSVGTARFIYNYTLAKQEENYKNGGK | 91 | UUUCACAAGCAAGAAAAUGCUAA |
| | | FINDGVIRKELTQLKKSELTWLNEVSNNITKQSVKDACKAYKRFFKN | | UAUGUAGGAUUCGUUGUAUCCGA |
| | | LANKPKFKSRKKSKPSFYNDPIKLKIKEKKVLIEKVGWIKINEQIPINV | | AUUUAAGCCUUUGGAGAAUCAUA |
| | | KYNNPRITYDNKYWYLSVGIEVDKKQEGLTNISLGIDLGLKELAICSD | | UCAAACGAAAGUAGCUACGGCAA |
| | | GKVFKNINKTKKIKKLEKRLKQKQRQISRKYEINKIKKEGGKRYQFIK | | AAUCGGAUUCAAUAAAGAAGGAA |
| | | TKNIKKLENTTKLIYRKLANIRNNYIHQVTTSIVKTKPYRIVIEDLNVS | | AUAAACAAAUUUUUAUAUUUUUA |
| | | GMMKNKHLSDSVKKQCFHKFRQYITYKSELNGIELVVADRFYPSSK | | UAGAUUUUUGGCAACGG |
| | | TCSKCGFIKKDLKLKDRVYRCPHCGAVIDRDLNASLNLSMYKLA | | |
| 35 | RHF50970.1 | MLKAFKTEIAPTKEQRDKIIRSIGVARFLYNQYISYNRRLYKMYRRG | 92 | UAGAGAAGUUCUGAUAUAUGUAC |
| | | MLDERQKYFVTAVDFDKYVNHKLKQELPWIDQCGSKARKKALVNA | | CGAUGGCUAGUCGGGAACAGGCU |
| | | EQAFRRFFAGISRFPTFKKKTNQDVKLYFPRNNKGDWKIWRHKLMIP | | CUUAUCCUUGCAUUGCAAGGAGC |
| | | TLKQVKLKEYGYLPVGMTATSGTVSYVAGRFYVSVVVDIDAKSKY | | GUGAAGCCAACGCCUUCGGAGUG |
| | | NKDLDSCYAIQTEGIGIDLGVKDLAVVSDGRVFENINKSSKVKRLEK | | UUAUACAAACGUCAGUAGCCUGU |
| | | RLRREQRRLSRKYVGKVDTVSANIEKQKLKVQKLHQRITRIREDYEN | | CUUUCGGACGGCGAGGGCGAACA |
| | | KTVHEIVKQKPRFITVEDLNVKGMMKNRHLAKAVAAQRFNHLLSKL | | CGAUGAACAAGGAAUAUUUCUAA |
| | | KRKAKIIGIELREVDRFYPSSKTCHACGHVHRGLKLKDRVYICPECG | | GAUACAUACAUUGAGGGUUAUGU |
| | | YRMDRDYNASLNLRDAKEYRVV | | UCUUAGUAGCAG |
| 36 | RHF52474.1 | MRNLLKSFKTELRLTEGQRDKVRRSLGICRFLANQYIARNIWLYRLY | 93 | UUCACGCUCUCACAUUUGAUAAA |
| | | QRGVLDEKQRRFVTANDFDKYVNHVLKKKFPWIDQCGSKARKKIL | | UGUACCGAUGGCUAGUCGGAAAC |
| | | VNAEQAFRRFFRGQAHFPRFKRKNQQNVKLYFPKNNKGDWTIWRH | | AGGCUGUUGCCUUGCCUAGCAAG |
| | | KIQIPTIGKVRLKEFGYLPQGVTIKNGHVSYKAGRYYVSVTAEIEEMS | | GAAUACAGCCAACGCCUUUGGAG |
| | | RYNNDLEASYHPRSDGIGVDLGVKSLAIVSDGRIFENINRTSRARRLE | | UGUACAAGAACUUGUGAGUAGUC |
| | | RRLRREQRRLSRKYEMRKKKGGSTATASANIEKKKLSVQRLHRRLA | | UCAGGCUUGCCUGGGCAAAAGCA |
| | | NLRRNHENQVIHALVEQKPRFITVEDLNVTGMMKNRHLSKAVVQQ | | UACACGAUGAAUAAGGAAUUUUU |
| | | RFYTFREKLRRKAEIIGIEVRIADRFYPSSKTCHACGHVHSGLKLKDR | | CUAAGACAUGCACAUUUUUGUCU |
| | | TYVCPACGYKKDRDFNAALNLRDTKNYRIA | | AUGUUUUUAGUAGCAG |
| 37 | RHM55346.1 | MIRSIRVQLLPNNKQKTKLFQFAGAARYAYNWALDKQMDNFREGR | 94 | AAAGAAGUCCAUGCAGAUAUGUA |
| | | KLQTDHELRKAFTLLRHSEENKWLLDISNNVTKQAIKDLCIAYKNFF | | CCGAUUCGUUAGUCGGGAACAGA |
| | | HKQKQPIYIKYSSKKITHYNRIGKSLTVYDMNGHPKFRSKKNGDFRF | | CGUGCCUUCUUGCGAUGGUAAGG |
| | | YQDNVKIQFTGTHVKLESLAGSRRKNRQRLNWVRLAERNRIPVRAK | | AGUGUCAUGUCAAAGCCUCCGGA |
| | | YTNPRIAFDGERWWLSVGIQTARKLKPLRGLSFVCKKRRYARSEGIG | | GCAUUAUAUCAAACGUGAGUAGC |
| | | IDLGIKDLAVLSDATKVGNINKTRAIRKLKKKRRRLQRQVSQKYQM | | UUGUCCUUCGGGAUGGCAAAAAC |
| | | NKKGDSYCKTSNLIKSEKRLLRINHRLANIRQNHVHQATTSIIKRKPS | | GAAUGCGAUGAACGAGGAAUGAA |
| | | FVCLEDLNVRGMMKNRYLSEKIQEQNLYEFRRQIEYKAHWAKIPVV | | ACGUAAAAGUCAGUUUUUCAUCA |
| | | IADRWYPSSKTCVICGHVKKELNLLERTYICPVCGNIIDRDFQAALNL | | AUAGACUAUGGCUUUUUAUAAGU |
| | | KRYGEAEQVKLANY | | UUUCAGUAACGG |
| 38 | RHQ99195. | MEKGFKVQIFPNKEQKELMFKSFGCARFAYNWALAKEKENYEKGG | 95 | ACUGUAAACUAAAUGUGUACCGA |
| | | KFISDMDLRKEFTQLKKKEEFKWLNEINNNVTKQAIKDLCKAYKNFF | | UACGUUAGUCGGGAAUUUAAGCC |
| | | KKKAEHPRFKSKKKSKKSFYNDVYKIKFDDYKVRIESIGWMDMAEN | | UUUGGAGAAGUAUACAAACUAUA |
| | | RIPRNCKYQNPKISFDGVKFWLSITCIVEDEENNLPKTEPIGIDMGI | | GUAGCUAUUCUUUGAAUGGUGAA |
| | | KTLMTCSNGMNFNRVSTKKEKKKLKRLQKKASRHYDKMMKTKSSDK | | AUAGGCUUUGGUGAAUUAGGAAA |
| | | SKNLLKLETLIRKQYQRISNVRLNNIHQATTKLVKLNPKAIVVEDLN | | UAGAGUUUGUAGUUUUUUAUAAG |
| | | VKGMIKNKYLSEKIADCSFYEIYRQLEYKCENKGMEFIVADRFYPSS | | UAAUUUAUAGAAAUUUAUAGAUU |
| | | KICSNCGHKKEKLSLSERTYRCECCGNVIDRDFNASLNLKNLAYN | | UUAUGUAACGG |
| 39 | RHR68682.1 | MVKAIKVMLIPNNIQKTKMFQYAGASRFAYNWALAREIENYEKGGK | 96 | CACUAAAACGUUAGUGCAAAUAU |
| | | FISDAELRKEFTKLRHSDEYAWLLSISNNVTKQAIKDACTAYKNFFK | | GUACGGGUACGUUAAUUCGGAAU |
| | | GLQKFPRFKPKKRSIPKFYQDNIKIQFSNTHVKFEGFSSSRKRNKQKL | | UUACGCCUAUGGAGAGUACAAGA |
| | | NWVRLAEHGRIPTDAKYMNPRISFDGLNWWISVCVEFPDCIEKLND | | ACUUGUGAGUAGAUAGAUAUUUA |
| | | DGIGIDLGIKDLAICSDGAKYKNINKSQKVKKLEKQKRRLQRSISRSY | | UAUCAUCAAAAGCAUACUCGUUG |
| | | EKNKKGESYCKTNNVIKKEKLLLKRNHRLTNIRKNYLNQTTSEIVSR | | AAGUAGGAAUGAAACAUAGAAGU |
| | | KPRFICIEDLNVSGMMKNRHLSKAVQNQGFFEFRKQLEYKCSDKGIQ | | UUAUAACUUUUUAUAAGUUUUCA |
| | | LIVADWFYPSSKLCSCCGNIKKDLKLSDRIYKCDCGNVIDRDFQASIN | | GUAACGG |
| | | LKAYGERVAS | | |
| 40 | RHR71212.1 | MWKLLKSFKTEINPSEEQKVKIHKTIGTCRFIYNFYLAHNKELYNNG | 97 | CUAAAACGCAAACGUAAGUAUGU |
| | | EKFMSSNRFRVWLNNEYLSEHPEYSWIKEAYSKAVTQSVNNGQTAF | | ACCGAAGGCUAUUUUCGGGAAUU |
| | | TRFFNHESAFPKFKKKGRSDVKMYFVKNNPKDCRCERHRINIPSLGW | | UACGACUGCGGAGUGUACAAGAA |
| | | VRIKEKGYIPTTKDGYVVKSGTVSMKADRYYVSVLVEISDNKIADNS | | CUUGUGAGUAGAUAUGAUUUCGG |
| | | NAGIGIDLGLKDFAIVSNGKTYKNINKSARLKKLEKQFIREQRCLSRK | | UCAAUCAAAAACAUACACGGUGA |
| | | YENLKKGEVTQRANIQKQKLKVQKLHHKIDNIRTDYINKTIAEIVKT | | AGCAGUAAGUAGAACUCGUGAGA |
| | | KPSYITIEDLNVNGMMKNRHLSKAVASQKFYEFRTKLQIKCNENGIE | | GUCUACGUUAUCUCGAUGUGAGU |
| | | LRIVDRWYPSSKTCHCCGAIKKDLKLSDRIFKCSCGYVEDRDLNAAL | | AUAUUUACACAUAUUUUGAGUGG |
| | | NLRDAITYEVA | | CAG |
| 41 | RGG64615.1 | MLTTKKVRLKLTPEQEIQFRKSCGVARWAYNYLLSEKQRVYDEYIS | 98 | UUCACUAUAAAGAAUUUUAUAAC |
| | | NGKTGKKTISEGEVRKYINNVLKPTTHQWLKEVGSNVMKQAVKDA | | UAUGUACCUAUCGUUACUGGGGA |
| | | DNAYKNFFNGLSKRPKFKSKKSSKQSFYVNYESLTRINGGFKGEKLG | | AUUUAAGUCUACAGAGUGUUAUA |
| | | FVKTSEPLPKLADGEKYANPRITFDGKYWYLSVVYNIEPKSVQLTNE | | ACAAAUGAGAGUAGCUUAGGCAA |
| | | SLGIDLGVKNLAICSNGVTYKNINKSKRVKALKRTLKREQRKLSRKI | | AAUCAGACACGAUGAAGUAGAAA |
| | | ECNIIGYANNRKPIFRTPLQGCKNIQKQIHYIKLINRKINSIRNNHL | | GUCUAAUUCGUGAGAAUAGACAU |
| | | HQATAEIVKTKPFQIVMETLNITGMMKNKHLAKAIAEEKLFEFKRQI | | GGUGUAGAUAUUUAUAAAUUAUA |
| | | KYKAEMYGIKVVEVPTFYPSSKTCSVCGCINTNLKLSDRVYHCDSCG | | AUAGAUUUGUACAUAUUUAUCGU |
| | | ITLDRDLNAAINLANYKVI | | AACGG |
| 42 | RHU26542.1 | MRKLLKSFKTEINPTVEQKIKINKTIGTCRYVYNFYLGHNKALYDKG | 99 | UGAACGCAAACGUAAGUAUGUAC |
| | | EKFMTGKSFSVWLNNEYVPDNPDKIWIKEAYSKAVKKSIEDGCTAFT | | UGCGGGCUAUCGCAGGAAUUUAC |
| | | RFFKHQSAFPNFKRKGKSDVKMYFVKNNPKDCRCERHRLNIPTLGW | | GACUGUGGAGUGUACAAGAACUU |
| | | VRMKEKGYIPTTKDGWKIKSGTVSIKADRYYVSVLVEIPDAKIANNS | | GUGAGUAGCGUAUUGUUUACAAU |
| | | NDGIGIDLGLKDLAIVSNGKTYKNINKSARVKKLEKKLRREQRGLSR | | CGCCAAAGCAUACACAAUGAAGC |
| | | KYENSKKGESTQKNIQKQKLKVQRLHHKIDNIRTDYINKSIAEIVKTK | | AGUAAGAAGUAUCCGCAAGGACU |
| | | PSYITIEDLNVSGMMKNRHLSKAVASQKFYEFRTKLKAKCDENGIEL | | UCAAUUUCUCCAUGUGUUUGAGU |
| | | RVVDRWYPSSKVCHCCGAIKKDLKLSDRIYRCDCGYVEDRDFNAAL | | AUAUUUAAACAUAUUUUGAGUGG |
| | | NLRDALTYEVA | | CAG |
| 43 | RGH37793.1 | MWKLLKSFKTEINPSEEQKVKIHKTIGTCRFIYNFYLAHNKELYDNG | 100 | CUAAAACGCAAACGUAAGUAUGU |
| | | EKFMSSNKFRVWLNHEYLSEHPEYSWIKEVYSKAVTQAVNNGQTAF | | ACCGAAGGCUAUUUUCGGGAAUU |
| | | ARFFNHESAFPNFKKKGKSDVKMYFVKNNPKDCRCERHRINIPSLG | | UACGACUAUGGAGUGUACAAGAA |
| | | WVRIKEKGYIPTTKDGYVIKSGTVSMKADRYYVSVLVEILNNKIANN | | CUUGCGAGUAGAUAUGAUUUCGG |
| | | SNAGIGIDLGLKDFAIVSNGKTYKNINKSARLKKLEKQLIREQRSLSR | | UCAAUCCAAAGCAUACACGAUGA |
| | | KYENLKKRESTQKTNIQKQRLKVQKLHHRIDNIRTDYINKTIAEIVKT | | AAUAGUAAGUAAUGUUCGUGAGA |
| | | KPSYITIEDLNVKGMMKNRHLSKAVASQKFYEFKTKLQTKCNENGIE | | ACUUACAUUAUCUCGAUGUGAGC |
| | | LRVVDRWYPSSKTCHCCGAVKKDLKLSDRMFKCSCGYVEDRDFNA | | AUAUUUAAUCACAUUUUGAGUGG |
| | | ALNLRDAITYEVA | | CAG |
| 44 | GUT_ | MIKSIKVRLNPNNKQSTKLFQYAGCARFAYNWAISREQDNYKQGNK | 101 | UACGUUAGUCAGGAAUUUAUGCC |
| | GENOME | FLSDSELRKEFTQLKKQSEYQWLNEVSNNVTKQAIKDACNTYKRFF | | UAUGGAGUGUACAAGAACUUGCG |
| | 08471128_ | KGQCKYPKFKSKKHSTPSFYQDNIKIQFTDTHVKVESFSMSKKQNKQ | | AGUAGAUUAUGAGAAAUCAUGUC |
| | 1717_ | KLNWIKLCEKGRIPTGCKYMNPRFTYDGLYWYVSIGIEVNDNTTLPS | | AAAAGCAUAUACGAUGAAGUAGG |
| | 2925_+ | NEGIGIDLGIKHLAICSDGNTYKNINKTQTVKKLEKKKRRLQRSISRK | | AAUGAAACAUAAAGGUUUAUAAC |
| | | YEKNKKGANYCKTSNIIKREKELLKLNHRLTNVRQNYLHQTTSEIVK | | UUUUUAUAAGUUUUCAGUAACGG |
| | | REPSFICIEDLNVSGMMKNKHLSKAVQQQGFYEFRRQIEYKAMWNNI | | |
| | | PVVIADRFFPSSKLCSCCGSIKKDLKLSDRIYKCECGNVIDRDYQAAL | | |
| | | NLKQYGENVLKQQSVAYHFQVITDMY | | |
| 45 | GUT_ | MLIPNNVQKTKLFQYAGASRFAYNWALAREKETYEKGGKFISDSEL | 102 | GACUGAAACGUUAAUGCAAAUAU |
| | GENOME | RKEFTRLRHSEEYAWLLNISNNVTKQAIKDACTAYQNFFKGLQKFPR | | GUACGGAUACGUUAGUCCGGAAU |
| | 11227339_ | FKSRKRSMPKFYQDNVKIQFSNTHVKFEGFSSSRKANKQKRNWVRL | | UUACGCCUAUGGAGAGUACAAGA |
| | 14283_ | AEHGRIPTDVKYRNPRISFDGLNWWISVCVEFPDSKETLNEDGVGID | | ACUUGUGAGUAGAUAGAUAUUUA |
| | 15425_+ | LGIKDLAVCSDAVKYKNINKSQKVKKLEKQKRRLQRSISRSYEKNK | | UAUCAUCAAAAGCAUACUCGUUG |
| | | KGESYCKTNNVIKKEKLLLKRNHRLTNIRKNYLNQTISEIVNRKPRF | | AAGUAGGAAUGAAACAUAAAAGU |
| | | ICIEDLNVRGMMKNRHLSKAVQEQGFFLFRKQLEYKCSDKGVQLIVA | | UUAUAACUUUUUAUAAGUUUUCA |
| | | DRFYPSSKLCSCCGNIKKDLKLSDRVYRCECGNRIDRDFQASINLKA | | GUAACGG |
| | | YGEQVAS | | |
| 46 | SRR735 | MSSNQFRVWLNNEYISKHPECFWIKEAYSKAVTQAVNNGQAAFTRF | 103 | AUAAACGCAAACGUAGGUAUGUA |
| | 2028_38 | FKHQSSFPRFKKKNRSDVKMYFVKNNPKDCRCQRHRINIPSLGWVRI | | CCGAAGGCUAUUUCGGGAAUUUA |
| | 0524_48 | KEKGYIPTTKDGYVIKSGHVSIKAGRYYVSALVEIPNNKIANSSNEGI | | CGACUAUGGAGUGUACAAGAACU |
| | 115_816 | GVDLGIKDFAVVSNGKVYKNINRSARLKKLEKQLIREQRSLSRRYEN | | UGUGAGUAGAUAUGAUUUCGGUC |
| | 1829_− | LKKGESTQKANIQKQRLKVQKLHHRIDNIRTDHINKTIAEIVKTKPSY | | AAUCCAAAGCAUACACGAUGAAG |
| | | ITIEDLNISGMMKNRYLSKAVASQKFYEFRIKLQAKCNENGIELRVV | | UAGUAAGUAGAACUCGUGAGAGU |
| | | DRWYPSSKTCHCCGAIKKDLKLSDRIFKCDCGYTEDRDLNAALNLR | | CUACGUUAUCUCGAUGUGAGUAU |
| | | DAMTYEVA | | AUUUACACAUAUUUUGAGUGGCA |
| | | | | G |
| 47 | GUT_ | MLKSFKTEIDPTPEQKVKINKTIGTCRYVYNVYLSHNKALYDKGEKF | 104 | AUAAACGCAAACAUAAGUAUGUA |
| | GENOME | MTGKSFSVWLNNEYIPNNPDKAWIKEVSSKAIKKSIEDGCTAFRRFF | | CCGAAGGCUAUUUCGGGAAUUAA |
| | 129951100_ | KHQSSFPSCKKKGRSDVKMYFVKNNPKDCKCERHRISIPTLGWMRM | | CGACUGUGGAGUGUACAAGAACU |
| | 5873_ | KEKGYIPTTKDGYAIKSGTVSMKAGRYYVSALVEVPNVNVRNNTND | | UGUGAGUAGCGUAUUUUUUAUUU |
| | 7024_+ | GVGIDLGLKDLAIVSNGKIYKNINKSARLKKFEKRLRREQRCLSRKY | | CUUGCAAUCGCCAAAACAUACAC |
| | | DNRKKGESTQKNIQKQRLKVQRLHHKIENIRTDHINKTIAEIVKTKPS | | AAUGAAGCAGUAAGAAGAAUCCG |
| | | YITIEDLNVSGMMKNRHLSKAVASQKFYEFKIKLKAKCEDNGIELRV | | UGAGGACUUCAAUUUCUCGAUGU |
| | | VDRFYPSSKLCHCCGRIKKDLKLSDRIYKCECGYIEDRDYNAALNLR | | GUUUGUGUAUAUUUAAUCACAUU |
| | | DAVTYEVA | | UUGAGUGGCAG |
| 48 | WP_ | MTGKSFSVWLNNEYIPNNPEKVWIKEAYSKAVKKSIEDGYTAFTRFF | 105 | UGAAAGCAAACGUAAGUAUGUAC |
| | 005609868.1 | KHQSAFPNFKKKGKSDVKMYFVKNNPKDCRCERHRLNIPTLGWVRI | | UGCGGGCUAUCGCAGGAAUUUAC |
| | | KEKGYIPTTKDGWKIKSGTLSIKAGKYYVSVLVEIPDAKIANNSNDGI | | GACUGUGGAGUGUACAAGAACUU |
| | | GIDLGLKDLAVVSNGKTYKNINKSARVKKLEKKLRREQRCLSRKYE | | GUGAGUAGCGAAUUGUUUACAAU |
| | | NIKEGESTQKNIQKQKLKVQRLHHKIDNIRTDYINKSIAKIVKTKPSYI | | CGCCAAAGCAUACACAUUGAAGC |
| | | TIEDLNVSGMMKNRHLSKAVASQKFYEFRTKLKAKCDENGIELRVV | | AGUAAGAAGUAUCCGCAAGGACU |
| | | DRWYPSSKICHCCGAIKKDLKLSDRIYRCDCGYIEDRDLNAALNLRD | | UCAAUUUCUCGAUGCGUUUGAGU |
| | | ASTYEIA | | AUAUUUCAACACAUUUUGAGUGG |
| | | | | CAGA |
| 49 | WP_ | MLKSFKTEISPTIEQKIKINKTIGTCRYVYNFYLDHNKTLYDNGEKFM | 106 | UCAACGCAAACGUAAGUAUGUAC |
| | 012741946.1 | TGKSFSVWLNNEYIPNNPDKIWIKEAYSKAVKKSIEDGCTAFTRFFK | | UGCGGGCUAUCGCAGGAAUUUAC |
| | | HQSAFPNFKKKGKSDVKMYFVKNNTKDCRCERHRLNIPTLGWVRIK | | GACUGUGGAGUGUACAAGAACUU |
| | | EKGYIPTTKDGWKIKSGTVSVKADRYYVSVLVEIPDVKIANNSNGGI | | GUGAGUAGCGUAUUGUUUACAAU |
| | | GIDLGLKDLAIVSNGKTYKNINKSTRIKKLEKKLRREQRCLSRKYENL | | CGCCAAAGCAUACACAAUGAAGC |
| | | KKGESTQKNIQKQKLKVQRLHHKIDNIRTDYINKSIAEIVKTKPSYIT | | AGUAAGAAGUAUCCGCAAGGACU |
| | | IENLNVSGMMKNRHLSKAVASQKFYEFRTKLKAKCDENGIELRVVD | | UUAAUUUCUCGAUGUGUUUGAGA |
| | | RWYPSSKICHCCGAIKKDLKLSDRIYHCDCGYVEDRDFNAALNLKD | | AUAUUUAAACACAUUUUGAGUGG |
| | | ALTYEVA | | CAGA |
| 50 | BBH26708.1 | MLKSFRTEIHPTPEQKVKINKIIGTCRYIYNFYLSNNKALHDQGEAFM | 107 | ACAAGCAAAUGUAAGUAUGUACC |
| | | SGKAFSVWLNNEYIPDNPDKAWIKEVYSKAVKKSIEDGCLAFTRFFK | | GAUGGCUUAGUCGGGAAUUUACG |
| | | HQSGFPKFKKKGKSDVKMYFVKNNPKDCICERHRIKIPALGWVRLK | | ACUGUGGAGUGUACAAGAACUUG |
| | | EKGYIPTTKDGWKIKSGTVSMKADRYYVSVVVEIPDTKPFCDSGEGI | | UGAGUAGUAUGUCACUUGUGACU |
| | | GIDLGLKDFAIVSNGKTYKNINKSAKLKKLEKQLRRAQRCLSRKYEN | | ACAAAAGCAUACACGAAGAAGCA |
| | | LKKGESTQKNIQKQRLKVQKLYQRIDNIRTDYMNKTIAEIVKTKPSYI | | GUAAGUAAUGUUCGUGAGAACUU |
| | | TIEDLNVKGMMKNRHLSKAVASQKFYEFRTKLKNKCDENGIELRVV | | ACAUAUCUCGAUGUGUUUGAGUA |
| | | DRWYPSSKLCHCCGAIKKDLKLSDRIYKCNCGYIADRDYNASLNLR | | UAUUUAAACACAUUUUGAGUAGC |
| | | DAKTYKIA | | AGA |
| 51 | GUT_ | MLKSFKTEINPTEEQKVKIRKTIGTCRYIYNFYLAHNKELYDKGEKF | 108 | AUAAAACGCAAACGUAGGUAUGU |
| | GENOME | MSGKSFSVWLNNEYLPQNPDKLWIKEVSSKSVKRSIENGCIAFTRFF | | ACCGAAGGCUGUUUCGGGAAUUU |
| | 2398042_ | KHQSAFPNFKKKGKSDVKMYFVKNNPKDCRCERHRINIPSLGWVRI | | ACGACUAUGGAGUGUACAAGAAC |
| | 85784_ | KEKGYILTTKDGYAIKSGHVSIKADRYYVSVLVEIPNNKIANNSNEGI | | UUGUGAGUAGAUAUGAUUUCGGU |
| | 86938_+ | GIDLGLKDFAIVSNGKTYKNINKSAKLKKLEKQLIKEQRSLSRKYENL | | CAAUCCAAAGCAUACACGAUGAA |
| | | KKGESTQKTNIQKQRLKVQKLHHRIDNIRTDYINKTIAEIVKTKPSYI | | GUAGUAAGUAGAACUCGUGAGAG |
| | | TIEDLNISGMMKNKHLSKAVASQKFYEFRIKLQAKCKENGIELRVVDR | | UCUACGUUAUCUCGAUGUGAGUA |
| | | WFPSSKTCHCCGAVKKDLKLSDRIFKCSCGYVEDRDFNAALNLRDA | | UAUUUAAUCACAUUUUGAGUGGC |
| | | ATYEVA | | AG |
| 52 | GUT_ | MLRSFKTEINPSEEQKVKIHKTIGTCRFIYNFYLAHNKELYDNGEKFM | 109 | AUAAACGCAAACGUAGGUAUGUA |
| | GENOME | SSNRFRVWLNNEYLSEHPEYLWIKEAYSKAVTQSVNNGQTAFTRFF | | CCGAAGGCUAUUUCGGGAAUUUA |
| | 04782036_ | NHESAFPKFKKKGRSDVKMYFVKNNPKDCRCERHRINIPSLGWVRIK | | CGACUGUGGAGUGUACAAGAACU |
| | 4781632_− | EKGYIPTTKDGYVIKSGTVSIKADRYYVSVLVELPDNKTADNSNEGI | | UGUGAGUAGAUAUGAUUUCGGUC |
| | | GIDLGLKDFATVSNGKTYKNINKSAKLKKLEKQLIREQRSLSRKYEN | | AAUCCAAAGCAUACACGAUAAAG |
| | | LQKGESTQRANIQKQKLKVQKLHHRIDNIRTDYINKTIAEIVKTKPSY | | CAGUAAGUAGAACUCGUGAGGGU |
| | | ITIEDLNVKGMMKNRHLSKAVASQKFYEFRIKLQAKCTENGIELRVA | | CUACAUUAUCUCGAUGUGAGUAU |
| | | DRWYPSSKTCHCCKNIRKDLKLSDRIFKCDCGYIEDRDLNAALNLRD | | AUUUAAUCACAUUUUGAGUGGCA |
| | | AMTYKVA* | | G |
| 53 | GUT_ | MRKLLKSFKTEINPTEEQKVKIRKTIGTCRFIYNFYLAHNKELHKNGE | 110 | AUAAACGCAAACGUAGGUAUGUA |
| | GENOME | KFMSSNKFRVWLNNEYLPNHPEYSWIKEAYSKAVTQAVNNGQTAF | | CCGAAGGCUAUUUCGGGAAUUUA |
| | 0012134_ | TRFFNHKSSFPNFKKKGKSDVKMYFVKNNPKDCRCERHRINIPSLGW | | CGACUAUGGAGUGUACAAGAACU |
| | 160075_ | VRIKEKGYIPTTKDGYVIKSGHVSIKADRYYVSVLIEIPNVKIANNSN | | UGUGAGUAGAUAUGAUUUCGGUC |
| | 16123 | AGIGIDLGLKDFAIVSNGKTYKNINKSAKLKKLEKQLIREQRSLSRKY | | AAUCCAAAGCAUACACGAUGAAA |
| | 8_+ | ENLKKGESAQKPNIQKQRLKVQKLHHRIDNIRTDYINKTIAEIVKTKP | | UAGUAAGUAAUGUUCGUGAGAAC |
| | | SYITIEDLNISGMMKNKHLSKAVASQKFNEFRTKLQAKCNENGIELR | | UUACAUUAUCUCGAUGUGAGUAU |
| | | VVDRWFPSSKTCHCCKNIKKDLKLSDRIFKCDCGYIEDRDFNAALNL | | AUUUAAUCAUAUUUUGAGUGGCA |
| | | RDATTYEVA | | G |
| 54 | GUT_ | MLKSFKTEINPTEEQKVKIRKTIGTCRYIYNFYLAHNKELHDNGEKF | 111 | AUAAACGCAAACGUAGGUAUGUA |
| | GENOME | MSGKSFSVWLNNEYLPKNPDKLWIKEVSSKSVKRSIENGCVAFTRFF | | CCGUGGGCUUACACGGGAAUUAA |
| | 02284545_ | KHQSAFPNFKKKGKSDVKMYFVKNNPKDCRCERHRMNIPSLGWVR | | CGACUGUGGAGUGUACAAGAACU |
| | 3955_ | MKEKGYVPTTKDGYVIKSGHVSIKADRYYVSVLIEIPNSKTADNSNE | | UGUGAGUAGAUAUGAUUUUGGUC |
| | 5109_+ | GIGIDLGLKDFAIVSNGKTYKNINKSAKLKKLEKQLIREQRSLSRKYE | | AAUCCAAAGCAUACACGAUGAAA |
| | | NLKEGESTQRANIQKQKLKVQKVHHKIDDIRTDYINKTIAEMVKTKP | | CAGUAAGUAAAACUUGUGAGAGU |
| | | SYITIEDLNVSGMMKNKHLSKAVASQKFYEFRTKLQAKCKENGIELR | | UUACAUUAUCUCGAUGUGAGUAU |
| | | VVDRWYPSSKTCHCCGAIRKDLKLSDRIFECDCGYIEDRDFNAALNL | | AUUUACACACAUUUUGAGUGGCA |
| | | RDATTYEVA | | G |
| 55 | GUT_ | MLKSFKTEINPTEEQKVKIRKTIGTCRYIYNFYLAHNKELHDNGEKF | 112 | AUAAACGCAAACGUAGGUAUGUA |
| | GENOME | MSGKSFSVWLNNEYLPKNPDKLWIKEVSSKSVKRSIENGCVAFTRFF | | CCGUGGGCUUACACGGGAAUUAA |
| | 112567131_ | KHQSAFPNFKKKGKSDVKMYFVKNNPKDCRCERHRMNIPSLGWVR | | CGACUGUGGAGUGUACAAGAACU |
| | 1855_ | MKEKGYVPTTKDGYVIKSGHVSIKADRYYVSVLIEIPNNKIANHSKE | | UGUGAGUAGAUAUGAUUUUGGUC |
| | 3009 | GIGIDLGLKDFAIVSNGKTYKNINKSAKLKKLEKQLIREQRSLSRKYE | | AAUCCAAAGCAUACACGAUGAAA |
| | | NLKKGESTQRANIQKQKLKVQKLHHKIDDIRTDYINKTIAEMVKTKP | | CAGUAAGUAAAACUUGUGAGAGU |
| | | SYITIEDLNVSGMMKNKHLSKAVASQKFYEFRTKLQAKCKESGIELR | | UUACAUUAUCUCGAUGUGAGUAU |
| | | VVDRWYPSSKTCHCCGAIKKNLKLSDRIFQCDCGYIEDRDFNAALNL | | AUUUACAUACAUUUUGAGUGGCA |
| | | RDATTYEVA | | G |
| 56 | GUT_ | MLKSFKTEINPTEEQKVKIRKTIGTCRYIYNFYLAHNKELHDNGEKFK | 113 | GGACGCAAAUGUAAGUAUGUACC |
| | GENOME | SGKSFSVWLNNEYLPQNPDKLWIKEVSSKSVKHSIENGCVAFTRFFK | | GAAGGCUAUUUCGGGAAUUAACG |
| | 03448415_ | HQSAFPNFKKKGRSDVKMYFVKNNPKDCRCERHRVNIPSLGWVRIK | | ACUGUGGAGUGUACAUGGAUCUA |
| | 4023_ | EKGYIPTTKEGYIIKSGHVSMKAGRYYVSALIEIPDNKTADLSNEGIGI | | CGAGUAGAUAUGAUUUCGGUCAA |
| | 5177_− | DLGLKDFAIVSNGKTYKNINKSARLKKLEKRLVREQRCLSRKYENLK | | UCAAAAGUAUACACAAGGAAGCA |
| | | KGESTQRANIQKQKLKVQKLHHKIDNIRTDYINKTIAEIVKTKPSYITI | | GUAAGUAAGAUUCGUGAGAACUU |
| | | EDLNVSGMMKNKHLSRAVASQKFYEFRIKLKAKSRENGIELRVADR | | ACAUUAUCUCGAUGUGAUAAGUA |
| | | WYPSSKICHCCGMIKKDLKLSDRIFRCHCGYVEDRDFNAALNLRGV | | UAUUUAAUCACAUUUUGAGUGGC |
| | | KTYKIA | | AG |
| 57 | GUT_ | MRTLLKSFKTEINPTEEQKVKIRKTIGTCRYIYNFYLAHNKELHENGK | 114 | GGACGCAAAUGUAAGUAUGUACC |
| | GENOME | KFMSGKRFSVWLNNEYLPKNPDKLWIKEVSSKSVKRSIENGCVAFTR | | GAAGGCUAUUUCGGGAAUUAACG |
| | 04053731_ | FFKHQSDFPNFKKKGKSNVKMYFVKNNPKDCRCERHRINIPSLGWV | | ACUGUGGAGUGUACAUGGAUCUA |
| | 7690_ | RIKEKGYVPTTKDGYVIKSGHVSIKADRYYVSVLVELPDNKTADNSN | | CGAGUAGAUAUGAUUUCGGUCAA |
| | 8853_+ | EGIGIDLGLKDFAIVSNGKTYKNINKSTKLKKLEKQLIREQRSLSRKY | | UCAAAAGUAUACACAAGGAAGCA |
| | | ENLKKGESTQRANIQKQKLKVQKLHHKIDNIRTDYINKTIAEIVKTKP | | GUAAGUAAGAUUCGUGAGAACUU |
| | | SYITIEDLNVSGMMKNKHLSRAVASQKFYEFRIKLKAKSRENGIELR | | ACAUUAUCUCGAUGUGAUAAGUA |
| | | VADRWYPSSKICHCCGMIKKDLKLSDRIFRCHCGYVEDRDFNAALN | | UAUUUAAUCACAUUUUGAGUGGC |
| | | LRGVKTYKIA | | AG |

For RNA sequences, the “U” in a sequence (such as any one of SEQ ID NOs: 58-114) may be shown as “T” in the sequence listing.

The sequences of the proteins of additional 42 RAD (IS607 TnpB) systems and corresponding RNAs are described below. For RNA sequences, the “U” in the following sequences may be shown as “T” in the sequence listing.

No. 58
(Numbering continues sequentially after Table 2):
> ERR3307045_63958_30277_4897_6000_−
(SEQ ID NO: 150)
MVYKIEISPTQEQKSIIFRTFDATRLVYNYFIEVNKARHENGYPYMGNLE
FSKTFNNYMSRLEWVRDKSLKISSKAVKEAMKDADKAYQRFFKGISRYPR
FKSRREPRKSYYLCGSIHVKRHAVRLPNLGWVLLKEKNYIPAGSGVVSCR
LIYEGGRFYVAILTQDNNSVNNQLSNDGLGVDVGIRFLATDSDKDVYPNI
NKSSRVKHLKKRLKREQRKLVRRNKKSLRREKTRQIINRIHYKISCICID
HKNKVVSALVKKKPSYITVETLNIKGMLKNRHLSRVIHEQGLHDFMTRLA
NKARQYGIELRRVSQYFASSKLCSTCGAKNDPKHKTFWVCTSCGTRHDRD
INASLNLLNATQYTIDK*
Associated RNA sequence
(SEQ ID NO: 192)
UAAAACCAUAGUUAUAAGGACUUCACAUGACCUAAAACAUUAUAGUACAA
UGAAUAAUGAGACCAAUUAACCCGUUAUGUCUCCAAAAACUACCGUUGGU
UCUACCGUGGGUUGGCAGGUAAGUUACGCACGUGGACUGUUACAUCAAAC
CGUAGUAGACACUCCUUGCAGUUUCGAGACGGGACAGGAAGAAGCGUGAA
AAAGCUCAACCAGAUAAUGAUAAUAUGCGUAUGGUUGAGUUACUGGCAUG
GAACAG
No. 59:
> GUT_GENOME151180_45_9178_10326_+
(SEQ ID NO: 151)
MIRGYRIKIFPNEEQKKMIHKSFGCSRFIYNWCIDRISENYNETKKTLSS
IELQKEIVILKKQIEYSWLNEVSANMLKQVTIDCSNAYKRWFKLIKQNNN
IKGKPRYKSRKSKQNCPTRTDRMTFNGRYVHLEKIGVVKLSKVKIDLDGK
LMNARLNFDGIDYWLSFSVEYKDVQLEEKPKAEPIGIDLGLKTLVYCSNG
NIYKKPHTKMIDKKIKHLQRRISKIYQPMIDYCKETRTKFSTLKKSNNLT
KLEKELRKYQIRKTNILDSNIHRITSDLIKINPEKIVIEDLNVKGMMSNH
KLARSIQSSKFYEIRRQLMYKCKNNNIELIVADRFYPSSKTCSSCGKIKD
DLKLKDRVYRCPQCGAVIDRDLNAAINLSRIS*
Associated RNA sequence
(SEQ ID NO: 193)
UUUAUAUAAAAUAGUGAGACUGCUCUCACGAAUUUAAGCCUAUGGAGAGU
UAAAACCACUUGAGUAGUUGCGACGAAAAAGAAUUCGAAGAAGUAGGAAA
UAUUAGAAUAUACAACUUUGUAUAAGUUUAAUAUAGCAG
No. 60:
> ERR2017626_1022590_12863_8034_9173_+
(SEQ ID NO: 152)
MNDVIRSYKIKLYPTKEQEQLMHKHIGCCRYIWNYMINLQCSNYESGNKY
MSAFTMINLLKPLKNDGEHKWLCEVSNQSLATVCQDVDKAYQRLFKKQAK
KPKFKSKKRSKQAFPVRGTIWFKDNVVTIEKLGKVKFKSDFEFPQGRGNK
FTNTRISEINGKWMLSFGMECENQAPELNDYSMGIDLGVKDLAIAECNGQ
IVFHNINKSKKMRALNKKLKHLQRAVSRKYEANKQGKKYIKTKNIEREEQ
KVRNIHNRISNIRNNYIHQCTHALISMLPKRVVVEDLNVKGMMKNKYLSR
AIQEQCFGEFLRQMQYKCEWNGIEFVMADRFYPSSKTCSCCGVVKTNLKL
SDRIYRCESCGLVIDRDYNAAINLSRYVA*
Associated RNA sequence
(SEQ ID NO: 194)
AUAGAGAGGCUAUAACCUCAAGGUGUCGUUGCACCUUUAAGCUGUGGAGU
GUUAUAUAAACUUGAGUAGGAUUACCCAAAAGAGGACGCUGAGAAGCAGU
AAGUUAGAUUGGCUAACACAACGGAAAAGGAAUGAUUUAUAUGGUUAAAA
UCUCGCAGGAGGAAGCUUUUAAGAUUAGAAAGAAGUUUCCGGGAACGCAU
AUAACGG
No. 61:
> GUT_GENOME142162_18_64466_65659_−
(SEQ ID NO: 153)
MIKAIKVRLYPTKKQEVLMFKSAGISRFSYNWGLAFLNKYYEENNKFLQI
DKLRKEFTRLRNNNEFSWLKEVSSEIPQQALKDLGESFKKFFKKESLYPR
FKKKGKCEVSFFHLNNKFVVKENYIKLEKIGYVKMKDEGRLPIGNYKKDK
IKVLNPRVKFNGKYWYLSLAMEVEEKTNIKLTNESVGVDLGIKDLAIVSN
IDEPFKNINKSKEVKRLNKKLRKLQRQVSRKYDMLKSGNTFQKGEKLVKT
KNIIKVEKQIKLVYKKLSDIRLNHIHQTTNTIVKTKPCRIVVEDLNVRGL
LKNKHLSKSISEQCFNKFINILEYKCKWNKIEFVKADRFYPSSKMCSCCG
TIKKDLKLKDRTYVCSNEKCNLVIDRDKNASINLANYKSNYKCGYLN*
Associated RNA sequence
(SEQ ID NO: 195)
CAUAGAAAAUCAAAUAAAAAGAUUGUGCUAAUAUGUAGACAUUGACGAGG
UCGAAUUUAAGCCUUUGGACUGCUACAUUAAACUAGAGUAGAAAUUGAAU
UUCAAAAUAGAGCAGGAGGAAAAAGGAAAUAAAACUAUAAAAAAUAUAAA
GUAUUUAUAUAAAUUUAUAGAGUUUAUACGUCAG
No. 62:
> NC 013216_1_421908_422903_+
(SEQ ID NO: 154)
MYAGTCVTSIFKLIKKGISKAYLLCLQMILTNGLTMSILNSKDLSGLRVY
HLKHVRSLLSMRRGLLKDSLKNWVNSIPTIGWMRLKEFGYIPYGVTVKSG
TVSYKAGRYYVSVIVDIQLVGVEKKCSEGIGVDLGVKVLATVSSGIKFKN
INKTSKVKKLERRLKREQRKLSRKYHMRKQRGGTATALRCANIDKQRLKV
QKLHQRLTNIQTDYVNKTVNELVKTKPEYITVENLNVSGMMKNRHLSKAI
AQQKFYEFNIKLKSKTQWHNIELRVVDRFYPSSKICSCCGHIKWDLKLSD
RIYICDNCGFVEDRDVNASINLRNAKQYKIS*
Associated RNA sequence
(SEQ ID NO: 196)
UUAACAAAGUUCUUGUAUAUGUACCGAUGGCUAGUCGGGAAUUUAAGACU
GUGGAGUGUUACAUCAAAUGCGAGUAGCUUUGUGGCAAAAGCAGACAUUA
UGAAGCAGUAAAACUCUCUUGAUAUAGAUUUUUGUCUAUAUUUAAAGUAG
CAG
No. 63:
> NC_004557_1_WP_035125047.1
(SEQ ID NO: 155)
MIKALNIRLYPTQEQAALMYRHIGCMRFVYNWALARQIDSYKHSGKKLSI
AELGKELTVLKNTEGYEWLYEVSNATLKEGIRDLDKAYGNFFKGRGFPKF
KSKKKSEPKFYSRYEKAYFKDNLVNLEKIGKVKCKVDYDIDLTTIKKFKN
PRVKFNGRVWVLSVGIEIPAKKVKLNDFSLGVDLGIKQLAITNIDNLDTL
NINKTNKVKKLNKKLKKLQRQCSKKYIINKKGGSYQKTKNIGKLELKIKK
LHSKLKNIRLNHTHQTTSKMVKAKPYRVVMEDLKVSNMIKNKHLSKAISE
QGFNIFINQMKYKCQKYGIEFIQVSTFYPSSKTCSHCGTIKKDLKLSDRT
YKCDCGFTCDRDKNASYNLANYGLEISI*
Associated RNA sequence
(SEQ ID NO: 197)
AAAGAUAUUCUAAAUUGGGUAAUCGUUACAACCCAUUACCUUGCUAGGUA
ACAAAGCCUAUGGAGUGUUAUAGCAAACGAAAGUAGCUUAGGCAAAAUCG
GAUAUUAUGAAGUAGGAAAGAAACAAAAAUCUUUAAUUUAUAGAUUUUUA
UAGGUUUUUGGUAACGG
No. 64:
> GUT_GENOME096560_11_244135_245259_−
(SEQ ID NO: 156)
MMKAYKIKLNPNNKQRTLLERSAGVSRWAYNWALEQKKKRYEETGKTVSQ
ADLRKRLTKLKQTDDYQWLFQFSNNITKQAIKDSDEAFKRFFKGQARYPR
FKSKRKSKWSFYNDNHKVKRSDTHIRLEKIGWIRFAETGYLPIEFKPLSY
RVSREGLHWFVSITVEIPDSSDYSTPVNEPIGIDLGIKTLATVSDGSVYV
NINKKHEVRQMEKRLRRLQRQISRKYKMNKEGSRYRKTCNIIKTEKEIAR
LQTRLNHIRTNYIHQMTSEIVRTKPSHIVMEDLNVRGMMKNKHLADHIAK
CHFREIRRQLTYKANQSGVSLVVADRWYPSSRICCECGVMDKTLKLSDRK
MKCECGNRIDRDMNASINLSRLAV*
Associated RNA sequence
(SEQ ID NO: 198)
UCACGAACAAGACACGGCUAAUAGGUACCGUGAGUUACACGGGAAUGAAC
GCCCUCGGAGUGUUACAGCAAACGAGAGUAGUAUCUGAGACAGAUACAAA
AUCGGACACGGUGAACAGGGAAGAAAACAAAAAAUCUCUUUUACAGGUUU
UUAUAAGUUUUUCGUAACGG
No. 65:
> GUT_GENOME083533_37 11359_12597_−
(SEQ ID NO: 157)
MFRGRKIRLLPTKEQELLFWKSAGTARWAYNYFLAENEAAYRAYLKRGKT
GPRRISADDVRKYINNNLKKTTHQWLKEVSNNVMKMAVKKADYAVKKFLK
GLAGKPKFKTKHRTKPSFYVNYASLARKNGGFHGERLGFIRTAEPLPRLP
KGKRYSDPWISFDGKYWYLGLSYEVSASEAKELSGDTIGIDLGIKTLATC
YVSGTKKKMTFQNINRTKKVRRLEKKLRREQRKQSRKLVANTARHKSIQR
EGKTIGRKPIWKRPLHECLNLKRQRRKIALIHRRLANIRQNHVHQTTATI
VKTKPSRVVLENLNVKGLMKNKHLAKAIAGAKFYEFRRQMIYKCEEHGIQ
VIIADRFYPSSKTCSYCGYLKKNLKLNDRVYVCPSCGKAIDRDLNAAINL
ANYFQSDTACTA*
Associated RNA sequence
(SEQ ID NO: 199)
AACUUAGAGAAAUUCUCAGACAGGUACCUAUCGCUACUAGGGAACCGGCU
UUCCUAAAAAGAUUGUCUAAGCCUGCGGAGCGUUAUAUCAAACGUGAGUA
GCCAUCGAGCUUGCUUGAGGCCAAAACGGACGCGAUGAACCAGGAAGAAA
ACAUCGCAAGAGUUUUCGAAGUAUAACUCUGUAUGAUAAUUUAUACAGAU
UUAUCGUAGCGA
No. 66:
> NC_022878_1_WP_023518930.1
(SEQ ID NO: 158)
MSHLKIDKYNKEIEGDKDVSGVQNRNSSNRRTKKIINRTIGVARFVKNFY
IYHNQEVYKSGGKFVSGIDFSKWLNNDYIPNHPDKLWIKAVYAKSTKHAI
METEKAFRNFFQGKANFPKFKKKKHQDVSMYFVKNDKKTIIRCERHRIKV
PSLNWVKLNEKGYIPKHDDKHSIKSGTISQQANRYYLSVIVEQDCTAQVL
NDTPTEGVGIDLGLKDFAILSTGKTYKNINKSGKIRKIEKQLKRQQRKLS
RKYESEKKRVNEKQSKRGEATRKNIEKQVLKVQKLHKRLTDIRRDRVNKI
VLELVKTKPAYITIEDLNVKGMMKNRHLSKAVREQSFYAFRKKLADKCKV
STIELRLADRFYPSSKLCHECGHKKVALKLSERTYRCENCGYVADRDYNA
SMNLKDVKEYNVIA*
Associated RNA sequence
(SEQ ID NO: 200)
GCACGCAACUUUGUAUAUGUACCGAUGGCUUAGUCGGGAAUCAACGACUG
UGGAGAACGAGACAAACCGUAGUAGUGUCGAAGUGACGAGGCGGCGUUCG
GUGAAACAGUAAUUCUACCUACGUGAGUGGUAGAGAAAUCUCGAUAUAGG
UAUUUUUGUCUAUAUUUUGUGUAGCAG
No. 67:
> NZ_CP020815_1_WP_012574395.1
(SEQ ID NO: 159)
MIRTYKVMLLPNNKQKTKLFQCAGVARWAYNFALAQQQENDKQGGKFLSD
GELRKRLTQLKQTKEYSWLNQFSNNITKQAIKDAYQAYKNFFEGRAKFPK
FKTKKRTRPSFYQDTSKIKITDTHVKLEKLTPSKKKNRQKFNWIRLAERG
RIPHGAHIKYVNPRIVFDGLHWFLTVGVEEAEVKHGTYNEGIGIDLGVKS
FATVSNGMTFPNIHHMPQVKKLEKQMKRMQRKISRKYEMNKIQTERGEFR
YRKTENIKKLEFLVLKKRRRLKNTQLNYTHHITTTLVKTKPAYVVMEDLN
TSGMLKNRKLSKAIQQQTFHEFKRQMTYKCAWHGIELIVADRFYPSSKKC
SRCGHVKEKLSLSERTYRCEACGIEMDRDLNASINLKNYAK
Associated RNA sequence
(SEQ ID NO: 201)
AGCACAACAUGUACCCAUCCGUUAGUGGGGAAGUGAAGCCUUCGGAGCGC
CAAGGAAACGAGAGUAGCUUCGGCAAAAUCGGGCGCGAUGAACAAGGAAG
GAAACAUAGACUUUUAUAGGUUUCUUAUAAAGUCUUGUAAGUUUUCUGUA
ACGG
No. 68:
> NZ_CP024701_1_WP_099988772.1
(SEQ ID NO: 160)
MYYKALKIEIKLTEEQKIQVNKTIGTERFIYNEYIKYNQEQYELGNKFVS
ANDFSKYINNIYLPNNPDKKWIKDVSSKSVKQAMIYGEKAFKNFFKGLSS
FPVFKKKGKNELGAYFVKNNKTDFEFYRHKIKIPTLKFVRVKEYGYIPKN
ANIKSGTITKIADRYFLSLIIEIEDTVKATNTNSKGLGVDLGIKDTAICS
NGKVFKNINKTKKVKKLKKKLKREQRKMSRSVEYSKSKKIKLKECKNFNK
KKLKVQKLFYRLNCIRDDYNNKIVDEITRAKLKYIAIEDLKVSNMMKNKH
LSKAIQEQNFYAIRTKLINKCKERNIELRVVNTFYPSSKTCSCCGSIKKN
LKLNDRIYKCCNCGLEIDRDYNASINLEKAKIYKVIA*
Associated RNA sequence
(SEQ ID NO: 202)
CAAGUUAAAAUAUCUAUAUGUACCGAUGGCUAGUCGGGAAUUUACGACUA
UGGAGAGUACAAGGAACUAUGAGUAGAUACUAAGUAUUGAAAAUAUACUC
GUUGAAGUAGUAACAUUCUAGUGUAUUAAAUAUGUUUUAAUACAUUUAUA
GUAGCAG
No. 69:
> GUT_GENOME228548_94_18418_19641_+
(SEQ ID NO: 161)
MEFEEQPIILFKGGLKILKSYKTEIDPTEEQTKKIKKTIGTCRFIYNFYL
AKNKEIYEKSNQSMSAFSFMKWLNNEYLKENPDKLWIKEVSTVSIQKATQ
NATDAFRRFFKGQNRFPKFKKKNKQDVKMYFCRQGKNDSIKCERHRIKIP
TLGWVKLKEKGYIPTNPNTHIIRKGTVSYKAGRYYVSVLVEEMQPMKERL
NDFGIGIDLGVKEFATLSNGTIYQNINKTNRVKDLEKKLNRERRKWSRKY
KSYLRRKELDENCTKENLLKQQIKMQKVFHQLDNARTDYVNKCISEIVKT
KPSYITIEDLDVMRMMKNKHLSMEVQKQKFFEFRTKLADKCREYGIELRV
VDRWYPSSKTCHNCGCIKHNLKLSERVYKCPECGFKIDRDLNAALNLRDS
KIYKIIP*
Associated RNA sequence
(SEQ ID NO: 203)
CUAAUCUGUAAAUAUGUACCGAUGGUUUGUCGGGAAUUUACGACUGUGGA
GUAUACUAGAACUUGUGAGUAGCGUGUUAUUCGUAACCGCAAAAGCAUAU
ACGAAGAAGCAGUAAGAGGUAUCUGUAAAACCCUCAAUUCUCAAUACUUA
UGUAUUUUAGAGAAACAG
No. 70:
> NC_010001_1_WP_012201916.1
(SEQ ID NO: 162)
MKRLQRAYKVELKPTNQQIQKINQCLGVCRWLYNAYLAKNQSLYQDFKEG
KLNKKKAFMSANDFDKYINNDIKVLDDCKWINDCGSKARKKAICNAEFAY
KKFFKGEAQFPRFKKKGKSKVGLYFPKNNKTDFKIERHKINIPTLKWVRL
KEFGYIKANANVISGTVTEQAGKYYISIVCEVEEVKNSLPKTEPLGLDLG
IKDFAIFSNEIVKKNINKTSRVKKLEKKLKREQVRLSRKYECLKKQNKIR
KGETTRQNIRKQIVKVQRLQQRLANIRTDYINKCVSEIVGQKPSSITIED
LNVKGMMKNRHLSKAVAGQKFYEFRIKLTNKCKQNDIELRIVDRWYPSSK
LCSCCGNVKKDLKLSDRIYVCKECGLVIDRDYQASINLREAKTYMIA*
Associated RNA sequence
(SEQ ID NO: 204)
CCACGUUACUGUAAGUAUGUACCCAAGGCUAUUUGGGGAAUUUACGCCCU
CGGAGUGUUAUAGCAAAUGAGAGUAGUGUAUUAUAACAAUCACCAAAUCA
GACACGUUGAACAGGGAAUAAUCUCGAUAUGGGUAGAUUUGUCCAUAUUU
UGAGUGGCAG
No. 71:
> NC_010003_1_WP_081429131.1
(SEQ ID NO: 163)
MMRLLKSYKTEINPTLEQEQQIKKTLGVCRFIYNFYIAHNKEIYEKERRF
VSGMEFSVWLNNEFIPNSQEFNWIKDVSSKAVKQSIMNGERAFKKFFKGK
SKFPRFKKKKNNDVKAYFPRNNKTDWTIERHRVKIPTLGFVRLKEKGYIP
TNAKVGSGSVSFEAGRYYVSVLVEEDSLTAVEKGIKQDNISFSEGIGIDV
GIREFVVASNGIKKKNINKSKEIKKLEKKLKREQRRLSKKYENKKKGGTA
TNSAKNIDKQVLKVQKLHQKLKNKRHDYVNKVVCELVKTKPEYFTIEDLN
VKGMMKNRHLSKAIAKQNFHYFKIRLENKCKHLGIQLRIVSRFYPSSKIC
SHCGNIKKDLRLSDRVYICEECGSKIDRDLNAAINLKNAKEYAIAC*
Associated RNA sequence
(SEQ ID NO: 205)
AAUUGCUUUGCAUAUGUGUACCGAUGGCCAGUCGGGAAUUCACGCCUGUG
GAGUGUCAAACAAACCGUAGUAGAUAUAUAUCGAGGCGGGACACGUUGAA
GCAGGAAACUUCUGGAUAUAGGCAUGUUUGUCUAUGUUUUCAGUGGCAG
No. 72:
> GUT_GENOME249453_33_14687_15901_−
(SEQ ID NO: 164)
MVWSLVKYKLEREVRKMIKTIRVMLVPNNKQKAKLFQYANTARFAYNWAL
GREKENHENRGKFICEGDLRKEFTQLKKKEEYLWLKKISNNVPKQAIRDA
CTAYQNFFNGVSKAPRFKSKKHSIPKFYQDTKKIKFTDTHVKVEGFATSK
RHNKQKLNWIRLAEHCRIPTDCKHFNPRIKYDGLNWYMTVGIEYKDKESI
PSNEGIGIDLGIKDLAICSDKNKYKNINKIQKVKKLEKRKRRLQRCISRR
YEKNKNGECYCKTSNIKRKEKELLKVSRKLANIRLNYLHQITTEIIKRKP
SFICIEDLNVKGIMKNKHLSKAIQQQCFGEFRRQIEYKSKWNNTKVIIAD
RFFPSSRLCSCCNKIKKDLNLSDRIYRCECGNVIDRDYQAAINLKNYGEM
KISV*
Associated RNA sequence
(SEQ ID NO: 206)
AACACGAAAAAGUUAAUACAGAUGUGUACGAAUACGUUAGUUCGGAAUUU
ACGCCUAUGGAGUGUACAAGGAACUGUAAGUAGUAACUAAAUUUAUUUAG
UACAAAAGCAUACACGAUGAAAUAGGAAUGAAACAUAAAAGUUUAUAACU
UGUUAUAAGUUUUCAGUAACGG
No. 73:
> GUT_GENOME046003_7_13445_14386_−
(SEQ ID NO: 165)
MRYYLIGEFAEKIGKAIQTLRNWHKNNTFKPAYVTAAGTRYYSQEQLNNF
LGLKIKTKIKDKKALIEKVGWIKINEQIPSDIKYSNPRITYDNKYWYISV
GIEVNKKQENLTDISLGIDLAICSNGQVFKNINKTIKVRKLEKRLKQKQR
QISRKYEMNKIKKEGGVRCQFIKTKNIEKLENTTKLIHRKLANIRNNYLH
QVTTSIVKNKPYRIVIEDLNVSGMMKNKHLSDSVRKQCFYKFRQYLTYKT
ELYGIKLVIADRFYPSSKTCSQYSSIKKDLKLKDIVYRCPHCGAVIDRDY
NAALNLSMYKSNM*
Associated RNA sequence
(SEQ ID NO: 207)
GACACGUUGUAUCCAAAUUUAAGCCUUUGGAGAAUCAUAUCAAACGAAAG
UAGCUAUGGCAAAAUCGGAUUCUAUGAAGAAGGAAGUAAACAAAUUUAUA
UAUUGUUAUAGAUUGUUGGCAACGG
No. 74:
> GUT_GENOME043660_1_131655_132716_−
(SEQ ID NO: 166)
MYNYMLDLQKKRYEAGEKHLSAFSMIKLITPLKQQEGYEWLCRVSTTSMQ
RVCQDLANAYENFFSKRAKFPRFKSRKRSRASYPVRETVYFIDGKTQIEK
IGKVVYQTNYDLPQGRGHKFSNPRISNNNGKWILTVGIECESQAPELTDK
PMGIDVGVKDLAIVAFGEDQIVFHNINKSKRVRNLSRKKRKVQRTISRKY
RTNGSYSKTKGIEKYERILKTIHYKLANIRSNYIHQSTHKLVSMLPKVVC
VEDLNVTGMMKNRHLSRAIQEQCLGEFLRQMEYKCAWNGIELVKVDRFYP
SSRTCSCCGEIKEDLKLSDRTYVCPHCGLVIDRDYNAALNLMKYAASQTR
AAA*
Associated RNA sequence
(SEQ ID NO: 208)
GGGAAUCGUUGCACCCCUGAAUGCUGUGGAGUGUCAUACAAACCCGAGUA
GCUAAGGCCAAAGGGGACACAAUGAAGCAGUGAUUGGAAUUACCAAACGC
AACGGUACAACGUGCUGGAUGUAGAGGUCAUGGCUAAGAUUCUCUAUCGG
GAAGCUCGUGGCAUCUCCAGCAAGACAGAACAGGCCUGCGUCGGCUGGGU
GAUCUGUAACAG
No. 75:
> NZ_CP014176_1_WP_039636020.1
(SEQ ID NO: 167)
MINLIKALNIRLYPTKEQIILMYKHIGCMRFVYNWALSKQIENYKINNKK
LSVSELAKELTILKNTKGYEWMYEISNATLKESIRDLDKAYKNFFNGSSF
PKFKSKKKSNHVFYTRYDRIYFKEDFVNLEKIGKVKCRVDYDIDLATITK
FQNPRIHFNGRVWVLSVGIETSVEKAKLNDFSLGIDLGITQLAITNIDNL
DTKNINKTNSVKKLNKRLKRLQRQCSRKYEKNKEGGSYQKTKNIVRLEKK
IKKLHSKLKNIRLNHIHQSTSKMVKAKPFRVVMEDLKVSNMMKNKHLSKA
IAEQGFNKFINQMKYKCEKYGIEFIQVPILYPSSKTCSHCGYIKKDLKLS
DRIYKCSCGFSCDRDKNASYNLANYGLV*
Associated RNA sequence
(SEQ ID NO: 209)
UCAAUUAAAAGAUUAACUAAAUUGGGGAAUCGUUAUAACCCACUACCUUA
UUAUUAGGUAGCAAAGUCUAUGGAGUGUUAUAUCAAACGAAAGUAGCUUA
GGCGAAAUCGGACAUUAUGAAGUAGGAAAUAAACAAAGCUAUAAACUUAU
AUAAUCUUUUUAUAGAGUUUUUAGUAUUUUGGUAACGG
No. 76:
> NC_014614_1_WP_013360293.1
(SEQ ID NO: 168)
MIKSIKIRLKPTKEQEILMFKSVGVARFAYNWGLAKWEEMYKKGLKPSKA
KIRKEFNNSVKKSDDFKWLYEVSGQITSQAFTDLEYAYKNFFNKTSDKPK
FKTKKKSKKSFYVRYDALKFKDAKVNIEKIGKVKYSSNYNIPQLDNYVNP
RCHFDGKYWYLTFGFEHSENKAKLDYNLSIGIDLGIKELAYVSCFNKPIK
NINKSVKVKKLKKRLIRLQKQVSRKYEANKSGDRYNKTSNIIKLEKQIKL
LHRKLNNIRNNHLHQTTKMIVKLNPYRIVMEDLNVTGMMKNKHLSKAIAE
QKFYEFIRQIKYKCEFNNIEFLQVDRFYPSSKTCSSCSGIKQDLKLKDRI
YHCDKCGLTMDRDKNASINLANYKIV*
Associated RNA sequence
(SEQ ID NO: 210)
UUUUGAAAAAUUAUACUAUAUGUAUCCUGCGUUGUGGGAGAAUUUAAGUC
CUUGGAGAGUUACACCAAACUAAAGUAGAGUGAGAAAUCAUUCAAAAUAG
AACUCGAUGAAUAGGAAAUGUUAUUUGGUAACAAAUAAUGUAAGUAAAAU
UUAUAUAUUUAUAAGUUUUAUGCAACGG
No. 77:
> GUT_GENOME243333_13_9207_10436_+
(SEQ ID NO: 169)
MLRGKKIRLLPTPEQEILFWKSAGTSRWAYNYFLARNEEQYKTYLANGRK
GKKSISEGVIRKEINNQLKPITHQWLKEVGSNVMKQAVKEAGRAMKKFLS
GAAGRPKFKTKRRTTPRFYVNYESLTRKNGGFHGEKIGFVRTAEPLPKIP
KGCHYSNPWISFDGKYWYIGFGYEVPDRLNVELTGDSIGIDLGVKELAVC
YAFGERKKTVFKNINKTKEVHRLEKKLRREQRRQSRKFTANIDHYETVQR
NEKKTGRRPIWKRPIRECRNIERQRQRIILLHRRLKNIRENHIHQTTAAI
VKTKPSRIVMEDLNVKGMMKNRYLAKEIASEKFYEFRRQMEYKCEPYGIQ
LVFADRFYPSSKTCSHCGHIKKDLKMNNRTYICPACGHVIDRDLNAAINL
AKYFKSEIA*
Associated RNA sequence
(SEQ ID NO: 211)
CAUUUCGAGAAGCACUUAGAUAUGUACCUAUCGCUACUGGGGAAUUUAAG
CCUGUGGAGCGUUAUAUCAAACGAAAGUAGCUGCCUGCUUCGGCAGGGCA
AAAUCGAACGCAAUGAAACAGGAAGGAUGUUUGUGAAAACAUUCAAAGUG
UAAAUUUGUAUAGUAUAAAAUAGAUAUUUACAGAUUUAUCGUAGCGG
No. 78:
> GUT_GENOME228909_131_318_1547_−
(SEQ ID NO: 170)
MIVGKKIRLHPTSEQETLFWKSAGTARWAYNYFLIRQEEVYQEYLVNGQK
GRRGISEGTVRKEINRVLKPTTHAWLKDIGSNVMKQAVKTADKAWKKYLK
GLAWKPKFKSKKMTKPNFYVNYESLSRKNGGFHGEKIGFVRTAEPLPKLP
HKKHYLNPWISFDGKYWYLGFSYEVEAHRESLTGDHIGIDLGVKSLAVLY
SVDTGQSKVIKNINKSRKVLRLEKKLKRAERKRTRQLKSNVLRYEPIQRD
GKRPGRKPVWKRPVPECKNIEKQRRKIKLIYRKLKNIRVNHLHQATTAIV
KTKPSRVVLETLNIQWMMKNKHLAKAISKAKLYEFRRQIEYKCQKCGIEV
VFADKFFPSSKQCSSCGEIHHELTLSDRMFSCSCGLHIDRDLNAAINLAR
YTPSKIAHN*
Associated RNA sequence
(SEQ ID NO: 212)
GAGCAGUUUCGAUAUGUACCUAUCGCUGCUGGGGAAUUUAAGCCUGCAGA
GCAUUAUAUCAAACGAGAGUAGCUUGCAAUGCGUUGUGGCAAAAUCGGAU
GCGAUGAAGCAGGAAGAGUGCUCGUGAGAGCAGUCAACGUAUUGAUUUAU
GAUAUAUAUCGGUAAAUCAAUCGCAGCGG
No. 79:
> ref_RGS74140_1
44001_45231_+QRVW01000004_1
(SEQ ID NO: 171)
>MLRGKKIRLLPTPKQEVLFWKSAGTARWAYNYFLGENQAVYKAYLEHGK
VGPKRISEGAIRKHINNELKKTTHQWLKEVGSNVMKMAVKEADRALKRFL
KGIAGKPRFKTKHKTKPSFYVNYESLVRKNGGFHGEKIGFVRTRETLPKL
PKGNHYCNPWISFDGKYWYLSFSYEVPEQETEKLTGDSMGIDLGIKVLAT
CYASGERKATVFKSVNKTKEVRRLEKKLRREQRKQSRKILANTARYETVQ
RKGKKPGRKPIWKRPLRECQNIEKGRRKIALIHRRLANIRQNHIHQTTAA
IVKTKPSRIVMEDLNVKGMMKNKHLAKGIAGEKFYEFRRQMTYKCENNGI
QLVFADRFYPSSKTCSCCGHVKENLKLNDRKYVCQACGTTIDRDLNAAIN
LANYFQSETA
Associated RNA sequence
(SEQ ID NO: 213)
AAUACAGAGAAGCACUCAGAUAUGUACCUAUCGCUACUGGGGAAUUCAAG
CCUGUGGAGCGUUACAUCAAACGCGAGUAGCCAUCGAGCUUGCUCGGGGG
CAAAAACGGACGCAAUGAAACAGGAAGAAAAUAUCGUGAGAAUUUUCAUC
GUAUAAAUUUGUGCGAAUUAUAUUAUAUUUAUACAGAUUUAUCGUAGCGG
No. 80:
> GUT_GENOME029159_118_4969_5874_−
(SEQ ID NO: 172)
MPAQPIKTFSRVYNGTQGLRRKRNQCLNSIKIQFRDTHVKFEGFSFSRKA
NKQKLNWVKLAEHGRIPTDAKYRNPRVSFDGLNWWISVCVEFPDCRDRSN
HDGIGIDLGIKNLAICSDGNTYKNINKSQTVKKLEKCKRRLQRSVSRKYE
HNKKGGSYCKTNNIVKKEKLLLKVNHRLANIRKDYLNQTTSEIVNRKPRF
ICIEDLNVSGMIKNRHLSKAVQNQGFFEFRKQLEYKCNDRGIQLIVADRF
YPSSKRCSCCGKIKKDLRLSDRTYKCECGNVIDRDFQASLNLKAYGEQFA
S*
Associated RNA sequence
(SEQ ID NO: 214)
CACUUAAAUGUUAAUGCAAAUAUGUACGGAUGCGUUAAUUCGGAAUUUAC
GCCUAUGGAGAGUACAAGAACUUGUGAGUAGAUAGGUAUUUAUGCCAUCA
AAAGCAUACUCGUUGAAGUAGGAAUGAAACAUAAAAGUUUAUAACUUUUU
AUAAGUUUUCAGUAACGG
No. 81:
> NC_010371_1_WP_012289953.1
(SEQ ID NO: 173)
MLITYKTEIKPNPEQVIKIKQTMGVCRFIYNFYIVENQKAYKNNMPFITA
FNFSKWLNNVFIPNNLNYSWIRDVSSKSVKQSIVCAERAFKRFFKKESKF
PRFKKKNKSNVKMYFVRNNKGDCLCERHRIKIPTLGWIKIKEKGYIPTSK
DDYVVKSGAISYKAGRYYISCLVDIEEPPKQILKPFGLGIDLGLEKFAVI
SNGNVYKNINKSKKIIKLEKKLRHEQKCLSRKYEQLKNNKKGESTHKNIL
KQIAKVQKTHLKLSNIRTDYINKIINELVKTKPAYIVIENLNISGMIKNR
YLSKSISNQRFYEFRQKLFNKCNFHGIELRIADRFYPSSKTCHSCGSIKK
DLKLSDRIYKCPTCGEIIDRDLNASLNLRDTTSYKIA*
Associated RNA sequence
(SEQ ID NO: 215)
UAAAAAAAGCUAUAUAAGAUAUGUACCGAUGGCUAGUCGGGAAUUGACGA
CUGUGGAGUGUACAAGAACUUGUUAGUAGCAAAAUCUUUGCAAAAGCAUA
CACAAUGAAGCAGUAAUUUAUCUCGAUGUAGACUUAUUUGUCUAUGUUUU
GAGUAGCAG
No. 82:
> NZ_CP009687_1_WP_044824965.1
(SEQ ID NO: 174)
MKKMIKGIKVMLIPNNKQNTKLFQYAGASRFAYNWGLAKQQENYKNSGKF
INDGELRKEFTQLKKQEGYEWLNNISNNVTKQAIKDACNAYKRFFKGESK
FPRFKSRKKSAPKFYQDVEKIQFNGTHVKFEGFSTSKKKNKQKLNWVRLA
EYNRIPFGENEKYINPRVSFDGLNWWVSVGIEHEDEVKQPTNEGIGIDLG
IKDLAICSDIDKPYKNINKTSKVKKIKKKLRRLQRKISKKYEMNKEGSRY
KKTCNIIKLENKLKKVHHQLKSIRHNYIHQTTCEIVNREPMFIVLEDLAV
SNMMKNKHLAKAIGEQCFYEFYRQIEYKALWNNVKFIIADRFYPSSKMCS
VCGAIDKDLKLSDRTYICECGNKIDRDKNASVNLKNFGELAI*
Associated RNA sequence
(SEQ ID NO: 216)
UCACUUAAAAGAUAUUGCUAAUAUGUACUCAUACGUUAAUGAGGAAUUUA
UGCCUUUGGAGUGUUAUAUCAAACGAAAGUAGCAUAGGCAAAAUCGGACA
CGAUGAACAAGGAAAGAAACACAAAAGUUUAUUUUAUAACUUUUUAUAAG
UUUUCUGUAACGG
No. 83:
> GUT_GENOME100288_478_819_1994_−
(SEQ ID NO: 175)
MLKSYKTEIDPTPEQKQHISRTIGVCRYVYNFYLAHNQEIYEVEKRFVSG
MDFSKWLNNEFIPNNPDFHWIKEVSSKSVKQSIMDAERAFKNFFKGKSRF
PRFKKKARSDVKMYFVKTDAKVIIKCERHRIKIPTLGWVRLKEKGYIPTN
PKTHIIKSGAVSCKAERYYVSVLVEEQEPQKPVLNDFGIGIDLGLKDFAV
CSNGKTYKNINKSSQIRKLEKKLKREQRSLSRKYESYKKLNKNMKGVATR
QNIQKQKLKVQELHQRLDNIRTDHIKKVVSELAKTKPMWITIEDLNISGM
MKNRHLSKAIAQQKLFEFRTKLLAKCNSYGIELRIVDRFYPSSKTCHNCG
SVKSDLKLSDRTYHCCECGYAEDRDYNASLNLRDCRTYKIA*
Associated RNA sequence
(SEQ ID NO: 217)
ACAAGCUAAUGUAGGUAUGUACCGUAGGCUAUACGGGAAUUUACGCCUGU
GGACUAUACAAGAACUUGUGAGUAGUCUUAGGACAAAAGCAUAUAGGUUG
AAGCAGGAAUUUUCUCAAUAUGGGUGUAUUUGUCCAUAUUUUGAGUAGCA
G
No. 84:
> GUT_GENOME089678_61_13091_14269_−
(SEQ ID NO: 176)
MLITKKIRLKPTTEQEILFRKSAGVSRWAYNFFLGENKRIYREYIDNGKV
GKKSISEGDVRKYINNVLKPTTHSWLKEVGSNVMKQGVKDANLALQRYFK
DLSGKPKFKSKHKDRPSFYVNYESLSRKQGGFQGEKIGFVKTSESLPKLN
KNEKYSNPRITFDGKYWYLSVGYEAKENTVELTDESLGIDLGVKDLAICS
NGKVYKNINKTQRVKNLEKKLKREQRKLSRKIENNIQSYKSNRSPVYKRP
LKECQNVQKQNRLIRNIHRKLANIRQNYLHQTTTEIVKTKPSRIVMESLN
VSEMMKNKHLSKAIQQQKFYEFKRQIEYKSKLNGIEFIQVDKFYPSSKTC
SCCGNIKKDLKLSDRMYKCDVCGLVIDRDYNASINLANYEVI*
Associated RNA sequence
(SEQ ID NO: 218)
AUUCACUUAAAAGAAUUAUAUAACUAUGUACCUAUCGCUAUUAGGGAAUU
UACGUCUAUGGAGUGUCAUAACAAAUGAAAGUAGCUACGGCAAAAUCAGA
CACGUUGAAGUAGAAAGUCUUAAUCGUGAGAUAAGACACAGUAUAGAUUU
AUAUAGAUUAGUUGUAAUUUAUAUAUAUUUAUCAUAGCGG
No. 85:
> GUT_GENOME190643_17_8068_9183_−
(SEQ ID NO: 177)
MILAKKVRLCPSELQEEKLWQSVGTARFIYNWALNRQEENYKGGGKFIPD
SVLRKEITQLKKTDLSWLNEVSNNVAKQAVKDACDSYKRFFKGLSGKPRF
KSRKRSKKSFYHDNVKLKVKDNKLVKIEKIGWIKTNEQLSIGAKYSNPRI
SYDNKYWYLSVGIEQEEIQEELTDASLGIDLGLKNLAICSDGTVYKNINK
TCTVKKIEKKLKRLQRQVSRKYEKNKKGKEYVKTKNIIKLEKQIQQTHRR
LANIRNNYLHQTTTSIVKTKPYRVVIEDLAVSNMMKNKHLSDAIRKQGFY
EFRRQLEYKCNFRGIKLVVADRFYPSSKICSQCGEIKKDLKLSDRVYKCN
CGLVIDRDLNASINLSKYKLA*
Associated RNA sequence
(SEQ ID NO: 219)
UAUCAUUAAUAAGAUAAUGCUAAUAUGUAGGAUGCGUUGUAUCCGAAUUU
ACGCCCUUGGAGAGUUAUAUCAAACGAAAGUAGGUUGUUAUUUAUAAUUU
CCAAAAUCGAAUUCGUAGAACAGGGAAUUAAACAAGAUUUAUAGAUAUUU
AUAGAUUUUUGGCAACGG
No. 86:
> ERR1539620_45264_57524_39821_41020_−
(SEQ ID NO: 178)
MNEEDKGDQERMLKSYKTEINPTMEQIHKINKTIGTCRFVYNFYLARNKE
VYEKEKKFVSGMEFSKWLNNVYLKENPEYVWIKEVSTKSVKQSIMNSERS
FKRFFKGQSGFPKFKKKGKSDVKMYFVKTNSQTIITCERHRIKISTLGWV
RLKEKGYIPWDSNTYIIKSGTVSCRAGRYYVSVLVEQPERERLQLQDFGF
GIDLGVKNFAVISNGLVKKNINKTNKVKKLEKQLKREQRCLSRKYESLKK
NNKKGESSRENIQKQILKVKRLYCRIDNIRTDYINKTIAEIVKTKPSYIA
VEDLNVSGMMKNRHLSKAVASQKFYEFRVKLEAKCKEYGIELRIVDRWYP
SSKLCHECGCIRKDLKLSDREYVCECGYHADRDYNASLNLRDATSYKVA*
Associated RNA sequence
(SEQ ID NO: 220)
ACAAGCAUUUGCAGGUAUGUACCGAUGGCUAGUCGGGAAUUGACGACUGU
GGAGUGUACAAGAACUUGUGAGUAGUAUCUGUAUUUACUCAGGGCGAAAG
CAUAUACAGAGAAGCAGUAAGAAAUGUUCGCAAGAACUUUCAAUAUCUCG
AUAUGGAUAUCUUUGUUCAUAUUUUGAGUAGCAG
No. 87:
> NZ_CP013243_1_4129900_4131015_+WP_072587064.1
(SEQ ID NO: 179)
MILAKKIRIIPNMEQEQQLWKSVGTARFIYNWTLARQEGNYKNGGKFIKD
GDLRKEITIMKQTEEYKWLKEVSNNVAKQAVKDGCNAYKRFFKGFADKPR
FKSRKKSRPSFYNDNLKLKVKPKMALIEKVGWIKTAEQVPMNIKYTNPRV
SFDGKYWYLSVGVEKENSKVELTGESIGIDVGIKDLAICSNGMTFKNINK
TRLVKKLEKRLRRLQRKISKKYELNKEGRKFVKTSNIIKLEKQIRLLHRR
LSNIRNNYLHQATTKIVKNKPSRVVMETLNIKGMMKNRHLSKAIAQQGLY
EFKRQLQYKCEFYRIEFVEADKWYPSSKICSECGHKKTKLSLSERTYICE
ECGVVIDRDFNASINLSRYSA*
Associated RNA sequence
(SEQ ID NO: 221)
CUACUUAAACAGUUAUGUUUGAGGUGUAGGAUUCGUUGUAUCCGAAGUUA
AGCCCUCGGAGUGCUAUACAAACCAAAGUAGCUACGGCAAAAUGGAGUAC
GUUGAACAGGGAAUUAAACAAGCUUUAUAGAGUUUUAUAAGGUUUUGGCA
ACGG
No. 88:
> CP029458_1_2205965_2207104_−AYF55140.1
(SEQ ID NO: 180)
MKMILLKKVRLYPTGIQEQKLWQSVGTARFIYNWTLNKQQENYKNGGKFI
KDSDLRKEITLMKKTDEYKWLSEVSNNVAKQAVKDCCNAYKNFFKGLSDK
PRFKSRKKSKPSFYNDNVKLKVKTKLVNIEKVGWIKTKEQIPINVKYTNP
RISFDGKYWYITVGVEQEQSTIELTNKSIGIDVGIKDLAICSNGMTFKNI
NKSKEVKRLKKVLKRKQRKVSRKYDMNKKIEGGENRCQYKKTNNIIKLEK
EIRLLHRRLRNIRNNHIHQATNKIVKTKPSRVVMETLNIKGMLKNKHLSK
AISEQCLYDFKTKMQYKCKLYGIEFIEADKWYPSSKTCSCCGAIKKDLKL
SDRVYKCKCGLVIDRDLNASINLSRYKLA
Associated RNA sequence
(SEQ ID NO: 222)
UAUCACUUAAAAAGAUAAUGCUAAUAUGUACCAUUCGUUGUAUGGGAAUU
UAAGCCCUUGGACUGUUAUACAAACCAAAGUAGCCUCGGCAAAAUGGAAC
AGGUGGAACAGGGAAUUAAACAAGGUUUAUAGAUAUUUAUAGAUUUUUGG
CAACGG
No. 89:
> CP013700_1_58380_59501_−AUM93701.1
(SEQ ID NO: 181)
MILGKKVRLYPTKDHEQKLWQSVGTARFIYNWTLARQEESYKNGGKFISD
NDLRKELTILKKTELNWLNEVSNNVAKQAVKDGCEAYKKFFKGLADRPRF
KSRRKSKPSFYNDTSKLKVKENSVLIEKVGWINIKKNSIPMNCKYSNPRI
SFDGKYWFIAVGIEKEKPIVELTTESIGIDVGVKDLAICSNGMTFKNINK
ARVVKQLEKRLRRLQRRVSRKYLKNKEGSKFVKTSNIIKIEKQIKLLHRK
LANIRSNYNHQATNKIVKTKPSRVVMETLNIKGMMKNKYLSKAIAKQCLY
EFKKQMQYKCEFNGIKFVEADKWYPSSKTCSECGHIKPKLSLSERTYICE
ECGCVIDRDYNASINLSRYELVG
Associated RNA sequence
(SEQ ID NO: 223)
UCACUUAAAAGAUACUACUAAUAUGUACCAUGUGUUGUAUGGGAAUUUAA
GCCCUCGGAGUGUUAUAGCAAACGAAAGUAGCUUUAUAGCAAAAUCGGAC
ACGUUGAACAGGGAAUUAAACAAGCUUUAUAGAGUUCUAUAAUGUUUUGG
CAACGG
No. 90:
> SRR5892202_233507_16022_11195_12361_+
(SEQ ID NO: 182)
MIILLKSFKTEINPTAEQIIKINKTIGVCRFIYNFYIAHNKELYENGEKF
ISGRRFNVWLNNEYLPNNPDKMWIKDVYTKAVAKSMDNACVAFTRFFMCK
SKFPRFKKKNKSDVKMYFVKNNKTDCLSERYRIKVPTLGWVRLKEKGYIP
TSKDGFIIRSGTISHKAGRYYISVLVDIEKQDTEMSNNFGIGIDLGLKSL
AVCSDGSTYCNINKTYNIRKVEKILKREQRKLSRKVISIKKGESTRKNFV
KQRLKVQKLYQRLDNIKTDYINKTISKVVKTKPSYIVIEDLNVSGMMKNR
HLSKAVAQQKFFEFKTKLISKCKENNIELRVVDRFYPSSKLCHNCDNVKK
DLKLSDRIYKCSECGYVEDRDINASLNLRDAKTYTIIQ*
Associated RNA sequence
(SEQ ID NO: 224)
AUAUAGUGUAUAAGUAUGUACCAAUGGCUAGUUGGGAAUUUACGACUGUG
GAGUAUAGAAGAACUUGUGAGUAAUAUAUUCAACUUUGUUGAAUUAUGAA
AGCAUAUACGUUGAAGCAGUAAGUAAAAAUCGUGAGGUUUUACAAUUCUC
AUUAUAGACAUUAGGUCUAUAAUUUGAGUAGCAG
No. 91:
> NC_014171_1_3506713_3507831_−WP_000978855.1
(SEQ ID NO: 183)
MMLAKKVRIKPNKGQELQLWKSVGTARWAYNWTLAKQEENYKNGGKFISD
VFLRKELTVLKQTEEYAWLYDVSNNIAKQAIKDACEAYKRFFKGLADKPR
FKSRRKAKLSFYNDNVKLKVKEKQVLIEKVGWVKTSEKLPVGVKYSNPRV
SFDGKYWYLSVGIEQEFTKEKLTAVSLGIDIGIKELAYCSDGQRKNNINK
TKMVRQVEKRLRRLQRQVSCKYEMNKEGNRFVKTSNIIKIEKEIRQLHRR
LSNIRKNHLHQSTSEIVKTKPFRIVMETLHMKGLMKNRYLAKAIAKQCLY
EFKRQIQYKCEKYGIEFVEADTWYPSSKMCSSCGSINKELKLSDRVYKCT
CGHEMDRDLNAAINLSRYELAS*
Associated RNA sequence
(SEQ ID NO: 225)
CAUCUAAAAAAGUUACGGCUAAUAUGUAGGAUUCGUUGUAUCCGAAUUUA
CGCCCUCGGAGUGUUACAUCAAACGAAAGUAGCUUCUGUGAAAUCGGACA
CGUAGAACAGGGAAAUAAACAAAAUUUAUAGAUUUUUAUAAGUUUAUGGC
AACGG
No. 92:
> NC_017209_1_13469_14587_−WP_000604588.1
(SEQ ID NO: 184)
MILVKKVRLKPNREQEMQLWKSTGIARWAYNWALSRQEENYKQGGKFISD
GTLRKELTQLKQTEEYAWLYDVSNNVTKQAVKDLCEAYKRFFKGVVDKPR
FKSRRHSKPSFYNDNVKLKVMKNMVLIGKVGWVKTSECIPVVNKYSNPRI
SFDGKYWYLAVGIEQEIIHQELTNASIGIDVGIKELAFCSNGMKFKNINK
TKVVKQVEKRLRRLQRQVSRKYEINKEGSCFVKTSNIIKLEKKIQHLHRR
LTNIRDNHIHQVTCAIVKTKPCRVVMETLNIKGMMKNKHLSKAIAQQKLY
DFKMKMKYKCEKYGIEFVEADKWFPSSKQCSNCGEVNKHLTLSDRTYHCK
CGHHIDRDLNASINLSRYQLVN
Associated RNA sequence
(SEQ ID NO: 226)
CACUAAAACGUUAUUGCUAAUAUGUAGGAUUCGUUGUAUCCGAAUUUACG
CCCUUGGAGUGUUAUGUCAAACGAAAGUAGCUUCGGCAAAAUCGGACACG
UAGAGUAGGGAAGUAAACAGACUUUAUAAAGUUUUAUAAGGUUUUGGCAA
CGG
No. 93:
> NZ_CP012098_1_2114035_2115228_+WP_077326813.1
(SEQ ID NO: 185)
MMVVKKMIKTIRVMLIPNNKQNTKLFRYANTARFAYNWALGREKENYKNG
GKFLSDSDLRKEFTQLKKTEEYSWLKEVSNNVTKQAIKDACHAYKRFFKG
CSKFPKFKSRKFSIPSFYQDNVKIQFSDTHVKIEGFAASKKKNKQKINWI
RLAEKNRIPTDCNYSNPPIRYDGINWWITVGIEYEDSVTVPSNDGIGIDL
GIKDLAICSDGNKYKNINKTKKVKKLEKQKRRLQRSISRSYENNKQGKEY
CKTKNVIKKEKLLLILNHRLTNIRHDHLHHITSEIVKREPSFICIEDLNV
KGMMKNRHLSKAVQQQGFYEFRRQMEYKSKWNNIQAIIADQFFPSSKLCS
CCGKIKKDLKLSERIYKCECGNVIDRDLQAALNLKKYGEDVLKRSVA
Associated RNA sequence
(SEQ ID NO: 227)
UAUAUUCAAGUGAUUACAGAUAUGUACUGAUACGUUAGUCAGGAAUUAAC
GCCUAUGGAGAGUACAGGAACUUGUGAGUAGUACAUGAAUUGAUUCGUUA
CAAAAGCAUACUCGUUGAAGUAGGAAUGAAACAUAAAGGUUUAUAACUUU
UUAUAAGUUUUCGGUAACGG
No. 94:
>IS607_orfB_GUT_GENOME170581_2_14431_15588_−
(SEQ ID NO: 186)
MYKSFKTEINPTPTQKAKIDKTIGTCRFVYNFYLAHNIELFNAGEDLMTG
KMFSVWLNNEYLPNNPEYNWIKDTYSKAIKKSIEDGFTSFKRFFKHESDF
PRFKKKGKSNVKMYFVKNNKTDCKCERHRIKIPTLGWIRIKEKGYIPTEK
QGWNIKSGTVSYKAGRYYVSVLAEIPDIEYEKTSNEGVGIDLGIKDFSVI
SNGNVHENINKSHKIRKLEKKLRREQRCFSRKYENLKKEGEPTQKKNIEK
QKLKVQKIYHKMDNIRTDYINKTIAEIVKTKPSYITIEDLNVKGMMKNKH
LSKAIASQKFCEFRRKLKIKCDNQNIELRIVDKFYPSSKLCHHCDSIKKD
LKLSDRVYRCKCGYIEDRDRNAALNLKDAQSYVVA*
Associated RNA sequence
(SEQ ID NO: 228)
AAGAUAGAAAAGAAUUUUAAGCAUGUACCAAAGGCUAAUUUGGGAAUUAA
CGACUGCGGAGUGUACAAGAACUUGUUAUUAAUGCCUUAUUAAAGACAUG
AAAGCAUACACGUUGAAACAGUAAGAAGUAUCUGUGAAGACUUCAAUUUC
UCAAUAUGUUAAGUAUAUUAAUAUGAUUGAGUGGCAG
No. 95:
> NZ_CP011114_1_2448296_2449408_−WP_025697990.1
(SEQ ID NO: 187)
MILTKKVRIRPALEQEQQLWRSVGTARWVYNWTLSRQEENHKHGGKFLSD
GDLRKELTQLKHTEEYAWLCNVSNNVAKQAVKDACDAYKKMFKGLAKKPK
FKSRKKSRPSFYNDTAKLKVKDQVVLLEKVGWMKTVEQLPIGVSYSNPRV
TFDGKYWYLSVGINQEQVQQKLTDEVVGIDVGVKELAVCSNGMVFKNINK
SAAVRKTEKRLRRLQRKVSRKYEMNKEGSRFVKTSNIVKVEKSIRLLHRR
LTNIRTNHIHQATSAVAKTKPRAVVMETLNVSGMMKNRHLSQAIANQKLN
EFIRQMKYKCEKRGATFMQADRWFPSSKCCSGCGQVKTHLKLSDRIYECD
CGLKIDRDLNAAINLAQLAV
Associated RNA sequence
(SEQ ID NO: 229)
CAUCUUAAAGCUUACGCCAAUGUGUAGGAUUCGUUGCAUCCGAAUUUACG
CCUGUAGAGUGUUAUACCAAACGUAAGUAGCUUCGGCAAAAGCGGGCACG
UUGAAGCAGGAAGCAAACAAGAGUUGUGUAGACUUGUACAAUCUCUUGGC
AACGG
No. 96:
> CP035280_1_2081523_2082695_−QAT40560.1
(SEQ ID NO: 188)
MKKLKKAYKTEIKPTKEQIIKIRRTIGVSRFIYNFYIAHNKEVYEKEKKF
VSGMDFSRWLNNEYIPNNQDKVWIKKVSSKAVKQAIMNGEKAFKKFFKGE
AGFPKFKKKKNQDIKAYFPKNNETDWTIERHRIKIPTLGWIGLKEYSYIP
ANGRITSGTVSQKADRYYVSVLVDEDIKINNKRCSEGIGVDLGLKDFAIC
SNGITKKNINKTKTVKKAEKKLKREQRKLSRKYESLKLRNKKEKGEATRQ
NIQKQIVKVQKLHQRLTNIRTDYINKTVSELIKQKPRFITIEDLNVRGMM
KNRHLAKAVAQQKFYEFRIKLISKSKQDGIEVRIVDRFYPSSKTCSCCGR
IKKDLKLSDRVYKCDNCNTSIDRDLNASINLANAKEYKIA
Associated RNA sequence
(SEQ ID NO: 230)
UUAAACAAGCGCUUAUAUGUGUACCGAAGGCUAUUUCGGGAAUUUACGCC
UGUGGAGUGCUAUACAAACUGUAGUAGCUUAGGCAAGGCAGAGUACAAUG
AAACAGGAAUCUUCUCAGUAUGGAUACAUUUGACCAUAUUUUGAGUAGCAG
No. 97:
> NC_017078_1_59860_61161_−WP_014431089.1
(SEQ ID NO: 189)
MIRSVRIRLLPNNKQRTKLFQFAGASRFAYNWALDKQMENFREGRKLQSD
YDLRKEFTVLRNSEEYLWLLGISNNVTKQAIKDLCIAYKNFFHKQRQKGY
VKYSPKKLAHFARIARTPTIYDMNGHPKFRSKKNGDFRFYQDNVKIQFTA
THVKLENIAGSRKKNRQRLNWIRLAEKGRIPTKGRYTQPRITFDGENWWL
SVGIRTSYTLKPMRGLNFSRKQPGRFCTEGVGIDLGIKDLAIISDSTVVK
NINKSHTIRNIKKKRRRLQRQVSRKYQMNKKGERYCKTSNLIKSEKRLLR
INHRLADIRANHVHQATRIIINRKPRFICLEDLNVQGMMKNKHLSEKVQE
QNFYEFRRQIEYKAHWARIPVVIADRWFPSSKTCVECGYINKDLKLSDRT
YVCPVCGNVIDRDFQASLNLKRYGETELSKSAS
Associated RNA sequence
(SEQ ID NO: 231)
CACCGACAAGUCAAUGCAGAUAUGUACCGAUUCGUUAGUCGGGAAUUUAA
GCCUCUGGAGCGUUAUAUCAAACGUGAGUAGUCUGUUCUUCGGGACGGCA
AAAGCGGACGCAGUGAAUGAGGAAUGGAACGUAAAAGUUAACUUUCUACG
GAAGGUUUAUGACUUUUUAUAAGUUUUCAGUAACGG
No. 98:
>IS607_orfB_GUT_GENOME088555_122_512_1693_+
(SEQ ID NO: 190)
MMIKTIRVMLVPNNKQKTKLFQYANTARFAYNWALGREQENYKNGGKFIS
DGDLRKEFTQLKKKEEYAWLNYVSNNVTKQAIKDACEAYRDFFKGYTKFP
RFKSKKHSVPKFYQDNVKIQFTDTHVKVEGFATSRKKNKQKLNWIRLAEH
GRIPTEKVKYMNPRIKYDGLNWYITVGIEYEDSTTLSTNEGIGIDLGIKD
LTICSDGNTYKNINKTHKIKKLEKRKRRLQRSISRRYEKNKKGGSYCKTS
NIIKSEKELLKLNHRLTNIRQNYLHQTTTEIVKTRPSFIVLEDLNVKGMM
KNKHLSKSVQQQCFGEFRRQIEYKSAWNNIQVIIADRFFPSSKLCSYCGN
IKKDLKLSDRIYRCECGNVIDRDYQAALNLKKYGEMALEQSVA*
Associated RNA sequence
(SEQ ID NO: 232)
CACAUUCAAGUUAAUACAGAUAUGUACGGAUUCGUUAGUUCGGAAUUUAA
GCCUAUGAAGUGUACAAGAACUUGUGAGUAGUAUCUGGGUUUAUUCAGUA
CAAAAGCAUACACGACGAAGUAGGAAUGAAACAUAAAAGUUUAUAACUUU
UUAUAAGUUUUCAGUAACGG
No. 99:
> CP030117_1_439976_441088_+AWX54000.1
(SEQ ID NO: 191)
MILAKKVRLKPTPEQERQLWKSVGTARWAYNWTLAKQEENYRNGGKFLLD
GDLRKELTQLKQSEEYSWLYEVSNNITKQAVKDACDAYKKFFKKLAEKPT
FKSRRRSKPSFYNDNVKLKVKQNHVLIEKVGWVTTSEQLPTDVNYTMPRI
SFDGKYWYLSVGVEQEFIQPELSGESLGIDVGVKELAVCSNGMTFKNINK
TKVMKKAEKRLRRLQRKVSRKYEMNKEGSRFVKTCNIVKVEKHIRLLHRR
LTNIRTNHSHQATSAIVKTKPSTVVMETLNIKGMMRNRHLSKAIAQQKLY
EFKRQLQYKCEKYGIRFMEADKWYPSSKTCSDCGEVKKDLKISDRIFKCV
CGLVLDRDVNASINLAKLAS
Associated RNA sequence
(SEQ ID NO: 233)
CCCCAAUAAGUUACCGCUAAUCUGUAGGAUUCGUUGUAUCCGAAUUUACG
CCUGUGGAGUGUCCGAUCAAAUGCGAGUAGCUUCGGCCAAAGCAGACACA
GAGAAGCAGGAAGCAAACAAAAGAUGUGUAGACUUUUACAAUCUUUUGGC
AACGG

### EXAMPLES

The present disclosure is further described by the following examples, which are not to be construed as limiting the scope of the present disclosure.

### Example 1: Discovery and Acquisition of the Gene Sequences of RAD (IS607 TnpB) Systems

Non-redundant, high-quality genome sequences of 1,520 gut strains isolated and cultured from healthy human fecal samples were downloaded from NCBI (project number PRJNA482748). 19,165 complete bacterial genome sequences were downloaded from the NCBI FTP site (ftp://ftp.ncbi.nih.gov/genomes/Bacteria/). 26,097 bacterial genome sequences were downloaded from the IMG database (https://img.jgi.doe.gov/). 12,715 microbial genome sequences assembled from marine metagenomes were collected from the literature (Maria G Pachiadaki et al., Cell. 2019 Dec. 12; 179(7):1623-1635.e11. doi: 10.1016/j.cell. 2019.11.017; Charting the Complexity of the Marine Microbiome through Single-Cell Genomics). 204,938 microbial genome sequences isolated from the human gastrointestinal tract were downloaded from the MGnify database. 141,115 unassembled metagenome samples covering multiple species (plants, insects, birds, etc.) were downloaded from MGnify database. 96,709 unassembled metagenome samples from multiple tissue sites of humans were downloaded from HumanMetagenomeDB. Further assembling was performed to obtain genome sequences.

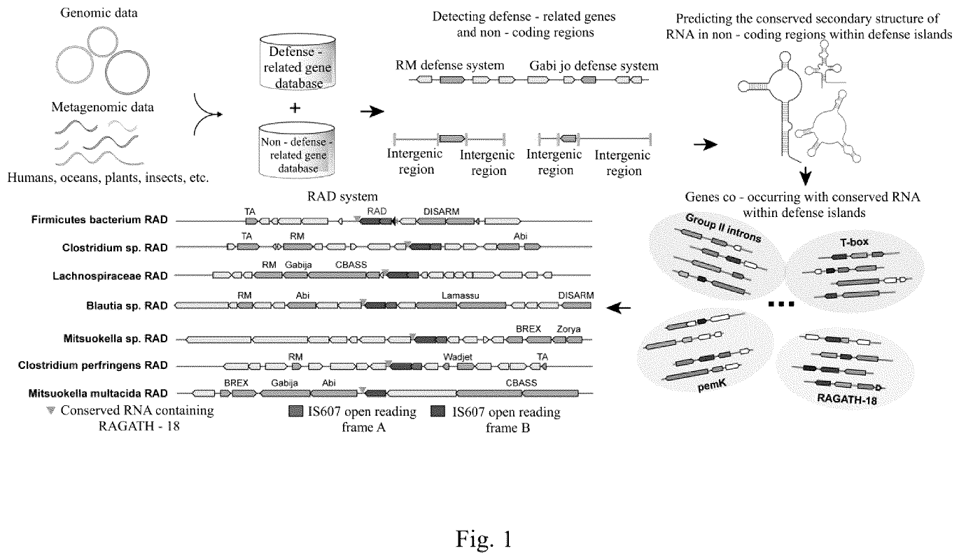

The inventors analyzed the intergenic region (IGR) proximal to defense-related genes one by one by enumeration, and predicted the conserved secondary structure by Rfam (FIG. 1). The prediction method refers to Rfam 14: expanded coverage of metagenomic, viral, and microRNA families. Hundreds of RNAs with conserved secondary structure were discovered, and gene family analysis was performed on 10 upstream or downstream proteins to explore other functional elements that cooperate with these defense-related RNA candidate proteins. This analysis identified a RAGATH-18-associated DNase system (RAD) comprising RAGATH-18-associated RNA and an IS607 element. The effector protein of RAD (IS607 TnpB) is the second coding sequence ORFB of IS607, encoding approximately 387 amino acids in size.

The inventors selected the source strains and effector proteins of a total of 99 (Table 1) RAD (IS607 TnpB) systems as representatives for subsequent analysis. Based on the effector proteins of the 99 RAD systems, the inventors had obtained the DNA sequence information of candidate proteins as well as the corresponding amino acid sequences and the sequence information of RAGATH-18-associated RNA, from a plurality of genomic data mentioned above. The candidate protein sequences and the sequences of the corresponding RAGATH-18-associated RNA are shown in Table 2, along with the sequences below Table 2. According to the strain ID, protein ID and amino acid information listed herein, the corresponding DNA information of the proteins can be obtained in the above database.

### Example 2: Expression and Purification of RAD (IS607 TnpB) Protein-Nucleic Acid Complex

The DNA coding sequences of the obtained candidate proteins were sent to a gene synthesis company (Jinweizhi Biotechnology Co., Ltd.) for gene synthesis, and Pgex-6p1 universal vector was selected as the expression vector. The gene synthesis company synthesized 99 expression plasmids PGEX-RAD 1-99 (containing coding sequences of the candidate protein of each of SEQ ID NOs: 1-57 listed in Table 2 and SEQ ID NOs: 150-191 listed subsequently, respectively and the corresponding RNA coding sequences). The insertion sites for all expression cassettes were BamHI and EcoR1 sites, and the structure of the expression cassettes was Rad protein-6HIS-RNA-N20-HDV (wherein Rad protein was each of SEQ ID NOs: 1-57 and SEQ ID NOs: 150-191, such as FbRAD1 protein of SEQ ID NO: 6).

The sequence of an exemplary plasmid PGEX-6P1-FbRAD1-N20-HDV is shown below, wherein the RAD protein (i.e., FbRAD1 protein) is underlined and in bold; 6HIS is underlined; the RNA coding sequence is italicized and underlined; N20 is in bold; and HDV is italicized.

(SEQ ID NO: 115)
ATGAGGAAATTGCTGAAGAGCTTCAAGACGGAAATAAATCCAACAGAAGAGCA
GAAAGTCAGGATTCGTAAAACAATAGGTACTTGCAGATTTATCTATAACTTCTATCTTGCT
CATAATAAGGAGCTTCATGAAAGTGGCAAAAAGTTTATGAGCAGCAGCCAATTCAGAGTA
TGGCTCAACAATGAATATCTTCCGAGCCATCCGGAATATTCCTGGATCAAGGAAGCTTAT
TCAAAATCGGTAACACAGGCAGTAAATGATGGACAGACCGCATTTACAAGATTCTTTAAT
CACAAGAGCGCCTTTCCAAAGTTTAAAAAGAAAGGCAAGTCTGATGTGAAAATGTATTTT
GTAAGGAATAATCCAAAAGATTGCAGCTGCGAAAGACACAGAATCAAGATTCCTTCACTT
GGCTGGGTCCGTATCAAAGAGAAAGGATATATTCCCACTACAAAAGATGGATATGTAATT
AAGAGCGGTCATGTCTCAATAAAAGCTGATAGATACTATGTTTCAGTTCTTATAGAAATC
CCAGACAAAACGTCAGCTAGTAATTCCAGTGAAGGAATTGGCATTGACCTTGGATTAAAG
GACTTTGCGATTGCTTCTAATGGAAAGACTTATAAGAACATCAACAAATCTGCTAAATTAA
AGAAGCTTGAAAAAAAGCTTATTCGAGAACAGAGAAGTCTTTCTCGAAAGTATGAAAATA
TAAAGAAAGGAGGGTCCACTCAAAAAAGAAATATACAAAAACAAAGGCTTAAGATACAGA
AACTTCATCATAGAATCGACAATATTCGTACTGATTACATCAATAAGATAATCGCAGAGAT
AGTGAAAACCAAGCCATCTTATATAACGATTGAAGATCTAAACATATCAGGGATGATGAA
GAACAGACATCTTTCAAAGGCAGTTGCTTCGCAGAAGTTTTATGAATTTAAGACAAAGCT
TCAAGCTAAATGCAGGGAGAACGGAATCGAACTTCGAGTTGTAGATAGATGGTTCCCATC
ATCTAAGACTTGTCACTGCTGTAAAAGTATCAAGAAAGATTTAAAGCTTTCAGATAGACTT
TTCAGATGTGATTGTGGATATATCGAAGACAGAGATTTCAATGCTGCACTAAATCTGAGA
GATGCTACAACTTACGAAGTTGCAAGCAGCCATCACCATCATCACCACTAAATGAACGCAAAC
GTAAGTATGTACCGAAGGCTATTTCGGGAATTTACGACTATGGAGTGCACTAGAACTTGTGAGTAGAT
ATGATTTCGGTCAATCCAAAGCATGCACAGTGACATAGTAAGTAATGTTCGTGAGAACTTACATTATCT
CGATGTGAGTATATTTAATCACATTTTGAGTGGCAGCTGATGGTCCATGTCTGTTAGGCCGGCATG
GTCCCAGCCTCCTCGCTGGCGCCGGCTGGGCAACATGCTTCGGCATGGCGAATGGGACCCG

The expression plasmids of other RAD proteins were synthesized by the gene synthesis company in a similar manner to PGEX-6P1-FbRAD1-N20-HDV, except that the coding sequence of FbRAD1 was replaced with a coding sequence of other RAD proteins and the RNA sequence for FbRAD1 was replaced with a RNA sequence for other RAD systems, thereby obtaining the expression plasmids of all 99 candidate proteins, PGEX-RADs 1-99 (including PGEX-6P1-FbRAD1-N20-HDV). All plasmids were verified by the gene synthesis company to be correctly cloned.

Plasmid transformation experiment: the competent E. coli C43 (DE3) cells stored in a −80° C. refrigerator were taken out and placed on ice. After thawing, 100 ng of each of the expression plasmids PGEX-RADs 1-99 was added to 50 μL of E. coli cells respectively, mixed and incubated on ice for 30 minutes. The cells were then heat-shocked in a 42° C. water bath for 90 seconds and immediately placed on ice for 2 minutes, which were aseptically operated in an ultra-clean workbench. 500 μL of LB liquid culture medium without antibiotics was added to the cells. The cells were cultured with shaking in a shaker at the constant temperature of 37° C. for 45-60 minutes, then evenly spread on LB plates containing 100 μg/mL antibiotic ampicillin. The plates were inverted and cultured overnight in an incubator at 37° C., and single colonies were seen on the second day.

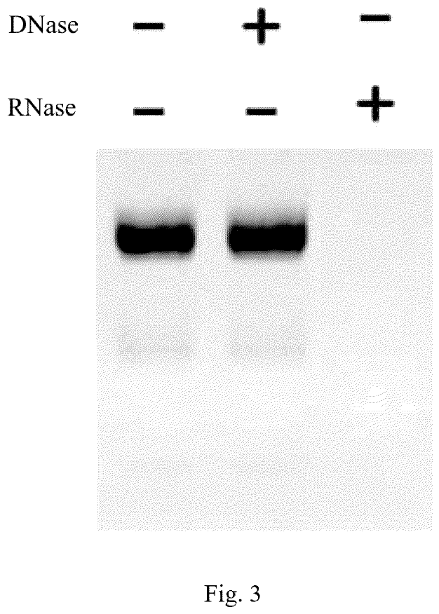

The expression and purification of RAD (IS607 TnpB) systems: positive clones were picked and inoculated into 5 mL of LB liquid medium containing antibiotic ampicillin. The cells were cultured while shaking in a shaker at 37° C. for 12 hours. Then, cells were transferred to LB liquid medium containing antibiotic ampicillin, and cultured in a shaker at a constant temperature of 37° C. to the OD600 of about 0.6. The culture was cooled to 18° C., and IPTG was added to a final concentration of 0.6 mmol/L in order to induce the expression for 16 hours. The bacterial cells were collected on the second day. Using a gravity column with column-material of GST-Sepharose, and utilizing the affinity effect between the GST tag and GST-Sepharose to extract protein from the cell lysate. The bacterial solution cultured overnight was centrifuged at 4° C. and 4000 rpm for 10 minutes. Then the resuspension solution was added at 20 mL GST resuspension solution per 1 L of bacterial solution and the bacterial cells were resuspended by shaking thoroughly with a vortex shaker. During resuspension, the protease inhibitor PMSF was added at a final concentration of 2 mmol/L, and the resuspension solution comprises 25 mmol/L Tris (pH 8.0), 1 mol/L NaCl, and 3 mmol/L DTT. Ultrasonication was used to fully lyse the resuspended bacterial cells. The whole ultrasonication process should be carried out on ice. In order to prevent the probe from overheating, it was necessary to pause for 3 seconds every 3 seconds of operation. The ultrasonication was performed for 1 minute, generally for 5-8 times, until the bacterial solution was no longer viscous and was homogenized. The lysed bacterial solution was dispensed into high-speed centrifuge tubes precooled at 4° C., and centrifuged at 15,000 rpm and 4° C. for 40-50 minutes. Then the supernatant was removed. The supernatant was loaded into a gravity column filled with 2 mL of column-material GST Sepharose, and allowed to completely flow through the column material. Salt (NaCl) gradient concentration wash was used to remove non-specifically bound proteins and nucleic acids. 40 mL of GST Washing buffer I (25 mmol/L Tris (pH 8.0), 1.5 mol/L NaCl, 3 mmol/L DTT) was firstly used to wash the column, followed by washing with 15 mL of GST Washing buffer II (25 mmol/L Tris (pH 8.0), 0.5 mol/L NaCl, 3 mmol/L DTT), and finally 10 mL of GST Washing buffer III (25 mmol/L Tris (pH 8.0), 0.3 mol/L NaCl, 3 mmol/L DTT) was used to wash the column, with all Washing buffer being pre-cooled at 4° C. 5 mL of GST Washing buffer III was load into the column, followed by sealing the gravity column. 80 μL of Prescission protease was added for digestion overnight at 4° C. Then the column was resuspended 3 to 4 times, each with 30-minute interval. Another 5 mL of GST Washing buffer III was added on the second day and the effluent was collected. After concentration to 2 mL, the solution was centrifuged at 15000 rpm and 4° C. for 5 minutes in preparation for the next step of purification. The gel filtration chromatography column (Superdex200 100/300) was connected to an FPLC Flash Protein Liquid Chromatography workstation as per the instruction of the device. The gel filtration chromatography column (Superdex200 100/300) was equilibrated with a pre-cooled gel filtration chromatography buffer (10 mmol/L Tris-HCl pH 8.0, 150 mmol/L NaCl, 3 mmol/L DTT, 2 mmol/L MgCl₂). The column was equilibrated for at least one or more column volumes, until the ultraviolet absorption at 280 nm was stable and no longer changed. 2 mL of sample was injected into the loading loop of the workstation and the preset program of the workstation was run. After completion of the program, the purity of the proteins was observed by 10% SDS-PAGE, and binding with nucleic acid was observed by 10% denaturing Urea-PAGE gel stained with EB. For the FbRAD1 complex, FIG. 3 shows the results of 10% denaturing Urea-PAGE gel stained with EB (far left of FIG. 3). It was confirmed by PAGE that the actual molecular weights of RAD protein and RNA of all 99 RAD nucleic acid complexes was consistent with the expected molecular weights, and the RAD nucleic acid complexes were correctly expressed. The following table shows the experimental data for several exemplary complexes.

| | | Expected | | Expected | |
|---|---|---|---|---|---|
| | | Protein | Experimentally | RNA | Observed RNA |
| Protein name | ID | size | observed size | size | size |
| FbRAD1/ISFba1 | RHS75625.1 | 45.0 kDa | about 45 kDa | 187 nt | about 180 nt |
| CbRAD1/ISCba1 | RHR46218.1 | 45.4 kDa | about 45 kDa | 186 nt | about 180 nt |
| RiRAD/ISRin | RGR69673.1 | 44.9 kDa | about 45 kDa | 187 nt | about 180 nt |
| ErRAD/ISEre1 | RHB06265.1 | 44.9 kDa | about 45 kDa | 187 nt | about 180 nt |
| WcRAD | RGR87203.1 | 43.5 kDa | about 44 kDa | 155 nt | about 160 nt |
| BsRAD/ISBsp4 | RHV03091.1 | 45.3 kDa | about 45 kDa | 184 nt | about 180 nt |
| CbRAD2/ISClsp2 | RHR42937.1 | 45.0 kDa | about 45 kDa | 187 nt | about 180 nt |
| CbRAD3/ISClsp3 | RGD88909.1 | 45.3 kDa | about 45 kDa | 186 nt | about 180 nt |
| CcRAD/ISCco | RGT87174.1 | 45.4 kDa | about 45 kDa | 168 nt | about 160 nt |
| FbRAD2/ISFba2 | RGH01875.1 | 45.5 kDa | about 45 kDa | 168 nt | about 160 nt |
| FbRAD3/ISFba3 | RHU26542.1 | 44.8 kDa | about 45 kDa | 187 nt | about 180 nt |
| FbRAD4/ISFba4 | RGH37793.1 | 45.2 kDa | about 45 kDa | 187 nt | about 180 nt |

Nucleic acid identification: The RAD-nucleic acid complex with a final concentration of 5 mg/ml was treated in a 10 μL reaction system using DNase or RNase with a final concentration of 0.1 mg/ml, respectively. The results are shown in FIG. 3. The nucleic acid on the complex was completely degraded only by RNase, indicating that the nucleic acid bound by RAD protein was RNA. All 99 RAD-nucleic acid complexes were validated, and the results demonstrated that the nucleic acids bound to RAD proteins in the 99 RAD nucleic acid complexes were RNAs.

RNA sequencing: The purified complex was sent to a gene sequencing company (Shanghai Personal Biotechnology Co., Ltd) for processing to obtain sequence data information (FIG. 18).

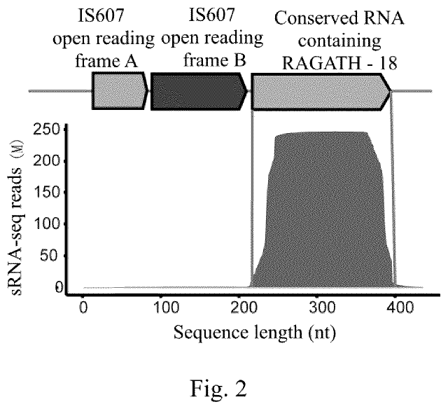

According to the sequencing results, the inventors found that the RNA bound to the RAD protein was a non-coding RNA containing RAGATH-18, which is downstream of the second coding frame of IS607 (as shown in FIG. 2), indicating that the RAD system was able to express and form a protein-RNA complex. This RNA is unique to this system, and there is no relevant study involving the function between this segment of non-coding RNA and the upstream protein.

### Example 3: Determination of PAM Sequences and the Enzymatic Digestion Activity

The construction of a substrate for enzymatic digestion: construction of a PAM substrate plasmid library: a plasmid library covering any combination of 7 bases. Primers PAM-I-F and PAM-I-R were used for PCR amplification (amplification time 3 minutes, primer annealing temperature of 58° C., number of amplification cycles being 30) of the recombinant fragments. Primers PAM-V-F and PAM-V-R were used for PCR amplification (amplification time 3 minutes, primer annealing temperature of 58° C., number of amplification cycles being 30) of the recombinant vector PUC19. Using the recombinase system (available from Vazyme Biotech Co., Ltd., Cat. No. Vazyme. C112-01), the fragment and vector PUC19 were recombined and transformed into E. coli DH5a competent cells. The cells were directly expanded and cultured, and the plasmid library was extracted using a gel extraction kit (Cat. No. CW2302; Cowin Biotech Co., Ltd.) according to the manufacturer's instructions.

The PAM plasmid library engineered based on the PUC19 plasmid is shown below, with N representing any of the four types of bases of A, T, C, or G, and the targeted sequence underlined.

(SEQ ID NO: 118)
gcgcccaatacgcaaaccgcctctccccgcgcgttggccgattcattaatgcagctggcacgacaggtttcccgactggaaagcgggcag
tgagcgcaacgcaattaatgtgagttagctcactcattaggcaccccaggctttacactttatgcttccggctcgtatgttgtgtggaattgtga
gcggataacaatttcacacaggaaacagctatgaccatgattacgccaagcttgcatgcctgcaggcctggacatccgcgagcacagctaaggcc
aNNNNNNNCTGATGGTCCATGTCTGTTActcacaccgaccggcacagcatcaagaagcgggtaccgagctcgaattcactggccgtcgttttaca
acgtcgtgactgggaaaaccctggcgttacccaacttaatcgccttgcagcacatccccctttcgccagctggcgtaatagcgaagaggcccgca
ccgatcgcccttcccaacagttgcgcagcctgaatggcgaatggcgcctgatgcggtattttctccttacgcatctgtgcggtatttcacaccgc
atatggtgcactctcagtacaatctgctctgatgccgcatagttaagccagccccgacacccgccaacacccgctgacgcgccctgacgggcttg
tctgctcccggcatccgcttacagacaagctgtgaccgtctccgggagctgcatgtgtcagaggttttcaccgtcatcaccgaaacgcgcgagac
gaaagggcctcgtgatacgcctatttttataggttaatgtcatgataataatggtttcttagacgtcaggtggcacttttcggggaaatgtgcgc
ggaacccctatttgtttatttttctaaatacattcaaatatgtatccgctcatgagacaataaccctgataaatgcttcaataatattgaaaaag
gaagagtatgagtattcaacatttccgtgtcgcccttattcccttttttgcggcattttgccttcctgtttttgctcacccagaaacgctggtga
aagtaaaagatgctgaagatcagttgggtgcacgagtgggttacatcgaactggatctcaacagcggtaagatccttgagagttttcgccccgaa
gaacgttttccaatgatgagcacttttaaagttctgctatgtggcgcggtattatcccgtattgacgccgggcaagagcaactcggtcgccgcat
acactattctcagaatgacttggttgagtactcaccagtcacagaaaagcatcttacggatggcatgacagtaagagaattatgcagtgctgcca
taaccatgagtgataacactgcggccaacttacttctgacaacgatcggaggaccgaaggagctaaccgcttttttgcacaacatgggggatcat
gtaactcgccttgatcgttgggaaccggagctgaatgaagccataccaaacgacgagcgtgacaccacgatgcctgtagcaatggcaacaacgtt
gcgcaaactattaactggcgaactacttactctagcttcccggcaacaattaatagactggatggaggcggataaagttgcaggaccacttctgc
gctcggcccttccggctggctggtttattgctgataaatctggagccggtgagcgtgggtctcgcggtatcattgcagcactggggccagatggt
aagccctcccgtatcgtagttatctacacgacggggagtcaggcaactatggatgaacgaaatagacagatcgctgagataggtgcctcactgat
taagcattggtaactgtcagaccaagtttactcatatatactttagattgatttaaaacttcatttttaatttaaaaggatctaggtgaagatcc
tttttgataatctcatgaccaaaatcccttaacgtgagttttcgttccactgagcgtcagaccccgtagaaaagatcaaaggatcttcttgagat
cctttttttctgcgcgtaatctgctgcttgcaaacaaaaaaaccaccgctaccagcggtggtttgtttgccggatcaagagctaccaactctttt
tccgaaggtaactggcttcagcagagcgcagataccaaatactgttcttctagtgtagccgtagttaggccaccacttcaagaactctgtagcac
cgcctacatacctcgctctgctaatcctgttaccagtggctgctgccagtggcgataagtcgtgtcttaccgggttggactcaagacgatagtta
ccggataaggcgcagcggtcgggctgaacggggggttcgtgcacacagcccagcttggagcgaacgacctacaccgaactgagatacctacagcg
tgagctatgagaaagcgccacgcttcccgaagggagaaaggcggacaggtatccggtaagcggcagggtcggaacaggagagcgcacgagggagc
ttccagggggaaacgcctggtatctttatagtcctgtcgggtttcgccacctctgacttgagcgtcgatttttgtgatgctcgtcaggggggcgg
agcctatggaaaaacgccagcaacgcggcctttttacggttcctggccttttgctggccttttgctcacatgttctttcctgcgttatcccctga
ttctgtggataaccgtattaccgcctttgagtgagctgataccgctcgccgcagccgaacgaccgagcgcagcgagtcagtgagcgaggaagcgg
aaga.

2700 bp double-stranded DNA substrate: a 2700 bp double-stranded DNA fragment was amplified by PCR using primers DS2700-F and DS2700-R, using the PAM substrate plasmid library as template. The amplification time was 3 minutes, the primer annealing temperature was 58° C., and the number of amplification cycles was 30. The PCR products were separated by 1% agarose gel electrophoresis, and the product was recovered. 2700 bp mismatched double-stranded DNA substrate (the base complementarity of CTGATGGTCCATGTCTGTTA (SEQ ID NO: 116) was changed one base by one base to form a mismatch), and the construction method was the same as described above. 5′FAM-labeled single-stranded DNA substrates: 5FAM-55TAM-F, 5FAM-55TAM-R, 5FAM-55NOTAM-F, 5FAM-55NOTAM-R, 5FAM-NTSSDNA was synthesized by a gene synthesis company (Comate Bioscience Co., Ltd.), in which the specific sequences of primers and the sequences of single-stranded DNA substrates are shown below.

### 2700 bp Double-Stranded DNA Substrate (AGGAG)

The 2700 bp double-stranded DNA substrate employed in the in vitro enzymatic digestion experiment was derived from the pUC19 plasmid. This DNA substrate was amplified using primers DS2700-F and DS2700-R, after which it was obtained and recovered.

If the underlined sequence below is replaced with GAGGG, it becomes a GAGGG substrate.

(SEQ ID NO: 119)
5′-tgagtgataacactgcggccaacttacttctgacaacgatcggaggaccgaaggagctaaccgcttttttgcacaacatgggggatcatgta
actcgccttgatcgttgggaaccggagctgaatgaagccataccaaacgacgagcgtgacaccacgatgcctgtagcaatggcaacaacgttgcg
caaactattaactggcgaactacttactctagcttcccggcaacaattaatagactggatggaggcggataaagttgcaggaccacttctgcgct
cggcccttccggctggctggtttattgctgataaatctggagccggtgagcgtgggtctcgcggtatcattgcagcactggggccagatggtaag
ccctcccgtatcgtagttatctacacgacggggagtcaggcaactatggatgaacgaaatagacagatcgctgagataggtgcctcactgattaa
gcattggtaactgtcagaccaagtttactcatatatactttagattgatttaaaacttcatttttaatttaaaaggatctaggtgaagatccttt
ttgataatctcatgaccaaaatcccttaacgtgagttttcgttccactgagcgtcagaccccgtagaaaagatcaaaggatcttcttgagatcct
ttttttctgcgcgtaatctgctgcttgcaaacaaaaaaaccaccgctaccagcggtggtttgtttgccggatcaagagctaccaactctttttcc
gaaggtaactggcttcagcagagcgcagataccaaatactgttcttctagtgtagccgtagttaggccaccacttcaagaactctgtagcaccgc
ctacatacctcgctctgctaatcctgttaccagtggctgctgccagtggcgataagtcgtgtcttaccgggttggactcaagacgatagttaccg
gataaggcgcagcggtcgggctgaacggggggttcgtgcacacagcccagcttggagcgaacgacctacaccgaactgagatacctacagcgtga
gctatgagaaagcgccacgcttcccgaagggagaaaggcggacaggtatccggtaagcggcagggtcggaacaggagagcgcacgagggagcttc
cagggggaaacgcctggtatctttatagtcctgtcgggtttcgccacctctgacttgagcgtcgatttttgtgatgctcgtcaggggggcggagc
ctatggaaaaacgccagcaacgcggcctttttacggttcctggccttttgctggccttttgctcacatgttctttcctgcgttatcccctgattc
tgtggataaccgtattaccgcctttgagtgagctgataccgctcgccgcagccgaacgaccgagcgcagcgagtcagtgagcgaggaagcggaag
agcgcccaatacgcaaaccgcctctccccgcgcgttggccgattcattaatgcagctggcacgacaggtttcccgactggaaagcgggcagtgag
cgcaacgcaattaatgtgagttagctcactcattaggcaccccaggctttacactttatgcttccggctcgtatgttgtgtggaattgtgagcgg
ataacaatttcacacaggaaacagctatgaccatgattacgccaagcttgcatgcctgcaggcctggacatccgcgagcacagctaaggccaggg
aggagCTGATGGTCCATGTCTGTTActcacaccgaccggcacagcatcaagaagcgggtaccgagctcgaattcactggccgtcgttttacaacg
tcgtgactgggaaaaccctggcgttacccaacttaatcgccttgcagcacatccccctttcgccagctggcgtaatagcgaagaggcccgcaccg
atcgcccttcccaacagttgcgcagcctgaatggcgaatggcgcctgatgcggtattttctccttacgcatctgtgcggtatttcacaccgcata
tggtgcactctcagtacaatctgctctgatgccgcatagttaagccagccccgacacccgccaacacccgctgacgcgccctgacgggcttgtct
gctcccggcatccgcttacagacaagctgtgaccgtctccgggagctgcatgtgtcagaggttttcaccgtcatcaccgaaacgcgcgagacgaa
agggcctcgtgatacgcctatttttataggttaatgtcatgataataatggtttcttagacgtcaggtggcacttttcggggaaatgtgcgcgga
acccctatttgtttatttttctaaatacattcaaatatgtatccgctcatgagacaataaccctgataaatgcttcaataatattgaaaaaggaa
gagtatgagtattcaacatttccgtgtcgcccttattcccttttttgcggcattttgccttcctgtttttgctcacccagaaacgctggtgaaag
taaaagatgctgaagatcagttgggtgcacgagtgggttacatcgaactggatctcaacagcggtaagatccttgagagttttcgccccgaagaa
cgttttccaatgatgagcacttttaaagttctgctatgtggcgcggtattatcccgtattgacgccgggcaagagcaactcggtcgccgcataca
ctattctcagaatgacttggttgagtactcaccagtcacagaa-3′

TABLE 4
The sequences of enzymatic digestion substrates and primers

| | | | SEQ |
|---|---|---|---|
| Name | Description | Sequence | ID NO |
| PAM-I- | Forward primer for the construction | 5- | 120 |
| F | of PAM plasmid library fragments | cctggacatccgcgagcacagctaaggccaNNNN | |
| | | NNNCTGATGGTCCATGTCTGTTAC | |
| | | TC-3 | |
| PAM-I- | Reverse primer for the construction | 5- | 121 |
| R | of PAM plasmid library fragments | cttcttgatgctgtgccggtcggtgtGAGTAACA | |
| | | GACATGGACCATCAG-3 | |
| PAM-V- | Forward primers for the | 5- | 122 |
| F | construction of PAM plasmid | ggcacagcatcaagaagcgggtaccgagctcgaattc | |
| | library vectors | a-3 | |
| PAM-V- | Reverse primers for the | 5- | 123 |
| R | construction of PAM plasmid library | agctgtgctcgcggatgtccaggcctgcaggcatgca | |
| | vectors | agcttg-3 | |
| N20- | Forward primers for the | 5- | 124 |
| TAR-F | construction of PGEX-6P1- | CTCCTCGCTGGCGCCGGCTGGGC | |
| | FbRAD1-N20-HDV plasmid | AACATGCTTCGGCATGGCGAATG | |
| | containing a 20 bp targeted | GGACCCGGAATTCCCGGGTCGAC | |
| | sequence | TCGA-3 | |
| N20- | Forward primers for the | 5- | 125 |
| TAR-R | construction of PGEX-6P1- | CCAGCCGGCGCCAGCGAGGAGGC | |
| | FbRAD1-N20-HDV plasmid | TGGGACCATGCCGGCCTAACAGA | |
| | containing a 20 bp targeted | CATGGACCATCAGCTGCCACTCA | |
| | sequence | AAATGTGATTAAAT-3 | |
| DS2700- | Forward primer for amplification | 5-tgagtgataacactgcggccaacttac-3 | 126 |
| F | of the 2700 bp double-stranded DNA | | |
| DS2700- | Reverse primers for amplification | 5-ttctgtgactggtgagtactcaaccaag-3 | 127 |
| R | of the 2700 bp double-stranded DNA | | |
| 5FAM- | 55 nt long, 5′-end FAM-modified | 5FAM- | 128 |
| 55TAM- | forward single-stranded DNA | ggccaccattaggagCTGATGGTCCATGT | |
| F | containing AGGAG and the 20 nt | CTGTTACTCacaccgaccggcacagc-3 | |
| | targeted sequence | | |
| 5FAM- | 55 nt long, 5′-end FAM-modified | 5FAM- | 129 |
| 55TAM- | reverse single-stranded DNA | gctgtgccggtcggtgtGAGTAACAGACAT | |
| R | containing AGGAG and the 20 nt | GGACCATCAGctcctaatggtggcc-3 | |
| | targeted sequence | | |
| 5FAM- | 55 nt long, 5′-end FAM-modified | 5FAM- | 130 |
| 55NOT | forward single-stranded DNA | ggccaccattTTGATCTGATGGTCCAT | |
| AM-F | containing no AGGAG and | GTCTGTTACTCacaccgaccggcacagc-3 | |
| | containing the 20 nt targeted | | |
| | sequence | | |
| 5FAM- | 55 nt long, 5′-end FAM-modified | 5FAM- | 131 |
| 55NOT | reverse single-stranded DNA | gctgtgccggtcggtgtGAGTAACAGACAT | |
| AM-R | containing no AGGAG and | GGACCATCAGATCAAaatggtggcc-3 | |
| | containing the 20 nt targeted | | |
| | sequence | | |
| 5FAM- | 50 nt long, 5′-end FAM-modified | 5FAM- | 132 |
| NTSSDNA | forward single-stranded DNA | cggtatttcacaccgcatatggtgcactctcagtacaat | |
| | without AGGAG and the 20 nt | ctgctctgatg-3 | |
| | targeted sequence | | |
| 20 | a single-stranded DNA of 20 nt | 5-TAACAGACATGGACCATCAG-3 | 133 |
| activator | complementary to 20 nt targeting | | |
| | sequence of the RAD system′s | | |
| | RNA, for activation of trans- | | |
| | cleavage activity | | |
| Adapter- | The forward primer of the adapter | 5- | 134 |
| F | sequence linked to the enzymatic | ATAAGGAGCTGGAGAACCTGGAG | |
| | digestion product in the TAM motif | ATCAATCTGCGGAAGGAGATCGC | |
| | identification experiment, for | CAAGGCCTTCAAGGGCAACGAGG | |
| | subsequent high-throughput | GCTACAAGTCCT-3 | |
| | sequencing PCR amplification | | |
| Adapter- | The reverse primer of the adapter | 5- | 135 |
| R | sequence linked to the enzymatic | GGACTTGTAGCCCTCGTTGCCCTT | |
| | digestion product in the TAM motif | GAAGGCCTTGGCGATCTCCTTCCG | |
| | identification experiment, for | CAGATTGATCTCCAGGTTCTCCAG | |
| | subsequent high-throughput | CTCCTTAT-3 | |
| | sequencing PCR amplification | | |
| TAMSEQ- | The high-throughput sequencing | 5- | 136 |
| F | forward primer for TAM motif | GGAGTGAGTACGGTGTGCATAAG | |
| | identification | GAGCTGGAGAACCTG-3 | |
| TAMSEQ- | The high-throughput sequencing | 5- | 137 |
| R | reverse primer for TAM motif | GAGTTGGATGCTGGATGGGAGCG | |
| | identification | GATAACAATTTCACACAGG-3 | |

In vitro enzymatic digestion experiment: 5 nM of each of the RAD protein-nucleic acid complexes was added into 20 μL of reaction system (25 mM Tris 8.0, 50 mM NaCl, 2 mM DTT, 5 mM MgCl₂), and different substrates (200 ng of PAM substrate plasmid library or 200 ng of 2700 bp double-stranded DNA substrate or 20 ng 5′ FAM-labeled single-stranded DNA substrate) were added for different reactions. The reaction time ranged from 30 seconds to 2 hours, and the reaction temperature was 37° C. The reaction was stopped by adding equal volumes of 2× loading buffer (8 M urea, 0.05% bromophenol blue, 0.05% xylene blue, 1×TBE).

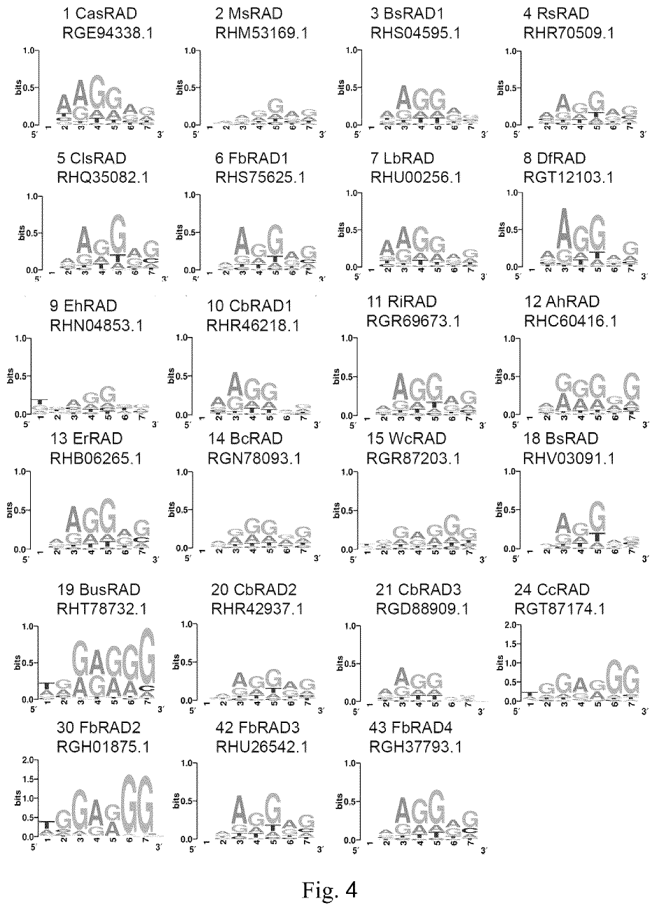

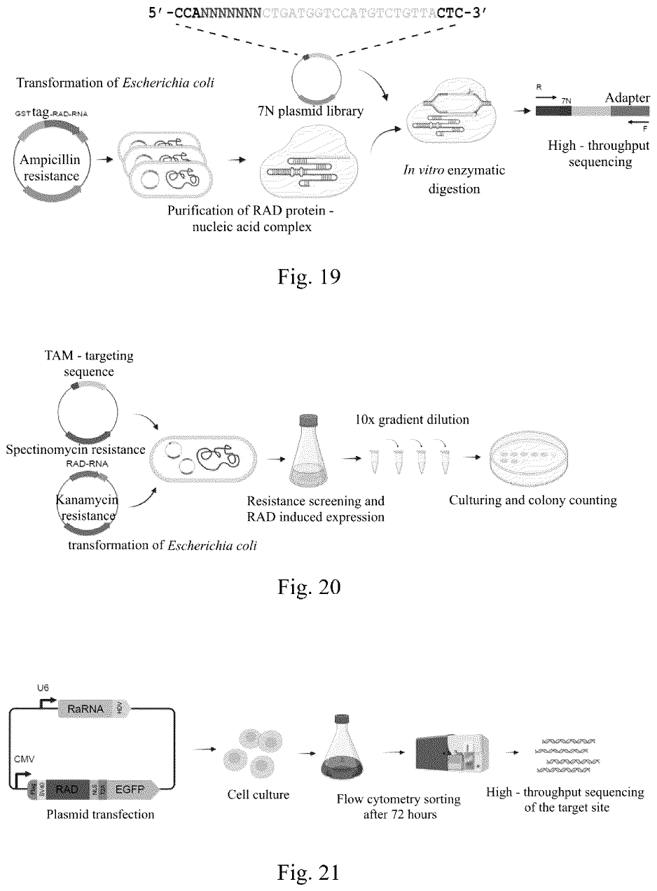

High-throughput sequencing of TAM sequences: the RAD system protein-nucleic acid complex and PAM substrate plasmid library were digested according to the above in vitro enzymatic digestion experiment, and then the reaction product was obtained and recovered. Then the recovered product was ligated into the adapter sequence (see in Table 4, Adapter-F/R) using TA cloning, and the ligated product sequence was amplified using primers TAMSEQ-F/R. The amplification time was 30 seconds, and the primer annealing temperature was 58° C., with 20 amplification cycles. The resulting samples were sent to a sequencing company for high-throughput sequencing analysis (FIG. 19). The obtained data were counted statistically to obtain the motif sequence (TAM) at the 5′ end of the RAD systems (FIG. 4 and Table 5). The results showed that the TAM of different RAD systems numbered 1-57 showed a preference of AGGAG, and different RAD proteins also showed different preferences, such as GAGGG. The RNA proteins numbered 58-99 showed TAM preferences as shown in Table 5.

TABLE 5
Motif sequences (TAM) at the 5′ end of the RAD systems

| Number | Protein ID | TAM |
|---|---|---|
| 58 | ERR3307045_63958_30277_4897_6000_− | NGGAG |
| 59 | GUT_GENOME151180_45_9178_10326_+ | NGGNN |
| 60 | ERR2017626_1022590_12863_8034_9173_+ | AGGAG |
| 61 | GUT_GENOWE142162_18_64466_65659_− | NGGAG |
| 62 | NC_013216_1_421908_422903_+ | GAGGG |
| 63 | WP_035125047.1 | GGGGG |
| 64 | GUT_GENOME096560_11_244135_245259_− | GAGGG |
| 65 | GUT_GENOME083533_37_11359_12597_− | NNGGG |
| 66 | WP_023518930.1 | NNAGG |
| 67 | WP_012574395.1 | GGGGG |
| 68 | WP_099988772.1 | AGGNN |
| 69 | GUT_GENOME228548_94_18418_19641_+ | NTAAA |
| 70 | WP_012201916.1 | GGGGG |
| 71 | WP_081429131.1 | AGGAG |
| 72 | GUT_GENOME249453_33_14687_15901_− | NGGNN |
| 73 | GUT_GENOME046003_7_13445_14386_− | NGGNN |
| 74 | GUT_GENOME043660_1_131655_132716_− | GGGGG |
| 75 | WP_039636020.1 | GGGGG |
| 76 | WP_013360293.1 | NGAGG |
| 77 | GUT_GENOME243333_13_9207_10436_+ | NNGGN |
| 78 | GUT_GENOME228909_131_318_1547_− | NNGGN |
| 79 | RGS74140.1 | NNGGN |
| 80 | GUT_GENOME029159_118_4969_5874_− | GAGGG |
| 81 | WP_012289953.1 | AGGAG |
| 82 | WP_044824965.1 | GGGGG |
| 83 | GUT_GENOME100288_478_819_1994_− | NGGNN |
| 84 | GUT_GENOME089678_61_13091_14269_− | NNGGN |
| 85 | GUT_GENOME190643_17_8068_9183_− | GGGGN |
| 86 | ERR1539620_45264_57524_39821_41020_− | NNGGG |
| 87 | WP_072587064.1 | NNGGN |
| 88 | AYF55140.1 | GAGGG |
| 89 | AUM93701.1 | NNGGN |
| 90 | SRR5892202_233507_16022_11195_12361_+ | AGGNN |
| 91 | WP_000978855.1 | GGGGG |
| 92 | WP_000604588.1 | NNGGN |
| 93 | WP_077326813.1 | NGGAG |
| 94 | GUT_GENOME170581_2_14431_15588_− | NNGGG |
| 95 | WP_025697990.1 | AGGAG |
| 96 | QAT40560.1 | AGGAG |
| 97 | WP_014431089.1 | NNAGG |
| 98 | GUT_GENOME088555_122_512_1693_+ | GAGGG |
| 99 | AWX54000.1 | NNGGN |

### In Vitro Cleavage Activity of the RAD Systems

Construction of double-stranded DNA substrate: after obtaining the sequence of AGGAG, the inventors constructed a double-stranded DNA substrate that can be specifically recognized by RAD proteins (the sequence is shown above in the 2700 bp double-stranded DNA substrate). Specifically, the 2700 bp double-stranded DNA substrate sequence used in the in vitro enzymatic digestion experiment was derived from PUC19 plasmid, and was obtained and recovered by an amplification with primers DS2700-F and DS2700-R.

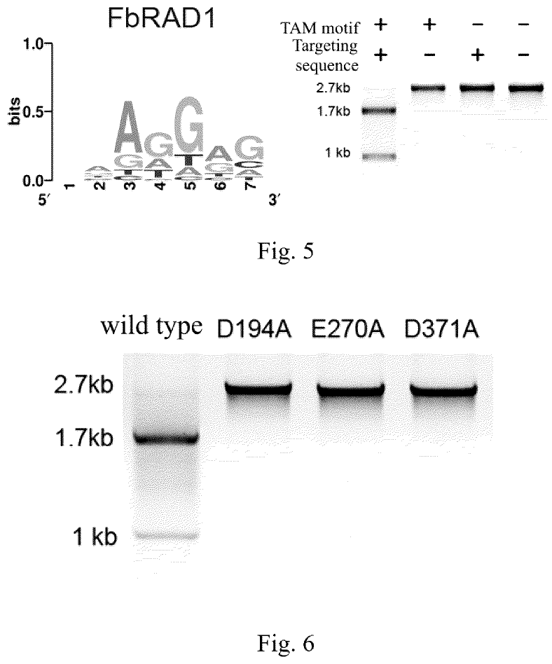

Determination of positions at which cleavage occurs: The FbRAD1 (RHS75625.1, SEQ ID NO: 6) system derived from the Firmicutes bacterium AM43-11BH was used as the experimental object. It was found that only when the TAM sequence and the targeted sequence existed at the same time, the RAD system (comprising the RAD protein SEQ ID NO: 6, its associated RNA sequence SEQ ID NO: 63 and the targeting sequence) could specifically cleave the double-stranded DNA substrate (comprising AGGAG and the targeted sequence), as shown in FIG. 5, where the left panel shows the analysis of the TAM sequence, and the right panel shows the results of cleavage. It shows that the RAD system has the same basic mechanism as conventional gene editing tools, and can function to specifically cleave double-stranded DNA in vitro.

The RAD system protein-nucleic acid complex and the 2700 bp double-stranded DNA substrate were enzymatically digested in vitro according to Example 4 above to obtain a reaction product. The PAM substrate plasmid library and 2700 bp double-stranded DNA substrate were separated into product bands using 1% agarose gel electrophoresis, and the 5′FAM labeled single-stranded DNA substrate was separated into product bands using 15% denaturing Urea-PAGE gel. The recovered products were sent to the sequencing company for Sanger sequencing and high-throughput sequencing, and the sequencing maps and quantitative statistical data of different cleavage positions were obtained, respectively.

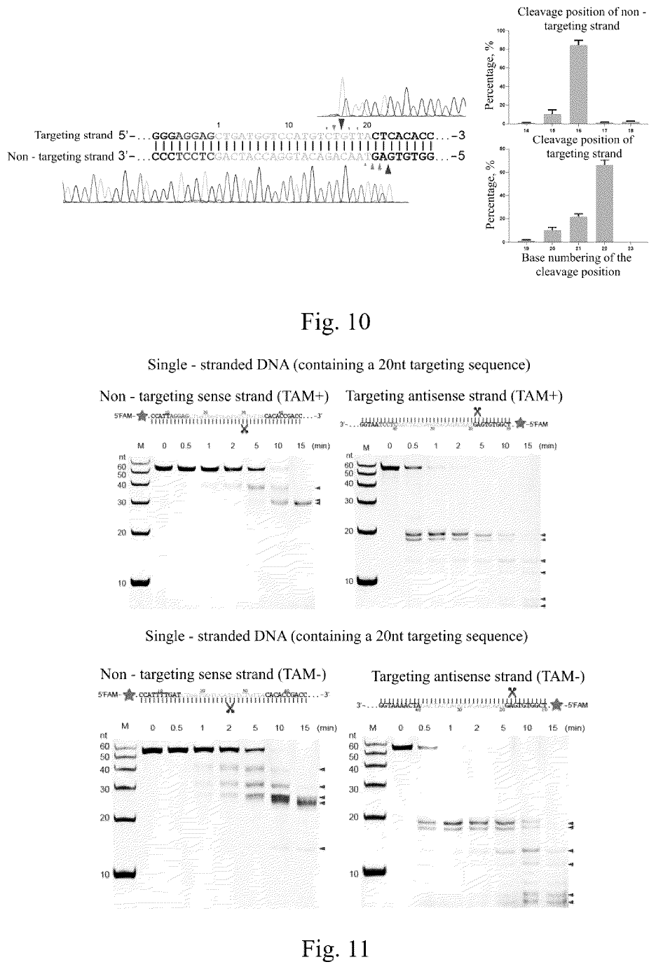

Using high-throughput sequencing and Sanger sequencing, it was found that RAD protein cleaved the double-stranded substrate DNA to produce a sticky end nick. The cleavage mainly occurred at the base position 22 of the targeted strand and the base position 16 of the non-targeted strand, respectively (FIG. 10) (for the experimental procedures, see in vitro enzymatic digestion experiment). A small amount of cleavage was produced at other positions. This experiment again proved that RAD protein had specific double-stranded DNA endonuclease activity.

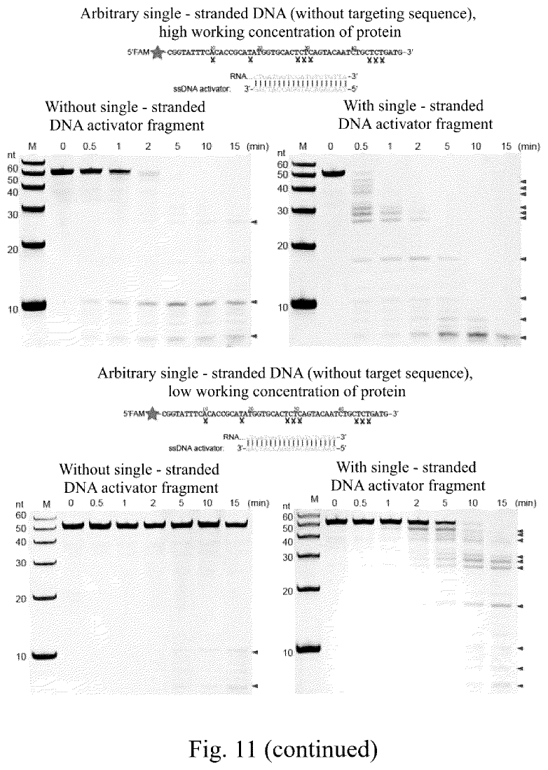

In vitro single-stranded enzymatic digestion experiment: the 5′ end of single-stranded DNA was fluorescently labeled with FAM to perform an in vitro enzymatic digestion experiment, as described in the above in vitro enzymatic digestion experiment. As a result, as shown in FIG. 11, the RAD system can specifically cleave not only double-stranded DNA, but also single-stranded DNA. The cleavage of the single-stranded DNA did not require the TAM sequence. The activity of cleaving single-stranded DNA that matches the RNA targeting sequence was significantly higher than that for non-matching DNA. Furthermore, the single-stranded DNA was cleaved without matching the targeting sequence. The 20-base single-stranded DNA matching the end of the RNA (20activator in Table 4) was added for activation of the RAD protein. Subsequently, the activity of the RAD protein to cleave the non-specific single-stranded DNA was significantly increased, indicating that it has the trans-cleavage activity of single-stranded DNA. This single-stranded DNA cleavage activity can be developed as an identification tool for single-stranded DNA viruses as follows. A sequence from a target virus is constructed into the RNA of the RAD system. A single-stranded DNA substrate is used which produces chemical or fluorescent signals after cleavage. When the RAD system recognizes the target virus, the trans-cleavage activity is activated, and the substrate is cleaved followed by generation of a signal, thereby identifying the target virus.

### Determination of Conditions of Cleavage

A series of in vitro enzymatic digestion experiments were carried out under different conditions, and the experimental processes were the same as above. Suitable conditions for temperature, divalent cation type, divalent cation concentration, targeting sequence length, and salt concentration were obtained.

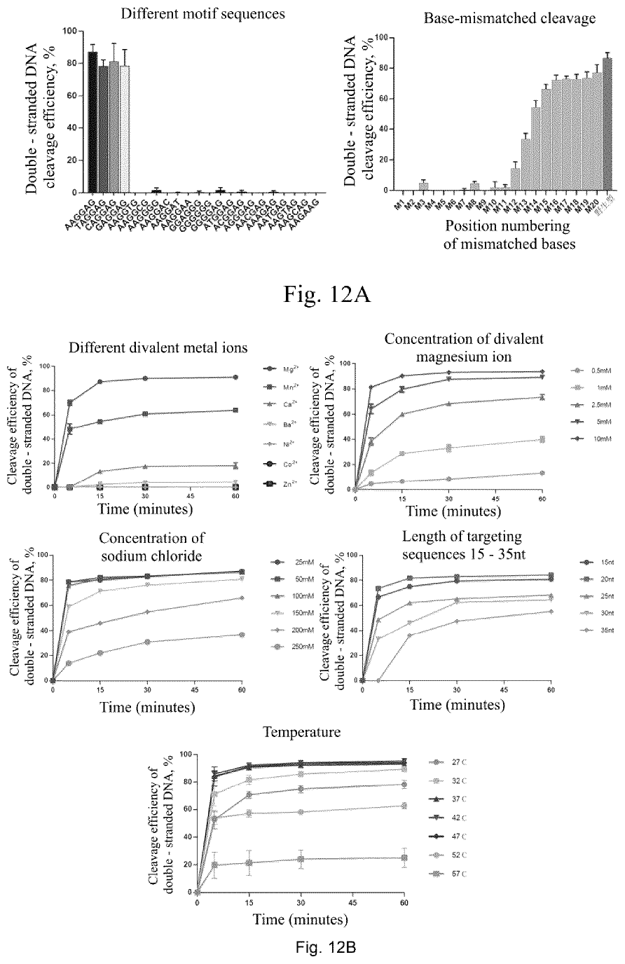

The results are shown in FIG. 12B. The results showed the suitable conditions of in vitro enzymatic digestion for the RAD system containing FbRAD1. The cleavage activity was considerably high at 42° C. in presence of 5 to 10 mM MgCl₂ and 25 to 100 mM NaCl, and the activity was highest with a targeting sequence length of 20 bases.

### Effects of Motifs and Mismatched Sequences

Based on the constructed enzymatic digestion substrate and in vitro enzymatic digestion experiments as described above, 5 nM of FbRAD1 protein-nucleic acid complex and 200 ng of 2700 bp double-stranded DNA substrate were added to 20 μL reaction system (25 mM Tris 8.0, 50 mM NaCl, 2 mM DTT, 5 mM MgCl₂) and were reacted at 37° C. for 30 minutes. DNA cleavage with different TAM sequences was verified. It was found that AGGAG was the most suitable TAM for FbRAD1 protein which recognizes AGGAG sequence, and a change of any base would greatly reduce the cleavage activity or even result in loss of the cleavage activity (FIG. 12A).

Based on the constructed enzymatic digestion substrates and in vitro enzymatic digestion experiments as described above, the 2700 bp mismatched double-stranded DNA substrate was used. Mismatch cleavage experiments showed that a base mismatch in positions 14 to 20 at the 3′ end of the targeting sequence reduced cleavage activity, but the effect was little. However, a mismatch at positions 1-13 greatly reduced the cleavage activity. The results of this experiment showed that the RAD system had a low tolerance to a base mismatch at positions 1-13, indicating the specificity of the RAD system was strong. As conventional CRISPR/Cas9 and Cas12a can still result in cleavage in the presence of the mismatch at positions 6 to 13, their specificities are poorer than that of the RAD system (FIG. 12B).

### Example 4: Analysis of RAD Complex Domain

Through a domain analysis, it was found that RAD protein contained a RuvC domain with three active centers, which in the FbRAD1 system were aspartic acid at amino acid position 194, glutamic acid at position 290, and aspartic acid at position 371. The three amino acids were highly conserved in all RAD proteins. In the domain analysis, firstly, the structurally resolved Cas proteins with RuvC were collected, and three RuvC domain sequences were picked up, respectively, to perform three iterative distal homology searches in the NCBI database, with evalue of 1e-4. Then, multiple-sequence alignment of the sequence of each domain was performed together with its homologous sequences. Then, hmm was constructed based on the results of the multiple-sequences alignment, using the commonly used tool hmmscan with the threshold set to evalue=1e-10, HMMER web server: interactive sequence similarity searching.

The wild-type sequence is FbRAD1. The mutants D194A, E290A, and D371A with amino acid inactive mutations to alanine of the three active centers in this example were prepared using the amino acid point mutation kit from Vazyme Biotech Co., Ltd. according to the instructions of the kit. Expression and purification of the RAD (IS607 TnpB) protein-nucleic acid complex were shown in Example 1. The double-stranded DNA cleavage of RAD (IS607 TnpB) protein-nucleic acid complexes of wild-type, mutants D194A, E290A, and D371A were determined as described in Example 3.

Functional verification of active centers: as shown in FIG. 6, the RAD protein lost the activity to cleave double-stranded DNA after the inactive mutations to alanine of the amino acids at the three active centers, indicating that the RuvC domain of the RAD protein itself exerted DNA endonuclease activity (FIG. 6).

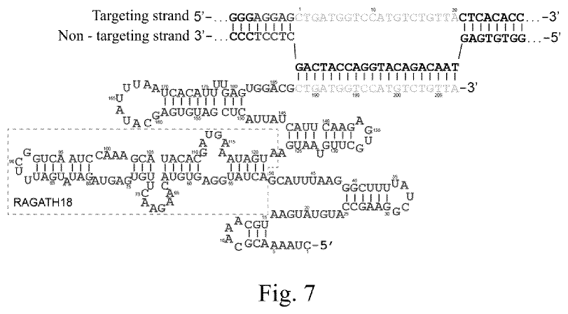

Structural analysis: using the RNA secondary structure prediction website RNA folder web server, it was found that the RNA of RAGATH-18 formed the longest stem-loop structure in the entire complex, as shown in FIG. 7.

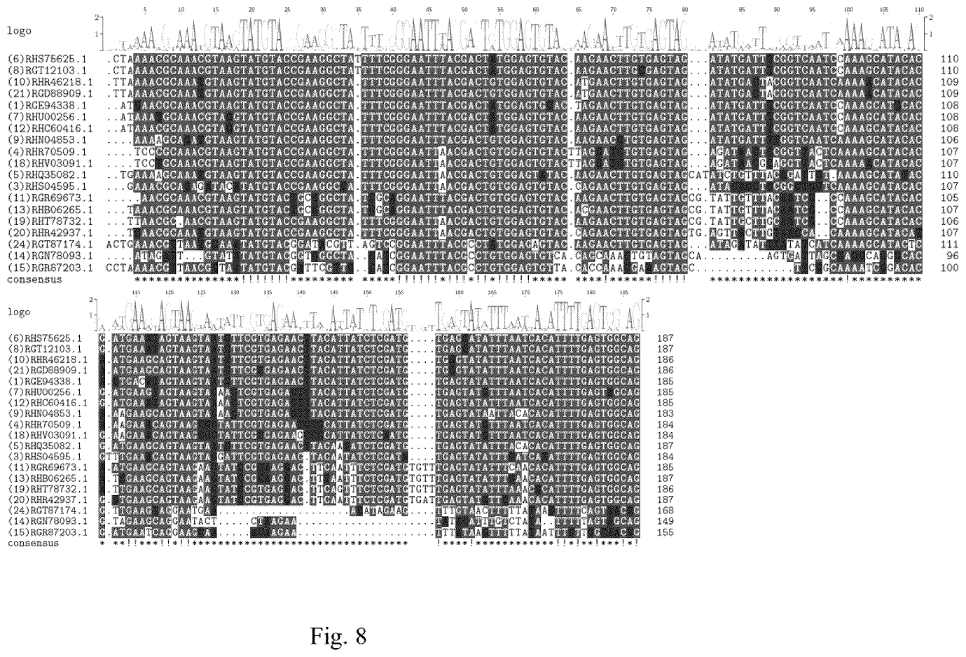

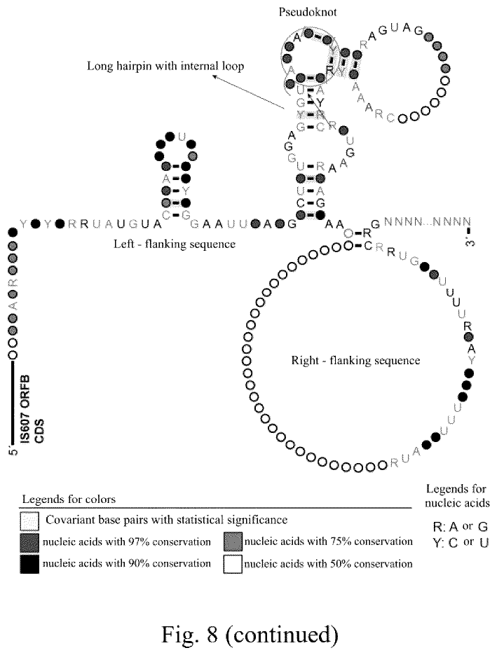

As shown in FIG. 8, RNA multiple-sequence alignment analysis on a variety of RAD systems with DNase activity was performed in the MAFFT version 7 website, using FbRAD1 (RHS75625.1) as the alignment reference. The inventors found that the average length of RAGATH18 RNA was 75 bp, containing a predicted long hairpin with an inner loop and a pseudo-knot, and the both left and right flanking sequences of RAGATH18 were also conserved. The left flanking sequence was about 50 bp in length containing a predicted stem loop, and the right flanking sequence was about 60 bp in length. The RNA sequences of RAGATH-18 with the predicted stem loop (FIG. 8) were highly conserved in RAD systems. The results indicated that RAGATH-18 RNA was necessary in the RAD system, and also indicated that the RAD system with RAGATH-18-associated RNA had DNase activity.

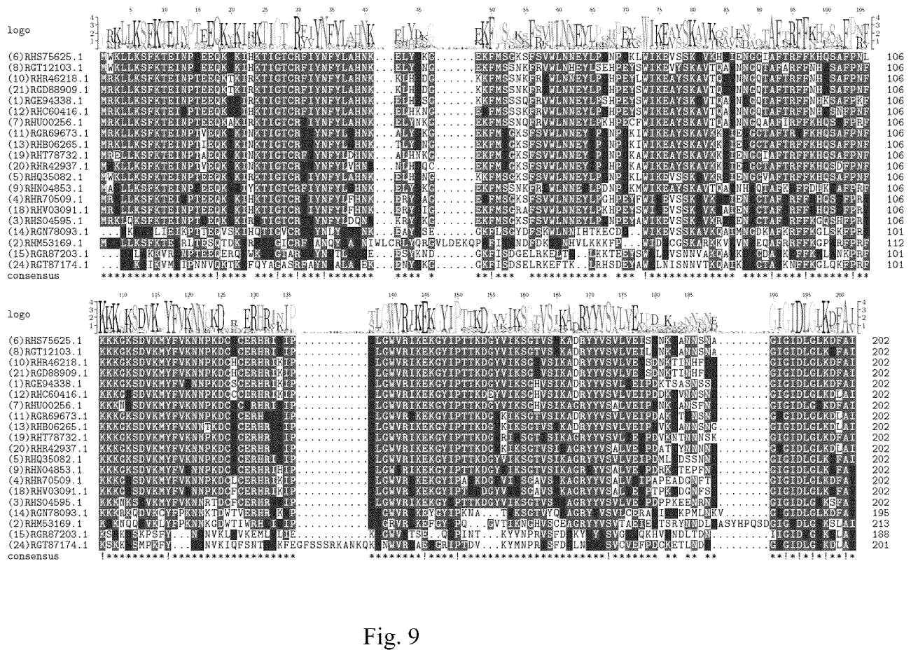

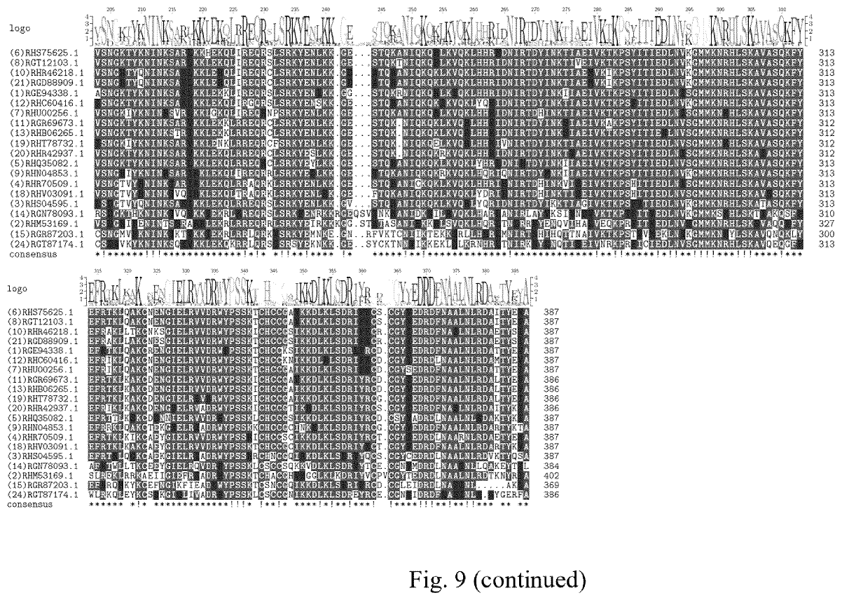

As shown in FIG. 9, multiple-sequence alignment analysis on 20 types of RAD systems with DNase activity was performed in the MAFFT version 7 website, using FbRAD1 (RHS75625.1) as the alignment reference. We found that in different strains of different genera, the following amino acids in proteins of the RAD system are highly conserved: Q18, G27, R30, N34, F50, W73, A92, F97, P104, K107, Y117, E145, P150, 5173, G192, D194, G196, K198, A201, 5204, N211, 1212, N213, R227, Q229, S233, R234, E237, G242, N249, K252, N266, V280, K283, P284, E290, L292, N293, G296, M297, M298, K299, L303, 5304, K305, Q310, K322, P338, S339, 5340, C343, C346, L353, L355, R358, C362, C364, D369, R370, D371, A374, N377 and L378.

Based on the above results of sequence alignment analysis, we can infer that RAD proteins with these conserved amino acids also have DNase activity, because the RAD system is widely found in nature.

### Example 5: Intracellular Cleavage Activity in Prokaryotes

Addition of targeting sequence: a 20-base sequence (CTGATGGTCCATGTCTGTTA, SEQ ID NO: 116) was directly added to the 3′ end of the RNA sequence (SEQ ID NO: 63) bound by RAD protein FbRAD1 to serve as a targeting sequence. The 20-base sequence was complementary to the sequence of the PAM plasmid library and can target the PAM plasmid library. The addition was performed as described below. The expression plasmid was amplified by PCR using primers N20-TAR-F and N20-TAR-R (primer sequences are shown in Table 4), using each of PGEX-RAD 1-57 plasmids synthesized in Example 2 (for example, PGEX-6P1-FbRAD1-N20-HDV) as a template. The PCR amplification time was 6 minutes, the primer annealing temperature was 58° C., and the number of amplification cycles was 30. Then, the PCR product was digested with Dpn1 enzyme.

Constructing E. coli intracellular expression vectors: the digested product was ligated to the linear vector of the universal vector PET-28A by homologous recombination using the recombinase system from Vazyme Biotech Co., Ltd., and the recombinant product was transformed into E. coli DH5α competent cells. On the second day, single colonies were picked up and sequenced for identification, in order to obtain the RAD system expression plasmids pET-28a RAD 1-57 containing targeting sequences. An exemplary plasmid sequence, pET-28a-FbRAD1-N20-HDV, is shown below. This exemplary plasmid is a plasmid for the FbRAD1 system, which is a vector for the intracellular editing experiment in E. coli. The RAD system was inserted at the Nco1 and Xho1 sites. The structure of FbRAD1 protein-6HIS-RNA-N20-HDV was shown below, where FbRAD1 protein is in bold and underlined, 6HIS is underlined, RNA is in bold, N20 is in italic, and HDV is in italic and in bold:

(SEQ ID NO: 117)
ATGAGGAAATTGCTGAAGAGCTTCAAGACGGAAATAAATCCAACAGAAGAGCA
GAAAGTCAGGATTCGTAAAACAATAGGTACTTGCAGATTTATCTATAACTTCTATCTTGCT
CATAATAAGGAGCTTCATGAAAGTGGCAAAAAGTTTATGAGCAGCAGCCAATTCAGAGTA
TGGCTCAACAATGAATATCTTCCGAGCCATCCGGAATATTCCTGGATCAAGGAAGCTTAT
TCAAAATCGGTAACACAGGCAGTAAATGATGGACAGACCGCATTTACAAGATTCTTTAAT
CACAAGAGCGCCTTTCCAAAGTTTAAAAAGAAAGGCAAGTCTGATGTGAAAATGTATTTT
GTAAGGAATAATCCAAAAGATTGCAGCTGCGAAAGACACAGAATCAAGATTCCTTCACTT
GGCTGGGTCCGTATCAAAGAGAAAGGATATATTCCCACTACAAAAGATGGATATGTAATT
AAGAGCGGTCATGTCTCAATAAAAGCTGATAGATACTATGTTTCAGTTCTTATAGAAATC
CCAGACAAAACGTCAGCTAGTAATTCCAGTGAAGGAATTGGCATTGACCTTGGATTAAAG
GACTTTGCGATTGCTTCTAATGGAAAGACTTATAAGAACATCAACAAATCTGCTAAATTAA
AGAAGCTTGAAAAAAAGCTTATTCGAGAACAGAGAAGTCTTTCTCGAAAGTATGAAAATA
TAAAGAAAGGAGGGTCCACTCAAAAAAGAAATATACAAAAACAAAGGCTTAAGATACAGA
AACTTCATCATAGAATCGACAATATTCGTACTGATTACATCAATAAGATAATCGCAGAGAT
AGTGAAAACCAAGCCATCTTATATAACGATTGAAGATCTAAACATATCAGGGATGATGAA
GAACAGACATCTTTCAAAGGCAGTTGCTTCGCAGAAGTTTTATGAATTTAAGACAAAGCT
TCAAGCTAAATGCAGGGAGAACGGAATCGAACTTCGAGTTGTAGATAGATGGTTCCCATC
ATCTAAGACTTGTCACTGCTGTAAAAGTATCAAGAAAGATTTAAAGCTTTCAGATAGACTT
TTCAGATGTGATTGTGGATATATCGAAGACAGAGATTTCAATGCTGCACTAAATCTGAGA
GATGCTACAACTTACGAAGTTGCAAGCAGCCATCACCATCATCACCACTAAATGAACGCAA
ACGTAAGTATGTACCGAAGGCTATTTCGGGAATTTACGACTATGGAGTGCACTAGAACTT
GTGAGTAGATATGATTTCGGTCAATCCAAAGCATGCACAGTGACATAGTAAGTAATGTTC
GTGAGAACTTACATTATCTCGATGTGAGTATATTTAATCACATTTTGAGTGGCAGCTGATG
GTCCATGTCTGTTAGGCCGGCATGGTCCCAGCCTCCTCGCTGGCGCCGGCTGGGCAACATGCTTC
GGCATGGCGAATGGGACCCG

Intracellular verification of DNA interference in E. coli: 57 RAD systems in Table 2 were cloned into PET28a (+) expression vectors at Nco1 and Xho1 cleavage sites by homologous recombination, and a spectinomycin-resistant plasmid PCS101ORI (pTargetF-aggag-n20-psc101ori) containing TAM sequence and a targeted sequence (CTGATGGTCCATGTCTGTTA, SEQ ID NO: 116) was constructed. The sequence of the plasmid is shown below.

pTargetF-aggag-n20-psc101ori (Targeted plasmid having spectinomycin resistance used in E. coli
intracellular editing experiments). The motif sequence is underlined, and the targeted sequence
is in bold)
(SEQ ID NO: 138)
catgttctttcctgcgttatcccctgattctgtggataaccgtattaccgcctttgagtgagctgataccgctcgccgcagccgaacgaccgag
cgcagcgagtcagtgagcgaggaagcggaagagcgcctgatgcggtattttctccttacgcatctgtgcggtatttcacaccgcatatgctgga
tccttgacagctagctcagtcctaggtataatactagtattgtcaccagactggcgtcgttttagagctagaaatagcaagttaaaataaggct
agtccgttatcaacttgaaaaagtggcaccgagtcggtgctttttttgaattctctagagtcgacctgcagaagcttagatctattaccctgtt
atccctactcgagttcatgtgcagctccataagcaaaaggggatgataagtttatcaccaccgGGGAGGAGCTGATGGTCCATGTCTGTTAact
atttgcaacagtgccgttgatcgtgctatgatcgactgatgtcatcagcggtggagtgcaatgtcatgagggaagcggtgatcgccgaagtatc
gactcaactatcagaggtagttggcgtcatcgagcgccatctcgaaccgacgttgctggccgtacatttgtacggctccgcagtggatggcggc
ctgaagccacacagtgatattgatttgctggttacggtgaccgtaaggcttgatgaaacaacgcggcgagctttgatcaacgaccttttggaaa
cttcggcttcccctggagagagcgagattctccgcgctgtagaagtcaccattgttgtgcacgacgacatcattccgtggcgttatccagctaa
gcgcgaactgcaatttggagaatggcagcgcaatgacattcttgcaggtatcttcgagccagccacgatcgacattgatctggctatcttgctg
acaaaagcaagagaacatagcgttgccttggtaggtccagcggcggaggaactctttgatccggttcctgaacaggatctatttgaggcgctaa
atgaaaccttaacgctatggaactcgccgcccgactgggctggcgatgagcgaaatgtagtgcttacgttgtcccgcatttggtacagcgcagt
aaccggcaaaatcgcgccgaaggatgtcgctgccgactgggcaatggagcgcctgccggcccagtatcagcccgtcatacttgaagctagacag
gcttatcttggacaagaagaagatcgcttggcctcgcgcgcagatcagttggaagaatttgtccactacgtgaaaggcgagatcaccaaggtag
tcggcaaataagatgccgctcgccagtcgattggctgagctcataagttcctattccgaagttccgcgaacgcgtaaaggatctaggtgaagat
cctttttgataatctcatgaccaaaatcccttaacgtgagttttcgttccactgagcgtcagaccccgtagaaaagatcaaaggatcttctcag
atccttccgtatttagccagtatgttctctagtgtggttcgttgtttttgcgtgagccatgagaacgaaccattgagatcatacttactttgca
tgtcactcaaaaattttgcctcaaaactggtgagctgaatttttgcagttaaagcatcgtgtagtgtttttcttagtccgttatgtaggtagga
atctgatgtaatggttgttggtattttgtcaccattcatttttatctggttgttctcaagttcggttacgagatccatttgtctatctagttca
acttggaaaatcaacgtatcagtcgggcggcctcgcttatcaaccaccaatttcatattgctgtaagtgtttaaatctttacttattggtttca
aaacccattggttaagccttttaaactcatggtagttattttcaagcattaacatgaacttaaattcatcaaggctaatctctatatttgcctt
gtgagttttcttttgtgttagttcttttaataaccactcataaatcctcatagagtatttgttttcaaaagacttaacatgttccagattatat
tttatgaatttttttaactggaaaagataaggcaatatctcttcactaaaaactaattctaatttttcgcttgagaacttggcatagtttgtcc
actggaaaatctcaaagcctttaaccaaaggattcctgatttccacagttctcgtcatcagctctctggttgctttagctaatacaccataagc
attttccctactgatgttcatcatctgaacgtattggttataagtgaacgataccgtccgttctttccttgtagggttttcaatcgtggggttg
agtagtgccacacagcataaaattagcttggtttcatgctccgttaagtcatagcgactaatcgctagttcatttgctttgaaaacaactaatt
cagacatacatctcaattggtctaggtgattttaatcactataccaattgagatgggctagtcaatgataattactagtccttttcctttgagt
tgtgggtatctgtaaattctgctagacctttgctggaaaacttgtaaattctgctagaccctctgtaaattccgctagacctttgtgtgttttt
tttgtttatattcaagtggttataatttatagaataaagaaagaataaaaaaagataaaaagaatagatcccagccctgtgtataactcactac
tttagtcagttccgcagtattacaaaaggatgtcgcaaacgctgtttgctcctctacaaaacagaccttaaaaccctaaaggcttaagtagcac
cctcgcaagctcggttgcggccgcaatcgggcaaatcgctgaatattccttttgtctccgaccatcaggcacctgagtcgctgtctttttcgtg
acattcagttcgctgcgctcacggctctggcagtgaatgggggtaaatggcactacaggcgccttttatggattcatgcaaggaaactacccat
aatacaagaaaagcccgtcacgggcttctcagggcgttttatggcgggtctgctatgtggtgctatctgactttttgctgttcagcagttcctg
ccctctgattttccagtctgaccacttcggattatcccgtgacaggtcattcagactggctaatgcacccagtaaggcagcggtatcatcaaca
acgccagcaacgcggcctttttacggttcctggccttttgctggccttttgctca

Two plasmids, spectinomycin-resistance plasmid pTargetF-aggag-n20-psc101ori and each of RAD system expression plasmids pET-28a RAD 1 to 57, were co-transformed into E. coli C43 (DE3), and the method for transformation was the same as that in Example 2. 100 ng of each of the two plasmids was added simultaneously to (E. coli C43 (DE3)) competent cells. The cells were cultured overnight on a LB plates containing both 100 μg/mL of spectinomycin and kanamycin at 30° C. The single colonies were picked up on the plates with the two antibiotics and cultured in 2 mL of LB medium containing spectinomycin and kanamycin and 0.6 mM IPTG at 30° C., 220 rpm for 12 hours overnight. On the second day, the bacterial solution was diluted serially to 10⁻⁶ in ten-fold dilutions. Then, 5 μL of the bacterial solution was dropped on plates with the two antibiotics, and cultured at 30° C. for 24 hours. The growth was observed and imaged for counting. Once DNA-specific cleavage occurs, significant growth differences can be observed on the screening plates with kanamycin and spectinomycin, and the cells in which cleavage takes place will die. FIG. 20 shows a flow chart showing the DNA cleavage by RAD protein in E. coli and the step of screening.

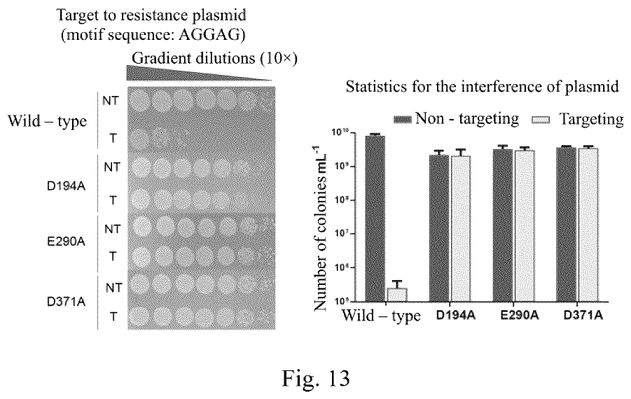

Results and FIGS. 13, 14, and 15 show that the RAD systems successfully produce specific DNA cleavage in E. coli.

The DNA sequence to be interfered with was a spectinomycin resistance sequence, which was an exogenous sequence (PUC19-TARGET1-AGGAG, PUC19-TARGET1-GAGGG, see Table [[4]]6). Under the conditions where the TAM sequence was AGGAG or GAGGG, a total of 12 RAD systems were found to successfully achieve cleavage. The 12 RAD (IS607 TnpB) systems were from 7 different species, and were (6) FbRAD1/ISFba1 (RHS75625.1), (10) CbRAD1/ISCba1 (RHR46218.1), (11) RiRAD/ISRin (RGR69673.1), (13) ErRAD/ISEre1 (RHB06265.1), (15) WcRAD (RGR87203.1), (18) BsRAD/ISBsp4 (RHV03091.1), (20) CbRAD2/ISClsp2 (RHR42937.1), (21) CbRAD3/ISClsp3 (RGD88909.1), (24) CcRAD/ISCco (RGT87174.1), (30) FbRAD2/ISFba2 (RGH01875.1), (42) FbRAD3/ISFba3(RHU26542.1), and (43) FbRAD4/ISFba4 (RGH37793.1), respectively.

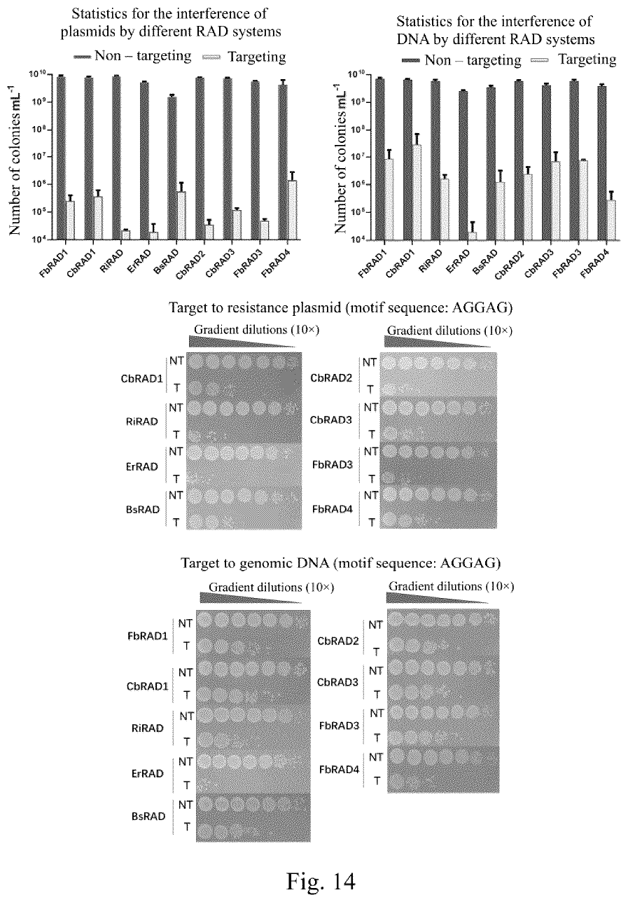

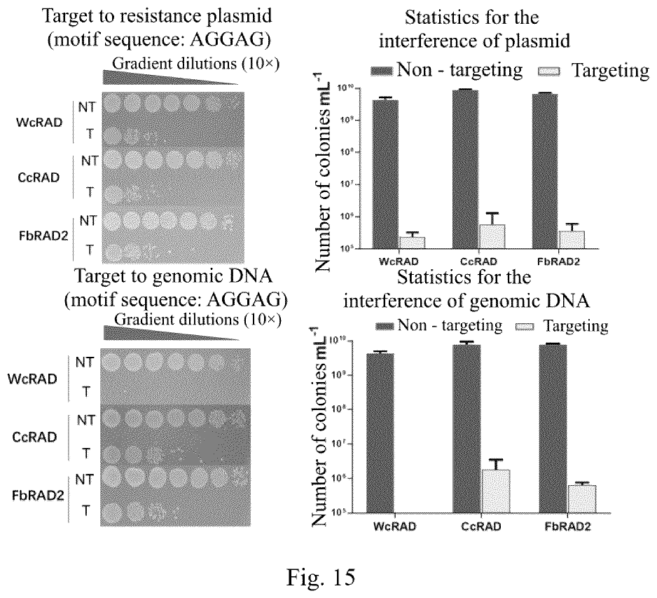

In order to cleave a DNA in the endogenous genome of E. coli, the targeting sequences were replaced against the genome of E. coli. The DNA sequence to be interfered with was a genome sequence of E. coli, which is an endogenous sequence. (ECOLI-TARGET-AGGAG and ECOLI-TARGET-GAGGG, see Table 6; the motifs and targeted sequences in pTargetF-aggag-n20-psc101ori described above were replaced with the sequences shown in Table 6). Plates with kanamycin were used to screen single colonies and were observed after 10-fold dilution titration (FIGS. 14, 15). FIGS. 14 and 15 show the DNA interference results in E. coli for different RAD systems when the TAM sequence is AGGAG or GAGGG, respectively. The colonies formed after titration show that the growths of the targeted group (T group) (i.e., FbRAD1, CbRAD1, RiRAD, ErRAD, and BsRAD) were significantly inhibited as compared to the non-targeted control group (NT group), showing a difference exceeding 104 in more than one system.

Verification of the enzymatic digestion activity after a mutation in the active centers: DNA interference no longer occurred after the inactive mutations to alanine of the amino acids of three active centers in FbRAD1, aspartic acid at position 194, glutamic acid at position 290, and aspartic acid at position 371 (FIG. 13).

TABLE 6
Sequences of targets in prokaryote experiments

| | | | SEQ ID |
|---|---|---|---|
| Name | Description | Sequence | NO |
| PUC19- | the targeted sequence of the | aggagCTGATGGTCC | 139 |
| TARGET1- | spectinomycin resistance plasmid for in | ATGTCTGTTA | |
| AGGAG | vitro enzymatic digestion experiments | | |
| | and E. coli intracellular experiments, the | | |
| | TAM motif was AGGAG, | | |
| PUC19- | the targeted sequence of the | gagggCTGATGGTC | 140 |
| TARGET1- | spectinomycin resistance plasmid for E. | CATGTCTGTTA | |
| GAGGG | coli intracellular experiments, the TAM | | |
| | motif was GAGGG, | | |
| ECOLI- | For E. coli intracellular experiments, the | aggagAGGTAGGGC | 141 |
| TARGET- | genome sequence of E. coli was targeted, | AAACCGCCCCT | |
| AGGAG | and the TAM motif was AGGAG. | | |
| ECOLI- | For E. coli intracellular experiments, the | gagggCAAAGGCCT | 142 |
| TARGET- | genome sequence of E. coli was targeted, | CCCGGAAGTTC | |
| GAGGG | and the TAM motif was GAGGG. | | |

### Example 6: In Vivo Gene Editing in Humans

Individual proteins in each of the RAD systems in Table 2 were fused to a nuclear localization signal and green fluorescent protein, and the CMV promoter was used to regulate the expression. On the same plasmid, the U6 promoter was used to regulate the expression of RNA in each of the RAD systems, and the RNA contained the 20-base genome-targeting sequence at the end.

The exemplary plasmid sequence pcdna3.1-u6-FbRAD1-cmv-3flag-SV40-FbRAD1-NLS-2a-EGFP is shown below, which is a plasmid used in intracellular editing in human 293F cells. The U6 promoter sequence is in bold, the RNA sequence associated with RAD protein FbRAD1 is italic and in bold, the CMV sequence is in bold and underlined, the 3flag sequence is italic, the SV40 sequence is italic, in bold and underlined, the RAD protein FbRAD1 is italic, in bold and double underlined, the NLS sequence is double underlined, the EGFP sequence is italic and underlined, and the 2a sequence is in bold and double underlined.

(SEQ ID NO: 143)
gagggcctatttcccatgattccttcatatttgcatatacgatacaaggctgttagagagataattggaattaatttgactgta
aacacaaagatattagtacaaaatacgtgacgtagaaagtaataatttcttgggtagtttgcagttttaaaattatgttttaaa
atggactatcatatgcttaccgtaacttgaaagtatttcgatttcttggctttatatatcttgtggaaaggacGAAACACCGCT
AAAACGCAAACGTAAGTATGTACCGAAGGCTATTTTCGGGAATTTACGACTATGGAGTGTACAAGAACTTGTGAGTAGATATGA
TTTCGGTCAATCCAAAGCATACACGATGAAATAGTAAGTAATGTTCGTGAGAACTTACATTATCTC
GATGTGAGCATATTTAATCACATTTTGAGTGGCAGCTGATGGTCCATGTCTGTTAGGCCGGCATG
GTCCCAGCCTCCTCGCTGGCGCCGGCTGGGCAACATGCTTCGGCATGGCGAATGGGACTTTTTT
GTTTTAGAGCTAGAAATAGCAAGTTAAAATAAGGCTAGTCCGTTTTTAGCGCGTGCGCCAATTC
TGCAGACAAATGGCTCTAGAGGTACCCGATGTACGGGCCAGATATACGCGTTGACATTGATTA
TTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTTTTG
GCAGTACATCAATGGGCGTGGATAGCGGTTTGACTCACGGGGATTTCCAAGTCTCCACCC
CATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATA
TAAGCAGAGCTCTCTGGCTAACTAGAGAACCCACTGCTTACTGGCTTATCGAAATTAATACGA
CTCACTATAGGGAGACCCAAGCTGGCTAGCGTTTAAACTTAAGCTTGGTACCGAGCTCGGATC
Cgccaccatggactataaggaccacgacggagactacaaggatcatgatattgattacaaagacgatgacgataagatggcc
ccaaagaagaagcggaaggtcggtatccacggagtcccagcagcc
ATGTGGAAATTGCTAAAGAGCTTCAAGACGGAAATAAATCCGTC
AGAGGAACAGAAAGTCAAGATTCATAAGACGATAGGAACTTGTAGATTCATTTATAATTTCTATCT
TGCTCATAACAAAGAACTTTACGATAAAGGCGAAAAGTTTATGAGTGGTAAAAGTTTTAGTGTTT
GGCTGAACAATGAGTATCTTCCACAAAATCCAGACAAGTTATGGATTAAGGAAGTCAGTTCAAAA
TCTGTAAAGCACTCAATTGAAAATGGTTGTATCGCATTTACCAGATTTTTCAAACATCAAAGTGCT
TTTCCGAATTTAAAAAAGAAAGGCAAATCTGATGTAAAAATGTATTTTGTAAAGAATAATCCAAAA
GATTGTCGTTGCGAAAGACACAGAATCAATATTCCGTCACTTGGTTGGGTTCGCATCAAAGAAAA
AGGATATATTCCAACAACTAAAGACGGATATGTGATTAAAAGCGGCACGGTCTCAATGAAAGCTG
ACAGATACTATGTTTCAGTTCTTGTAGAAATTTCAAATAACAAGATAGCCAATAATTCCAATGCAG
GAATTGGCATTGACCTTGGATTAAAGGATTTTGCCATTGTTTCTAATGGTAAAACTTATAAGAACA
TTAACAAATCTGCAAGATTAAAGAAACATGAAAAACAGCTTATTCGAGAACAGAGAAGTCTTTCT
CGAAAGTATGAAAATTTAAAGAAAGGAGAGTCCACTCAAAAAGCAAATATACAAAAACAAAGGCT
TAAGGTACAGAAACTTCATCATAGAATGGATAATATTCGTACTGATTACATCAATAAAACAATCGC
AGAGATAGTAAAAACCAAACCATCTTATATTACGATTGAAGATTTGAATGTAAAAGGTATGATGA
AAAACAGACATCTTTCAAAAGCTGTTGCTTCACAGAAGTTTTATGAATTTAGAACAAAGCTTCAAG
CTAAATGCAATGAAAATGGAATCGAACTTCGAGTAGTAGACAGATGGTATCCATCATCTAAAACC
TGTCACTGCTGTGGCGCTGTCAAAAAAGATTTAAAGCTTTCAGATAGAATATTTAAATGCAGTTG
CGGTTATGTCGAAGACAGAGATTTCAATGCTGCACTTAATTTGAGAGATGCGATAACTTACGAAGTTGCA
aaaaggccggcggccacgaaaaaggccggccaggcaaaaaagaaaaaggaattcggcagtgga
gagggcagaggaactctcctaacatgcggtgacgtcgaggagaatcctggccca
gtgagcaagggcgaggagctgttcaccggggtggtgcccatcctggtcgagctggacggcgacgtaa
acggccacaagttcagcgtgtccggcgagggcgagggcgatgccacctacggcaagctgaccctgaagttcatctgcaccaccg
gcaagctgcccgtgccctggcccaccctcgtgaccaccctgacctacggcgtgcagtgcttcagccgctaccccgaccacatga
agcagcacgacttcttcaagtccgccatgcccgaaggctacgtccaggagcgcaccatcttcttcaaggacgacggcaactaca
agacccgcgccgaggtgaagttcgagggcgacaccctggtgaaccgcatcgagctgaagggcatcgacttcaaggaggacggca
acatcctggggcacaagctggagtacaactacaacagccacaacgtctatatcatggccgacaagcagaagaacggcatcaagg
tgaacttcaagatccgccacaacatcgaggacggcagcctccagctcgccgaccactaccagcagaacacccccatcggcgacg
gccccgtgctgctgcccgacaaccactacctgagcacccagtccgccctgagcaaagaccccaacgagaagcgcgatcacatc
gtcctgctggagttcgtgaccgccgccgggatcactctcggcatggacgagctgtacaaggaattctaaCTCGAG

Other corresponding plasmid constructs can be obtained by replacing the nucleic acid sequences encoding FbRAD1 protein and associated RNA in the exemplary sequence pedna3.11-u6-FbRAD1-cmv-3 flag-sv40-FbRAD1-NLS-2a-EGFP with the nucleic acids encoding each of the other effector proteins and corresponding RNAs.

Cell transfection: human 293F cells were selected as the transfection objects. In 10 mL cell transfection system, the cell density was 7×10⁵ cells/mL, and the culture medium was SMM 293TII produced by Sino Biological, Inc., Beijing. 10 μg of the plasmid was added to 0.5 ml of OPTI-MEM at room temperature for 5 minutes, and 50 μL of PEI was added to 0.5 mL of OPTI-MEM at room temperature for 5 minutes. The two solutions were mixed and incubated at room temperature for 25 minutes, and then the mixture was added to 10 mL of cells. The cells were cultured at 5% CO₂, 120 rpm in a cell culture shaker for 72 hours.

Flow cytometry sorting and high-throughput sequencing: cells containing a green fluorescent signal were sorted by a FACSAria Fusion flow cytometer from BD Company, and cells which died or failed in transfection and expression were removed. A universal column-type genome extraction kit available from Cowin Biotech Co., Ltd. was used to extract the whole genome of cells, and the products were directly sent to a sequencing company for high-throughput sequencing. Then, the results were analyzed for gene editing efficiency.

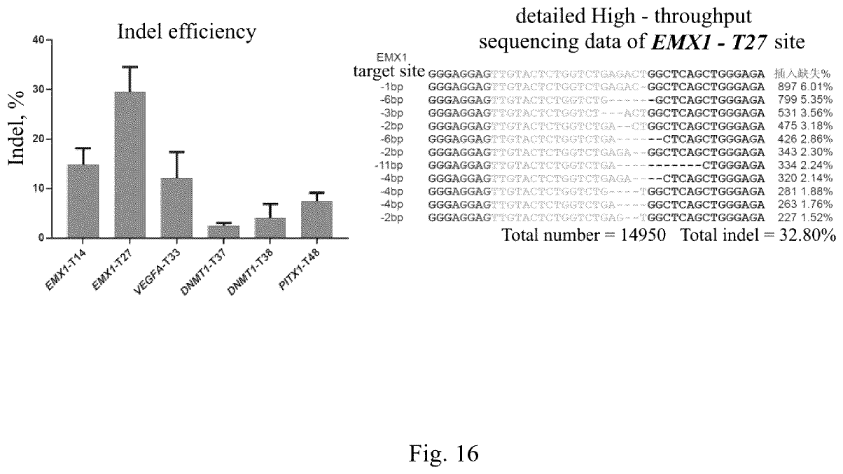

By constructing a eukaryotic expression vector (pcdna3.1-u6-FbRAD1-cmv-3flag-sv40-FbRAD1-NLS-2a-EGFP) and cell transfection experiments as well as high-throughput sequencing analysis, the inventors successfully verified that the FbRAD1 system can achieve specific DNA editing in human cells. Editing occurred at EMX1, VEGFA, DNMT1, and PITX1 gene loci (see Table 7 for sequence information), and the editing efficiency at the EMX1 locus reaches up to 32.8%. This efficiency is similar to the intracellular editing activities originally revealed for the widely used Cas9 and Cas12a. This study also provides a basis for the RAD system to be further optimized into an efficient gene editing tool (FIG. 16). FIG. 21 shows a flow chart showing the DNA editing by RAD protein in 293F cells and steps of detection.

TABLE 7
gene locus information of EMX1, VEGFA, DNMT1, PITXI.

| | | |
|---|---|---|
| EMX1- | Targeted site for intracellular editing | aggagAAGGCCAAGTGGTCCCAGGC (SEQ ID |
| T14 | of human 293F cells | NO: 144) |
| EMX1- | Targeted site for intracellular editing | aggagTTGTACTCTGGTCTGAGACT (SEQ ID |
| T27 | of human 293F cells | NO: 145) |
| VEGFA- | Targeted site for intracellular editing | aggagGAGGGCAGAATCATCACGAA (SEQ ID |
| T33 | of human 293F cells | NO: 146) |
| DNMT1- | Targeted site for intracellular editing | aggagTCTGATGTCGGGTTATACAC (SEQ ID |
| T37 | of human 293F cells | NO: 147) |
| DNMT1- | Targeted site for intracellular editing | aggagACAAAGTTCCTGACATTCTC (SEQ ID |
| T38 | of human 293F cells | NO: 148) |
| PITX1- | Targeted site for intracellular editing | aggagAAGGCCAGGTGACAGAGGGT (SEQ ID |
| T48 | of human 293F cells | NO: 149) |

Using the above experimental method, different RAD systems were expressed in 293F cells to verify whether they all have an intracellular editing function in mammalian cells. 9 RAD systems were found to be capable of intracellular editing, and these systems were derived from eight different species. They were: (6) FbRAD1/ISFba1 (RHS75625.1), (11) RiRAD/ISRin (RGR69673.1), (13) ErRAD/ISEre1 (RHB06265.1), (21) CbRAD3/ISClsp3 (RGD88909.1), (43) FbRAD4 (RGH37793.1), (51) DfRAD/ISDfo4 (GUT_GENOME239804_2_85784_86938+), (52) ArRAD/ISAre (GUT_GENOME047820_36_478_1632_-), (54) HuRAD/ISHun (GUT_GENOME022845_45_3955_5109_+) and (55) AfRAD/ISAfa (GUT_GENOME112567_131_1855_3009_-) (FIG. 17).

The RAD systems in a total of 16 strains from 11 different species have an intracellular activity to cleave DNA and are representatives of RAD systems. This confirms that the DNA cleavage activity in RAD systems is ubiquitous, providing a theoretical basis for further research on RAD systems. More RAD systems can be developed and optimized as gene editing tools.

RAD protein has a smaller protein size (about 387 amino acids). Compared with effector proteins of gene editing tools that have been widely studied currently, such as Cas9 and Cas12a effectors (generally greater than 1,000 amino acids), the RAD protein has the smallest size (384-387 amino acids), which suggests the value of further development into new gene editing tools. The smaller size is more advantageous in delivering vector constructs than traditional large proteins. The inventors have characterized the fundamental features of the RAD systems and constructed a gene-editing platform founded on these systems. This work lays a foundation for further optimizing the RAD systems into a more precise and efficient gene-editing tool.

The present invention described and claimed herein is not limited to the scope of the specific aspects disclosed herein, as these aspects are intended to be illustrative of several aspects of the disclosure. Any equivalent aspect is intended to be within the scope of the disclosure. Indeed, various modifications of the disclosure in addition to those shown and described herein will become obvious to those skilled in the art from the above description. Such modifications are also intended to fall within the scope of the appended claims. In case of conflict, this disclosure, including definitions, prevails.

1. An endonuclease complex comprising an RNA-guided endonuclease and a guide RNA, wherein the RNA-guided endonuclease is IS607 TnpB protein and the guide RNA comprises RAGATH-18-associated RNA.

2. The endonuclease complex of claim 1, wherein the RNA-guided endonuclease comprises a RuvC domain, and the RNA-guided endonuclease comprises an amino acid selected from the group consisting of Q8, G27, R30, N34, F50, W73, A92, F97, P104, K107, Y117, E145, P150, S173, G192, D194, G196, K198, A201, S204, N211, I212, N213, R227, Q229, 5233, R234, E237, G242, N249, K252, N266, V280, K283, P284, E290, L292, N293, G296, M297, M298, K299, L303, 5304, K305, Q310, K322, P338, S339, 5340, C343, C346, L353, L355, R358, C362, C364, D369, R370, D371, A374, N377 and L378 at an amino acid position corresponding to the amino acid sequence of SEQ ID NO: 6; optionally wherein the gene of interest is a single-stranded or double-stranded DNA, and optionally comprises a motif selected from the group consisting of NGGAG, NGGNN, AGGAG, GAGGG, GGGGG, NNGGG, NNAGG, AGGNN, NTAAA, NGAGG, NNGGN or GGGGN.

3. Polynucleotides comprising a polynucleotide encoding the RNA-guided endonuclease in claim 1 and a polynucleotide encoding the RNA molecule in claim 1.

4. A vector comprising the polynucleotides of claim 3, and optionally comprising an expression control sequence; wherein the RNA-guided endonuclease and the guide RNA are under the control of the same or different regulatory elements, optionally a promoter.

5. A viral particle, optionally an adeno-associated viral particle, comprising the endonuclease complex of claim 1, polynucleotides comprising a polynucleotide encoding the RNA-guided endonuclease in claim 1 and a polynucleotide encoding the RNA molecule in claim 1 or a vector comprising the polynucleotides.

6. A method for specifically cleaving DNA comprising contacting the endonuclease complex of claim 1 with the DNA,
optionally, the method comprises introducing polynucleotides comprising a polynucleotide encoding the RNA-guided endonuclease in claim 1 and a polynucleotide encoding the RNA molecule in claim 1, a vector comprising the polynucleotides or a viral particle into a cell, wherein the viral particle comprises the endonuclease complex of claim 1, the polynucleotides or the vector.

7. The method of claim 6, wherein the method is performed in vivo or in vitro.

8. The method of claim 6, wherein the method is performed under conditions selected from the group consisting of 0.5 to 10 mM of a divalent metal ion selected from the group consisting of Mg²⁺, Mn²⁺ and Ca²⁺, 25 to 250 mM Na⁺, a reaction temperature between 27 and 57° C. and a reaction time between 3 and 60 minutes.

9. A method for gene editing comprising contacting the endonuclease complex of claim 1 with a genomic DNA of a cell for the gene editing.

10. A kit comprising one or more of the following: the endonuclease complex of claim 1, polynucleotides comprising a polynucleotide encoding the RNA-guided endonuclease in claim 1 and a polynucleotide encoding the RNA molecule in claim 1, a vector comprising the polynucleotides, or a viral particle comprising the endonuclease complex, the polynucleotides or the vector.

11. A method for inactivating or identifying a virus, comprising contacting the virus with the endonuclease complex or the endonuclease in claim 1, optionally wherein the endonuclease comprises an amino acid sequence selected from the group consisting of SEQ ID NOs: 1-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 1-57 and SEQ ID NOs: 150-191.

12. A method for treating infection with a virus, comprising administering the endonuclease complex of claim 1 to a patient infected with the virus.

13. A viral vector comprising the polynucleotides of claim 3 or the vector comprising the polynucleotide molecule, optionally wherein the viral vector is an adeno-associated viral vector.

14. A gene therapy method for treating a disease comprising using the viral vector of claim 13.

15. A cell comprising one or more of the following: the endonuclease complex of claim 1, polynucleotides comprising a polynucleotide encoding the RNA-guided endonuclease in claim 1 and a polynucleotide encoding the RNA molecule in claim 1, a vector comprising the polynucleotides, or a viral particle comprising the endonuclease complex, the polynucleotides or the vector; optionally, the cell is a eukaryotic cell or a prokaryotic cell, optionally, the eukaryotic cell is a fungal cell, a plant cell or an animal cell.

16. The endonuclease complex of claim 1, wherein the RNA-guided endonuclease comprises an amino acid sequence selected from the group consisting of SEQ ID NOs: 6, 1-5, 7-57 and SEQ ID NOs: 150-191, or an amino acid sequence having at least 30%, 40%, 50%, 60%, 70%, 80%, 85%, 90%, 95%, 96%, 97%, 98%, 99% or 100% identity to any one of SEQ ID NOs: 6, 1-5, 7-57 and SEQ ID NOs: 150-191;
the guide RNA comprises an RNA-guided endonuclease-associated RNA, and optionally further comprises a targeting RNA;
the RNA-guided endonuclease-associated RNA comprises the sequence of any one of SEQ ID NOs: 63, 58-62, 64-114, and SEQ ID NOs: 192-233 or a sequence having at least 30%, 40%, 50%, 60%, 70%, 80%, 85%, 90%, 95%, 96%, 97%, 98%, 99% or 100% identity to the sequence of any one of SEQ ID NOs: 63, 58-62, 64-114, and SEQ ID NOs: 192-233;
the targeting RNA is at the 3′ end of the RNA-guided endonuclease-associated RNA and is at least 70%, 80%, 85%, 90%, 95%, 96%, 97%, 98%, 99% or 100% identical or complementary to a targeted sequence in a gene of interest, optionally the targeting RNA has a length of 12-40 bp;
optionally, the endonuclease system comprises an endonuclease of SEQ ID NO: N or a sequence having at least 30%, 40%, 50%, 60%, 70%, 80%, 85%, 90%, 95%, 96%, 97%, 98%, 99% or 100% identity to SEQ ID NO: N paired with an RNA guide sequence comprising SEQ ID NO: N+57 or a sequence having at least 30%, 40%, 50%, 60%, 70%, 80%, 85%, 95%, 96%, 97%, 98%, 99% or 100% identity to SEQ ID NO: N+57, wherein N is an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57; or the endonuclease system comprises an endonuclease of SEQ ID NO: M or a sequence having at least 30%, 40%, 50%, 60%, 70%, 80%, 85%, 90%, 95%, 96%, 97%, 98%, 99% or 100% identity to SEQ ID NO: M paired with an RNA guide sequence comprising SEQ ID NO: M+42 or a sequence having at least 30%, 40%, 50%, 60%, 70%, 80%, 85%, 95%, 96%, 97%, 98%, 99% or 100% identity to SEQ ID NO: M+42, wherein the M is an integer of 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, or 191.

17. The endonuclease complex of claim 1, wherein the endonuclease complex comprises an RNA-guided endonuclease comprising SEQ ID NO: 6 and a guide RNA comprising SEQ ID NO: 63; an RNA-guided endonuclease comprising SEQ ID NO: 11 and a guide RNA comprising SEQ ID NO: 68; an RNA-guided endonuclease comprising SEQ ID NO: 13 and a guide RNA comprising SEQ ID NO: 70; an RNA-guided endonuclease comprising SEQ ID NO: 21 and a guide RNA comprising SEQ ID NO: 78; an RNA-guided endonuclease comprising SEQ ID NO: 43 and a guide RNA comprising SEQ ID NO: 100; an RNA-guided endonuclease comprising SEQ ID NO: 51 and a guide RNA comprising SEQ ID NO: 108; an RNA-guided endonuclease comprising SEQ ID NO: 52 and a guide RNA comprising SEQ ID NO: 109; an RNA-guided endonuclease comprising SEQ ID NO: 54 and a guide RNA comprising SEQ ID NO: 111; or an RNA-guided endonuclease comprising SEQ ID NO: 55 and a guide RNA comprising SEQ ID NO: 112.

18. The method of claim 6, wherein the DNA is DNA of a eukaryotic cell, a prokaryotic cell or a virus;
and/or, the DNA is a single-stranded or double-stranded DNA;
and/or, the targeted sequence of the DNA comprises a motif selected from the group consisting of NGGAG, NGGNN, AGGAG, GAGGG, GGGGG, NNGGG, NNAGG, AGGNN, NTAAA, NGAGG, NNGGN or GGGGN, and the DNA at the 3′ end of the motif is at least 70%, 80%, 85%, 90%, 95%, 96%, 97%, 98% or 99% identical or complementary to the targeting RNA.

19. The method of claim 9, wherein the gene editing results in an insertion and/or a deletion in the genomic DNA;
and/or, the gene editing is performed in a mammalian cell to treat or prevent a disease, or to introduce a desired trait.
